(19) United States
(12) Patent Application Publication
(10) Pub. No.: US 2024/0237649 A1
(43) Pub. Date: Jul. 18, 2024

(54) CRYSTALLINE FORMS OF 5-FLUORO-4-IMINO-3-METHYL-1-TOSYL-3,4-DIHYDROPYRIMIDIN-2-ONE, AND MIXTURES, COMPOSITIONS AND METHODS OF USE THEREOF

(71) Applicants: Enrico MODENA, Bologna (IT); Stefano Luca GIAFFREDA, Bologna (IT); Cristina IANNI, Bologna (IT); Chiara PARISE, Bologna (IT); Rotem SELLA-EREZ, Tel-Aviv (IL); Judith ARONHIME, Rehovot (IL); Gal SUEZ, Dimona (IL); Hanan SERTCHOOK, Gedera (IL); Adama Makhteshim Ltd., Beer Sheva (IL)

(72) Inventors: Stefano Luca Giaffreda, Imola, Bologna (IT); Enrico Modena, Bologna (IT); Cristina Ianni, Bologna (IT); Chiara Parise, Bologna (IT); Rotem Stella-Erez, Tel-Aviv (IL); Judith Aronhime, Rehovot (IL); Gal Suez, Dimona (IL); Hanan Sertchook, Gedera (IL)

(73) Assignee: Adama Makhteshim Ltd., Beer Sheva (IL)

(21) Appl. No.: 18/557,524

(22) PCT Filed: May 4, 2022
(86) PCT No.: PCT/IB2022/054132

§ 371 Date: Oct. 26, 2023

Related U.S. Application Data
(60) Provisional application No. 63/184,071, filed on May 4, 2021.
(60) Provisional application No. 63/184,074, filed on May 4, 2021.

(51) Int. Cl. A01N 43/54; A01N 25/22; A01N 25/30; A01P 3/00; C07D 239/47

(52) U.S. Cl. CPC A01N 43/54; A01N 25/22; A01N 25/30; A01P 3/00; C07D 239/47

(57) ABSTRACT

The present invention provides crystalline forms of 5-fluoro-4-imino-3-methyl-1-tosyl-3,4-dihydropyrimidin-2(1H)-one, and uses and processes of preparation thereof. The present invention also provides combinations, mixtures and compositions comprising the crystalline forms of 5-fluoro-4-imino-3-methyl-1-tosyl-3,4-dihydropyrimidin-2(1H)-one, and uses and processes of preparation thereof.

This application claims benefit of U.S. Provisional Application No. 63/184,074, filed May 4, 2021, and U.S. Provisional Application No. 63/184,071, filed May 4, 2021, the entire content of each of which is hereby incorporated by reference herein.

Throughout this application various publications are referenced. The disclosures of these documents in their entireties are hereby incorporated by reference into this application in order to more fully describe the state of the art to which this invention pertains.

### BACKGROUND

Fungicides are compounds, of natural or synthetic origin, which act to protect plants against damage caused by fungi. Current methods of agriculture rely heavily on the use of fungicides. In fact, some crops cannot be grown usefully without the use of fungicides. Using fungicides allows a grower to increase the yield and the quality of the crop, and consequently, increase the value of the crop. In most situations, the increase in value of the crop is worth at least three times the cost of the use of the fungicide.

5-fluoro-4-imino-3-methyl-1-tosyl-3,4-dihydropyrimidin-2(1H)-one is a fungicide which provides control of a variety of pathogens in economically important crops including, but not limited to, the causal agent of leaf blotch in wheat, Septoria tritici, (SEPTTR) and diseases caused by fungi of the classes Ascomycetes and Basidiomycetes.

Uses of N3-substituted-N1-sulfonyl-5-fluoropyrimidinone derivatives as fungicides were described in U.S. Pat. No. 8,263,603, issued Sep. 11, 2012, the content of which is incorporated herein by reference in its entirety. Methods of preparation of 5-fluoro-4-imino-3-methyl-1-tosyl-3,4-dihydropyrimidin-2(1H)-one were described in U.S. Pat. No. 9,850,215, issued Dec. 26, 2017 and U.S. Pat. No. 9,840,476, issued Dec. 12, 2017, the contents of each of which are incorporated herein by reference in their entirety.

U.S. Pat. No. 8,263,603 also described fungicidal compositions for the control or prevention of fungal attack comprising N3-substituted-N1-sulfonyl-5-fluoropyrimidinone derivatives and a phytologically acceptable carrier material, and methods of use thereof.

Fungicidal compositions are frequently applied under various conditions and/or with other additives such as adjuvant and fertilizer. Therefore, fungicidal compositions must exhibit excellent chemical stability and a high level of physical stability during the preparation, storage and application process.

Often in agriculture, the compositions are diluted with water prior to use. Liquid compositions are much easier to dilute and disperse in water.

Sometimes, the biological activity and efficacy of the fungicide is limited for various reasons such as rapid drifting, limited penetration into leaves and high surface tension/low spreading. The efficacy of the active compound can be influenced and enhanced by adding adjuvant(s).

Adjuvants are inert chemicals which are added for increasing performance of the active ingredient and composition thereof. Adjuvants affect the condition for absorption of the active ingredient and the delivery properties thereof which leads to increased efficacy and enhanced activity of the active ingredient. For example, an adjuvant can enhance the efficacy of active ingredients; e.g. modifies properties of the spray solution to improve deposition on the leaf of the fungicide.

The use of adjuvant suitable for the active ingredient and composition thereof often determines whether or not the active ingredient can be used and can act in its full efficacy after application. The adjuvant is required to increase the reservoir of “available” material for uptake on the leaf surface. Such adjuvants are often non-ionic surfactants or various types of oil.

U.S. Pat. No. 8,263,603 disclosed that the compositions comprising N3-substituted-N1-sulfonyl-5-fluoropyrimidinone derivatives described therein may additionally contain adjuvant surfactants to enhance deposition, wetting and penetration of the compounds onto the target crop and organism.

### SUMMARY

The present invention provides a crystalline form of 5-fluoro-4-imino-3-methyl-1-tosyl-3,4-dihydropyrimidin-2(1H)-one (compound A), which has the following structure:

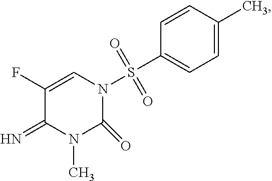 [SMILES: Cc1ccc(S(=O)(=O)n2cc(F)c(=N)n(C)c2=O)cc1]

wherein:
   a. the crystalline form is Form III having powder X-ray diffraction pattern comprising peaks at 4.5, 5.1, 9.1, 10.1, 22.4, 23.5±0.2 deg 2-theta;
   b. the crystalline form is Form IIIa having powder X-ray diffraction pattern comprising peaks at 4.5, 5.0, 9.3, 10.0, 22.3, 22.6±0.2 deg 2-theta;
   c. the crystalline form is Form 02 having powder X-ray diffraction pattern comprising peaks at 7.5, 14.3, 15.8, 22.4, 24.7±0.2 deg 2-theta; or
   d. the crystalline form is Form 01 having powder X-ray diffraction pattern comprising peaks at 5.1, 6.4, 7.5, 10.3, 16.1±0.2 deg 2-theta.

The present invention also provides a combination comprising any one of the crystalline forms described herein and at least one agriculturally acceptable carrier.

The present invention also provides a combination comprising an amount of compound A in two or more different forms and at least one agriculturally acceptable carrier, wherein at least one form of compound A is the crystalline form of the present invention.

The present invention also provides a composition comprising any one of the combinations described herein.

The present invention also provides a method for treating a plant or locus against fungal infection, wherein the method comprises:
i) obtaining an effective amount of the crystalline form, combination, or composition of the present invention; and
ii) applying the amount of the crystalline form, combination, or composition to the plant or locus so as to thereby treat the plant or locus against fungal infection.

The present invention also provides a method for treating a plant or locus against fungal infection, wherein the method comprises:
i) obtaining an effective amount of a compound having the following structure:

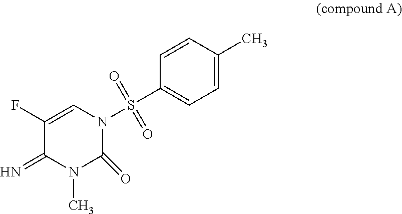 [SMILES: Cc1ccc(S(=O)(=O)n2cc(F)c(=N)n(C)c2=O)cc1]

in one or more forms, wherein at least one form of compound A is the crystalline Form III, IIIa, 01 or 02 of the present invention, and
ii) applying the amount of compound A to the plant or locus so as to thereby treat the plant or locus against fungal infection.

The present invention also provides a process for preparing the crystalline form of the present invention, wherein the process comprises:
a) combining an amount of a compound having the following structure:

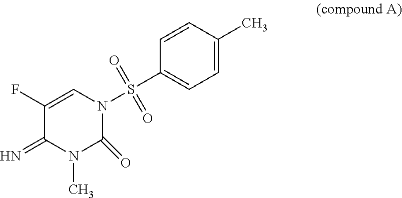 [SMILES: Cc1ccc(S(=O)(=O)n2cc(F)c(=N)n(C)c2=O)cc1]

and a suitable solvent to prepare a mixture;
b) optionally removing any precipitated solid from the mixture of step a); and
c) obtaining the crystalline form.

The present invention also provides a process of manufacturing a fungicidal composition comprising obtaining the crystalline form of the present invention and combining the crystalline form with at least one agriculturally acceptable carrier so as to thereby manufacture the fungicidal composition.

The present invention provides a stable, liquid composition comprising:
(a) a fungicidally effective amount of a compound of Formula I:

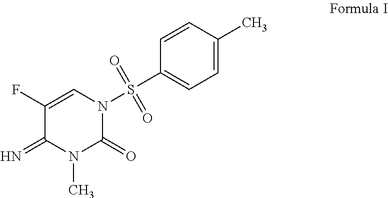 [SMILES: Cc1ccc(S(=O)(=O)n2cc(F)c(=N)n(C)c2=O)cc1]

in one or more forms, wherein at least one form is:
a. the crystalline form Form III having powder X-ray diffraction pattern comprising peaks at 4.5, 5.1, 9.1, 10.1, 22.4, 23.5±0.2 deg 2-theta;
b. the crystalline form Form IIIa having powder X-ray diffraction pattern comprising peaks at 4.5, 5.0, 9.3, 10.0, 22.3, 22.6±0.2 deg 2-theta;
c. the crystalline form Form 02 having powder X-ray diffraction pattern comprising peaks at 7.5, 14.3, 15.8, 22.4, 24.7±0.2 deg 2-theta; or
d. the crystalline form Form 01 having powder X-ray diffraction pattern comprising peaks at 5.1, 6.4, 7.5, 10.3, 16.1±0.2 deg 2-theta.
and
(b) a liquid carrier.

The present invention provides a stable, liquid composition comprising:
(a) a fungicidally effective amount of a compound of Formula I:

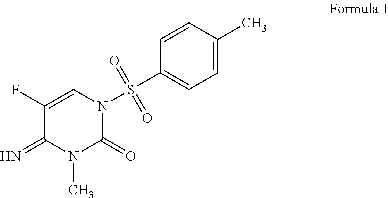 [SMILES: Cc1ccc(S(=O)(=O)n2cc(F)c(=N)n(C)c2=O)cc1]

in one or more forms, wherein at least one form is:
a. the crystalline form Form III having powder X-ray diffraction pattern comprising peaks at 4.5, 5.1, 9.1, 10.1, 22.4, 23.5±0.2 deg 2-theta;
b. the crystalline form Form IIIa having powder X-ray diffraction pattern comprising peaks at 4.5, 5.0, 9.3, 10.0, 22.3, 22.6±0.2 deg 2-theta;
c. the crystalline form Form 02 having powder X-ray diffraction pattern comprising peaks at 7.5, 14.3, 15.8, 22.4, 24.7±0.2 deg 2-theta; or
d. the crystalline form Form 01 having powder X-ray diffraction pattern comprising peaks at 5.1, 6.4, 7.5, 10.3, 16.1±0.2 deg 2-theta.
and
(b) a liquid carrier,
wherein:
(i) the composition comprises (a) a fungicidally effective and substantially pure amount of the compound of Formula I or a fungicidally effective amount of a mixture containing the compound of Formula I, wherein 95% or more by weight of the mixture is the compound of Formula I, and (b) the liquid carrier, and/or
(ii) 95% or more of the amount of the compound of Formula I is in the form of Form III, Form IIIa, Form 01, Form 02, or a mixture thereof.

The present invention provides a fungicidal mixture comprising the following components:
(a) a fungicidally effective and substantially pure amount of a compound of Formula I or a fungicidally effective amount of a mixture containing a compound of Formula I:

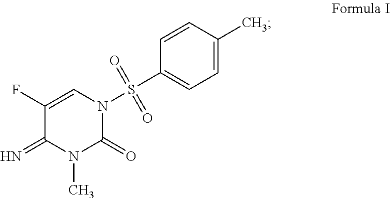 [SMILES: Cc1ccc(S(=O)(=O)n2cc(F)c(=N)n(C)c2=O)cc1]

and
(b) an adjuvant selected from the group consisting of:
   (i) polyalkylene oxide alkyl ether;
   (ii) siloxane polyalkyleneoxide copolymer;
   (iii) esters of fatty acid;
   (iv) vinylpyrrolidones and derivatives thereof;
   (v) sugar-based surfactants;
   (vi) lignins;
   (vii) terpenes; and
   (viii) any combination of (i), (ii), (iii), (iv), (v),
   (vi) and (vii),
      wherein the compound of Formula I is in one or more forms and at least one form is Form III, Form IIIa, Form 01, or Form 02.

The present invention also provides a method of controlling and/or preventing (i) fungal pathogen attack on a plant, or (ii) plant and/or soil fungal disease, wherein the method comprises applying a fungicidally effective amount of a compound having Formula (I):

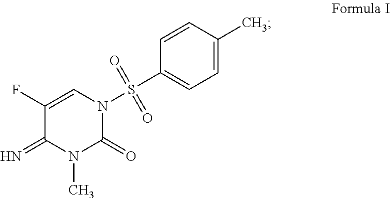 [SMILES: Cc1ccc(S(=O)(=O)n2cc(F)c(=N)n(C)c2=O)cc1]

and at least one adjuvant to soil, plant, root, foliage, seed, locus of the fungus, and/or a locus in which the infestation is to be prevented so as to thereby control and/or prevent fungal pathogen attack on the plant or plant and/or fungal disease, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants,
(vi) lignins;
(vii) terpens; and
(viii) any combination of (i), (ii), (iii), (iv), (v), (vi) and (vii),
wherein the compound of Formula I is in one or more forms and at least one form is Form III, Form IIIa, Form 01, or Form 02.

The present invention also provides a method for improving biological activity of a compound of Formula I against fungal pathogen, the method comprising applying a compound of Formula I:

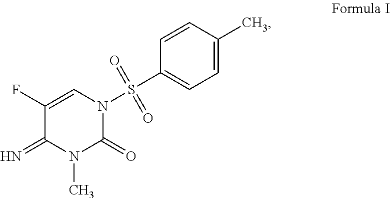 [SMILES: Cc1ccc(S(=O)(=O)n2cc(F)c(=N)n(C)c2=O)cc1]

in the presence of at least one adjuvant, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
so as to thereby improve biological activity of the compound of Formula I,
wherein the compound of Formula I is in one or more forms and at least one form is Form III, Form IIIa, Form 01, or Form 02.

The present invention also provides a method for increasing stability of a liquid composition comprising a compound of Formula I:

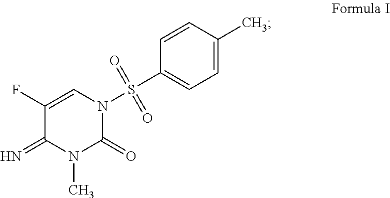 [SMILES: Cc1ccc(S(=O)(=O)n2cc(F)c(=N)n(C)c2=O)cc1]

and a liquid carrier, wherein the method comprises:
a) selecting a liquid carrier wherein the solubility of the compound of Formula I in the liquid carrier is less than 5000 ppm,
b) maintaining the pH value of the composition in the range of 5 to 7.5,
c) maintaining the water content of the composition to less than 0.2% by weight based on the total weight of the composition,
d) adding (i) at least one stabilizing surfactant having crystal growth inhibiting property or (ii) a stabilizing system having a crystal growth inhibiting property to the liquid composition, and/or
e) formulating the composition to have a viscosity of at least 500 cP,
so as to thereby increase stability of the composition comprising the compound of Formula I,
wherein the compound of Formula I is in one or more forms and at least one form is Form III, Form IIIa, Form 01, or Form 02.

The present invention also provides use of at least one stabilizing surfactant having structure of polyalkylene oxide polyaryl ether for controlling solubility and/or degradation of an amount of a compound of Formula I:

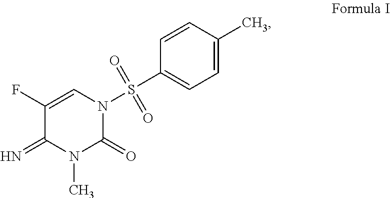 [SMILES: Cc1ccc(S(=O)(=O)n2cc(F)c(=N)n(C)c2=O)cc1]

wherein the compound of Formula I is in one or more forms and at least one form is Form III, Form IIIa, Form 01, or Form 02.

The present invention also provides a process for preparing a suspension concentrate (SC) composition comprising an amount of a compound of Formula I:

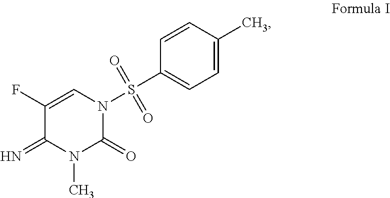 [SMILES: Cc1ccc(S(=O)(=O)n2cc(F)c(=N)n(C)c2=O)cc1]

wherein the process comprises the steps:
(1) mixing agriculturally acceptable inert additives and an aqueous liquid carrier to obtain a premix;
(2) adding the compound of Formula I to the premix obtained in step (1) to obtain a mixture; and
(3) milling the resulting mixture of step (2) to obtain the desired composition,
wherein the compound of Formula I is in one or more forms and at least one form is Form III, Form IIIa, Form 01, or Form 02.

The present invention also provides a process for preparing a suspension concentrate (SC) composition comprising an amount of a compound of Formula I:

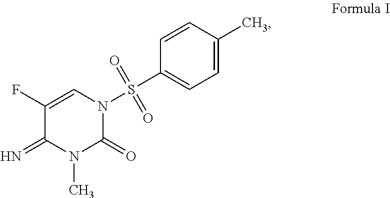 [SMILES: Cc1ccc(S(=O)(=O)n2cc(F)c(=N)n(C)c2=O)cc1]

wherein the process comprises the steps:
(1) mixing agriculturally acceptable inert additives and an aqueous liquid carrier to obtain a premix;
(2) adding the compound of Formula I to the premix obtained in step (1) to obtain a mixture; and
(3) milling the resulting mixture of step (2) to obtain the desired composition,
wherein the compound of Formula I is in one or more forms and at least one form is Form IIIa, Form 01, or Form 02.

The present invention also provides a process for preparing the suspoemulsion (SE) composition comprising an amount of a compound of Formula I:

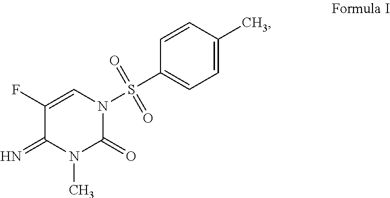 [SMILES: Cc1ccc(S(=O)(=O)n2cc(F)c(=N)n(C)c2=O)cc1]

wherein the process comprises the steps:
(1) mixing agriculturally acceptable inert additives and an aqueous liquid carrier to obtain a premix;
(2) adding the compound of Formula I and at least one adjuvant to the premix obtained in step (1) to obtain a mixture; and
(3) milling the resulting mixture of step (2) to obtain the desired composition,
wherein the compound of Formula I is in one or more forms and at least one form is Form III, Form IIIa, Form 01, or Form 02.

The present invention also provides a process for preparing an oil dispersion (OD) composition comprising an amount of a compound of Formula I:

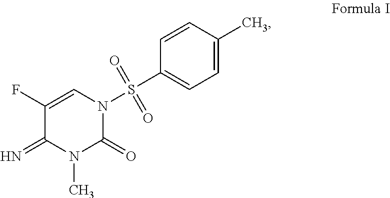 [SMILES: Cc1ccc(S(=O)(=O)n2cc(F)c(=N)n(C)c2=O)cc1]

wherein the process comprises the steps:
   (1) mixing agriculturally acceptable inert additives and a non-aqueous liquid carrier to obtain a premix;
   (2) adding the compound of Formula I to the premix obtained in step (1) to obtain a mixture; and
   (3) milling the resulting mixture of step (2) to obtain the desired composition,
wherein the compound of Formula I is in one or more forms and at least one form is Form III, Form IIIa, Form 01, or Form 02.

The present invention also provides a process for preparing an emulsifiable concentrate (EC) composition comprising an amount of a compound of Formula I:

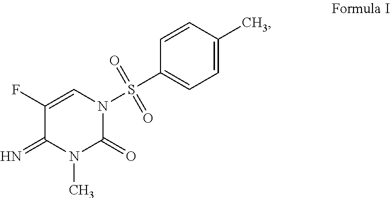 [SMILES: Cc1ccc(S(=O)(=O)n2cc(F)c(=N)n(C)c2=O)cc1]

wherein the process comprises the steps:
(1) mixing the agriculturally acceptable inert additives and a non-aqueous liquid carrier to obtain a premix;
(2) adding the compound of Formula I to the premix obtained in step (1) to obtain a mixture; and
(3) filtering the solution of step (2) to obtain the desired composition,
wherein the compound of Formula I is in one or more forms and at least one form is Form III, Form IIIa, Form 01, or Form 02.

The present invention also provides use of a compound having Formula (I):

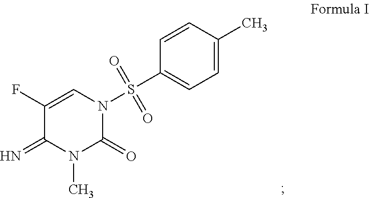 [SMILES: Cc1ccc(S(=O)(=O)n2cc(F)c(=N)n(C)c2=O)cc1 ; ";"]

and an adjuvant selected from the group consisting of:
(i) polyalkylene oxide alkyl ether;
(ii) siloxane polyalkyleneoxide copolymer;
(iii) esters of fatty acid;
   (i) vinylpyrrolidones and derivatives thereof;
   (ii) sugar-based surfactants;
   (iii) lignins;
   (iv) terpenes; and
   (v) any combination of (i), (ii), (iii), (iv), (v), (vi) and (vii),
for (a) controlling and/or preventing fungal pathogen attack on a plant and/or (b) controlling and/or preventing plant and/or soil fungal diseases,
wherein the compound of Formula I is in one or more forms and at least one form is Form III, Form IIIa, Form 01, or Form 02.

The present invention also provides a compound having Formula (I):

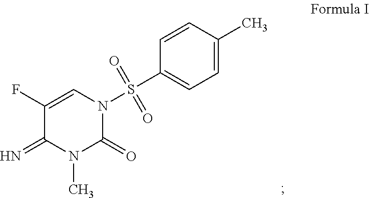 [SMILES: Cc1ccc(S(=O)(=O)n2cc(F)c(=N)n(C)c2=O)cc1 ; ";"]

and an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(i) sugar-based surfactants;
(ii) lignins;
(iii) terpenes; and
(iv) any combination of (i), (ii), (iii), (iv), (v), (vi) and (vii),
for use in (a) controlling and/or preventing fungal pathogen attack on a plant and/or (b) controlling and/or preventing plant and/or soil fungal diseases,
wherein the compound of Formula I is in one or more forms and at least one form is Form III, Form IIIa, Form 01, or Form 02.

The present invention also provides use of an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
for improving the biological activity of the compound of Formula (I):

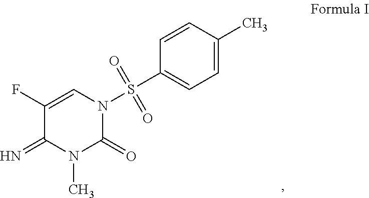 [SMILES: Cc1ccc(S(=O)(=O)n2cc(F)c(=N)n(C)c2=O)cc1 ; ","]

wherein the compound of Formula I is in one or more forms and at least one form is Form III, Form IIIa, Form 01, or Form 02.

The present invention also provides an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants,
(vi) lignins;
(vii) terpenes; and
(viii) any combination of (i), (ii), (iii), (iv), (v), (vi) and (vii),
for use in improving the biological activity of the compound of Formula (I):

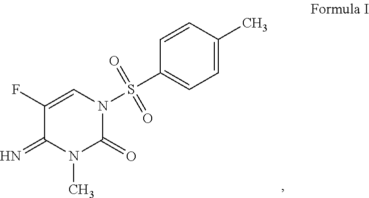 [SMILES: Cc1ccc(S(=O)(=O)n2cc(F)c(=N)n(C)c2=O)cc1 ; ","]

wherein the compound of Formula I is in one or more forms and at least one form is Form III, Form IIIa, Form 01, or Form 02.

The present invention provides a fungicidal mixture comprising the following components:
(a) a fungicidally effective and substantially pure amount of a compound of Formula I or a fungicidally effective amount of a mixture containing a compound of Formula I:

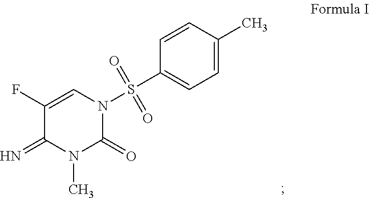 [SMILES: Cc1ccc(S(=O)(=O)n2cc(F)c(=N)n(C)c2=O)cc1 ; ";"]

(b) an adjuvant selected from the group consisting of:
   (i) polyalkylene oxide alkyl ether;
   (ii) siloxane polyalkyleneoxide copolymer;
   (iii) esters of fatty acid;
   (iv) vinylpyrrolidones and derivatives thereof;
   (v) sugar-based surfactants;
   (vi) lignins;
   (vii) terpenes; and
   (viii) any combination of (i), (ii), (iii), (iv), (v), (vi) and (vii),
wherein:
1) 95% or more of the amount of the compound of Formula I is in the form of Form III, Form IIIa, Form 01, Form 02, or a mixture thereof, and/or
2) the mixture of component (a) comprises 950 or more by weight of the compound of Formula I, wherein the compound of Formula I is in one or more forms and at least one form is Form III, Form IIIa, Form 01, or Form 02.

The present invention provides a method for the control and/or prevention of (i) fungal pathogen attack on a plant or (ii) plant and/or soil fungal disease, wherein the method comprises applying the composition or mixture described herein to soil, plant, root, foliage, seed, locus of the fungus, and/or a locus in which the infestation is to be prevented so as to thereby control and/or prevent fungal pathogen attack on a plant or plant and/or soil fungal disease.

The present invention also provides a method of controlling and/or preventing (i) fungal pathogen attack on a plant, or (ii) plant and/or soil fungal disease, wherein the method comprises applying a fungicidally effective amount of a compound having Formula (I):

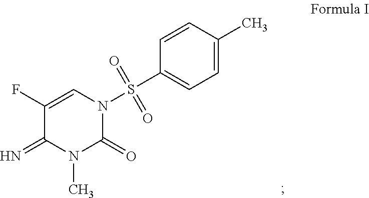 [SMILES: Cc1ccc(S(=O)(=O)n2cc(F)c(=N)n(C)c2=O)cc1 ; ";"]

and at least one adjuvant to soil, plant, root, foliage, seed, locus of the fungus, and/or a locus in which the infestation is to be prevented so as to thereby control and/or prevent fungal pathogen attack on the plant or plant and/or fungal disease, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants,
(vi) lignins;
(vii) terpens; and
(viii) any combination of (i), (ii), (iii), (iv), (v), (vi) and (vii),
wherein:
(1) 95% or more of the amount of the compound of Formula I is in the form of Form III, Form IIIa, Form 01, Form 02, and/or
(2) the method comprises applying a tank mix comprising the compound of Formula I wherein the compound of Formula I is added to the tank mix as the composition or mixture described herein.

The present invention also provides a method for improving biological activity of a compound of Formula I against fungal pathogen, the method comprising applying a compound of Formula I:

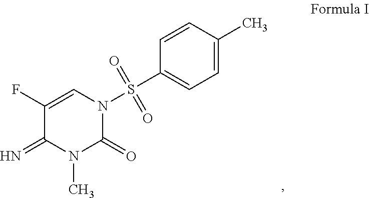 [SMILES: Cc1ccc(S(=O)(=O)n2cc(F)c(=N)n(C)c2=O)cc1 ; ","]

in the presence of at least one adjuvant, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
so as to thereby improve biological activity of the compound of Formula I,
wherein:
(1) 95% or more of the amount of the compound of Formula I is in the form of Form III, Form IIIa, Form 01, Form 02, or a mixture thereof, and/or
(2) the method comprises applying a tank mix comprising the compound of Formula I wherein the compound of Formula I is added to the tank mix in the form of the composition or mixture described herein.

The present invention also provides a method for increasing stability of a liquid composition comprising a compound of Formula I:

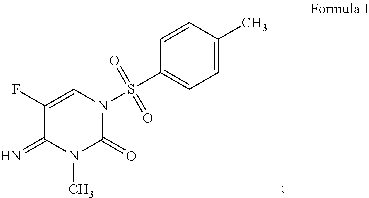 [SMILES: Cc1ccc(S(=O)(=O)n2cc(F)c(=N)n(C)c2=O)cc1 ; ";"]

and a liquid carrier, wherein the method comprises:
f) selecting a liquid carrier wherein the solubility of the compound of Formula I in the liquid carrier is less than 5000 ppm,
g) maintaining the pH value of the composition in the range of 5 to 7.5,
h) maintaining the water content of the composition to less than 0.2% by weight based on the total weight of the composition,
i) adding (i) at least one stabilizing surfactant having crystal growth inhibiting property or (ii) a stabilizing system having a crystal growth inhibiting property to the liquid composition, and/or
j) formulating the composition to have a viscosity of at least 500 cP,
so as to thereby increase stability of the composition comprising the compound of Formula I,
wherein 95% or more by weight of the amount of the compound of Formula I is in the form of Form III, Form IIIa, Form 01, Form 02, or a mixture thereof.

The present invention also provides a method for increasing stability of a liquid composition comprising a compound of Formula I:

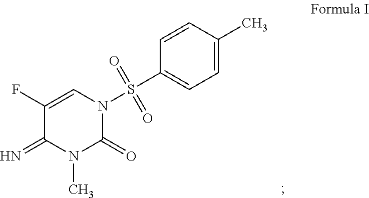 [SMILES: Cc1ccc(S(=O)(=O)n2cc(F)c(=N)n(C)c2=O)cc1 ; ";"]

and a liquid carrier, wherein the method comprises using a batch of the compound of Formula I that is substantially pure of the compound of Formula I or comprises 95% or more by weight of the compound of Formula I to prepare the stable, liquid composition, and wherein the compound of Formula I is in one or more forms and at least one form is Form III, Form IIIa, Form 01, or Form 02.

The present invention also provides use of at least one stabilizing surfactant having structure of polyalkylene oxide polyaryl ether for controlling solubility and/or degradation of an amount of a compound of Formula I:

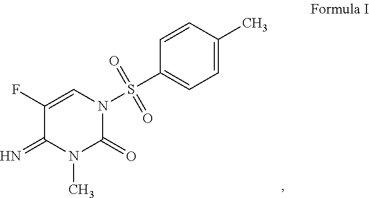 [SMILES: Cc1ccc(S(=O)(=O)n2cc(F)c(=N)n(C)c2=O)cc1 ; ","]

wherein 95% or more by weight of the amount of the compound of Formula I is in the form of Form III, Form IIIa, Form 01, Form 02, or a mixture thereof.

The present invention also provides a process for preparing a suspension concentrated (2C) composition comprising an amount of a compound of Formula I:

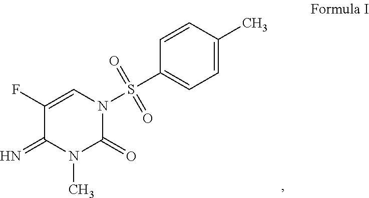 [SMILES: Cc1ccc(S(=O)(=O)n2cc(F)c(=N)n(C)c2=O)cc1 ; ","]

wherein the process comprises the steps:
   (1) mixing agriculturally acceptable inert additives and an aqueous liquid carrier to obtain a premix;
   (2) adding the compound of Formula I to the premix obtained in step (1) to obtain a mixture; and
   (3) milling the resulting mixture of step (2) to obtain the desired composition,
wherein:
(i) 950 or more by weight of the amount of the compound of Formula I is Form III, Form IIIa, Form 01, Form 02, or a mixture thereof, and/or
(ii) step (2) of the process comprises obtaining a batch of the compound of Formula I that is substantially pure or comprises 95% or more by weight of the compound of Formula I and adding the batch of the compound of Formula I to the premix obtained in step (1) to obtain a mixture, and wherein the compound of Formula I is in one or more forms and at least one form is Form III, Form IIIa, Form 01, or Form 02.

The present invention also provides a process for preparing a suspension concentrate (SC) composition comprising an amount of a compound of Formula I:

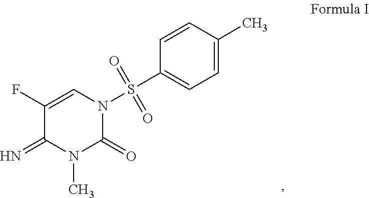 [SMILES: Cc1ccc(S(=O)(=O)n2cc(F)c(=N)n(C)c2=O)cc1 ; ","]

wherein the process comprises the steps:
   (4) mixing agriculturally acceptable inert additives and an aqueous liquid carrier to obtain a premix;
   (5) adding the compound of Formula I to the premix obtained in step (1) to obtain a mixture; and
   (6) milling the resulting mixture of step (2) to obtain the desired composition,
wherein:
(i) 95% or more by weight of the amount of the compound of Formula I is Form IIIa, Form 01, Form 02, or a mixture thereof, and/or
(ii) step (2) of the process comprises obtaining a batch of the compound of Formula I that is substantially pure or comprises 95% or more by weight of the compound of Formula I and adding the batch of the compound of Formula I to the premix obtained in step (1) to obtain a mixture, and wherein the compound of Formula I is in one or more forms and at least one form is Form IIIa, Form 01, or Form 02.

The present invention also provides a suspension concentrate (SC) composition prepared using the process described herein.

The present invention also provides a process for preparing the suspoemulsion (SE) composition comprising an amount of a compound of Formula I:

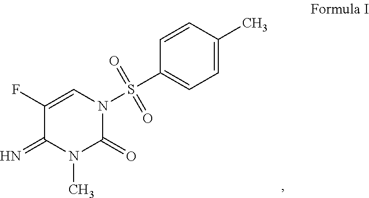 [SMILES: Cc1ccc(S(=O)(=O)n2cc(F)c(=N)n(C)c2=O)cc1 ; ","]

wherein the process comprises the steps:
(1) mixing agriculturally acceptable inert additives and an aqueous liquid carrier to obtain a premix;
(2) adding the compound of Formula I and at least one adjuvant to the premix obtained in step (1) to obtain a mixture; and
(3) milling the resulting mixture of step (2) to obtain the desired composition,
wherein:
(i) 95% or more by weight of the amount of the compound of Formula I is in the form of Form III, Form IIIa, Form 01, Form 02, or a mixture thereof, and/or
(ii) step (2) of the process comprises obtaining a batch of the compound of Formula I that is substantially pure of the compound of Formula I or comprises 95% or more by weight of the compound of Formula I and adding the batch of the compound of Formula I to the premix obtained in step (1) to obtain a mixture, wherein the compound of Formula I is in one or more forms and at least one form is Form III, Form IIIa, Form 01, or Form 02.

The present invention also provides a suspoemulsion (SE) composition prepared using the process described herein.

The present invention also provides a process for preparing an oil dispersion (OD) composition comprising an amount of a compound of Formula I:

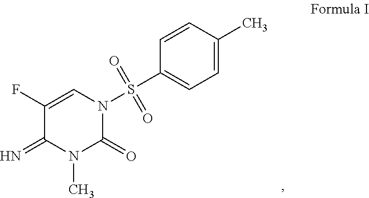 [SMILES: Cc1ccc(S(=O)(=O)n2cc(F)c(=N)n(C)c2=O)cc1 ; ","]

wherein the process comprises the steps:
   (1) mixing agriculturally acceptable inert additives and a non-aqueous liquid carrier to obtain a premix;
   (2) adding the compound of Formula I to the premix obtained in step (1) to obtain a mixture; and
   (3) milling the resulting mixture of step (2) to obtain the desired composition,
wherein:
(i) 95% or more by weight of the amount of the compound of Formula I is in the form of Form III, Form IIIa, Form 01, Form 02, or a mixture thereof, and/or
(ii) step (2) of the process comprises obtaining a batch of the compound of Formula I that is substantially pure of the compound of Formula I or comprises 95% or more by weight of the compound of Formula I and adding the batch of the compound of Formula I to the premix obtained in step (1) to obtain a mixture, and wherein the compound of Formula I is in one or more forms and at least one form is Form III, Form IIIa, Form 01, or Form 02.

The present invention also provides an oil dispersion (OD) composition prepared using the process described herein.

The present invention also provides a process for preparing an emulsifiable concentrate (EC) composition comprising an amount of a compound of Formula I:

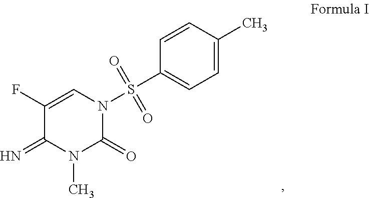 [SMILES: Cc1ccc(S(=O)(=O)n2cc(F)c(=N)n(C)c2=O)cc1 ; ","]

wherein the process comprises the steps:
   (1) mixing the agriculturally acceptable inert additives and a non-aqueous liquid carrier to obtain a premix;
   (2) adding the compound of Formula I to the premix obtained in step (1) to obtain a mixture; and
   (3) filtering the solution of step (2) to obtain the desired composition,
wherein:
(i) 95% or more of the amount of the compound of Formula I is in the form of Form III, Form IIIa, Form 01, Form 02, or a mixture thereof, and/or
(ii) step (2) of the process comprises obtaining a batch of the compound of Formula I that is substantially pure of the compound of Formula I or comprises 95% or more by weight of the compound of Formula I and adding the batch of the compound of Formula I to the premix obtained in step (1) to obtain a mixture, and wherein the compound of Formula I is in one or more forms and at least one form is Form III, Form IIIa, Form 01, or Form 02.

The present invention also provides an emulsifiable concentrate (EC) composition prepared using the process described herein.

The present invention also provides a composition comprising an admixture of a compound of Formula I:

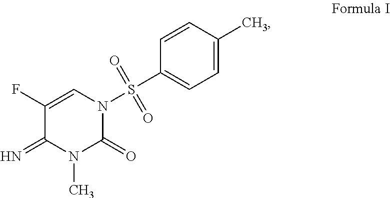 [SMILES: Cc1ccc(S(=O)(=O)n2cc(F)c(=N)n(C)c2=O)cc1]

and a liquid carrier,
wherein the composition is prepared using any one of the processes described herein.

The present invention also provides a method for (i) the control or prevention of fungal attack on a plant or (ii) protecting a plant from fungal attack comprising applying the composition or mixture described herein to a seed adapted to produce the plant.

The present invention also provides a method of treating a plant seed or seedling to produce a plant resistant to fungal attack comprising applying the composition or mixture described herein to the plant seed or seedling.

The present invention also provides a method of protecting a plant from fungal attack comprising applying the composition or mixture described herein to the seedling environment.

The present invention also provides a plant resistant to fungal attack wherein the seed of the plant is treated with the composition or mixture described herein.

The present invention also provides a plant seed or seedling adapted to produce a plant resistant to fungal attack, wherein the plant seed or seedling is treated with the composition or mixture described herein.

The present invention also provides a package comprising the composition or mixture described herein.

The present invention also provides a use of the mixture described herein for manufacturing a fungicidal composition.

The present invention also provides use of a compound having Formula (I):

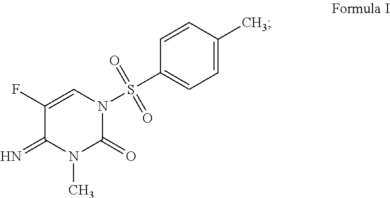 [SMILES: Cc1ccc(S(=O)(=O)n2cc(F)c(=N)n(C)c2=O)cc1]

and an adjuvant selected from the group consisting of:
(i) polyalkylene oxide alkyl ether;
(ii) siloxane polyalkyleneoxide copolymer;
(iii) esters of fatty acid;
(iii) vinylpyrrolidones and derivatives thereof;
(iv) sugar-based surfactants;
(v) lignins;
(vi) terpenes; and
(vii) any combination of (i), (ii), (iii), (iv), (v), (vi) and (vii),
for (a) controlling and/or preventing fungal pathogen attack on a plant and/or (b) controlling and/or preventing plant and/or soil fungal diseases,
wherein:
(i) 95% or more of the amount of the compound of Formula I is in the form of Form III, Form IIIa, Form 01, Form 02, and/or
(ii) the use comprises applying a tank mixture comprising the compound of Formula I wherein the compound of Formula I is added to the tank mixture as a substantially pure compound or a mixture comprising 95% or more by weight of the compound of Formula I, and wherein the compound of Formula I is in one or more forms wherein at least one form is Form III, Form IIIa, Form 01, or Form 02.

The present invention also provides a compound having Formula (I):

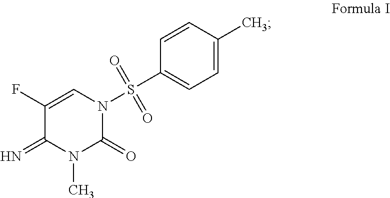 [SMILES: Cc1ccc(S(=O)(=O)n2cc(F)c(=N)n(C)c2=O)cc1]

and an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(i) sugar-based surfactants;
(ii) lignins;
(iii) terpenes; and
(iv) any combination of (i), (ii), (iii), (iv), (v), (vi) and (vii),
for use in (a) controlling and/or preventing fungal pathogen attack on a plant and/or (b) controlling and/or preventing plant and/or soil fungal diseases,
wherein:
(i) 95% or more of the amount of the compound of Formula I is in the form of Form III, Form IIIa, Form 01, Form 02, or a mixture thereof, and/or
(ii) the use comprises applying a tank mixture comprising the compound of Formula I wherein the compound of Formula I is added to the tank mixture as a substantially pure compound or a mixture comprising 95% or more by weight of the compound of Formula I, and wherein the compound of Formula I is in one or more forms wherein at least one form is Form III, Form IIIa, Form 01, or Form 02.

The present invention also provides use of an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
for improving the biological activity of the compound of Formula (I):

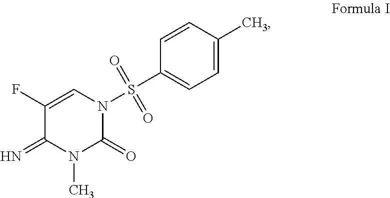 [SMILES: Cc1ccc(S(=O)(=O)n2cc(F)c(=N)n(C)c2=O)cc1]

wherein 95% or more by weight of the compound of Formula I is in the form of Form III, Form IIIa, Form 01, Form 02, or a mixture thereof.

The present invention also provides an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants,
(vi) lignins;
(vii) terpenes; and
(viii) any combination of (i), (ii), (iii), (iv), (v), (vi) and (vii),
for use in improving the biological activity of the compound of Formula (I):

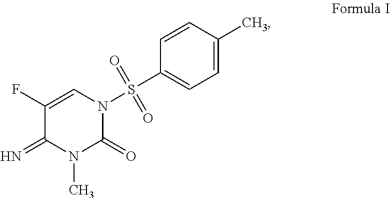 [SMILES: Cc1ccc(S(=O)(=O)n2cc(F)c(=N)n(C)c2=O)cc1]

wherein 9% 3 more by weight of the compound of Formula I is in the form of Form III, Form IIIa, Form 01, Form 02, or a mixture thereof.

The present invention also provides a process of preparing a stable, liquid composition comprising an admixture of a compound of Formula I:

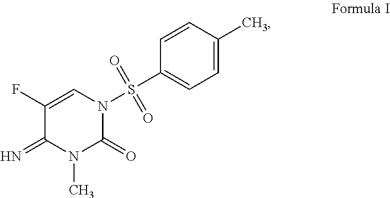 [SMILES: Cc1ccc(S(=O)(=O)n2cc(F)c(=N)n(C)c2=O)cc1]

and a liquid carrier,
wherein the process comprises the steps:
   (1) obtaining a batch of the compound of Formula I that is substantially pure of the compound of Formula I or comprises 95% or more by weight of the compound of Formula I, and
   (2) mixing the batch of the compound of Formula I from step (1) with the liquid carrier to obtain the composition,
wherein the compound of Formula I is in one or more forms wherein at least one form is Form III, Form IIIa, Form 01, or Form 02.

The present invention also provides a process of preparing a stable, liquid composition comprising a compound of Formula I:

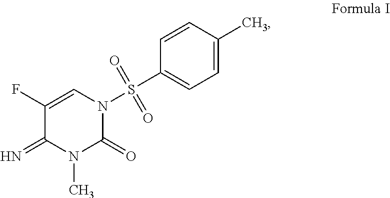 [SMILES: Cc1ccc(S(=O)(=O)n2cc(F)c(=N)n(C)c2=O)cc1]

and a liquid carrier,
wherein the process comprises the steps:
   1) obtaining a batch of the compound of Formula I wherein 95% or more of the compound of Formula I in the batch is in the form of Form III, Form IIIa, Form 01, Form 02, or a mixture thereof, and
(2) mixing the batch of the compound of Formula I from step (1) with the liquid carrier to obtain the composition.

The present invention also provides a composition comprising a compound of Formula I:

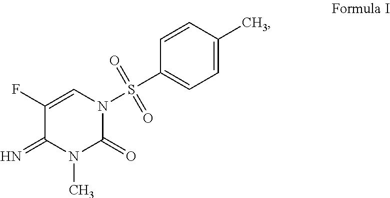 [SMILES: Cc1ccc(S(=O)(=O)n2cc(F)c(=N)n(C)c2=O)cc1]

and a liquid carrier,
wherein the composition is prepared using the process described herein.

### BRIEF DESCRIPTION OF THE DRAWINGS

FIG. 1: X-ray powder diffraction pattern of Form III.

FIG. 2: TGA of Form III.

FIG. 3: Differential Scanning Calorimetry (DSC) thermogram of Form III.

FIG. 4: X-ray powder diffraction pattern of Form IIIa.

FIG. 5: TGA of Form IIIa.

FIG. 6: X-ray powder diffraction pattern of form 02.

FIG. 7: TGA thermogram of form 02.

FIG. 8: Differential Scanning Calorimetry (DSC) thermogram of form 02.

FIG. 9: X-ray powder diffraction pattern of form 01.

FIG. 10: Differential Scanning Calorimetry (DSC) thermogram of form 01.

FIG. 11: Effect of Trycol® low (refers to 0.2 L/ha), Trycol® (refers to 0.4 L/ha) or Silwett as adjuvant on the activity of compound of Formula I. Comparison of the Area Under Disease Progress Curve (AUDPC) determined from the intensity of infection measured 21 dpi (days post infection) of Z. tritici strain Mg Tri-R6 moderately resistant to DMI fungicides and highly resistant to QoI fungicides of wheat plants cv. Alixan untreated or treated with compound of Formula I 450 suspension concentrate composition used at 2 rates (0.028 and 0.014 L/ha) alone (no adjuvant) or with adjuvant as tank mix. Values of the same timing of observation followed by the same letter are not significantly different according to the Newman and Keuls test (p<0.05).

FIG. 12. Effect of Trycol® low (refers to 0.2 L/ha), Trycol® (refers to 0.4 L/ha) or Silwett adjuvant on the activity of compound of Formula I. Comparison of the fungicide efficacy, obtained from AUDPC values, of compound of Formula I 450 suspension concentrate composition used at 2 rates (0.028 and 0.014 L/ha) alone (no adjuvant) or with an adjuvant (Trycol® low, Trycol® or Silwett®) as used at 1 rate towards Z. tritici strain Mg Tri-R6 moderately resistant to DMI and Highly resistant to QoI fungicides in controlled conditions.

FIG. 13. Effect of combination of two adjuvants, PVP and AGNIQUE BP420 as built-in composition on the activity of compound of Formula I. Comparison of the Area Under Disease Progress Curve (AUDPC) determined from the intensity of infection measured 21 dpi of Z. tritici strain Mg Tri-R6 moderately resistant to DMI fungicides and highly resistant to QoI fungicides of wheat plants cv. Alixan untreated or treated with compound of Formula I 450 suspension concentrate composition used at rate of 10 g a.i./ha alone (no adjuvant) or with the adjuvants. Values of the same timing of observation followed by the same letter are not significantly different according to the Newman and Keuls test (p<0.05).

FIG. 14. Effect of combination of two adjuvants, PVP and AGNIQUE® BP420 as built-in composition on the activity of compound of Formula I. Comparison of the fungicide efficacy, obtained from the AUDPC values, of suspension concentrate composition, of compound of Formula I at 10 g a.i./ha towards Zymoseptoria tritici strain Mg Tri-R6 moderately resistant to DMI and highly resistant to QoI fungicides in controlled conditions.

FIG. 15. Effect of three combinations of adjuvants, combination of PVP and AGNIQUE® BP420, combination of VP/VA and Silwett and combination of VP/VA and AGNIQUE® BP420 as built-in compositions (450 suspension concentrate composition) on the activity of compound of Formula I. Disease assessment (intensity of infection) on the first leaf of wheat plantlets cv. Alixan untreated or treated with suspension concentrate composition of compound of Formula I at 10 g a.i./ha 21 days post inoculation with pycnospores of the Zymoseptoria tritici strain Mg Tri-R6 moderately resistant to DMI and Highly resistant to QoI fungicides in controlled conditions. Values of the same timing of observation followed by the same letter are not significantly different according to the Newman and Keuls test (P<0.05).

FIG. 16. Effect of three combinations of adjuvants, combination of PVP and AGNIQUE® BP420, combination of VP/VA and Silwett® and combination of VP/VA and AGNIQUE® BP420 as built-in compositions on the activity of compound of Formula I compound of Formula I (450 suspension concentrate composition). Fungicide efficacy obtained from the intensity of infection determined 21 dpi and suspension concentrate composition of compound of Formula I at 10 g a.i./ha towards Zymoseptoria tritici strain Mg Tri-R6 moderately resistant to DMI and Highly resistant to QoI fungicides in controlled conditions.

FIG. 17. Comparison of the Area Under Disease Progress Curve (AUDPC) determined from the intensity of infection measured 21 and 28 dpi of Z. tritici strain Mg Tri-R6 moderately resistant o DMI fungicides and highly resistant to Qol fungicides of wheat plants cv. Alixan untreated or treated with the compound of Formula I 50 emusifiable concentrate compositions at 2 rates (0.25 and 0.125 L/ha) composition A alone (no adjuvant/additive) or with an adjuvant (Trycol®) and composition B alone|(no adjuvant/additive) or with an adjuvant (Trycol® low, Trycol®, Synergen® SOC) Values of the same timing of observation followed by the same letter are not significantly different according to the Newman and Keuls test (p<0.05).

FIG. 18. Comparison of the fungicide efficacy, obtained from AUDPC values, of the compound of Formula I 50 emusifiable concentrate compositions at 2 rates (0.25 and 0.125 L/ha) composition A alone (no adjuvant) or with an adjuvant (Trycol®) and composition B alone|(no adjuvant/additive) or with an adjuvant (Trycol® low, Trycol®, Synergen® SOC) towards Z. tritici strain Mg Tri-R6 moderately resistant to DMI and Highly resistant to QoI fungicides in controlled conditions.

FIG. 19. Disease assessment (intensity of infection) on the first leaf of wheat plantlets cv. Alixan untreated or treated with OD composition of Compound of Formula I Prototype A at 10 g a.i./ha and 20 g a.i./ha, 21 days post inoculation with pycnospores of the Zymoseptoria tritici strain Mg Tri-R6 moderately resistant to DMI and Highly resistant to QoI fungicides in controlled conditions.

FIG. 20. Disease assessment (% of infection) on potato late blight (Phytophthora infestans). Field trial assessment after six weekly applications.

FIG. 21. Concentration of the compound of Formula I after 2 weeks at 54° C. as a function of water content.

### DETAILED DESCRIPTION

### Definitions

Unless defined otherwise, all technical and scientific terms used herein have the meaning commonly understood by persons of ordinary skill in the art to which this subject matter belongs.

As used herein, the terms “compound A” or “compound of formula I” each refers to 5-fluoro-4-imino-3-methyl-1-tosyl-3,4-dihydropyrimidin-2(1H)-one, which has the following structure:

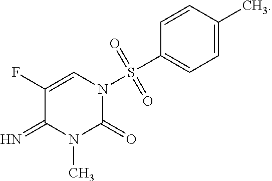 [SMILES: Cc1ccc(S(=O)(=O)n2cc(F)c(=N)n(C)c2=O)cc1]

The terms “compound A” and “compound of formula I” may be used interchangeably.

As used herein, the term “combination” means an assemblage of agrochemicals for application either by simultaneous or contemporaneous application. The combination may be in any physical form, e.g., blend, solution, alloy, or the like.

As used herein, the term “simultaneous” when used in connection with application of agrochemicals means that the agrochemicals are applied in an admixture, for example, a tank mix. For simultaneous application, the combination may be the admixture or separate containers each containing an agrochemical that are combined prior to application.

As used herein, the term “contemporaneous” when used in connection with application of agrochemicals means that an individual agrochemical is applied separately from another agrochemical or premixture at the same time or at times sufficiently close together that an activity that is additive or more than additive to the activity of either agrochemical alone at the same dose is achieved.

As used herein, the term “mixture” refers to, but is not limited to, a combination in any physical form, e.g., blend, solution, suspension, dispersion, emulsion, alloy, or the like.

As used herein, the term “tank mix” means one or more of the components of the combination, mixture or composition of the present invention, such as pesticide and/or additive and/or adjuvant, are added are mixed in a spray tank at the time of spray application or prior to spray application.

As used herein, the term “built-in” means that all components such as pesticide, adjuvant and other additives are in the same composition.

As used herein, the term “composition” includes at least one of the combinations or mixtures of the present invention with an agriculturally acceptable carrier.

As used herein, the term “effective” when used in connection with an amount of the active ingredient, combination, mixture or composition refers to an amount of the active ingredient, combination, mixture or composition that achieve a agriculturally beneficial level of control of the fungus, pathogen, and/or disease when applied to a plant, propagation material of the plant, soil or a locus.

As used herein, the term “effective amount” refers to an amount of the compound that, when applied, is sufficient to achieve a good level of control.

As used herein, the term “effective” when used in connection with a method for treating a plant or locus against fungal infection means that the method provides an agriculturally beneficial level of treatment without significantly interfering with the normal growth and development of the plant.

As used herein, the term “treating a plant or locus against fungal infection” includes, but is not limited to, protecting the plant or locus against fungal infection and/or controlling fungal infection of the plant or locus.

As used herein, the term “protecting the plant or locus against fungal infection” includes, but is not limited to, protecting the plant or locus against fungal attack, protecting the plant or locus from fungal disease, and/or preventing fungal infection of the plant or locus.

As used herein, the term “controlling fungal infection of the plant or locus” includes, but is not limited to, controlling fungal disease infecting the plant or locus, controlling a plant or soil disease caused by phytopathologic fungi, controlling fungal attack on the plant or locus, reducing fungal infection of the plant or locus, and/or curing plant or soil disease caused by phytopathologic fungi.

As used herein, the term “protectant application” means an application of one or more fungicide for preventing fungal infection of the plant or locus, wherein the fungicidal combination, mixture or composition is applied before infection occurs, before any disease symptoms are shown or when the disease pressure is low. Disease pressure may be assessed based on the conditions associated with disease development such as spore concentration and certain environmental conditions.

As used herein the term “curative application” means an application of one or more fungicide for controlling fungal infection of the plant or locus, wherein the fungicidal combination, mixture or composition is applied after an infection or after disease symptoms are shown.

As used herein, the phrase “agrochemically acceptable” means which is known and accepted in the art for use in agricultural/pesticidal use.

As used herein, the term “agriculturally acceptable carrier” means carriers which are known and accepted in the art for the formation of compositions for agricultural or horticultural use.

As used herein, the term “adjuvant” is broadly defined as any substance that itself is not an active ingredient but which enhances or is intended to enhance the effectiveness of the fungicide with which it is used. Adjuvants may be understood to include, spreading agents, penetrants, compatibility agents, and drift retardants.

As used herein, the term “excipient” refers to any chemical which has no significant pesticidal activity, such as surfactant(s), solvent(s), or adjuvant(s). One or more excipients can be added to any combination, mixture or composition disclosed herein.

As used herein, the term “stabilizing surfactant” is defined as any surfactant that increases the physical and/or chemical stability of the compound A when combined with compound A. In some embodiments, the stabilizing surfactant is effective for inhibiting crystal growth.

As used herein, the term “additive” is defined as any substance that itself is not a fungicide but is added to the composition such as sticking agents, surfactants, synergists, buffers, acidifiers, defoaming agents and thickeners.

As used herein, the term “thickener” refers to an agent that increases the viscosity of a liquid composition without essentially changing other properties of the composition.

As used herein, the term “w/w” means percentage by weight based on the total weight of the composition or mixture.

As used herein the term “plant” or “crop” includes reference to whole plants, plant organs (e.g. leaves, stems, twigs, roots, trunks, limbs, shoots, fruits etc.), plant cells, or plant seeds. This term also encompasses plant crops such as fruits. In yet another embodiment, the term “plant” may include the propagation material thereof, which may include all the generative parts of the plant such as seeds and vegetative plant material such as cuttings and tubers, which can be used for the multiplication of the plant. This includes seeds, tubers, spores, corms, bulbs, rhizomes, sprouts basal shoots, stolons, and buds and other parts of plants, including seedlings and young plants, which are to be transplanted after germination or after emergence from soil.

As used herein, the term “plant” includes reference to the whole plant, plant organ (e.g., leaves, stems, twigs, roots, trunks, limbs, shoots, fruits etc.), plant cells, and propagation material of the plant.

As used herein the term “plant” includes reference to agricultural crops include field crops (soybean, maize, wheat, rice, etc.), vegetable crops (potatoes, cabbages, etc.) and fruits (peach, etc.).

As used herein the term “propagation material” is to be understood to denote all the generative parts of the plant such as seeds and spores, seedlings, and vegetative structures such as bulbs, corms, tubers, rhizomes, roots stems, basal shoots, stolons and buds.

As used herein, the term “locus” includes not only areas where fungal infection may already be shown, but also areas where fungal infection have yet to show, and also to areas under cultivation. Locus includes, but is not limited to, soil and other plant growth medium.

As used herein, the term “substantially pure” refer to a purity of equal to or greater than 99.5%.

As used herein, the term “purity” when used in connection with compound A refers to the amount of compound A, which may be in any one or any combination of the known forms of compound A and crystalline Forms III, IIIa, 01 and 02 of the present invention, in the batch used to prepare a composition or mixture. For example, when it is described that the purity of compound A is 95% or more by weight, this means that the batch contains 95% or more by weight of the compound of compound A and 5% or less by weight of impurities.

As used herein, the term “stable” refers to chemical stability, physical stability, or both.

As used herein, the term “stable” when used in connection with chemical stability, e.g. chemically stable, means that the composition meets the chemical stability standards set forth by the Food and Agriculture Organization of the United Nations (FAO) in the Manual on Development and Use of FAO and WHO Specification for Pesticides (First Edition—Third Revision) (the “FAO/WHO Manual”) (available at http://www.fao.org/agriculture/crops/thematic-sitemap/theme/pests/jmps/manual/en/), the entire content of which is hereby incorporate by reference into the subject application. As described in the FAO/WHO Manual, a composition is stable if no significant degradation of the active ingredients in the composition is observable after 14 days of storage at a temperature of 54±2° C., after 4 weeks of storage at a temperature of 50±2° C., after 6 weeks of storage at a temperature of 45±2° C., after 8 weeks of storage at a temperature of 40±2° C., after 12 weeks of storage at a temperature of 35±2° C., or after 18 weeks of storage at a temperature of 30±2° C. The amount of degradation permitted before the degradation is considered to be significant depends on the concentration of the active ingredients in the composition. As described in the FAO/WHO Manual, for a composition comprising above 25 up to 100 g/L of active ingredients, degradation of up to 10% of the active ingredients is considered no significant degradation; for a composition comprising above 100 up to 250 g/L of active ingredients, degradation of up to 6% of the active ingredients is considered no significant degradation; for a composition comprising above 250 up to 500 g/L of active ingredients, degradation of up to 5% of the active ingredients is considered no significant degradation; and for a composition comprising above 500 g/L of active ingredients, degradation of up to 25 g/L of the active ingredients is considered no significant degradation.

As used herein, the term “stable” when used in connection with physical stability, e.g. physically stable, and when used in connection with a composition, means that the composition meets the physical stability standards set forth by the Collaborative International Pesticides Analytical Council (CIPAC). The CIPAC is an international, organization that promote international agreements on methods for the analysis of pesticides and physico-chemical test methods for compositions. Methods adopted by the CIPAC are published in the CIPAC Handbooks, available online at https://www.cipac.org/index.php/methods-publications, the entire content of each method is hereby incorporated by reference into the subject application.

As used herein, the term “liquid” means a liquid that is not a gas.

As used herein, when a property of a component in an admixture of components is described, the property is evaluated at the time the component is obtained or immediately prior to the formation of the admixture from the components, i.e. immediately prior to when the recited components are combined and made into the composition. For example, when it is described that one of the components in the admixture is a mixture comprising 95% or more by weight of a compound of Formula I as described herein, this means that when the mixture of Formula I is obtained or immediately prior the time the mixture is combined with another component, such as a liquid carrier, to form the admixture, the mixture contained 95% or more by weight of the compound of Formula I as described herein.

The concentrations of the compound of Formula I described in this application, given as w/w or w/v, refer to the weight of the batch of the compound of Formula I used to prepare the composition or mixture in relation to the total weight or volume of the composition or mixture. The batch of the compound of Formula I may comprise impurities, preferably no more than 5% by weight. Accordingly, when it is described that the concentration of the compound of Formula I in the composition is 50% by weight based on the total weight of the composition, the actual concentration of the compound of Formula I may range from 47.5% to 50% by weight based on the total weight of the composition depending on the purity of the compound of Formula I in the batch used.

As used herein, the term “water content” when used in connection with a composition, a mixture, or a component in the composition or mixture refers to the amount of free water in the composition, mixture or component of the composition or mixture and the water molecules chemically bound to another compound, such as water molecules in a hydrate.

As used herein, the term “low water content” when used in connection with a surfactant or carrier means that the surfactant or carrier solubilizes water in an amount of less than 25 g/L.

In some embodiments, the surfactant has a water content of less than 2.5% by weight based on the weight of the surfactant. In some embodiments, the surfactant has a water content of less than 2% by weight based on the weight of the surfactant. In some embodiments, the surfactant has a water content of less than 1.5% by weight based on the weight of the surfactant. In some embodiments, the surfactant has a water content of less than 1% by weight based on the weight of the surfactant. In some embodiments, the surfactant has a water content of less than 0.5% by weight based on the weight of the surfactant. In some embodiments, the water content is evaluated at the time the surfactant is obtained. In some embodiments, the water content is evaluated at the time immediately prior to addition of the surfactant to the admixture.

In some embodiments, the non-aqueous liquid carrier has a water content of less than 2.5% by weight based on the weight of the non-aqueous liquid carrier. In some embodiments, the non-aqueous liquid carrier has a water content of less than 2% by weight based on the weight of the non-aqueous liquid carrier. In some embodiments, the non-aqueous liquid carrier has a water content of less than 1.5% by weight based on the weight of the non-aqueous liquid carrier. In some embodiments, the non-aqueous liquid carrier has a water content of less than 1% by weight based on the weight of the non-aqueous liquid carrier. In some embodiments, the non-aqueous liquid carrier has a water content of less than 0.5% by weight based on the weight of the non-aqueous liquid carrier. In some embodiments, the water content is evaluated at the time the non-aqueous liquid carrier is obtained. In some embodiments, the water content is evaluated at the time immediately prior to addition of the non-aqueous liquid carrier to the admixture.

As used herein the term “ha” refers to hectare.

The term “a” or “an” as used herein includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one of skill in the art, that in some specific instances, an embodiment can alternatively be described using the language “consisting essentially of” or “consisting of.”

The term “about” herein specifically includes ±10% from the indicated values in the range. In addition, the endpoints of all ranges directed to the same component or property herein are inclusive of the endpoints, are independently combinable, and include all intermediate points and ranges.

It is understood that where a parameter range is provided, all integers within that range, and tenths thereof, are also provided by the invention as if the integers and tenths thereof are expressly described herein. For example, “from 1 g/ha to 500 g/ha” includes 1.0 g/ha, 1.1 g/ha, 1.2 g/ha, 1.3 g/ha, 1.4 g/ha, etc. up to 500 g/ha.

### Crystalline Forms of 5-Fluoro-4-Imino-3-Methyl-1-Tosyl-3,4-Dihydropyrimidin-2 (1H)-one

Solids exist in either amorphous or crystalline forms. In the case of crystalline forms, molecules are positioned in 3-dimensional lattice sites.

When a compound recrystallizes from a solution or slurry, it may crystallize with different spatial lattice arrangements, a property referred to as “polymorphism,” With the different crystal forms individually being referred to as a “polymorph”. Different polymorphic forms of a given substance may differ from each other with respect to one or more physical properties, such as solubility and dissociation, true density, crystal shape, compaction behavior, flow properties, and/or solid state stability. Solvates are crystalline solid adducts containing either stoichiometric or nonstoichiometric amounts of a solvent incorporated within the crystal structure. If the incorporated solvent is water, the solvates are also commonly known as hydrates.

Solvate or hydrate are also commonly known as “pseudopolymorph”.

The present invention provides crystalline forms of 5-fluoro-4-imino-3-methyl-1-tosyl-3,4-dihydropyrimidin-2(1H)-one (compound A), which has the following structure:

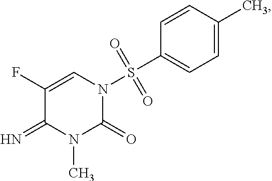 [SMILES: Cc1ccc(S(=O)(=O)n2cc(F)c(=N)n(C)c2=O)cc1]

wherein:
   a. the crystalline form is Form III having powder X-ray diffraction pattern comprising peaks at 4.5, 5.1, 9.1, 10.1, 22.4, 23.5±0.2 deg 2-theta;
   b. the crystalline form is Form IIIa having powder X-ray diffraction pattern comprising peaks at 4.5, 5.0, 9.3, 10.0, 22.3, 22.6±0.2 deg 2-theta;
   c. the crystalline form is Form 02 having powder X-ray diffraction pattern comprising peaks at 7.5, 14.3, 15.8, 22.4, 24.7±0.2 deg 2-theta; or
   d. the crystalline form is Form 01 having powder X-ray diffraction pattern comprising peaks at 5.1, 6.4, 7.5, 10.3, 16.1±0.2 deg 2-theta.

In one embodiment, the crystalline form is a polymorph of 5-fluoro-4-imino-3-methyl-1-tosyl-3,4-dihydropyrimidin-2(1H)-one.

In one embodiment, the crystalline form is an anhydrous crystalline form of 5-fluoro-4-imino-3-methyl-1-tosyl-3,4-dihydropyrimidin-2(1H)-one.

Forms III, IIIa, 01 and 02 exhibit distinct spectral characteristics as depicted by their X-ray diffraction patterns.

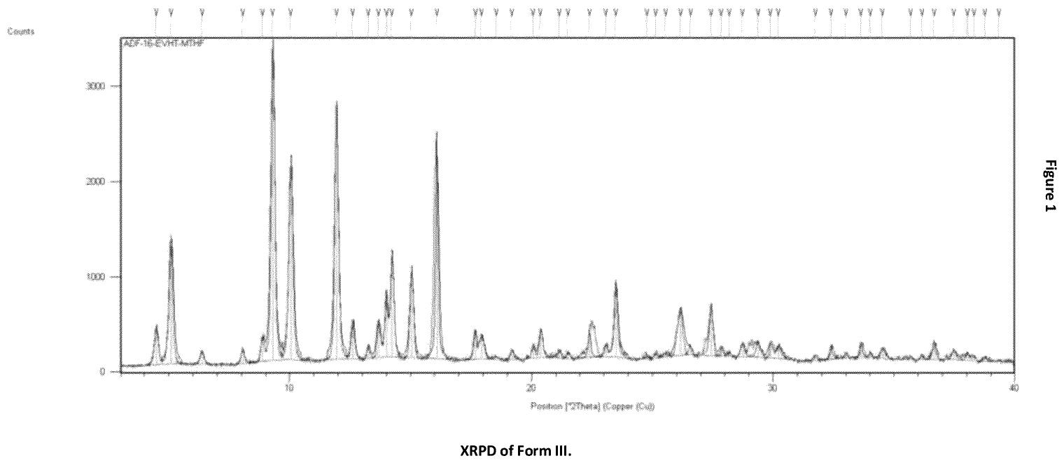

In one embodiment, the present invention provides a crystalline polymorphic form designated “Form III”. Form III exhibits an X-ray powder diffraction pattern as shown in FIG. 1. In one embodiment, Form III exhibits characteristic peaks at 4.5, 5.1, 9.1, 10.1, 22.4, 23.5±−0.2 deg 2-theta. In another embodiment, the X-ray powder diffraction pattern of Form III comprises characteristic peaks at 4.5, 5.1, 9.1, 10.1, 11.9, 14.2, 16.1, 22.4, 23.5±0.2 deg 2-theta.

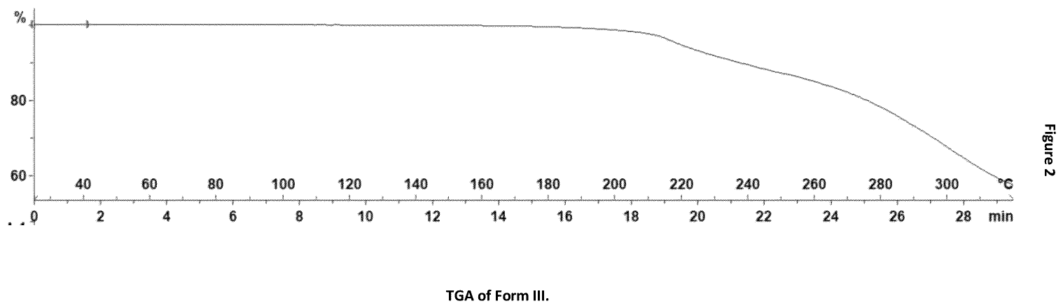

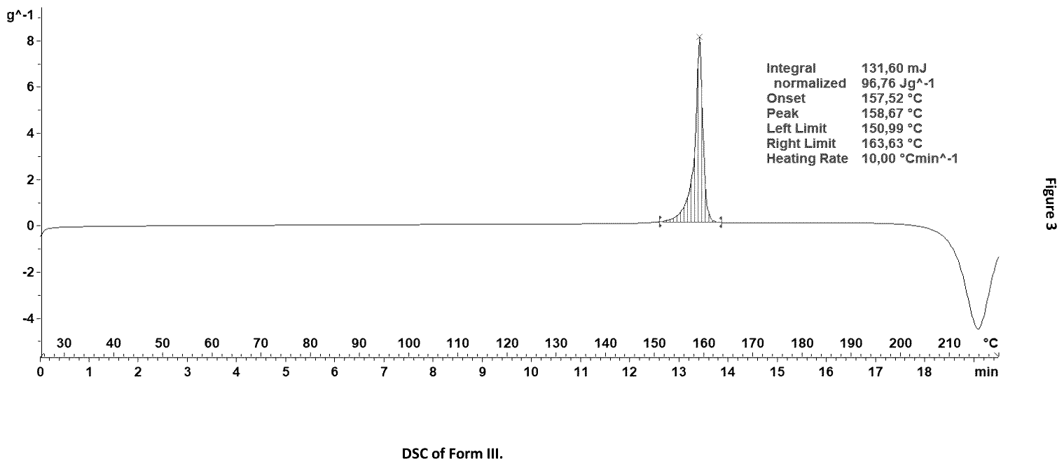

Form III is anhydrous based on TGA analysis (FIG. 2) which shows that no significant weight loss occurs up to 200 deg. The DSC melting event (FIG. 3) occurs between about 155-158 deg.

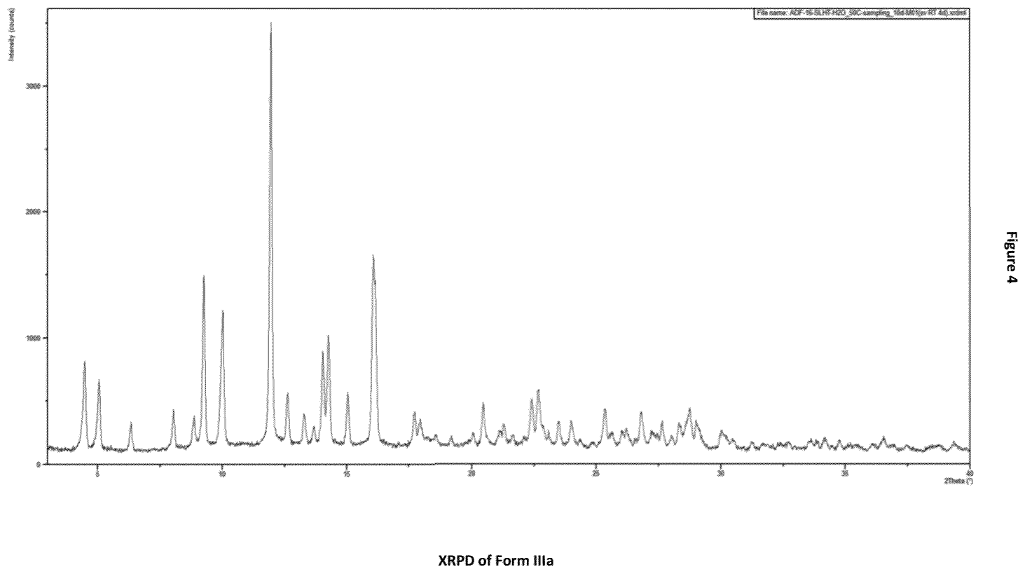

In one embodiment, the present invention provides a crystalline polymorphic form designated “Form IIIa”. Form IIIa exhibits an X-ray powder diffraction pattern as shown in FIG. 4. In one embodiment, Form IIIa exhibits characteristic peaks at 4.5, 5.0, 9.3, 10.0, 22.3, 22.6±0.2 deg 2-theta. In another embodiment, the X-ray powder diffraction pattern of Form IIIa comprises characteristic peaks at 4.5, 5.0, 9.3, 10.0, 11.9, 14.2, 16.0, 22.3, 22.6, 25.3±0.2 deg 2-theta.

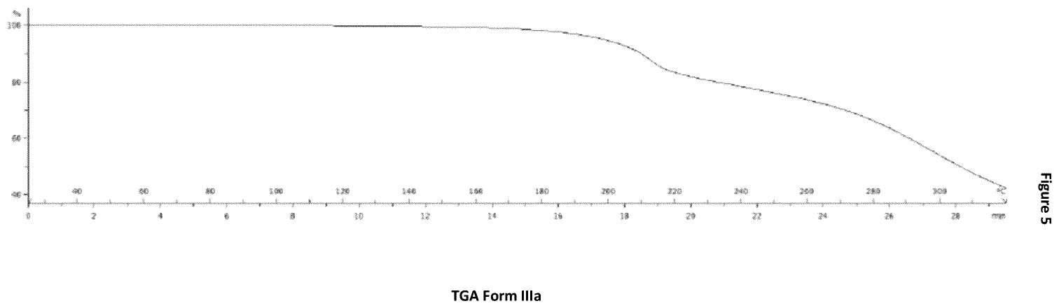

Form IIIa is anhydrous, based on TGA analysis (FIG. 5), which shows that no significant weight loss occurs up to 200 deg.

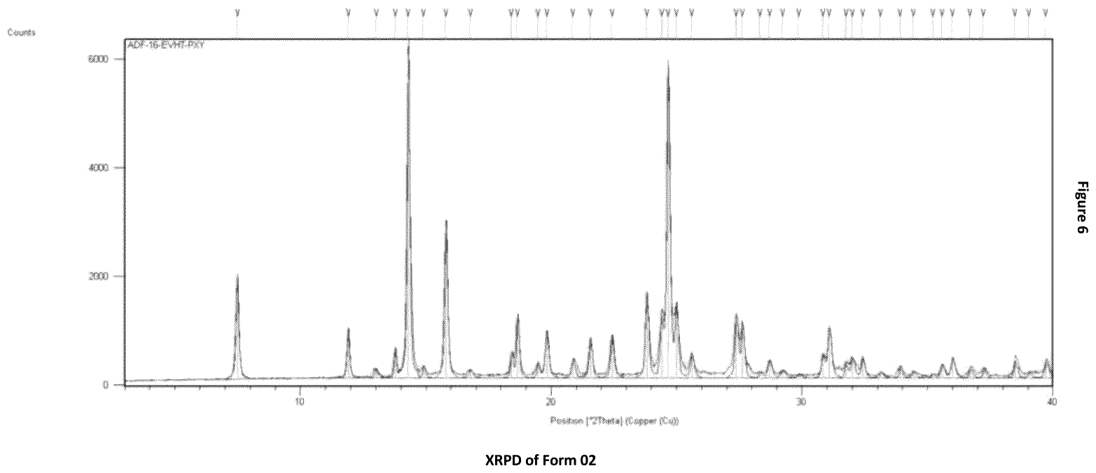

In one embodiment, the present invention provides a crystalline form designated “Form 02”. Form 02 exhibits an X-ray powder diffraction pattern as shown in FIG. 6. In one embodiment, Form 02 exhibits characteristic peaks at 7.5, 14.3, 15.8, 22.4, 24.7±0.2 deg 2-theta. In another embodiment, the X-ray powder diffraction pattern of Form 02 comprises characteristic peaks at 7.5, 11.9, 14.3, 15.8, 18.7, 22.4, 24.7, 27.4, 31.1±0.2 deg 2-theta.

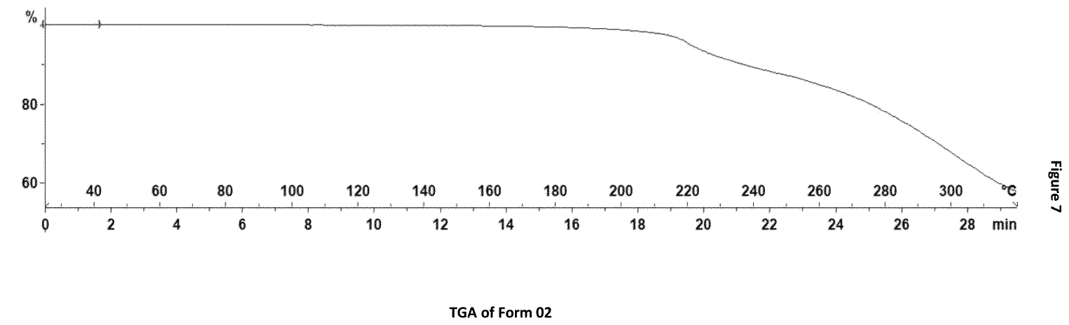

Form 02 is anhydrous based on TGA analysis, which shows that no significant weight loss occurs up to 200 deg (FIG. 7).

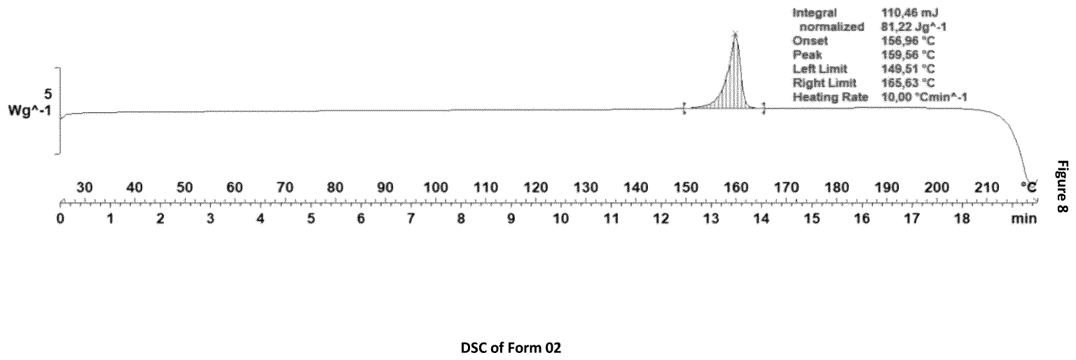

The DSC melting event y occurs between about 155-158 deg (FIG. 8).

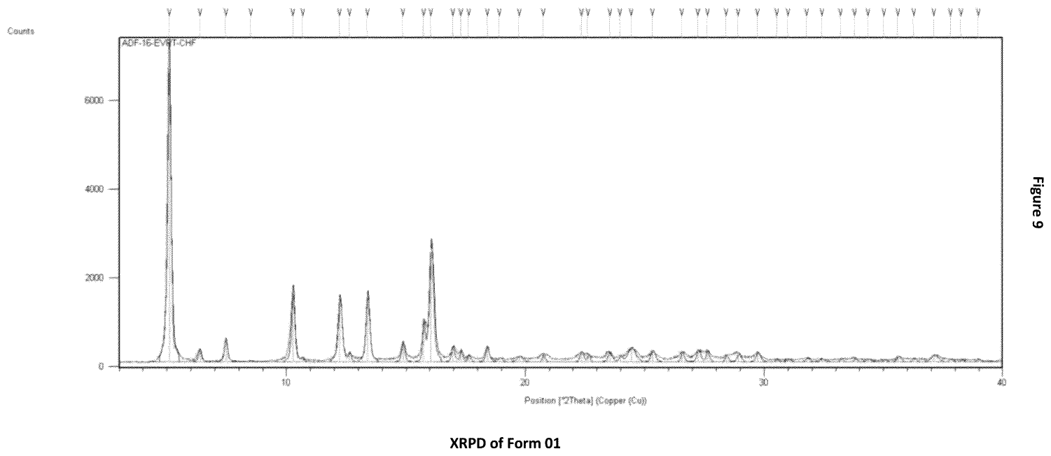

In one embodiment, the present invention provides a crystalline form designated “Form 01”. Form 01 exhibits an X-ray powder diffraction pattern as shown in FIG. 9. Form 01 exhibits characteristic peaks at 5.1, 6.4, 7.5, 10.3, 16.1±0.2 deg 2-theta. In another embodiment, the X-ray powder diffraction pattern of Form 01 comprises characteristic peaks at 5.1, 6.4, 7.5, 10.3, 12.2, 13.4, 14.9, 16.1, 18.4±0.2 deg 2-theta.

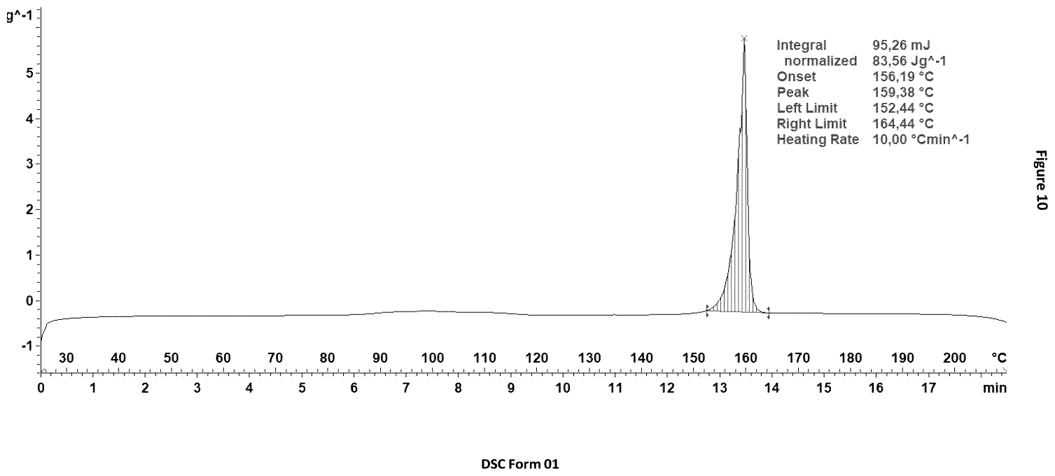

The DSC melting event y occurs between about 155-165 deg (FIG. 10).

Combinations of Crystalline Forms of Compound A, and Mixtures and Compositions Thereof

The present invention also provides a combination of two, three or four of crystalline Form III, IIIa, 01 or 02 described herein. In some embodiments, the combination comprises at least one agriculturally acceptable carrier.

The present invention also provides a combination of any one of crystalline Form III, IIIa, 01 or 02 described herein and at least one agriculturally acceptable carrier.

The present invention also provides a combination of an amount of compound A in two or more different forms and at least one agriculturally acceptable carrier, wherein at least one form of compound A is crystalline Form III, IIIa, 01 or 02 described herein.

The present invention also provides a combination of any one of crystalline Form III, IIIa, 01 or 02 described herein and at least one additional pesticide. In some embodiments, the pesticide is a fungicide.

In some embodiments, the combination is a mixture. In some embodiments, the mixture is a tank mix.

In some embodiments, the mixture is a fungicidal mixture.

In some embodiments, the crystalline Form III, IIIa, 02 or 01 is at least about 0.1% by weight of the total amount of compound A in the combination. In some embodiments, the crystalline form III, IIIa, 02 or 01 is at least about 1% by weight of the total amount of compound A in the combination. In some embodiments, the crystalline form III, IIIa, 02 or 01 is at least about 5% by weight of the total amount of compound A in the combination.

In some embodiments, the crystalline Form III, IIIa, 02 or 01 is at least about 50% by weight of the total amount of compound A in the combination.

In some embodiments, the crystalline Form III, IIIa, 02 or 01 is at least about 60% by weight of the total amount of compound A in the combination.

In some embodiments, the crystalline Form III, IIIa, 02 or 01 is at least about 70% by weight of the total amount of compound A in the combination.

In some embodiments, the crystalline Form III, IIIa, 02 or 01 is at least about 80% by weight of the total amount of compound A in the combination.

In some embodiments, the crystalline Form III, IIIa, 02 or 01 is at least about 90% by weight of the total amount of compound A in the combination.

In some embodiments, the crystalline Form III, IIIa, 02 or 01 is at least about 95% by weight of the total amount of compound A in the combination.

In some embodiments, the crystalline Form III, IIIa, 02 or 01 is at least about 98% by weight of the total amount of compound A in the combination.

In some embodiments, the crystalline Form III, IIIa, 02 or 01 is at least about 99% by weight of the total amount of compound A in the combination.

In some embodiments, the amount of compound A in the combination is substantially free of compound A in amorphous form.

In some embodiments, the amount of compound A in the combination is substantially free of crystalline Form 01 of the present invention.

In some embodiments, the amount of compound A in the combination is substantially free of crystalline Form 02 of the present invention.

In some embodiments, the amount of compound A in the combination is substantially free of crystalline Form 01 and 02 of the present invention.

In some embodiments, the combination comprises at least one additional pesticide. In some embodiments, the pesticide is a fungicide.

In some embodiments, the fungicide is a fungicidal sterol biosynthesis inhibitor. In some embodiments, the fungicide is a succinate dehydrogenase inhibitor. In some embodiments, the fungicide is a strobilurin fungicide. In some embodiments, the fungicide is a fungicidal multisite inhibitor. In some embodiments, the fungicide is a Qi inhibitor.

Suitable fungicidal sterol biosynthesis inhibitors, succinate dehydrogenase inhibitors, strobilurin fungicides, fungicidal multisite inhibitors, and Qi inhibitors are described hereinbelow.

In some embodiments, the components of the combination are applied contemporaneously.

In some embodiments, the components of the combination are applied sequentially.

In some embodiments, the components of the combination are applied separately. In some embodiments, the components of the combination are applied separately in separate compositions.

In some embodiments, the components of the combination are applied together. In some embodiments, the components of the combination are applied together in a single composition.

In some embodiments, the combination is a solid mixture.

In some embodiments, the combination is a liquid mixture.

The present invention provides a composition comprising any one of the combinations or mixtures of the present invention.

The present invention further provides a composition comprising any one or any combination of the crystalline Form III, IIIa, 01 or 02 of the present invention and at least one agriculturally acceptable carrier.

In some embodiments, the composition comprises a solution of any one or any combination of the crystalline Form III, IIIa, 01 or 02 of the present invention.

In some embodiments, the composition comprises a suspension of any one or any combination of the crystalline Form III, IIIa, 01 or 02 of the present invention.

In some embodiments, the composition is a fungicidal composition.

In some embodiments, the composition comprises at least one agriculturally acceptable carrier.

In some embodiments, the composition comprises at least one adjuvant.

In some embodiments, the composition comprises at least one excipient.

In some embodiments, the composition comprises at least one excipient for preparation of a tank mix.

In some embodiments, the composition is a solid composition.

In some embodiments, the composition is a liquid composition.

Solid forms of the compound A exist in either amorphous or crystalline forms.

In some embodiments, the composition comprises an amount of compound A wherein two or more forms of compound A are present and wherein at least one form is the crystalline Form III, IIIa, 01 or 02 of the present invention.

In some embodiments, the crystalline Form III, IIIa, 02 or 01 is at least about 50% by weight of the total amount of compound A in the composition.

In some embodiments, the crystalline Form III, IIIa, 02 or 01 is at least about 60% by weight of the total amount of compound A in the composition.

In some embodiments, the crystalline Form III, IIIa, 02 or 01 is at least about 70% by weight of the total amount of compound A in the composition.

In some embodiments, the crystalline Form III, IIIa, 02 or 01 is at least about 80% by weight of the total amount of compound A in the composition.

In some embodiments, the crystalline Form III, IIIa, 02 or 01 is at least about 90% by weight of the total amount of compound A in the composition.

In some embodiments, the crystalline Form III, IIIa, 02 or 01 is at least about 95% by weight of the total amount of compound A in the composition.

In some embodiments, the crystalline Form III, IIIa, 02 or 01 is at least about 98% by weight of the total amount of compound A in the composition.

In some embodiments, the crystalline Form III, IIIa, 02 or 01 is at least about 99% by weight of the total amount of compound A in the composition.

In some embodiments, the amount of compound A in the composition is substantially free of compound A in amorphous form.

In some embodiments, the amount of compound A in the composition is substantially free of crystalline Form 01 of the present invention.

In some embodiments, the amount of compound A in the composition is substantially free of crystalline Form 02 of the present invention.

In some embodiments, the amount of compound A in the composition is substantially free of crystalline Form 01 and 02 of the present invention.

In some embodiments, the concentration of compound A in the composition is greater than 0.1% by weight based on the total weight of the composition. In some embodiments, the concentration of compound A in the composition is greater than 1% by weight based on the total weight of the composition. In some embodiments, the concentration of compound A in the composition is greater than 5% by weight based on the total weight of the composition. In some embodiments, the concentration of compound A in the composition is greater than 10% by weight based on the total weight of the composition. In some embodiments, the concentration of compound A in the composition is greater than 25% by weight based on the total weight of the composition. In some embodiments, the concentration of compound A in the composition is greater than 50% by weight based on the total weight of the composition.

In some embodiments, the concentration of compound A in the composition is greater than 75% by weight based on the total weight of the composition. In some embodiments, the concentration of compound A in the composition is greater than 90% by weight based on the total weight of the composition.

In some embodiments, the composition further comprises at least one additional pesticide. In some embodiments, the pesticide is fungicide.

In some embodiments, the fungicide is a fungicidal sterol biosynthesis inhibitor.

In some embodiments, the sterol biosynthesis inhibitor is selected from the group consisting of prothioconazole, epoxiconazole, cyproconazole, myclobutanil, prochloraz, metconazole, difenoconazole, tebuconazole, tetraconazole, fenbuconazole, propiconazole, fluquinconazole, flusilazole, flutriafol, fenpropimorph, fenpropidin, ipconazole, triticonazole, spiroxamine, fenhexamid, fenpyrazamine, mefentrifluconazole, and any combination thereof.

In some embodiments, the sterol biosynthesis inhibitor is selected from the group consisting of prothioconazole, epoxiconazole, cyproconazole, myclobutanil, prochloraz, metconazole, difenoconazole, tebuconazole, tetraconazole, fenbuconazole, propiconazole, fluquinconazole, flusilazole, flutriafol, fenpropimorph, and any combination thereof.

In some embodiments, the sterol biosynthesis inhibitor is selected from the group consisting of epoxiconazole, cyproconazole, myclobutanil, metconazole, propiconazole, prothioconazole, fluquinconazole, flutriafol, and difenoconazole.

In some embodiments, the fungicide is a succinate dehydrogenase inhibitor.

In some embodiments, the succinate dehydrogenase inhibitor is selected from the group consisting of fluxapyroxad, benzovindiflupyr, penthiopyrad, isopyrazam, bixafen, boscalid, penflufen, fluopyram, inpyrfluxam, fluindapyr, pydiflumetofen, isofetamid, and any combination thereof.

In some embodiments, the succinate dehydrogenase inhibitor is selected from the group consisting of fluxapyroxad, benzovindiflupyr, penthiopyrad, isopyrazam, bixafen, boscalid, penflufen, fluopyram, and any combination thereof.

In some embodiments, wherein the succinate dehydrogenase inhibitor is selected from the group consisting of fluxapyroxad, benzovindiflupyr, penthiopyrad, isopyrazam, boscalid, fluopyram, and any combination thereof.

In some embodiments, the succinate dehydrogenase inhibitor is fluxapyroxad.

In some embodiments, the fungicide is a strobilurin fungicide.

In some embodiments, the strobilurin fungicide is selected from the group consisting of pyraclostrobin, fluoxastrobin, azoxystrobin, trifloxystrobin, picoxystrobin, kresoxim-methyl, dimoxystrobin, orysastrobin, and any combination thereof.

In some embodiments, the strobilurin fungicide is selected from the group consisting of pyraclostrobin, fluoxastrobin, azoxystrobin, trifloxystrobin, picoxystrobin, kresoxim-methyl, and any combination thereof.

In some embodiments, the strobilurin fungicide is azoxystrobin.

In some embodiments, the fungicide is a fungicidal multisite inhibitor.

In some embodiments, the fungicidal multisite inhibitor is selected from a group consisting of chlorothalonil, mancozeb, folpet, captan, metiram, maneb, propineb, copper hydroxide, copper octanoate, copper oxychloride, copper sulfate, copper sulfate (tribasic), mancopper, oxine-copper, copper bis(3-phenlsalicylate), copper zinc chromate, cuprous oxide, cupric hydrazinium sulfate, cuprobam, and any combination thereof.

In some embodiments, the fungicidal multisite inhibitor is selected from a group consisting of chlorothalonil, mancozeb, folpet, captan, and any combination thereof.

In some embodiments, the fungicidal multisite inhibitor is folpet or captan.

In some embodiments, the fungicide is a Qi inhibitor.

In some embodiments, the Qi inhibitor is a cyano imidazole. In some embodiments, the cyano imidazole is cyazofamid.

In some embodiments, the Qi inhibitor is a sulfamoyl triazole. In some embodiments, the sulfamoyl triazole is amisulbrom.

In some embodiments, the Qi inhibitor is a picolinamide. In some embodiments, the picolinamide is fenpicoxamid.

In some embodiments, the crystalline Form III, IIIa, 01, 02 or combination thereof of the present invention alone, or in combination with at least one additional fungicide is effective to treat a plant or locus against infection of a fungal pathogen.

In some embodiments, the composition of the present invention is effective to treat a plant or locus against infection of a fungal pathogen.

In some embodiments, the fungal pathogen is selected from Leaf Blotch of Wheat (Mycosphaerella graminicola; anamorph: Septoria tritici), Wheat Brown Rust (Puccinia triticina), Stripe Rust (Puccinia striiformis f. sp. tritici), Scab of Apple (Venturia inaequalis), Blister Smut of Maize (Ustilago maydis), Powdery Mildew of Grapevine (Uncinula necator), Barley scald (Rhynchosporium secalis), Blast of Rice (Magnaporthe grisea), Rust of Soybean (Phakopsora pachyrhizi), Glume Blotch of Wheat (Leptosphaeria nodorum), Powdery Mildew of Wheat (Blumeria graminis f. sp. tritici), Powdery Mildew of Barley (Blumeria graminis f. sp. hordei), Powdery Mildew of Cucurbits (Erysiphe cichoracearum), Anthracnose of Cucurbits (Glomerella lagenarium), Leaf Spot of Beet (Cercospora beticola), Early Blight of Tomato (Alternaria solani), and Net Blotch of Barley (Pyrenophora teres).

The compositions comprising or consisting essentially the crystalline forms of the present invention are prepared according to procedures which are conventional in the agricultural chemical art. See, for example, Foy, C. L. and Pritchard, D. W. (1996) Pesticide Formulation and Adjuvant Technology. CRC Press.

Concentrated compositions of the crystalline forms of the present invention can be dispersed in water or another liquid, for application, or the formulations can be dust-like or granular, which can then be applied without further treatment. The compositions are prepared according to procedures which are conventional in the agricultural chemical art, but are novel and important due to the crystalline form present therein.

Concentrated compositions of the crystalline form(s) of the present invention can be dispersed in water or another liquid for application, or can be dust-like or granular, which can be diluted before application.

The concentration of the crystalline form(s) of the present invention and/or combination of the crystalline form of the present invention with at least one other pesticide in the composition is usually from about 0.5% to about 90% by weight, more preferably about 25% to about 75% by weight, based on the total weight of the composition.

The compositions that are applied most often are aqueous suspensions or emulsions. Either such water-soluble, water-suspendable, or emulsifiable formulations are solids, usually known as wettable powders, or liquids, usually known as emulsifiable concentrates, aqueous suspensions, or suspension concentrates. The present disclosure contemplates all vehicles by which the compositions can be formulated for delivery and used as a fungicide.

As will be readily appreciated, any material to which the disclosed compositions can be added may be used, provided they yield the desired utility without significant interference with the activity of these compositions as antifungal agents.

Wettable powders, which may be compacted to form water-dispersible granules, comprise an intimate mixture of compound A in one or more forms, preferably wherein at least one form is Form III, Form IIIa, Form 01, or Form 02 described herein, a carrier and agriculturally acceptable surfactants. The concentration of compound A in the wettable powder is usually from about 10% to about 90% by weight, more preferably about 25% to about 75% by weight, based on the total weight of the composition. In the preparation of wettable powder compositions, the composition can be compounded with any of the finely divided solids.

The disclosed compositions may optionally include combinations that can comprise at least 1% by weight of one or more of the compositions with another pesticide. Such additional pesticides may be fungicides, insecticides, nematocides, miticides, arthropodicides, bactericides or combinations thereof that are compatible with the compositions of the present disclosure in the medium selected for application, and not antagonistic to the activity of the present compounds. Accordingly, in such embodiments the other pesticide is employed as a supplemental toxicant for the same or for a different pesticidal use. The pesticide and the composition can generally be mixed together in a weight ratio of from 1:100 to 100:1.

### Methods of Use

The present invention further provides a method for treating a plant or locus against fungal infection, wherein the method comprises:
i) obtaining an effective amount of a compound having the following structure:

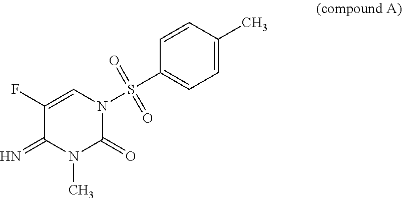 [SMILES: Cc1ccc(S(=O)(=O)n2cc(F)c(=N)n(C)c2=O)cc1]

in one or more forms, wherein at least one form of the compound is the crystalline Form III, IIIa, 01 or 02 described herein; and
ii) applying the amount of compound A to the plant or locus so as to thereby treat the plant or locus against fungal infection.

The present invention further provides a method for treating a plant or locus against fungal infection, wherein the method comprises:
i) obtaining an effective amount of any one of the crystalline forms, combinations, mixtures, or compositions described herein; and
ii) applying the amount of the crystalline form, combination, mixture, or composition to the plant or locus so as to thereby treat the plant or locus against fungal infection.

In some embodiments, the method is effective for controlling fungal infection of the plant or locus.

In some embodiments, controlling fungal infection comprises controlling fungal disease infecting the plant or locus. In some embodiments, controlling fungal infection comprises controlling a plant or soil disease caused by phytopathologic fungi. In some embodiments, controlling fungal infection comprises controlling fungal attack on the plant or locus. In some embodiments, controlling fungal infection comprises reducing fungal infection of the plant or locus. In some embodiments, controlling fungal infection comprises curing a plant or soil disease caused by phytopathologic fungi.

In some embodiments, the method is effective for protecting the plant or locus against fungal infection.

In some embodiments, protecting the plant or locus against fungal infection comprises protecting the plant or locus against fungal attack. In some embodiments, protecting the plant or locus against fungal infection comprises protecting the plant or locus from fungal disease. In some embodiments, protecting the plant or locus against fungal infection comprises preventing fungal infection of the plant or locus.

In some embodiments, the method is effective for treating plant roots against fungal infection. In some embodiments, the method is effective for controlling fungal attack on plant roots.

In some embodiments, the method is effective for treating plant seeds against fungal infection. In some embodiments, the method is effective for controlling fungal attack on plant seeds.

In some embodiments, the crystalline form, combination, mixture or composition is applied to a portion of a plant.

In some embodiments, the crystalline form, combination, mixture or composition is applied to plant roots. In some embodiments, the crystalline form, combination, mixture or composition is applied to propagation material of the plant. In some embodiments, the crystalline form, combination, mixture or composition is applied to plant seeds.

In some embodiments, the crystalline form, combination, mixture or composition is applied to plant seedling. In some embodiments, the crystalline form, combination, mixture or composition is applied to plant foliage.

In some embodiments, the crystalline form, combination, mixture or composition is applied to soil. In some embodiments, the crystalline form, combination, mixture or composition is applied to a locus where fungal infection is to be prevented.

In some embodiments, the crystalline form, combination, mixture or composition is applied to an area adjacent to a plant, soil in contact with a plant, soil adjacent to a plant, any surface adjacent to a plant, any surface in contact with a plant, a seed, and/or equipment used in agriculture.

In some embodiments, the crystalline form, combination, mixture or composition is applied to a locus of the plant, a locus in proximity to the plant, a locus of the fungi, or a locus in proximity to the fungi. In some embodiments, the crystalline form, combination, mixture or composition is applied to soil in which the plant is grown. In some embodiments, the crystalline form, combination, mixture or composition is applied to soil in which the plant is to be grown.

In some embodiments, the crystalline form, combination, mixture or composition is applied as a soil application. In some embodiments, the crystalline form, combination, mixture or composition is applied as a foliar application.

In some embodiments, the method is for the control of fungal attack on the roots and/or seeds and/or a plant, the method comprising applying the crystalline form, the combination, the mixture or the composition of the present invention, to the roots, seeds or foliage of plants, to a locus in which the fungal infection is to be prevented, and/or to the plant, so as to thereby control fungal infection on the roots and/or seeds and/or plant.

In some embodiments, the method comprises applying a mixture. In some embodiments, the mixture is a tank mix comprising any one or any combination of the crystalline forms of the present invention. In some embodiments, the tank mix comprises at least one excipient.

In some embodiments, the method comprises applying a composition. In some embodiments, the method comprises applying a composition, wherein the composition comprises a solution of any one or any combination of the crystalline forms of the present invention. In some embodiments, the method comprises applying a composition, wherein the composition comprises a suspension of any one or any combination of the crystalline forms of the present invention.

In some embodiments, the method is effective for treating the plant or locus against fungal infection without damaging the commercial value of the plant.

In some embodiments, the method comprises a protectant application of any one of the crystalline forms, combinations, mixtures or compositions disclosed herein. In some embodiments, the method comprises a curative application of any one of the crystalline forms, combinations, mixtures or compositions disclosed herein.

In some embodiments, the method comprises applying the crystalline form, combination, mixture or composition before existence of a fungal pathogen infection. In some embodiments, the method comprises applying the crystalline form, combination, mixture or composition before fungal disease symptoms are shown. In some embodiments, the method comprises applying the crystalline form, combination, mixture or composition when disease pressure is low.

In some embodiments, the method comprises applying the crystalline form, combination, mixture or composition after existence of a fungal pathogen infection. In some embodiments, the method comprises applying the crystalline form, combination, mixture or composition after fungal disease symptoms are shown.

In some embodiments, the crystalline form, combination, mixture or composition is applied at the time of planting. In some embodiments, the crystalline form, combination, mixture or composition is applied 1 to 60 day(s) after planting. In some embodiments, the crystalline form, combination, mixture or composition is applied 1 to 9 month(s) after planting.

In some embodiments, the crystalline form, combination, mixture or composition is applied once during a growth season. In some embodiments, the crystalline form, combination, mixture or composition is applied at least one time during a growth season. In some embodiments, the crystalline form, combination, mixture or composition is applied two or more times during a growth season.

In some embodiments, the amount of compound A applied is from 1 g/ha to 500 g/ha. In some embodiments, the amount of compound A applied is from 5 g/ha to 150 g/ha. In some embodiments, the amount of compound A applied is from 5 g/ha to 120 g/ha. In some embodiments, the amount of compound A applied is from 1 g/ha to 100 g/ha. In some embodiments, the amount of compound A applied is from 1 g/ha to 75 g/ha. In some embodiments, the amount of compound A applied is from 1 g/ha to 50 g/ha. In some embodiments, the amount of compound A applied is from 1 g/ha to 25 g/ha. In some embodiments, the amount of compound A applied is from 1 g/ha to 15 g/ha.

In some embodiments, the amount of compound A applied is 6.25 g/ha. In some embodiments, the amount of compound A applied is 10 g/ha. In some embodiments, the amount of compound A applied is 12.5 g/ha. In some embodiments, the amount of compound A applied is 20 g/ha. In some embodiments, the amount of compound A applied is 75 g/ha. In some embodiments, the amount of compound A applied is 100 g/ha. In some embodiments, the amount of compound A applied is 125 g/ha.

In some embodiments, the method comprises applying a fungicidal mixture to a locus of the fungus, to a locus in which the infestation is to be prevented, and/or to the plant, the mixture comprises: i) a fungicidally effective amount of the crystalline form, combination, mixture or composition of the present invention; and ii) at least one additional fungicide, so as to thereby control fungal attack on the plant.

In some embodiments, the fungicidal mixture provides greater than additive effect in controlling fungal attack on the plant compared to when the crystalline form and the additional pesticide(s), preferably fungicide(s), are applied alone at the same amounts.

In some embodiments, the method comprises applying at least one additional pesticide. In some embodiments, the pesticide is a fungicide.

In some embodiments, the fungicide is a fungicidal sterol biosynthesis inhibitor.

In some embodiments, the sterol biosynthesis inhibitor is selected from the group consisting of prothioconazole, epoxiconazole, cyproconazole, myclobutanil, prochloraz, metconazole, difenoconazole, tebuconazole, tetraconazole, fenbuconazole, propiconazole, fluquinconazole, flusilazole, flutriafol, fenpropimorph, fenpropidin, ipconazole, triticonazole, spiroxamine, fenhexamid, fenpyrazamine, mefentrifluconazole, and any combination thereof.

In some embodiments, the sterol biosynthesis inhibitor is selected from the group consisting of prothioconazole, epoxiconazole, cyproconazole, myclobutanil, prochloraz, metconazole, difenoconazole, tebuconazole, tetraconazole, fenbuconazole, propiconazole, fluquinconazole, flusilazole, flutriafol, fenpropimorph, and any combination thereof.

In some embodiments, the sterol biosynthesis inhibitor is selected from the group consisting of epoxiconazole, cyproconazole, myclobutanil, metconazole, propiconazole, prothioconazole, fluquinconazole, flutriafol, and difenoconazole.

In some embodiments, the fungicide is a succinate dehydrogenase inhibitor.

In some embodiments, the succinate dehydrogenase inhibitor is selected from the group consisting of fluxapyroxad, benzovindiflupyr, penthiopyrad, isopyrazam, bixafen, boscalid, penflufen, fluopyram, inpyrfluxam, fluindapyr, pydiflumetofen, isofetamid, and any combination thereof.

In some embodiments, the succinate dehydrogenase inhibitor is selected from the group consisting of fluxapyroxad, benzovindiflupyr, penthiopyrad, isopyrazam, bixafen, boscalid, penflufen, fluopyram, and any combination thereof.

In some embodiments, wherein the succinate dehydrogenase inhibitor is selected from the group consisting of fluxapyroxad, benzovindiflupyr, penthiopyrad, isopyrazam, boscalid, fluopyram, and any combination thereof.

In some embodiments, the succinate dehydrogenase inhibitor is fluxapyroxad.

In some embodiments, the fungicide is a strobilurin fungicide.

In some embodiments, the strobilurin fungicide is selected from the group consisting of pyraclostrobin, fluoxastrobin, azoxystrobin, trifloxystrobin, picoxystrobin, kresoxim-methyl, dimoxystrobin, orysastrobin, and any combination thereof.

In some embodiments, the strobilurin fungicide is selected from the group consisting of pyraclostrobin, fluoxastrobin, azoxystrobin, trifloxystrobin, picoxystrobin, kresoxim-methyl, and any combination thereof.

In some embodiments, the strobilurin fungicide is azoxystrobin.

In some embodiments, the fungicide is a fungicidal multisite inhibitor.

In some embodiments, the fungicidal multisite inhibitor is selected from a group consisting of chlorothalonil, mancozeb, folpet, captan, metiram, maneb, propineb, copper hydroxide, copper octanoate, copper oxychloride, copper sulfate, copper sulfate (tribasic), mancopper, oxine-copper, copper bis(3-phenlsalicylate), copper zinc chromate, cuprous oxide, cupric hydrazinium sulfate, cuprobam, and any combination thereof.

In some embodiments, the fungicidal multisite inhibitor is selected from a group consisting of chlorothalonil, mancozeb, folpet, captan, and any combination thereof.

In some embodiments, the fungicidal multisite inhibitor is folpet or captan.

In some embodiments, the fungicide is a Qi inhibitor.

In some embodiments, the Qi inhibitor is a cyano imidazole. In some embodiments, the cyano imidazole is cyazofamid.

In some embodiments, the Qi inhibitor is a sulfamoyl triazole. In some embodiments, the sulfamoyl triazole is amisulbrom.

In some embodiments, the Qi inhibitor is a picolinamide. In some embodiments, the picolinamide is fenpicoxamid.

In some embodiments, the disclosed crystalline form and/or mixture of disclosed crystalline forms with at least one other pesticide and/or the composition comprising the crystalline form of the present invention (including a mixture with at least one other pesticide) is applied to the roots, seeds or foliage of plants for the control of various fungi, without damaging the commercial value of the plants.

In some embodiments, the disclosed crystalline form and/or mixture of disclosed crystalline form with at least one other pesticide may be applied to the roots, seeds or foliage of plants for the control of various fungi, without damaging the commercial value of the plants.

In some embodiments, the additional pesticide and the crystalline form, combination, mixture or composition of the present invention are applied simultaneously.

In some embodiments, the additional pesticide and the crystalline form, combination, mixture or composition of the present invention are applied contemporaneously.

In some embodiments, the additional pesticide and the crystalline form, combination, mixture or composition of the present invention are applied sequentially.

In some embodiments, the additional pesticide and the crystalline form, combination, mixture or composition of the present invention are applied separately.

In some embodiments, the additional pesticide and the crystalline form, combination, mixture or composition of the present invention are applied together.

In some embodiments, the additional pesticide and the crystalline form, combination, mixture or composition of the present invention are applied together as a tank mix.

In some embodiments, the additional pesticide and the crystalline form, combination, mixture or composition of the present invention are formulated as a single composition.

In some embodiments, the additional pesticide and the crystalline form, combination, mixture or composition of the present invention are formulated as separated compositions.

PCT International Application No. PCT/US2014/072745 disclosed synergistic fungicidal mixtures and compositions comprising 5-fluoro-4-imino-3-methyl-1-tosyl-3,4-dihydropyrimidin-2 (1H)-one and at least one fungicidal sterol biosynthesis inhibitor, as well as uses thereof.

PCT International Application No. PCT/US2014/072747 disclosed synergistic fungicidal mixtures and compositions comprising 5-fluoro-4-imino-3-methyl-1-tosyl-3,4-dihydropyrimidin-2 (1H)-one and at least one succinate dehydrogenase inhibitor, as well as uses thereof.

PCT International Application No. PCT/US2014/072748 disclosed synergistic fungicidal mixtures and compositions comprising 5-fluoro-4-imino-3-methyl-1-tosyl-3,4-dihydropyrimidin-2 (1H)-one and at least one fungicidal multi-site inhibitor or strobilurin fungicide, as well as uses thereof.

PCT International Application No. PCT/IB2020/056828 disclosed combinations, mixtures and compositions comprising 5-fluoro-4-imino-3-methyl-1-tosyl-3,4-dihydropyrimidin-2(1H)-one and at least one fungicide (A) selected from the group consisting of fluindapyr, pydiflumetofen, mefentrifluconazole, inpyrfluxam, isofetamid and Qi inhibitor, as well as uses thereof.

The entire content of each of PCT/US2014/072745, PCT/US2014/072747, PCT/US2014/072748, and PCT/IB2020/056828 is hereby incorporated by reference into this application.

In particular, the crystalline forms of 5-fluoro-4-imino-3-methyl-1-tosyl-3,4-dihydropyrimidin-2(1H)-one of the present invention may be used as the 5-fluoro-4-imino-3-methyl-1-tosyl-3,4-dihydropyrimidin-2(1H)-one in the combinations, mixtures, compositions, methods and processes described in PCT/US2014/072745, PCT/US2014/072747, PCT/US2014/072748, and PCT/IB2020/056828.

The crystalline forms, combinations, mixtures or compositions disclosed herein may also be used as seed treatment to prevent or control plant disease as described in PCT International Application No. PCT/US2015/066756, the entire content of which is hereby incorporated by reference into this application.

The crystalline forms, combinations, mixtures or compositions disclosed herein may also be used for treating a plant against fungal pathogen infection and/or fungal disease as described in U.S. Provisional Application No. 63/142,447, the entire content of which is hereby incorporated by reference into this application.

The present invention provides a method for treating a plant against fungal pathogen infection and/or fungal disease comprising applying the crystalline form, combination, mixture or composition disclosed herein to a plant or a locus thereof so as to thereby treat the plant against fungal pathogen infection and/or fungal disease, wherein:
(1) the fungal pathogen is selected from group consisting of Pyricularia oryzae, Rhizoctonia solani, sclerotinia sclerotium, Pseudoperonospora cubensis, Venturia inequalis, Podospaera leucotricha, Botrytis cinerea, Sphaerotheca fuliginea, Pseudoperonospora cubensis, Alternaria solani, Cercospora beticola, Ramularia beticola, Ramularia areola, Erysiphe betae, Phakopsora pachyrhizi, Microsphaera diffusa, Mycosphaerella areola, Corynespora cassiicola, Colletotrichum dematium, Cercospora kikushi, Plasmopara viticola, Mycosphaerella fijiensis and any combination thereof,
(2) the fungal disease is selected from group consisting of blast, sheath blight, early blight, apple scab, downy mildew, black sigatoka, downy mildew, late season diseases of soybean, Asian soybean rust, anthracnose, powdery mildew, and any combination thereof, and/or
(3) the plant is selected from group consisting of soybean, rice, fruit plants, vegetable plants, sugar beet, rapeseed, grapevine, cotton and any combination thereof.

### Processes of Preparation of the Crystalline Forms of Compound A

The present invention also provides processes for preparing the crystalline Forms III, IIIa, 01 and 02.

In some embodiments, the process is for preparing the crystalline form of the present invention, the process comprising:
a) combining an amount of a compound having the following structure:

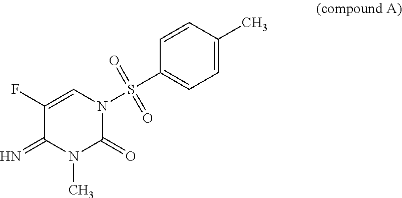 [SMILES: Cc1ccc(S(=O)(=O)n2cc(F)c(=N)n(C)c2=O)cc1]

and a suitable solvent to prepare a mixture;
b) optionally removing any precipitated solid from the mixture of step a); and
c) obtaining the crystalline form of the present invention.

In some embodiments, step a) comprises dissolving compound A in the solvent.

In some embodiments, step a) comprises dissolving compound A in the solvent by using mechanical means such as Vortex.

In some embodiments, step a) comprises suspending compound A in the solvent.

In some embodiments, step b) is performed.

In step b), the precipitated solid may be removed by any known method such as filtration or vortex.

In some embodiments, the crystalline form is obtained by crystallization. In some embodiments, the crystalline form is obtained by evaporation crystallization, by suspension crystallization or by cooling crystallization. In some embodiments, the crystalline form is obtained by evaporation crystallization. In some embodiments, the crystalline form is obtained by suspension crystallization. In some embodiments, the crystalline form is obtained by cooling crystallization.

Processes for preparing compound A used in step a) are described in PCT International Applications Nos. PCT/US2014/072566, PCT/US2014/072569, PCT/IB2020/058893 and PCT/IB2021/051957, the content of each of which is hereby incorporated by reference into this application.

In some embodiments, the process comprises preparing the compound using any one of the processes described in PCT/US2014/072566, PCT/US2014/072569, PCT/IB2020/058893 or PCT/IB2021/051957 and using the resulting reaction mixture containing the compound directly in step a).

In some embodiments, the amount of compound A used in step a) is in one or more forms selected from the group consisting of amorphous form, crystalline forms, hydrate forms, and any combination thereof.

Examples of crystalline forms and hydrate forms of the compound that may be used in step a) are described in PCT International Application No. PCT/IB2018/000875, the entire content of which is hereby incorporated by reference into this application.

In some embodiments, at least one of the forms of compound A used in step a) is an amorphous form.

In some embodiments, at least one of the forms of compound A used in step a) is a crystalline form.

In some embodiments, at least one of the forms of compound A used in step a) is a hydrate form.

In some embodiments, the crystalline form of the compound or one of the crystalline forms of the compound used in step a) is the crystalline polymorphic form designated “Form I” in PCT/IB2018/000875. Form I exhibits an X-ray powder diffraction pattern as shown in FIG. 1 of PCT/IB2018/000875, having characteristic peaks at 2-theta angles of 9.08, 10.98, 14.05, 17.51, 18.75, 21.63, 23.33, 24.70, 24.83, 25.37, 26.51 and 29.23. In some embodiments, the powder X-ray diffraction pattern of Form I comprises characteristic peaks at 2-theta angles of 14.05, 17.51, 18.75, 21.63 and 26.51. In some embodiments the powder X-ray diffraction pattern of Form I comprises characteristic peaks at 2-theta angles of 14.05, 17.51, 18.75 and 21.63.

In some embodiments, the crystalline form of the compound or one of the crystalline forms of the compound used in step a) is the crystalline polymorphic form designated “Form II” in PCT/IB2018/000875. Form II exhibits an X-ray powder diffraction pattern as shown in FIG. 4 of PCT/IB2018/000875, having characteristic peaks at 2-theta angles of 7.98, 9.20, 9.96, 11.88, 15.99, 18.49, 21.23, 22.33, 22.59, 26.73. In some embodiments, the X-ray powder diffraction pattern of Form II comprises characteristic peaks at 2-theta angles of 9.20, 9.96, 11.88, 22.33 and 22.59. In some embodiments, the X-ray powder diffraction pattern of Form II comprises characteristic peaks at 2-theta angles of 9.20, 11.88, 22.33 and 22.59.

In some embodiments, the hydrate form of the compound or one of the hydrate forms of the compound used in step a) is the crystalline hydrate form designated “Hydrate” in PCT/IB2018/000875. Hydrate exhibits an X-ray powder diffraction pattern as shown in FIG. 7 of PCT/IB2018/000875, having characteristic peaks at 2-theta 5.34, 7.48, 10.68, 16.05, 21.79, 22.99, 23.19, 24.95, 26.95, 27.63. In some embodiments, the powder X-ray diffraction pattern of Hydrate comprises characteristic peaks at 2-theta angles of 5.34, 7.48, 10.68, 16.05 and 21.79. In some embodiments the powder X-ray diffraction pattern of Hydrate comprises characteristic peaks at 2-theta angles of 5.34, 7.48, 10.68 and 16.05.

In some embodiments of the process for preparing crystalline Form IIIa, at least one form of compound A used in step a) is Form III of the present invention.

PCT International Application No. PCT/IB2018/000875 also disclosed processes for preparing the crystalline forms and hydrate forms described therein.

In some embodiments, the process comprises preparing the compound using any one of the processes described in PCT/IB2018/000875 and using the resulting reaction mixture containing the compound directly in step a).

In some embodiments, the compound is used for the preparation of the crystalline form directly in the solution it was prepared in.

In some embodiments, step a) uses a batch of compound A that is at least 95% pure of compound A, or at least 96% pure of compound A, or at least 97% pure of compound A, or at least 98% pure of compound A, at least 99% pure of compound A, or at least 99.5% pure of compound A.

In some embodiments, step a) uses a batch of compound A that is substantially pure of compound A.

In some embodiments, step a) uses a substantially pure amount of compound A.

In some embodiments, step a) uses a mixture containing compound A, wherein 95% or more by weight of the mixture is compound A. In some embodiments, step a) uses a mixture containing compound A, wherein 96% or more by weight of the mixture is compound A.

In yet another embodiment, the product of any of the disclosed processes can be isolated from the reaction mixture by any conventional techniques well-known in the art. Such isolation techniques can include, without limitation, one or more of the following: concentration, extraction, precipitation, cooling, filtration, crystallization, and centrifugation, followed by drying.

In yet another embodiment, the product of any of the disclosed processes can be optionally purified by any conventional techniques well-known in the art. Such purification techniques may include, without limitation, one or more of the following: precipitation, crystallization, slurrying, washing in a suitable solvent, filtration through a packed-bed column, dissolution in an appropriate solvent, and re-precipitation by addition of a second solvent in which the compound is insoluble, or any combination thereof.

### i) Crystalline Polymorph Form III

In some embodiments, the crystalline form of compound A prepared is crystalline polymorph Form III.

In some embodiments, the crystalline form of compound A obtained in step c) is crystalline polymorph Form III and the solvent in step a) is selected from the group consisting of isobutyl acetate, cyclohexanone, propylacetate, isopropylacetate, butylacetate, diethyl carbonate, 1,2 dimethoxyethane, 1,2 methyl ethyl ketone, anisole, toluene, 2-methyltetrahydrofuran, water, acetonitrile, and any combination thereof.

In some embodiments, the crystalline form of compound A obtained in step c) is crystalline polymorph Form III and compound A used in step a) is the crystalline polymorphic form designated “Form I” in PCT/IB2018/000875.

In some embodiments, the crystalline form of compound A obtained in step c) is crystalline polymorph Form III and compound A used in step a) is the crystalline hydrate form designated “Hydrate” in PCT/IB2018/000875.

In some embodiments, the crystalline form of compound A obtained in step c) is crystalline polymorph Form III and compound A used in step a) is a mixture of the crystalline polymorphic form designated “Form I” in PCT/IB2018/000875 and the crystalline hydrate form designated “Hydrate” in PCT/IB2018/000875.

In some embodiments, the crystalline polymorph Form III is obtained by suspension crystallization.

In some embodiments, the crystalline polymorph Form III is obtained by evaporation crystallization.

In some embodiments, the crystalline polymorph Form III is obtained by cooling crystallization.

In some embodiments, the crystalline form of compound A obtained in step c) is crystalline polymorph Form III and the process comprises:
a) suspending the amount of compound A in water to prepare a suspension mixture;
b) optionally removing any precipitated solid from the suspension mixture of step a); and
c) obtaining the crystalline polymorph Form III of the present invention by suspension crystallization.

In some embodiments, step b) is performed.

In some embodiments, the suspension concentration is about 1 mg/ml to 20 mg/ml. In some embodiments, the suspension concentration is about 10 mg/ml.

In some embodiments, the suspension is prepared at a temperature in the range of about 20° C. to about 80° C. for 2-3 days.

In some embodiments, the crystalline form of compound A obtained in step c) is crystalline polymorph Form III and the process comprises:
a) dissolving the amount of compound A in a solvent to prepare a solution mixture, wherein the solvent is selected from the group consisting of isobutyl acetate, cyclohexanone, propylacetate, isopropylacetate, butylacetate, diethyl carbonate, 1,2 dimethoxyethane, 1,2 methyl ethyl ketone, anisole, toluene, 2-methyltetrahydrofuran, and any combination thereof;
b) optionally removing any precipitated solid from the solution mixture of step a); and
c) obtaining the crystalline polymorph Form III of the present invention by evaporation crystallization.

In some embodiments, the process wherein the solution concentration is about 1 mg/ml to 20 mg/ml. In a preferred embodiment, the suspension concentration is about 10 mg/ml.

In some embodiments, the solution is prepared at a temperature in the range of about 20° C. to about 80° C. for 20 minutes to about 24 hours.

In some embodiments, the solution is prepared at a temperature of about 25° C. for 60 minutes.

In some embodiments, the solution is prepared at a temperature of about 75° C.

In some embodiments, step c) comprises evaporating the solution from step b) at about 50° C. to 60° C. until the solvent is completely evaporated, by visual inspection.

In some embodiments, the crystalline form of compound A obtained in step c) is crystalline polymorph Form III and the process comprises:
a) dissolving compound A in acetonitrile to prepare a solution;
b) optionally removing any precipitated solid from the solution of step a); and
c) obtaining the crystalline polymorph Form III of the present invention by cooling crystallization.

In some embodiments, the mixture of Form I and a hydrate form comprises at least 10% hydrate, at least 20% hydrate, at least 30% hydrate, at least 40% hydrate, at least 50% hydrate, at least 60% hydrate, at least 70% hydrate, at least 80% hydrate or at least 90% hydrate.

In some embodiments, the mixture of Form I and a hydrate form is 30% Form I and 70% hydrate.

In some embodiments, the solvent is acetonitrile.

In some embodiments, the solvent is acetonitrile and compound A is Form I.

In some embodiments, the process wherein the solution concentration is about 50 mg/ml to 130 mg/ml. In a preferred embodiment, the suspension concentration is about 70 mg/ml. In another preferred embodiment, the suspension concentration is about 125 mg/ml.

In some embodiments, the solution is prepared at a temperature in the range of about 50° C. to about 80° C. until compound A is dissolved.

In a preferred embodiment, the solution is prepared at about 60-65° C.

In some embodiments, the process is for preparing the crystalline polymorph Form III, the process comprising:
a) combining compound A with a solvent to prepare a mixture, wherein the solvent is selected from the group consisting of isobutyl acetate, cyclohexanone, propylacetate, isopropylacetate, butylacetate, diethyl carbonate, 1,2 dimethoxyethane, 1,2 methyl ethyl ketone, anisole, toluene, 2-methyltetrahydrofuran, water, acetonitrile, and any combination thereof;
b) optionally removing any precipitated solid from the mixture of step a); and
c) obtaining the crystalline polymorph Form III of the present invention.

### ii) Crystalline Polymorph Form IIIa

In some embodiments, the crystalline form of compound A prepared is crystalline polymorph Form IIIa.

In some embodiments, the crystalline form of compound A obtained in step c) is crystalline polymorph Form IIIa and the solvent in step a) is selected from the group consisting of water, methyl ethyl ketone, a mixture of water and DMF, and any combination thereof.

In some embodiments, compound A used in step a) is the crystalline polymorphic form of Form I, the crystalline polymorphic form of Form III, the Hydrate, or any mixture thereof.

In some embodiments, the crystalline form of compound A obtained in step c) is crystalline polymorph Form IIIa and compound A used in step a) is the crystalline polymorphic form designated “Form I” in PCT/IB2018/000875.

In some embodiments, the crystalline form of compound A obtained in step c) is crystalline polymorph Form IIIa and compound A used in step a) is the crystalline hydrate form designated “Hydrate” in PCT/IB2018/000875.

In some embodiments, the crystalline form of compound A obtained in step c) is crystalline polymorph Form IIIa and compound A used in step a) is crystalline polymorph Form III of the present invention.

In some embodiments, the crystalline form of compound A obtained in step c) is crystalline polymorph Form IIIa and compound A used in step a) is a mixture of the crystalline polymorphic form designated “Form I” in PCT/IB2018/000875 and the crystalline hydrate form designated “Hydrate” in PCT/IB2018/000875.

In some embodiments, the crystalline form of compound A obtained in step c) is crystalline polymorph Form IIIa and compound A used in step a) is a mixture of the crystalline polymorphic form designated “Form I” in PCT/IB2018/000875 and crystalline polymorph Form III of the present invention.

In some embodiments, the crystalline form of compound A obtained in step c) is crystalline polymorph Form IIIa and compound A used in step a) is a mixture of crystalline polymorph Form III of the present invention and the crystalline hydrate form designated “Hydrate” in PCT/IB2018/000875.

In some embodiments, the crystalline form of compound A obtained in step c) is crystalline polymorph Form IIIa and compound A used in step a) is a mixture of the crystalline polymorphic form designated “Form I” in PCT/IB2018/000875, the crystalline hydrate form designated “Hydrate” in PCT/IB2018/000875, and crystalline polymorph Form III of the present invention.

In some embodiments, the solvent is water.

In some embodiments, the solvent is a mixture of water and DMF.

In some embodiments, the solvent is methyl ethyl ketone.

In some embodiments, the crystalline polymorph Form IIIa is obtained by suspension crystallization.

In some embodiments, the crystalline form of compound A obtained in step c) is crystalline polymorph Form IIIa and the process comprises:
a) suspending the amount of compound A in water to prepare a suspension mixture;
b) optionally removing any precipitated solid from the suspension mixture of step a); and
c) obtaining the crystalline polymorph Form IIIa of the present invention by suspension crystallization.

In the above embodiment, compound A used in step a) is preferably the crystalline polymorphic form designated “Form I” in PCT/IB2018/000875, the crystalline hydrate form designated “Hydrate” in PCT/IB2018/000875, crystalline polymorph Form III of the present invention, or any mixture thereof.

In some embodiments, the suspension is prepared at a temperature in the range of about 20° C. to about 60° C. for 7 to 15 days before proceeding to step b).

In some embodiments, the suspension in step a) is stirred at about 50° C. for about 7 days before proceeding to step b).

In some embodiments, the suspension in step a) is stirred at room temperature for about 15 days before proceeding to step b).

In some embodiments, the suspension concentration is about 1 mg/ml to 20 mg/ml. In a preferred embodiment, the suspension concentration is about 10 mg/ml.

In some embodiments, the crystalline form of compound A obtained in step c) is crystalline polymorph Form IIIa and the process comprises:
a) suspending the amount of compound A in methyl ethyl ketone to prepare a mixture;
b) optionally removing any precipitated solid from the suspension mixture of step a); and
c) obtaining the crystalline polymorph Form IIIa of the present invention by suspension crystallization.

In the above embodiment, compound A used in step a) is preferably crystalline polymorph Form III of the present invention.

In some embodiments, the suspension in step a) is stirred at about 20° C. to 40° C. for about 3 to 5 days before proceeding to step b). Preferably, the suspension in step a) is stirred at about 25° C. for about 3 days before proceeding to step b).

In some embodiments, the suspension concentration is about 50 mg/ml to 100 mg/ml. In some embodiments, the suspension concentration is about 80 mg/ml.

In some embodiments, the crystalline form of compound A obtained in step c) is crystalline polymorph Form IIIa and the process comprises:
a) suspending the amount of compound A in a mixture of water and DMF to prepare a suspension mixture;
b) optionally removing any precipitated solid from the suspension mixture of step a); and
c) obtaining the crystalline polymorph Form IIIa of the present invention by suspension crystallization.

In the above embodiment, compound A used in step a) is preferably the crystalline polymorphic form designated “Form I” in PCT/IB2018/000875.

In some embodiments, the mixture of water and DMF used in step a) comprises 0.1%-5% v/v DMF. In some embodiments, the mixture of water and DMF used in step a) comprises 0.5%-2% v/v DMF. In a preferred embodiment, the mixture of water and DMF used in step a) comprises 1% v/v DMF.

In some embodiments, the suspension mixture of step a) is stirred at about 30° C. to 60° C. for about 1 to 8 days before proceeding to step b).

Preferably, the suspension mixture of step a) is stirred at about 50° C. for about 2-7 days before proceeding to step b).

In some embodiments, the suspension concentration is about 1 mg/ml to 20 mg/ml. In a preferred embodiment, the suspension concentration is about 10 mg/ml.

In some embodiments, the crystalline form of compound A obtained in step c) is crystalline polymorph Form IIIa and the process comprises:
a) suspending the amount of compound A in a solvent selected from water, methyl ethyl ketone or a mixture of water and DMF to prepare a suspension mixture;
b) optionally removing any precipitated solid from the suspension mixture of step a); and
c) obtaining the crystalline polymorph Form IIIa of the present invention.
iii) Crystalline Form 01 In some embodiments, the crystalline form of compound A prepared is crystalline Form 01.

In some embodiments, the crystalline form of compound A obtained in step c) is crystalline Form 01 and the solvent in step a) is chloroform.

In some embodiments, the crystalline Form 01 is obtained by evaporation crystallization.

In some embodiments, the crystalline form of compound A obtained in step c) is crystalline Form 01 and the process comprises:
a) dissolving the amount of compound A in chloroform to prepare a solution mixture;
b) optionally removing any precipitated solid from the solution mixture of step a); and
c) obtaining the crystalline Form 01 of the present invention by evaporation crystallization.

In some embodiments, compound A is in Form I.

In some embodiments, step b) is performed. In some embodiments, the precipitated solid is removed by filtration in step b).

In some embodiments, the solution mixture is prepared at a temperature in the range of about 15° C. to about 35° C. for 0.5 to 5 hours.

In some embodiments, the solution mixture in step a) is stirred at room temperature for 30 minutes to 5 hours before proceeding to step b).

In some embodiments, the solution in step a) is stirred at room temperature for about 60 minutes before proceeding to step b).

In some embodiments, the solution in step a) is stirred at about 25° C. for 60 minutes before proceeding to step b).

In some embodiments, the solution in step b) is left to evaporate at room temperature.

### iv) Crystalline Form 02

In some embodiments, the crystalline form of compound A prepared is crystalline Form 02.

In some embodiments, the solvent is p-xylene, mesitylene or a mixture thereof.

In some embodiments, the crystalline Form 02 is obtained by evaporation crystallization.

In some embodiments, the crystalline form of compound A obtained in step c) is crystalline Form 02 and the process comprises:
a) dissolving the amount compound A in p-xylene and/or mesitylene to prepare a solution mixture;
b) optionally removing any precipitated solid from the solution mixture of step a); and
c) obtaining the crystalline Form 02 of the present invention by evaporation crystallization.

In some embodiments, compound A is in Form I.

In some embodiments, the solution mixture of step a) is filtered prior to step b).

In some embodiments, the crystalline Form 02 is formed by evaporation crystallization.

In some embodiments, the solution is left until completely evaporated, by visual inspection.

In some embodiments, the solution mixture of step a) is stirred at about 25° C. for 60 minutes before proceeding to step b).

In some embodiments, the solution mixture is left to evaporate at about 50-60° C. in step c).

In some embodiments, the crystalline form of compound A obtained in step c) is crystalline Form 02 and the process comprises:
a) dissolving the amount of compound A in p-xylene and/or mesitylene at a temperature in the range of about 20° C. to about 30° C. for 1 to 10 hours to prepare a solution mixture;
b) filtering any precipitated solid from the solution mixture of step a); and
c) obtaining the crystalline Form 02 of the present invention by evaporation crystallization.

In some embodiments, the solution mixture in step a) is stirred at about 25° C. for 60 minutes before proceeding to step b).

In some embodiments, wherein the solution mixture is left to evaporate at about 50-60° C. in step c).

In the above embodiment, compound A used in step a) is preferably the crystalline polymorphic form designated “Form I” in PCT/IB2018/000875.

### Process for Preparing Compositions

The subject invention further provides a process of manufacturing a fungicidal composition.

In some embodiments, the process comprises obtaining the crystalline form of the present invention and combining the crystalline form with at least one agriculturally acceptable carrier so as to thereby manufacture the fungicidal composition.

In some embodiments, the process comprises adding an adjuvant to manufacture the fungicidal composition.

In some embodiments, the process comprises adding an excipient to manufacture the fungicidal composition.

In another embodiment, the process comprises obtaining the mixture of the present invention and combining the mixture with an adjuvant and/or an excipient so as to thereby manufacture the fungicidal composition.

In some embodiments, the process comprises adding at least one additional pesticide to manufacture the fungicidal composition.

### Stable Liquid Compositions of the Crystalline Forms of Compound A

Compound A, or the compound of Formula I, is a pro-pesticide derivative of N3-Me-5-FU comprising sensitive groups such as sulfonyl group and imine on positions N1 and C4 accordingly. These “groups” lead to highly sensitive unstable structures which require development of specific conditions for stabilizing the compound of Formula I in a liquid composition. In addition, the compound of Formula I has several crystal forms and has a tendency to form crystals which are less available and affect the penetration rate into the target.

Formulating compositions comprising active ingredient often requires adding an agriculturally acceptable inert additive. Such as surfactants, dispersants, emulsifiers, wetting agents, antifoams, solvents, co-solvent, light stabilizers, UV absorbers, radical scavengers and antioxidants, adhesives, neutralizers, thickeners, binders, sequestrates, biocides, buffers preservatives, and anti-freeze agents. The addition of an additive affects the solubility of the active ingredient and leads to chemically and physically unstable compositions.

Solvent and additives, which can be used for the compound of Formula I, should be neutral, i.e. without an active functional group which can affect the stability of and cause degradation of the compound of Formula I. Solvent and/or additive used for formulating the compound for Formula I should not be reactive towards the compound of Formula I.

Functional groups which can affect the stability of the compound of Formula I are groups containing N and/or O, such as S—O, OH and non-sterically hindered amide and amine. It was found that chemical stability of non-crystalline compound of Formula I in amide solvent depends on the substitute on the amide. The reactivity of the solvent and/or the additive is critical in formulating a stable composition comprising the compound of Formula I. Reactive nucleophilic groups are groups such as hydroxyl group with bond dissociation energies less than 120 Kcal/mol, a weak dissociate hydrogen bond, or an acidic functional group.

The concentration of water in the composition is another critical factor for chemical and/or physical stability.

For the abovementioned reasons, formulating compound of Formula I in a liquid composition is particularly challenging.

It was found that the stability of non-crystalline compound of Formula I in liquid carrier may be improved by controlling the solubility of the compound of Formula I in the liquid carrier, controlling the pH of the composition in water environment, controlling the water content of the composition, adding surfactants effective for preventing crystals growth, and/or controlling the viscosity of the composition.

Based on the foregoing, stable liquid compositions comprising crystalline forms of the compound of Formula I, as well as their methods of use and processes of preparation, are described below.

The present invention provides a stable, liquid composition comprising:
(a) a fungicidally effective amount of a compound of Formula I:

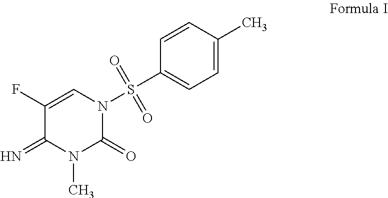 [SMILES: Cc1ccc(S(=O)(=O)n2cc(F)c(=N)n(C)c2=O)cc1]

in one or more forms, wherein at least one form is:
   a. the crystalline form Form III having powder X-ray diffraction pattern comprising peaks at 4.5, 5.1, 9.1, 10.1, 22.4, 23.5±0.2 deg 2-theta;
   b. the crystalline form Form IIIa having powder X-ray diffraction pattern comprising peaks at 4.5, 5.0, 9.3, 10.0, 22.3, 22.6±0.2 deg 2-theta;
   c. the crystalline form Form 02 having powder X-ray diffraction pattern comprising peaks at 7.5, 14.3, 15.8, 22.4, 24.7±0.2 deg 2-theta; or
   d. the crystalline form Form 01 having powder X-ray diffraction pattern comprising peaks at 5.1, 6.4, 7.5, 10.3, 16.1±0.2 deg 2-theta.
and
(b) a liquid carrier.

The present invention provides a stable, liquid composition comprising:
(a) a fungicidally effective amount of a compound of Formula I:

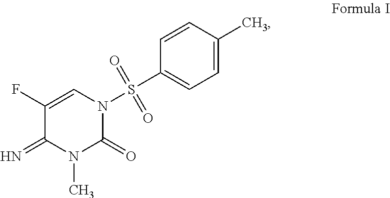 [SMILES: Cc1ccc(S(=O)(=O)n2cc(F)c(=N)n(C)c2=O)cc1]

and
(b) a liquid carrier,
wherein 95% or more of the amount of the compound of Formula I is in the form of Form III, Form IIIa, Form 01, Form 02, or a mixture thereof.

In some embodiments, 95% or more of the amount of the compound of Formula I is in the form of Form III.

The present invention provides a stable, liquid composition comprising an admixture of the following components:
(a) a fungicidally effective amount of a mixture containing a compound of Formula I:

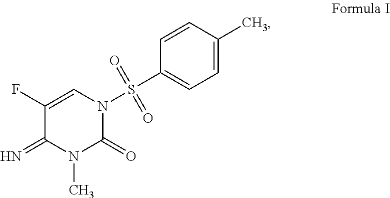 [SMILES: Cc1ccc(S(=O)(=O)n2cc(F)c(=N)n(C)c2=O)cc1]

wherein greater than or equal to 94% by weight of the mixture is the compound of Formula I, and
(b) a liquid carrier, wherein the compound of Formula I is in one or more forms and at least one form is Form III, Form IIIa, Form 01, or Form 02.

In some embodiments, 95% or more by weight of the mixture of component (a) is the compound of Formula I. In some embodiments, 96% or more by weight of the mixture of component (a) is the compound of Formula I. In some embodiments, 97% or more by weight of the mixture of component (a) is the compound of Formula I. In some embodiments, 98% or more by weight of the mixture of component (a) is the compound of Formula I. In some embodiments, 99% or more by weight of the mixture of component (a) is the compound of Formula I. In some embodiments, 99.5% or more by weight of the mixture of component (a) is the compound of Formula I. In some embodiments, 99.9% by weight of the mixture of component (a) is the compound of Formula I. In some embodiments, 99.99% by weight of the mixture of component (a) is the compound of Formula I.

In some embodiments, the solubility of the compound of Formula I in the liquid carrier is less than 5000 ppm. In some embodiments, the solubility of the compound of Formula I in the liquid carrier is less than 1000 ppm. In some embodiments, the solubility of compound of Formula I in the liquid carrier is in the range of 50 to 500 ppm. In some embodiments, the solubility of compound of Formula I in the liquid carrier is about 200 ppm. In some embodiments, the solubility of compound of Formula I in the liquid carrier is about 80 ppm.

In some embodiments, the composition comprises at least one stabilizing surfactant. In some embodiments, the composition comprises at least two stabilizing surfactants. In some embodiments, the composition comprises two stabilizing surfactants. In some embodiments, the composition further comprises a stabilizing system.

In some embodiments, the composition comprises at least one anionic stabilizing surfactant. In some embodiments, the composition comprises at least one non-ionic stabilizing surfactant. In some embodiments, the composition comprises two stabilizing surfactants. In some embodiments, the composition comprises a stabilizing system. In some embodiments, the composition comprises combination of a non-ionic stabilizing surfactant and an ionic stabilizing surfactant.

In some embodiments, the stabilizing surfactant(s) affects the solubility of the compound of Formula I in the liquid carrier.

In some embodiments, the pH of the composition is in the range of 5 to 7.5. In some embodiments, the pH of the composition is in the range of 6 to 7. In some embodiment, the pH of the composition is about 5. In some embodiments, the pH of the composition is about 5.5. In some embodiments, the pH of the composition is about 5.8. In some embodiments, the pH of the composition is about 6. In some embodiments, the pH of the composition is about 6.5. In some embodiments, the pH of the composition is about 7. In some embodiments, the pH of the composition is about 7.5.

In some embodiments, the pH of the composition is measured when the composition is in the presence of water. Water may be present in the composition as the liquid carrier. Water may also be present in the composition as a result of dilution or wetting.

In some embodiments, the pH of the composition is measured without further dilution and/or wetting. In some embodiments, the pH of the composition is measured after dilution and/or wetting.

In some embodiments, the pH value of the OD composition is measured by wetting the composition and the pH value is recorded as 1% (w/w) in aqueous suspension. In some embodiments, the pH value of the EC composition is measured by wetting the composition and the pH value is recorded as 1% (w/w) in aqueous suspension.

In some embodiments, the liquid carrier is water and the pH of the composition is measured without further dilution and/or wetting. In some embodiments, wherein the liquid carrier is a non-aqueous liquid carrier and the pH of the composition is measured after dilution and/or wetting.

In some embodiments, the composition comprises a pH adjuster.

The chemical stability of the composition is affected by the pH of the composition.

When the liquid carrier is non-aqueous, the amount of water in the composition should be less than 0.5% by weight based on the total weight of the composition, preferably, less than 0.2% by weight based on the total weight of the composition.

In some embodiments, the non-aqueous composition has a water content of less than 0.5% by weight based on the total weight of the composition. In some embodiments, the non-aqueous composition has a water content of less than 0.4% by weight based on the total weight of the composition. In some embodiments, the non-aqueous composition has a water content of less than 0.3% by weight based on the total weight of the composition. In some embodiments, the non-aqueous composition has a water content of less than 0.2% by weight based on the total weight of the composition. In some embodiments, the non-aqueous composition has a water content of less than or equal to 0.2% by weight based on the total weight of the composition. In some embodiments, the non-aqueous composition has a water content of less than 0.1% by weight based on the total weight of the composition. In some embodiments, the non-aqueous composition has a water content of less than or equal to 0.1% by weight based on the total weight of the composition. In some embodiments, the non-aqueous composition has a water content of 0.19%, 0.18%, 0.17%, 0.16%, 0.15%, 0.14%, 0.13%, 0.12%, 0.11%, 0.1%, 0.09%, 0.08%, 0.07%, 0.06%, 0.05%, 0.04% or 0.03% by weight based on the total weight of the composition.

In some embodiments, the non-aqueous composition has a water content of 0.09% or less by weight based on the total weight of the composition.

The present invention also provides a stable, liquid composition comprising:
(a) a fungicidally effective amount of a compound of Formula I or a fungicidally effective amount of a mixture containing a compound of Formula I:

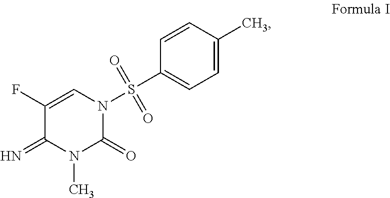 [SMILES: Cc1ccc(S(=O)(=O)n2cc(F)c(=N)n(C)c2=O)cc1]

and
(b) a non-aqueous liquid carrier,
wherein the composition has a water content of less than or equal to 0.09% by weight based on the total weight of the composition, and
wherein the compound of Formula I is in one or more forms and at least one form is Form III, Form IIIa, Form 01 or Form 02.

In some embodiments, the mixture containing the compound of Formula I has a water content of 0.3% or less by weight based on the total weight of the composition. In some embodiments, the mixture containing the compound of Formula I has a water content of 0.2% or less by weight based on the total weight of the composition. In some embodiments, the mixture containing the compound of Formula I has a water content of 0.1% or less by weight based on the total weight of the composition. In some embodiments, the mixture containing the compound of Formula I has a water content of 0.05% or less by weight based on the total weight of the composition. In some embodiments, the mixture containing the compound of Formula I is substantially free of water.

In some embodiments, the amount of the compound of Formula I has a water content of 0.3% or less by weight based on the total weight of the composition. In some embodiments, the amount of the compound of Formula I has a water content of 0.2% or less by weight based on the total weight of the composition. In some embodiments, the amount of the compound of Formula I has a water content of 0.1% or less by weight based on the total weight of the composition. In some embodiments, the amount of the compound of Formula I has a water content of 0.05% or less by weight based on the total weight of the composition. In some embodiments, the amount of the compound of Formula I is substantially free of water.

In some embodiments, the composition is an OD composition, and the water content of the OD composition is less than 0.5% by weight based on the total weight of the composition. In some embodiments, the composition is an EC composition, and the water content of the EC composition is less than 0.5% by weight based on the total weight of the composition.

An amount of water less than 0.5% by weight based on the total weight of the composition, preferably less than 0.2% by weight based on the total weight of the composition, can be achieved using methods including but not limited to drying component(s) of the composition prior to adding it to the composition and/or lowering the water content of the components in the composition (both active and non-active components). The water content of the composition may be also be controlled by using low water content surfactants, low water content carrier, water scavenger and/or drying agent. In some embodiments, the composition comprises a low water content surfactant. In some embodiments, the composition comprises a low water content carrier. In some embodiments, the composition comprises at least one water scavenger. In some embodiments, the composition comprises at least one drying agent. In some embodiments, the low water content surfactant, low water content carrier, water scavenger and/or drying agent are added to the composition after the composition is dried.

In some embodiments, the water scavenger is epoxylated soybean oil. In some embodiments, the water scavenger is selected from the group consisting of tetraethyl orthosilicate, Dynasylan® and a combination thereof. In some embodiments, the water scavenger is Dynasylan®. In some embodiments, the Dynasylan® is Dynasylan® P. In some embodiments, the water scavenger is tetraethyl orthosilicate.

In some embodiments, the amount of the water scavenger in the OD composition is less than 10% by weight based on the total weight of the composition. In some embodiments, the amount of the water scavenger in the OD composition is less than 7.5% by weight based on the total weight of the composition. In some embodiments, the amount of the water scavenger in the OD composition is less than 5% by weight based on the total weight of the composition. In some embodiments, the amount of the water scavenger in the OD composition is about 5% by weight based on the total weight of the composition.

In some embodiments, the amount of water scavenger in the composition is between about 0.5-7.5% by weight based on the total weight of the composition. In some embodiments, the amount of tetraethyl orthosilicate in the OD composition is 4-6% by weight based on the total weight of the composition. In some embodiments, the amount of tetraethyl orthosilicate in the OD composition is 5% by weight based on the total weight of the composition. In some embodiments, the amount of tetraethyl orthosilicate in the OD composition is less than 5% by weight based on the total weight of the composition.

These water scavengers reduce the water content of non-aqueous liquid composition to below 0.5% by weight which improves the composition's stability. These water scavengers can be used to reduce the water content of non-aqueous liquid composition to any water content level described in this application to improve the composition's stability.

Without water scavenger, the degradation of the compound of Formula I is usually between 5-10%. The additional of a water scavenger can reduce degradation of the compound of Formula I to 5% or less.

These water scavengers reduce the water content of the OD composition to below 0.5% by weight which improves the composition's stability. These water scavengers reduce the water content of the OD composition to below 0.2% by weight which improves the composition's stability. These water scavengers reduce the water content of the OD composition to below 0.1% by weight which improves the composition's stability.

In some embodiments, the water content of the non-aqueous composition is less than 0.5% by weight based on the total weight of the composition, and/or the water content of the amount of the compound of Formula I is not more than 0.3% by weight based on the weight of the amount of the compound of Formula I.

In some embodiments, the water content of the non-aqueous composition is less than 0.5% by weight based on the total weight of the composition, and the water content of the amount of the compound of Formula I is not more than 0.3% by weight based on the weight of the amount of the compound of Formula I.

In some embodiments, the water content of the non-aqueous composition is less than 0.5% by weight based on the total weight of the composition, and/or the water content of the amount of the compound of Formula I is not more than 0.2% by weight based on the weight of the amount of the compound of Formula I.

In some embodiments, the water content of the non-aqueous composition is less than 0.5% by weight based on the total weight of the composition, and the water content of the amount of the compound of Formula I is not more than 0.2% by weight based on the weight of the amount of the compound of Formula I.

In some embodiments, the water content of the OD composition is less than 0.5% by weight based on the total weight of the composition, and/or the water content of the amount of the compound of Formula I is not more than 0.3% by weight based on the weight of the amount of the compound of Formula I.

In some embodiments, the water content of the OD composition is less than 0.5% by weight based on the total weight of the composition, and the water content of the amount of the compound of Formula I is not more than 0.3% by weight based on the weight of the amount of the compound of Formula I.

In some embodiments, the water content of the OD composition is less than 0.5% by weight based on the total weight of the composition, and/or the water content of the amount of the compound of Formula I is not more than 0.2% by weight based on the weight of the amount of the compound of Formula I.

In some embodiments, the water content of the OD composition is less than 0.5% by weight based on the total weight of the composition, and the water content of the amount of the compound of Formula I is not more than 0.2% by weight based on the weight of the amount of the compound of Formula I.

These water scavengers reduce the water content of the EC composition to below 0.5% by weight which improves the composition's stability. These water scavengers reduce the water content of the EC composition to below 0.1% by weight which improves the composition's stability. These water scavengers reduce the water content of the EC composition to below 0.2% by weight which improves the composition's stability.

In some embodiments, the composition has a viscosity of at least 500 cP. In some embodiments, the composition has a viscosity of 500 cP-3000 cP. In some embodiments, the composition has a viscosity of 500 cP-2500 cP. In some embodiments, the composition has a viscosity of 800 cP-3000 cP. In some embodiments, the composition has a viscosity of 1600 cP-2200 cP. In some embodiments, the composition has a viscosity of equal to or less than 3000 cP.

In some embodiments, the composition has a viscosity of about 500 cP-1000 cP. In some embodiments, the composition has a viscosity of about 1000 cP-1500 cP. In some embodiments, the composition has a viscosity of about 1500 cP-2000 cP. In some embodiments, the composition has a viscosity of about 2000 cP-2500 cP. In some embodiments, the composition has a viscosity of about 2500 cP-3000 cP.

In some embodiments, the composition has a viscosity of about 500 cP, about 600 cP, about 700 cP, about 800 cP, about 900 cP, about 1000 cP, about 1100 cP, about 1200 cP, about 1300 cP, about 1400 cP, about 1500 cP, about 1600 cP, about 1700 cP, about 1800 cP, about 1900 cP about 2000 cP, about 2100 cP, about 2200 cP, about 2300 cP, about 2400 cP about 2500 cP, about 2600 cP, about 2700 cP, about 2800 cP, about 2900 cP, about 3000 cP.

In some embodiments, the liquid carrier is an aqueous liquid carrier. In some embodiments, the aqueous liquid carrier is water.

In some embodiments, the liquid carrier is a non-aqueous liquid carrier.

In some embodiments, the solubility of the compound of Formula I in the aqueous liquid carrier is less than 5000 ppm. In some embodiments, the solubility of the compound of Formula I in the non-aqueous liquid carrier is less than 5000 ppm.

In some embodiments, the compound of Formula I is in the form of solid particles. In some embodiments, the solid particles of the compound of Formula I is suspended in the aqueous liquid carrier. In some embodiments, the solid particles of the compound of Formula I is suspended in the non-aqueous liquid carrier.

In some embodiments, the compound of Formula I is dissolved in the non-aqueous liquid carrier.

When the solid particles of the compound of Formula I is suspended in the aqueous liquid carrier, the composition is suspension concentrate (SC).

When the SC composition comprising an aqueous liquid carrier further comprises a non-aqueous liquid component, the SC composition is a suspoemulsion (SE). When solid particles of the compound of Formula I is suspended in the aqueous liquid carrier and the composition further comprises a non-aqueous liquid component, the composition is a suspoemulsion (SE). The non-aqueous liquid component may be but is not limited to adjuvant, carrier of the adjuvant and/or any additive. In some embodiment, the non-aqueous liquid component is an adjuvant. The SC composition is an SE composition when the SC composition further comprises a non-aqueous liquid component in the aqueous liquid carrier.

When the solid particles of the compound of Formula I is suspended in the non-aqueous liquid carrier, the composition is oil dispersion (OD).

When the compound of Formula I is dissolved in the non-aqueous liquid carrier the composition is an emulsifiable concentrate (EC).

In some embodiments, the stable liquid composition is a suspension concentrate (SC) composition. An SC composition is aqueous. In some embodiments, the composition is a suspoemulsion. (SE). composition. In some embodiments, the composition is an oil dispersion (OD) composition. In some embodiments, the composition is an emusifiable concentrate (EC) composition.

Crystalline Form III is more stable when it is formulated in an oil dispersion (OD) composition than when it is formulated in a suspension concentrate (SC) composition.

In some embodiments, the stable liquid composition is a suspension concentrate (SC) composition comprising at least one stabilizing surfactant. In some embodiments, the stable liquid composition is a suspension concentrate (SC) composition comprising two stabilizing surfactants.

In some embodiments, the stable liquid composition is a suspoemulsion (SE) composition comprising at least one stabilizing surfactant. In some embodiments, the stable liquid composition is a suspoemulsion (SE) composition comprising two stabilizing surfactants.

In some embodiments, the stable liquid composition is a suspension concentrate (SC) composition having a pH in the range of 5 to 7.5.

In some embodiments, the stable liquid composition is a suspoemulsion (SE) composition having a pH in the range of 5 to 7.5.

In some embodiments, the stable liquid composition is an oil dispersion (OD) composition with a water content of less than 0.5% by weight based on the total weight of the composition. In some embodiments, the stable liquid composition is an oil dispersion (OD) composition with a water content of less than or equal to 0.2% by weight based on the total weight of the composition. In some embodiments, the stable liquid composition is an OD composition with a water content of less than 0.2% by weight based on the total weight of the composition. In some embodiments, the stable liquid composition is an oil dispersion (OD) composition with a water content of less than or equal to 0.1% by weight based on the total weight of the composition. In some embodiments, the stable liquid composition is an OD composition with a water content of less than 0.1% by weight based on the total weight of the composition. In some embodiments, the OD composition has a water content of 0.19%, 0.18%, 0.17%, 0.16%, 0.15%, 0.14%, 0.13%, 0.12%, 0.11%, 0.1%, 0.09%, 0.08%, 0.07%, 0.06%, 0.05%, 0.04% or 0.03% by weight based on the total weight of the composition.

In some embodiments, the stable liquid composition is an emulsifiable concentrate (EC) composition with a water content of less than 0.5% by weight based on the total weight of the composition. In some embodiments, the stable liquid composition is an emulsifiable concentrate (EC) composition with a water content of less than or equal to 0.2% by weight based on the total weight of the composition. In some embodiments, the stable liquid composition is an emulsifiable concentrate (EC) composition with a water content of less than 0.2% by weight based on the total weight of the composition. In some embodiments, the stable liquid composition is an emulsifiable concentrate (EC) composition with a water content of less than or equal to 0.1% by weight based on the total weight of the composition. In some embodiments, the stable liquid composition is an emulsifiable concentrate (EC) composition with a water content of less than 0.1% by weight based on the total weight of the composition. In some embodiments, the EC composition has a water content of 0.19%, 0.18%, 0.17%, 0.16%, 0.15%, 0.14%, 0.13%, 0.12%, 0.11%, 0.1%, 0.09%, 0.08%, 0.07%, 0.06%, 0.05%, 0.04% or 0.03% by weight based on the total weight of the composition.

In some embodiments, the composition comprises an aqueous liquid carrier and the aqueous composition has a viscosity of at least 500 cP. In some embodiments, the composition comprises an aqueous liquid carrier and the aqueous composition has a viscosity of equal to or less than 3000 cP.

In some embodiments, the stable liquid composition is a suspension concentrate (SC) composition and the SC composition has a viscosity of at least 500 cP. In some embodiments, the stable liquid composition is a suspension concentrate (SC) composition and the SC composition has a viscosity of 800 cP-3000 cP. In some embodiments, the stable liquid composition is a suspension concentrate (SC) composition and the SC composition has a viscosity of 1600 cP-2200 cP. In some embodiments, the stable liquid composition is a suspension concentrate (SC) composition and the SC composition has a viscosity of equal to or less than 3000 cP.

In some embodiments, the composition comprises a non-aqueous liquid carrier and the non-aqueous composition has a viscosity of at least 500 cP. In some embodiments, the composition comprises a non-aqueous liquid carrier and the non-aqueous composition has a viscosity of equal to or less than 3000 cP.

In some embodiments, the stable liquid composition is an oil dispersion (OD) composition and the OD composition has a viscosity of at least 500 cP. In some embodiments, the stable liquid composition is an oil dispersion (OD) composition and the OD composition has a viscosity of 500 cP-2500 cP. In some embodiments, the stable liquid composition is an oil dispersion (OD) composition and the OD composition has a viscosity of equal to or less than 2500 cP.

Viscosity may be measured using Collaborative International Pesticides Analytical Council (CIPAC) MT192—viscosity of liquids by rotational viscometer, the entire content of which is hereby incorporated by reference into this application. When viscosity is described in the subject application, the viscosity is measured using CIPAC MT192 using spindle 62 at 12 rpm or spindle 63 at 12 rpm.

In some embodiments, viscosity is measured using spindle 62 at 12 rpm. In some embodiments, viscosity is measured using spindle 63 at 12 rpm. In some embodiments, the composition has a viscosity of at least 500 cP when measured using CIPAC MT192 using spindle 62 at 12 rpm or using spindle 63 at 12 rpm. In some embodiments, the composition has a viscosity of 500 cP-3000 cP when measured using CIPAC MT192 using spindle 62 at 12 rpm or using spindle 63 at 12 rpm. In some embodiments, the composition has a viscosity of 500 cP-2500 cP when measured using CIPAC MT192 using spindle 62 at 12 rpm or using spindle 63 at 12 rpm. In some embodiments, the composition has a viscosity of 800 cP-3000 cP when measured using CIPAC MT192 using spindle 62 at 12 rpm or using spindle 63 at 12 rpm. In some embodiments, the composition has a viscosity of 1600 cP-2200 cP when measured using CIPAC MT192 using spindle 62 at 12 rpm or using spindle 63 at 12 rpm. In some embodiments, the composition has a viscosity of equal to or less than 3000 cP when measured using CIPAC MT192 using spindle 62 at 12 rpm or using spindle 63 at 12 rpm.

In some embodiments, the total amount of aqueous liquid carrier in the composition ranges from about 30% to about 70% by weight based on the total weight of the composition. In some embodiments, the total amount of aqueous liquid carrier in the composition ranges from about 40% to about 60% by weight based on the total weight of the composition. In some embodiments, the total amount of aqueous liquid carrier in the composition ranges from about 40% to about 50% by weight based on the total weight of the composition.

In some embodiments, the total amount of aqueous liquid carrier in the SC composition ranges from about 30% to about 70% by weight based on the total weight of the composition. In some embodiments, the total amount of aqueous liquid carrier in the SC composition ranges from about 40% to about 60% by weight based on the total weight of the composition. In some embodiments, the total amount of aqueous liquid carrier in the SC composition ranges from about 40% to about 50% by weight based on the total weight of the composition.

In some embodiments, the total amount of aqueous liquid carrier in the SE composition ranges from about 30% to about 70% by weight based on the total weight of the composition. In some embodiments, the total amount of aqueous liquid carrier in the SE composition ranges from about 40% to about 60% by weight based on the total weight of the composition. In some embodiments, the total amount of aqueous liquid carrier in the SE composition ranges from about 40% to about 50% by weight based on the total weight of the composition.

In some embodiments, the total amount of non-aqueous liquid carrier in the composition ranges from about 30 to about 80% by weight based on the total weight of the composition. In some embodiments, the total amount of non-aqueous liquid carrier in the composition ranges from about 40% to about 70% by weight based on the total weight of the composition. In some embodiments, the total amount of non-aqueous liquid carrier in the composition is about 50% by weight based on the total weight of the composition.

In some embodiments, the total amount of non-aqueous liquid carrier in the OD composition ranges from about 30 to about 80% by weight based on the total weight of the composition. In some embodiments, the total amount of non-aqueous liquid carrier in the OD composition ranges from about 40% to about 70% by weight based on the total weight of the composition. In some embodiments, the total amount of non-aqueous liquid carrier in the OD composition is about 50% by weight based on the total weight of the composition.

In some embodiments, the total amount of non-aqueous liquid carrier in the EC composition ranges from about 30 to about 80% by weight based on the total weight of the composition. In some embodiments, the total amount of non-aqueous liquid carrier in the EC composition ranges from about 40% to about 70% by weight based on the total weight of the composition. In some embodiments, the total amount of non-aqueous liquid carrier in the EC composition ranges from about 40% to about 80% by weight based on the total weight of the composition. In some embodiments, the total amount of non-aqueous liquid carrier in the EC composition is about 80% by weight based on the total weight of the composition.

In some embodiments, the concentration of the compound of Formula I in the stable liquid composition is 5 g/L to 750 g/L. In some embodiments, the concentration of the compound of Formula I in the stable liquid composition is 150 g/L to 750 g/L. In some embodiments, the concentration of the compound of Formula I in the stable liquid composition is 200 g/L to 250 g/L. In some embodiments, the concentration of the compound of Formula I in the stable liquid composition is 300 g/L to 750 g/L. In some embodiments, the concentration of the compound of Formula I in the stable liquid composition is 300 g/L to 400 g/L. In some embodiments, the concentration of the compound of Formula I in the stable liquid composition is 400 g/L to 500 g/L. In some embodiments, the concentration of the compound of Formula I in the stable liquid composition is 400 g/L to 600 g/L. In some embodiments, the concentration of the compound of Formula I in the stable liquid composition is 500 g/L to 600 g/L. In some embodiments, the concentration of the compound of Formula I in the stable liquid composition is 600 g/L to 700 g/L. In some embodiments, the concentration of the compound of Formula I in the stable liquid composition is 400 g/L. In some embodiments, the concentration of the compound of Formula I in the stable liquid composition is 450 g/L. In some embodiments, the concentration of the compound of Formula I in the stable liquid composition is 500 g/L. In some embodiments, the concentration of the compound of Formula I in the stable liquid composition is 550 g/L. In some embodiments, the concentration of the compound of Formula I in the stable liquid composition is 600 g/L. In some embodiments, the concentration of the compound of Formula I in the stable liquid composition is 660 g/L.

In some embodiments, the concentration of compound of Formula I in the composition is greater than 5% by weight based on the total weight of the stable composition. In some embodiments, the concentration of compound of Formula I in the composition is greater than 10% by weight based on the total weight of the stable composition. In some embodiments, the concentration of compound of Formula I in the composition is greater than 25% by weight based on the total weight of the stable composition. In some embodiments, the concentration of compound of Formula I in the composition is greater than 50% by weight based on the total weight of the stable liquid composition. In some embodiments, the concentration of compound of Formula I in the composition is less than 75% by weight based on the total weight of the stable composition. In some embodiments, the concentration of compound of Formula I in the composition is less than 90% by weight based on the total weight of the stable composition. In some embodiments, the concentration of compound of Formula I in the composition is between 20% to 30% by weight based on the total weight of the stable composition. In some embodiments, the concentration of compound of Formula I in the composition is about 25% by weight based on the total weight of the stable composition.

In some embodiments, the concentration of compound of Formula I in the SC composition is greater than 25% by weight based on the total weight of the stable composition. In some embodiments, the concentration of compound of Formula I in the SC composition is greater than 50% by weight based on the total weight of the stable liquid composition. In some embodiments, the concentration of compound of Formula I in the SC composition is 50% by weight based on the total weight of the stable liquid composition. In some embodiments, the concentration of compound of Formula I in the SC composition is 25%-75% by weight based on the total weight of the stable liquid composition. In some embodiments, the concentration of compound of Formula I in the SC composition is 50% by weight based on the total weight of the stable liquid composition. In some embodiments, the concentration of compound of Formula I in the SC composition is 35%-45% by weight based on the total weight of the composition. In some embodiments, the concentration of compound of Formula I in the SC composition is about 42% by weight based on the total weight of the composition.

In some embodiments, the concentration of compound of Formula I in the SE composition is greater than 25% by weight based on the total weight of the stable composition. In some embodiments, the concentration of compound of Formula I in the SE composition is greater than 50% by weight based on the total weight of the stable liquid composition. In some embodiments, the concentration of compound of Formula I in the SE composition is 50% by weight based on the total weight of the stable liquid composition.

In some embodiments, the concentration of the compound of Formula I in the OD composition is greater than 10% by weight based on the total weight of the stable composition. In some embodiments, the concentration of the compound of Formula I in the OD composition is greater than 25% by weight based on the total weight of the stable composition. In some embodiments, the concentration of compound of Formula I in the OD composition is greater than 50% by weight based on the total weight of the stable liquid composition. In some embodiments, the concentration of compound of Formula I in the OD composition is 50% by weight based on the total weight of the stable liquid composition. In some embodiments, the concentration of the compound of Formula I in the OD composition is 10%-50% by weight based on the total weight of the stable composition. In some embodiments, the concentration of the compound of Formula I in the OD composition is 15%-25% by weight based on the total weight of the stable composition. In some embodiments, the concentration of the compound of Formula I in the OD composition is 15%-25% by weight based on the total weight of the stable composition. In some embodiments, the concentration of the compound of Formula I in the OD composition is 20% by weight based on the total weight of the stable composition. In some embodiments, the concentration of compound of Formula I in the OD composition is between 20% to 30% by weight based on the total weight of the stable composition. In some embodiments, the concentration of compound of Formula I in the OD composition is about 25% by weight based on the total weight of the stable composition.

In some embodiments, the concentration of the compound of Formula I in the OD composition is 200 g/L to 250 g/L.

In some embodiments, the concentration of compound of Formula I in the EC composition is greater than 5% by weight based on the total weight of the stable liquid composition. In some embodiments, the concentration of compound of Formula I in the EC composition is greater than 10% by weight based on the total weight of the stable liquid composition. In some embodiments, the concentration of compound of Formula I in the EC composition is greater than 25% by weight based on the total weight of the stable liquid composition. In some embodiments, the concentration of compound of Formula I in the EC composition is 5%-25% by weight based on the total weight of the stable liquid composition. In some embodiments, the concentration of compound of Formula I in the EC composition is greater than 5%-10% by weight based on the total weight of the stable liquid composition. In some embodiments, the concentration of compound of Formula I in the EC composition is 5% by weight based on the total weight of the stable liquid composition. In some embodiments, the concentration of compound of Formula I in the EC composition is 1-10% by weight based on the total weight of the stable liquid composition. In some embodiments, the concentration of compound of Formula I in the EC composition is 4-5% by weight based on the total weight of the stable liquid composition. In some embodiments, the concentration of compound of Formula I in the EC composition is about 4.8% by weight based on the total weight of the stable liquid composition.

In some embodiments, greater than 95% of the amount of the compound of Formula I is in the form of Form III, Form IIIa, Form 01, Form 02, or a mixture thereof. In some embodiments, greater than 96% of the amount of the compound of Formula I is in the form of Form III, Form IIIa, Form 01, Form 02, or a mixture thereof. In some embodiments, greater than 97% of the amount of the compound of Formula I is in the form of Form III, Form IIIa, Form 01, Form 02, or a mixture thereof. In some embodiments, greater than 98% of the amount of the compound of Formula I is in the form of Form III, Form IIIa, Form 01, Form 02, or a mixture thereof. In some embodiments, greater than 99% of the amount of the compound of Formula I is in the form of Form III, Form IIIa, Form 01, Form 02, or a mixture thereof. In some embodiments, the amount of the compound of Formula I is substantially pure of Form III, Form IIIa, Form 01, Form 02, or a mixture thereof.

In some embodiments, 95% or more of the amount of the compound of Formula I is in the form of Form III, Form IIIa, Form 01, Form 02, or a mixture thereof. In some embodiments, 96% or more of the amount of the compound of Formula I is in the form of Form III, Form IIIa, Form 01, Form 02, or a mixture thereof. In some embodiments, 97% or more of the amount of the compound of Formula I is in the form of Form III, Form IIIa, Form 01, Form 02, or a mixture thereof. In some embodiments, 98% or more of the amount of the compound of Formula I is in the form of Form III, Form IIIa, Form 01, Form 02, or a mixture thereof. In some embodiments, 99% or more of the amount of the compound of Formula I is in the form of Form III, Form IIIa, Form 01, Form 02, or a mixture thereof.

In some embodiments, the compound of Formula I is in Form III.

In some embodiments, 95% or more of the amount of the compound of Formula I is in the form of Form III. In some embodiments, 96% or more of the amount of the compound of Formula I is in the form of Form III. In some embodiments, 97% or more of the amount of the compound of Formula I is in the form of Form III. In some embodiments, 98% or more of the amount of the compound of Formula I is in the form of Form III. In some embodiments, 99% or more of the amount of the compound of Formula I is in the form of Form III. In some embodiments, 99.5% or more of the amount of the compound of Formula I is in the form of Form III. In some embodiments, 100% of the amount of the compound of Formula I is in the form of Form III.

In some embodiments, the compound of Formula I is in Form IIIa.

In some embodiments, 95% or more of the amount of the compound of Formula I is in the form of Form IIIa. In some embodiments, 96% or more of the amount of the compound of Formula I is in the form of Form IIIa. In some embodiments, 97% or more of the amount of the compound of Formula I is in the form of Form IIIa. In some embodiments, 98% or more of the amount of the compound of Formula I is in the form of Form IIIa. In some embodiments, 99% or more of the amount of the compound of Formula I is in the form of Form IIIa. In some embodiments, 99.5% or more of the amount of the compound of Formula I is in the form of Form IIIa. In some embodiments, 100% of the amount of the compound of Formula I is in the form of Form IIIa.

In some embodiments, the compound of Formula I is in Form 01.

In some embodiments, 95% or more of the amount of the compound of Formula I is in the form of Form 01. In some embodiments, 96% or more of the amount of the compound of Formula I is in the form of Form 01. In some embodiments, 97% or more of the amount of the compound of Formula I is in the form of Form 01. In some embodiments, 98% or more of the amount of the compound of Formula I is in the form of Form 01. In some embodiments, 99% or more of the amount of the compound of Formula I is in the form of Form 01. In some embodiments, 99.5% or more of the amount of the compound of Formula I is in the form of Form 01. In some embodiments, 100% of the amount of the compound of Formula I is in the form of Form 01.

In some embodiments, the compound of Formula I is in Form 02.

In some embodiments, 95% or more of the amount of the compound of Formula I is in the form of Form 02. In some embodiments, 96% or more of the amount of the compound of Formula I is in the form of Form 02. In some embodiments, 97% or more of the amount of the compound of Formula I is in the form of Form 02. In some embodiments, 98% or more of the amount of the compound of Formula I is in the form of Form 02. In some embodiments, 99% or more of the amount of the compound of Formula I is in the form of Form 02. In some embodiments, 99.5% or more of the amount of the compound of Formula I is in the form of Form 02. In some embodiments, 100% of the amount of the compound of Formula I is in the form of Form 02.

In some embodiments, the compound of Formula I is a mixture of Form III, Form IIIa, Form 01, Form 02, or any combination thereof.

In some embodiments, the compound of Formula I is a mixture of Form III and Form IIIa.

In some embodiments, the weight ratio of Form III to Form IIIa in the mixture is between 20:1 to 1:20. In some embodiments, the weight ratio of Form III to Form IIIa in the mixture is between 10:1 to 1:10. In some embodiments, the weight ratio of Form III to Form IIIa in the mixture is between 5:1 to 1:5. In some embodiments, the weight ratio of Form III to Form IIIa in the mixture is between 4:1 to 1:4. In some embodiments, the weight ratio of Form III to Form IIIa in the mixture is between 2:1 to 1:2. In some embodiments, the weight ratio of Form III to Form IIIa in the mixture is 1:1.

In some embodiments, the compound of Formula I is a mixture of Form III and Form 01.

In some embodiments, the weight ratio of Form III and Form 01 in the mixture is between 20:1 to 1:20. In some embodiments, the weight ratio of Form III and Form 01 in the mixture is between 10:1 to 1:10. In some embodiments, the weight ratio of Form III and Form 01 in the mixture is between 5:1 to 1:5. In some embodiments, the weight ratio of Form III and Form 01 in the mixture is between 4:1 to 1:4. In some embodiments, the weight ratio of Form III and Form 01 in the mixture is between 2:1 to 1:2. In some embodiments, the weight ratio of Form III and Form 01 in the mixture is 1:1.

In some embodiments, the compound of Formula I is a mixture of Form III and Form 02.

In some embodiments, the weight ratio of Form III and Form 02 in the mixture is between 20:1 to 1:20. In some embodiments, the weight ratio of Form III and Form 02 in the mixture is between 10:1 to 1:10. In some embodiments, the weight ratio of Form III and Form 02 in the mixture is between 5:1 to 1:5. In some embodiments, the weight ratio of Form III and Form 02 in the mixture is between 4:1 to 1:4. In some embodiments, the weight ratio of Form III and Form 02 in the mixture is between 2:1 to 1:2. In some embodiments, the weight ratio of Form III and Form 02 in the mixture is 1:1.

In some embodiments, the compound of Formula I is a mixture of Form IIIa and Form 01.

In some embodiments, the weight ratio of Form IIIa and Form 01 in the mixture is between 20:1 to 1:20. In some embodiments, the weight ratio of Form IIIa and Form 01 in the mixture is between 10:1 to 1:10. In some embodiments, the weight ratio of Form IIIa and Form 01 in the mixture is between 5:1 to 1:5. In some embodiments, the weight ratio of Form IIIa and Form 02 in the mixture is between 4:1 to 1:4. In some embodiments, the weight ratio of Form IIIa and Form 01 in the mixture is between 2:1 to 1:2. In some embodiments, the weight ratio of Form IIIa and Form 01 in the mixture is 1:1.

In some embodiments, the compound of Formula I is a mixture of Form IIIa and Form 02.

In some embodiments, the weight ratio of Form IIIa and Form 02 in the mixture is between 20:1 to 1:20. In some embodiments, the weight ratio of Form IIIa and Form 02 in the mixture is between 10:1 to 1:10. In some embodiments, the weight ratio of Form IIIa and Form 02 in the mixture is between 5:1 to 1:5. In some embodiments, the weight ratio of Form IIIa and Form 02 in the mixture is between 4:1 to 1:4. In some embodiments, the weight ratio of Form IIIa and Form 02 in the mixture is between 2:1 to 1:2. In some embodiments, the weight ratio of Form IIIa and Form 02 in the mixture is 1:1.

In some embodiments, the compound of Formula I is a mixture of Form 01 and Form 02.

In some embodiments, the weight ratio of Form 01 and Form 02 in the mixture is between 20:1 to 1:20. In some embodiments, the weight ratio of Form 01 and Form 02 in the mixture is between 10:1 to 1:10. In some embodiments, the weight ratio of Form 01 and Form 02 in the mixture is between 5:1 to 1:5. In some embodiments, the weight ratio of Form 01 and Form 02 in the mixture is between 4:1 to 1:4. In some embodiments, the weight ratio of Form 01 and Form 02 in the mixture is between 2:1 to 1:2. In some embodiments, the weight ratio of Form 01 and Form 02 in the mixture is 1:1.

In some embodiments, the compound of Formula I is a mixture of Form III, Form IIIa and Form 01. In some embodiments, the compound of Formula I is a mixture of Form III, Form IIIa and Form 02. In some embodiments, the compound of Formula I is a mixture of Form IIIa, Form 01 and Form 02. In some embodiments, the compound of Formula I is a mixture of Form III, Form 01 and Form 02.

In some embodiments, the composition comprising non-aqueous liquid carrier is free of phosphoric acid. In some embodiments, the composition is free of phosphoric acid at 2% or 5%. In some embodiments, the composition comprises 2% or less by weight of phosphoric acid. In some embodiments, the composition comprises 5% or less by weight of phosphoric acid.

In some embodiments, the composition comprising non-aqueous liquid carrier is free of urea. In some embodiments, the composition is free of urea at 1% or 2%. In some embodiments, the composition comprises 1% or less by weight of urea. In some embodiments, the composition comprises 2% or less by weight of urea.

In some embodiments, the composition comprising non-aqueous liquid carrier is free of propyl gallate.

In some embodiments, the composition comprising non-aqueous liquid carrier is free of dimethyl sulfoxide (DMSO).

In some embodiments, the composition comprising non-aqueous liquid carrier is free of morpholine.

In some embodiments, the composition comprising non-aqueous liquid carrier is free of N-methyl pyrrolidone.

The present invention provides a stable suspension concentrate (SC) composition comprising:
(a) a fungicidally effective amount of a compound of Formula I:

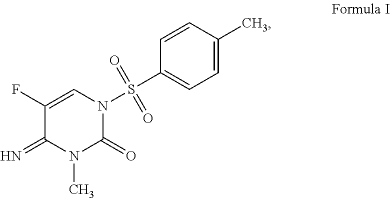 [SMILES: Cc1ccc(S(=O)(=O)n2cc(F)c(=N)n(C)c2=O)cc1]

in one or more forms, wherein at least one form is Form III, Form IIIa, Form 01, or Form 02,
(b) an aqueous liquid carrier, and
(c) at least one stabilizing surfactant,
   wherein the composition has a pH in the range of 5 to 7.5.

The present invention provides a suspoemulsion (SE) composition comprising:
(a) a fungicidally effective amount of a compound of Formula I:

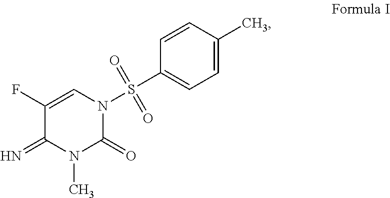 [SMILES: Cc1ccc(S(=O)(=O)n2cc(F)c(=N)n(C)c2=O)cc1]

in one or more forms, wherein at least one form III, Form IIIa, Form 01, or Form 02,
(b) an aqueous liquid carrier, and
(c) at least one stabilizing surfactant,
   wherein the composition has a pH in the range of 5 to 7.5.

The present invention provides an oil dispersion (OD) composition comprising:
(a) a fungicidally effective amount of a compound of Formula I:

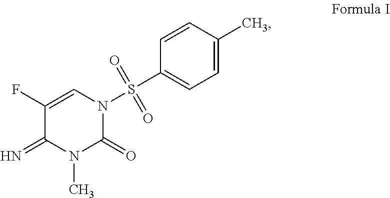 [SMILES: Cc1ccc(S(=O)(=O)n2cc(F)c(=N)n(C)c2=O)cc1]

in one or more forms, wherein at least one form is Form III, Form IIIa, Form 01, or Form 02,
(b) non-aqueous liquid carrier, and
(c) at least one stabilizing surfactant,
   wherein the water content in the composition is less than 0.5% by weight based on the total weight of the composition and/or the viscosity of the composition is at least 500 cP.

The present invention provides an emulsifiable concentrate (EC) composition comprising:
(a) a fungicidally effective amount of a compound of Formula I:

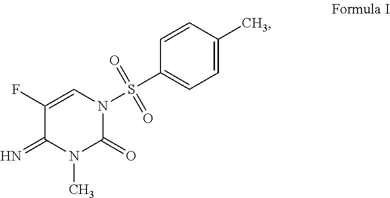 [SMILES: Cc1ccc(S(=O)(=O)n2cc(F)c(=N)n(C)c2=O)cc1]

in one or more forms, wherein at least one form is Form III, Form IIIa, Form 01, or Form 02,
(b) non-aqueous liquid carrier, and
(c) at least one stabilizing surfactant,
   wherein the water content in the composition is less than 0.5% by weight based on the total weight of the composition.

In some embodiments, the non-aqueous liquid carrier is used as an adjuvant.

The present invention also provides a suspension concentrate (SC) composition comprising:
(a) a fungicidally effective amount of a compound of Formula I:

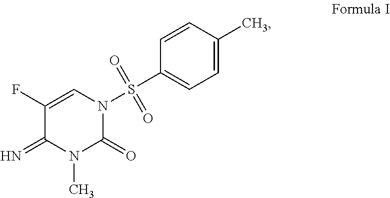 [SMILES: Cc1ccc(S(=O)(=O)n2cc(F)c(=N)n(C)c2=O)cc1]

in one or more forms, wherein at least one form is Form III, Form IIIa, Form 01, or Form 02, and
(b) an aqueous liquid carrier,
wherein the composition has one, two, three or four of the following features:
(i) the concentration of the compound of Formula I in the composition is 50% or less by weight based on the total weight of the composition,
(ii) the composition comprises siloxane polyalkyleneoxide copolymer at a concentration of 0.01-0.8% by weight based on the total weight of the composition,
(iii) the composition is free of the block copolymer of vinylpyrrolidone and vinyl acetate (VP/VA), and
(iv) the composition is free of magnesium aluminum silicate.

The present invention also provides an emulsifiable concentrate (EC) composition comprising:
(a) a fungicidally effective amount of a compound of Formula I:

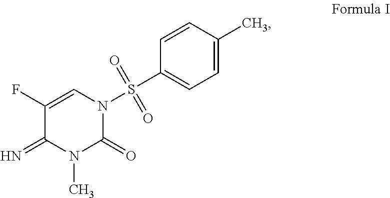 [SMILES: Cc1ccc(S(=O)(=O)n2cc(F)c(=N)n(C)c2=O)cc1]

in one or more forms, wherein at least one form s Form III, Form IIIa, Form 01, or Form 02,
and
(b) a non-aqueous liquid carrier,
wherein the composition has one, two, or three of the following features:
(ii) the non-aqueous liquid carrier is acetophenone,
(iii) the composition comprises at least one fatty alcohol alkoxy ether and/or at least one alkyl fatty acid ester, and
(iv) the concentration of the compound of Formula I in the composition is 50 g/L.

The present invention also provides an oil dispersion (OD) composition comprising:
(a) a fungicidally effective of a compound of Formula I

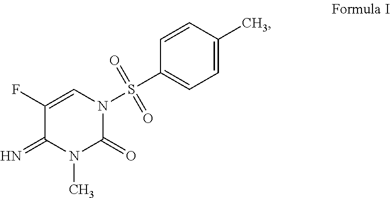 [SMILES: Cc1ccc(S(=O)(=O)n2cc(F)c(=N)n(C)c2=O)cc1]

in one or more forms, wherein at least one form is Form III, Form IIIa, Form 01, or Form 02,
and
(b) a non-aqueous liquid carrier.

The present invention also provides an oil dispersion (OD) composition comprising an admixture of the following components:
(a) a fungicidally effective and substantially pure amount of a compound of Formula I or a fungicidally effective amount of a mixture containing a compound of Formula I:

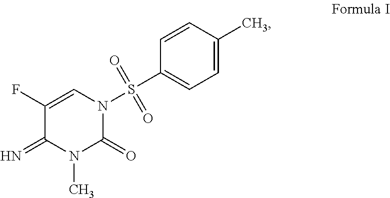 [SMILES: Cc1ccc(S(=O)(=O)n2cc(F)c(=N)n(C)c2=O)cc1]

in one or more forms, wherein at least one form is Form, Form IIIa, Form 01, or Form 02 and wherein 95% or more by weight of the mixture is the compound of Formula I, and
(b) a non-aqueous liquid carrier.

In some embodiments, 96% or more by weight of the mixture is the compound of Formula I. In some embodiments, 97% or more by weight of the mixture is the compound of Formula I.

In some embodiments, the compound of formula I is Form III. In some embodiments, the compound of formula I is Form IIIa. In some embodiments, the compound of formula I is Form 01. In some embodiments, the compound of formula I is Form 02.

In some embodiments, the non-aqueous composition has a water content of less than 0.5% by weight based on the total weight of the composition. In some embodiments, the non-aqueous composition has a water content of less than 0.4% by weight based on the total weight of the composition. In some embodiments, the non-aqueous composition has a water content of less than 0.3% by weight based on the total weight of the composition. In some embodiments, the non-aqueous composition has a water content of less than 0.2% by weight based on the total weight of the composition. In some embodiments, the non-aqueous composition has a water content of less than or equal to 0.2% by weight based on the total weight of the composition. In some embodiments, the non-aqueous composition has a water content of less than 0.1% by weight based on the total weight of the composition. In some embodiments, the non-aqueous composition has a water content of less than or equal to 0.1% by weight based on the total weight of the composition. In some embodiments, the non-aqueous composition has a water content of 0.19%, 0.18%, 0.17%, 0.16%, 0.15%, 0.14%, 0.13%, 0.12%, 0.11%, 0.1%, 0.09%, 0.08%, 0.07%, 0.06%, 0.05%, 0.04% or 0.03% by weight based on the total weight of the composition.

In some embodiments, the non-aqueous composition comprises water scavenger.

In some embodiments, the concentration of the compound of Formula I in the non-aqueous composition is 250 g/L.

The present invention also provides an oil dispersion (OD) composition comprising: an admixture of the following components:
(a) a fungicidally effective and substantially pure amount of a compound of Formula I or a fungicidally effective amount of a mixture containing a compound of Formula I:

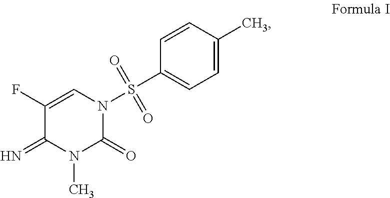 [SMILES: Cc1ccc(S(=O)(=O)n2cc(F)c(=N)n(C)c2=O)cc1]

in one or more forms, wherein at least one form is Form III, Form IIIa, Form 01, or Form 02
wherein 95% or more by weight of the mixture is the compound of Formula I, and
(b) a non-aqueous liquid carrier,
   wherein the composition has one, two, or more of the following features:
(i) the composition further comprises at least one fatty alcohol alkoxy ether and/or a fatty acid ester,
(ii) the composition comprises an effective amount of at least one water scavenger,
(iii) the water content is water content is less than 0.5% by weight based on the total weight of the composition, and
(iv) the concentration of the compound of Formula I in the composition is 250 g/L.

In some embodiments, the water scavenger is tetraethyl orthosilicate and/or epoxidized soybean oil. In some embodiments, the water scavenger is tetraethyl orthosilicate. In some embodiments, the water scavenger is epoxidized soybean oil.

In some embodiments, the amount of water scavenger in the composition is 5% by weight based on the total weight of the composition.

In some embodiments, the amount of tetraethyl orthosilicate in the composition is 5% by weight based on the total weight of the composition. In some embodiments, the amount of epoxidized soybean oil in the composition is 5% by weight based on the total weight of the composition.

In some embodiments, the epoxidized soybean oil is EPOXOL D65 (manufactured and sold by FACI SpA). In some embodiments, the epoxidized soybean oil is Agnique® ESO 81-G (manufactured and sold by BASF). In some embodiments, the amount of EPOXOL D65 in the composition is 5% by weight based on the total weight of the composition. In some embodiments, the amount of Agnique® ESO 81-G in the composition is 5% by weight based on the total weight of the composition.

The present invention also provides an oil dispersion (OD) composition comprising:
(a) a fungicidally effective amount of a compound of Formula I:

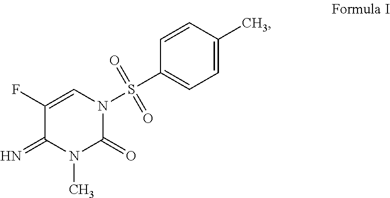 [SMILES: Cc1ccc(S(=O)(=O)n2cc(F)c(=N)n(C)c2=O)cc1]

in one or more forms, wherein at least one form is Form III, Form IIIa, Form 01, or Form 02, and
(b) a non-aqueous liquid carrier,
wherein the composition has one, two, or three of the following features:
(i) the composition further comprises at least one fatty alcohol alkoxy ether and/or a fatty acid ester,
(ii) the composition further comprises 5% by weight of a water scavenger based on the total weight of the composition, and
(iii) the concentration of the compound of Formula I in the composition is 250 g/L.

The present invention also provides an oil dispersion (OD) composition comprising:
(a) a fungicidally effective amount of a compound of Formula I:

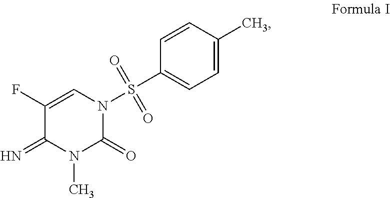 [SMILES: Cc1ccc(S(=O)(=O)n2cc(F)c(=N)n(C)c2=O)cc1]

in one or more forms, wherein at least one form is Form III, Form IIIa, Form 01, or Form 02, and
(b) a non-aqueous liquid carrier,
wherein the composition has one, two, or three of the following features:
(i) the composition further comprises at least one fatty alcohol alkoxy ether and/or a fatty acid ester,
(ii) the composition further comprises 5% by weight of tetraethyl orthosilicate based on the total weight of the composition, and
(iii) the concentration of the compound of Formula I in the composition is 250 g/L.

The present invention also provides an oil dispersion (OD) composition comprising:
(a) a fungicidally effective amount of a compound of Formula I:

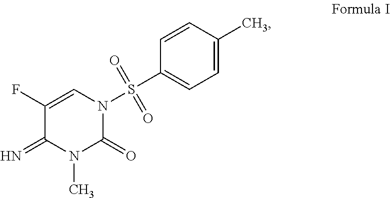 [SMILES: Cc1ccc(S(=O)(=O)n2cc(F)c(=N)n(C)c2=O)cc1]

in one or more forms, wherein at least one form is Form III, Form IIIa, Form 01, or Form 02, and
(b) a non-aqueous liquid carrier,
wherein the composition has one or two of the following features:
(i) the composition further comprises at least one fatty alcohol alkoxy ether and/or a fatty acid ester,
(ii) the composition comprises an amount of water less than 0.5% by weight based on the weight of the composition.

The present invention also provides an oil dispersion (OD) composition comprising:
(a) a fungicidally effective amount of a compound of Formula I:

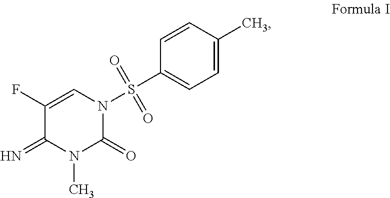 [SMILES: Cc1ccc(S(=O)(=O)n2cc(F)c(=N)n(C)c2=O)cc1]

in one or more forms, wherein at least one form is Form III, Form IIIa, Form 01, or Form 02, and
(b) a non-aqueous liquid carrier,
wherein the composition has one, two, or three of the following features:
(i) the composition further comprises at least one fatty alcohol alkoxy ether and/or a fatty acid ester,
(ii) the composition comprises an amount of water less than 0.5% by weight based on the total weight of the composition, and
(iii) the concentration of the compound of Formula I in the composition is about 250 g/L.

The present invention also provides an oil dispersion (OD) composition comprising:
(a) a fungicidally effective amount of a compound of Formula I:

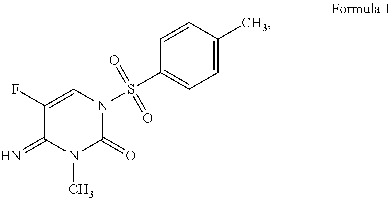 [SMILES: Cc1ccc(S(=O)(=O)n2cc(F)c(=N)n(C)c2=O)cc1]

in one or more forms, wherein at least one form is Form III, Form IIIa, Form 01, or Form 02, and
(b) a non-aqueous liquid carrier,
wherein the composition has one, two, or three of the following features:
(i) the composition further comprises at least one fatty alcohol alkoxy ether and/or a fatty acid ester,
(ii) the composition further comprises an effective amount of at least one water scavenger, wherein the water scavenger is tetraethyl orthosilicate at an amount of 5% by weight based on the total weight of the composition, and/or epoxidized soybean oil, and
(iii) the concentration of the compound of Formula I in the composition is 250 g/L.

The present invention also provides a SC composition comprising:
b) 40-45% by weight of the compound of Formula I based on the total weight of the composition, wherein the compound of Formula I is in one or more forms and at least one form is Form III, Form IIIa, Form 01, or Form 02,
c) 0.15-0.25% by weight of xanthan gum based on the total weight of the composition,
d) 0.5-0.6% by weight of disodium phosphate anhydrous based on the total weight of the composition,
e) 1.2-1.6% by weight of tristyryl phenol-polyethylene glycol ether based on the total weight of the composition.
f) 0.1-0.2% by weight of potassium dihydrogene phosphate based on the total weight of the composition,
g) 1-3% by weight of 1,2-propanediol based on the total weight of the composition,
h) 0.01-0.05% by weight of a biocide,
i) 1-2% by weight of polydimethylsiloxane emulsion based on the total weight of the composition,
j) 0.1-0.3% by weight of siloxane polyalkyleneoxide copolymer based on the total weight of the composition,
k) 3.5-4% by weight of tristyrylphenol ethoxylate phosphate ester based on the total weight of the composition,
l) 0.3-0.7% by weight of sodium diisopropylnaphthalene
sulphonate based on the total weight of the composition, and m) 45-50% by weight of water based on the total weight of the composition.

The present invention also provides a SC composition comprising:
a) 41.67% by weight of the compound of Formula I based on the total weight of the composition, wherein the compound of Formula I is in one or more forms and at least one form is Form III, Form IIIa, Form 01, or Form 02,
b) 0.19% by weight of xanthan gum based on the total weight of the composition,
c) 0.58% by weight of disodium phosphate anhydrous based on the total weight of the composition,
d) 1.42% by weight of tristyryl phenol-polyethylene glycol ether based on the total weight of the composition.
e) 0.14% by weight of potassium dihydrogene phosphate based on the total weight of the composition,
f) 1.96% by weight of 1,2-propanediol based on the total weight of the composition,
g) 0.0167% by weight of a biocide,
h) 1.5% by weight of polydimethylsiloxane emulsion based on the total weight of the composition,
i) 0.2% by weight of siloxane polyalkyleneoxide copolymer based on the total weight of the composition,
j) 3.75% by weight of tristyrylphenol ethoxylate phosphate ester based on the total weight of the composition,
k) 0.5% by weight of sodium diisopropylnaphthalene sulphonate based on the total weight of the composition, and
l) 48.575% by weight of water based on the total weight of the composition.

The present invention also provides a SC composition comprising:
a) 40-45% by weight of the compound of Formula I based on the total weight of the composition, wherein the compound of Formula I is in one or more forms and at least one form is Form III, Form IIIa, Form 01, or Form 02,
b) 0.3-0.7% by weight of sodium diisopropylnaphthalene sulphonate based on the total weight of the composition,
c) 3-5% by weight of 2,4,6-tris(1-phenylethyl)polyoxyethylenated phosphates based on the total weight of the composition,
d) 1.4-1.8% by weight of 2,4,6-tri-(1-phenylethyl)-phenol polyglycol ether with 54 EO based on the total weight of the composition,
e) 1-3% by weight of propylene glycol based on the total weight of the composition,
f) 0.2-0.3% by weight of polyalkyleneoxide modified heptamethyltrisiloxane based on the total weight of the composition,
g) 0.5-1.5% by weight of polydimethylsiloxane antifoam emulsion based on the total weight of the compositions,
h) 7-8% by weight of xanthan gum based on the total weight of the composition,
i) 0.1-0.2% by weight of potassium dihydrogen phosphate based on the total weight of the composition,
j) 0.5-0.6% by weight of disodium phosphate anhydrous based on the total weight of the composition,
k) 0.05-0.1% by weight of a biocide based on the total weight of the composition, and
l) 35-45% by weight of water based on the total weight of the composition.

The present invention also provides a SC composition comprising:
a) 42% by weight of the compound of Formula I based on the total weight of the composition, wherein the compound of Formula I is in one or more forms and at least one form is Form III, Form IIIa, Form 01, or Form 02,
b) 0.5% by weight of sodium diisopropylnaphthalene sulphonate based on the total weight of the composition,
c) 4% by weight of 2,4,6-tris(1-phenylethyl)polyoxyethylenated phosphates based on the total weight of the composition,
d) 1.6% by weight of 2,4,6-tri-(1-phenylethyl)-phenol polyglycol ether with 54 EO based on the total weight of the composition,
e) 2% by weight of propylene glycol based on the total weight of the composition,
f) 0.25% by weight of polyalkyleneoxide modified heptamethyltrisiloxane based on the total weight of the composition,
g) 1% by weight of polydimethylsiloxane antifoam emulsion based on the total weight of the compositions,
h) 7.4% by weight of xanthan gum based on the total weight of the composition,
i) 0.14% by weight of potassium dihydrogen phosphate based on the total weight of the composition,
j) 0.57% by weight of disodium phosphate anhydrous based on the total weight of the composition,
k) 0.08% by weight of a biocide based on the total weight of the composition, and
l) 40% by weight of water based on the total weight of the composition.

The present invention also provides an emulsifiable concentrate (EC) composition comprising:
a) 4.5-5% by weight of the compound of Formula I based on the total weight of the composition, wherein the compound of Formula I is in one or more forms and at least one form is Form III, Form IIIa, Form 01, or Form 02,
b) 7-8% by weight of C16-C18 alcohol ethoxylate propoxylate ether based on the total weight of the composition,
c) 22-25% by weight of tristyrylphenol ethoxylate based on the total weight of the composition,
d) 3-4% by weight of fatty alcohol alkoxylate blend in propylene glycol based on the total weight of the composition, and
e) 55-65% by weight of acetophenone based on the total weight of the composition.

The present invention also provides an EC composition comprising:
a) 4.74% by weight of the compound of Formula I based on the total weight of the composition, wherein the compound of Formula I is in one or more forms and at least one form is Form III, Form IIIa, Form 01, or Form 02,
b) 7.11% by weight of C16-C18 alcohol ethoxylate propoxylate ether based on the total weight of the composition,
c) 23.22% by weight of tristyrylphenol ethoxylate based on the total weight of the composition,
d) 3.79% by weight of fatty alcohol alkoxylate blend in propylene glycol based on the total weight of the composition, and
e) 61.14% by weight of acetophenone based on the total weight of the composition.

The present invention also provides an emulsifiable concentrate (EC) composition comprising:
a) 4-5% by weight of the compound of Formula I based on the total weight of the composition, wherein the compound of Formula I is in one or more forms and at least one form is Form III, Form IIIa, Form 01, or Form 02,
b) 22-24% by weight of tristyrylphenol ethoxylate based on the total weight of the composition,
c) 5-6% by weight of fatty alcohol alkoxylate blend in propylene glycol based on the total weight of the composition,
d) 5-6% by weight of C16-C18 alcohol ethoxylate propoxylate ether based on the total weight of the composition, and
e) 60-65% by weight of acetophenone based on the total weight of the composition.

The present invention also provides an emulsifiable concentrate (EC) composition comprising:
a) 4.8% by weight of the compound of Formula I based on the total weight of the composition, wherein the compound of Formula I is in one or more forms and at least one form is Form III, Form IIIa, Form 01, or Form 02,
b) 23% by weight of tristyrylphenol ethoxylate based on the total weight of the composition,
c) 5.5% by weight of fatty alcohol alkoxylate blend in propylene glycol based on the total weight of the composition,
d) 5.5% by weight of C16-C18 alcohol ethoxylate propoxylate ether based on the total weight of the composition, and
e) 61% by weight of acetophenone based on the total weight of the composition.

The present invention also provides an OD composition comprising:
a) 23-27% by weight of the compound of Formula I based on the total weight of the composition, wherein the compound of Formula I is in one or more forms and at least one form is Form III, Form IIIa, Form 01, or Form 02,
b) 2-4% by weight of polymer based on the total weight of the composition,
c) 2-4% by weight of alkoxylated fatty alcohol based on the total weight of the composition,
d) 5-7% by weight of sodium dioctyl sulfosuccinate based on the total weight of the composition,
e) 5-7% by weight of ethoxylate propoxylate alcohol based on the total weight of the composition,
f) 2-4% by weight of alkoxylated fatty alcohol based on the total weight of the composition,
g) 4-6% by weight of tetraethyl orthosilicate based on the total weight of the composition,
h) 0.1-1% by weight of hydrophilic fumed silica based on the total weight of the composition, and
i) 40-55% by weight of C18 methyl canolate ester based on the total weight of the composition.

The present invention also provides an OD composition comprising:
a) 25% by weight of the compound of Formula I based on the total weight of the composition, wherein the compound of Formula I is in one or more forms and at least one form is Form III, Form IIIa, Form 01, or Form 02,
b) 3% by weight of polymer based on the total weight of the composition,
c) 3% by weight of alkoxylated fatty alcohol based on the total weight of the composition,
d) 6% by weight of sodium dioctyl sulfosuccinate based on the total weight of the composition,
e) 6% by weight of ethoxylate propoxylate alcohol based on the total weight of the composition,
f) 3% by weight of alkoxylated fatty alcohol based on the total weight of the composition,
g) 5% by weight of tetraethyl orthosilicate based on the total weight of the composition,
h) 0.5% by weight of hydrophilic fumed silica based on the total weight of the composition, and
i) 48.5% by weight of C18 methyl canolate ester based on the total weight of the composition.

The present invention also provides an OD composition comprising:
a) 23-27% by weight of the compound of Formula I based on the total weight of the composition, wherein the compound of Formula I is in one or more forms and at least one form is Form III, Form IIIa, Form 01, or Form 02,
b) 2-4% by weight of polymeric dispersant based on the total weight of the composition,
c) 2-4% by weight of polymer based on the total weight of the composition,
d) 5-7% by weight of sodium dioctyl sulfosuccinate based on the total weight of the composition,
e) 5-7% by weight of ethoxylate propoxylate alcohol based on the total weight of the composition,
f) 2-4% by weight of alkoxylated fatty alcohol based on the total weight of the composition,
g) 4-6% by weight of tetraethyl orthosilicate based on the total weight of the composition,
h) 2-3% by weight of hydrophilic fumed silica based on the total weight of the composition, and
i) 40-50% by weight of C18 methyl canolate ester based on the total weight of the composition.

The present invention also provides an OD composition comprising:
a) 25% by weight of the compound of Formula I based on the total weight of the composition, wherein the compound of Formula I is in one or more forms and at least one form is Form III, Form IIIa, Form 01, or Form 02,
b) 3% by weight of polymeric dispersant based on the total weight of the composition,
c) 3% by weight of polymer based on the total weight of the composition,
d) 6% by weight of sodium dioctyl sulfosuccinate based on the total weight of the composition,
e) 6% by weight of ethoxylate propoxylate alcohol based on the total weight of the composition,
f) 3% by weight of alkoxylated fatty alcohol based on the total weight of the composition,
g) 5% by weight of tetraethyl orthosilicate based on the total weight of the composition,
h) 2.6% by weight of hydrophilic fumed silica based on the total weight of the composition, and
i) 46.4% by weight of C18 methyl canolate ester based on the total weight of the composition.

The present invention also provides an OD composition comprising:
a) 23-27% by weight of the compound of Formula I based on the total weight of the composition, wherein the compound of Formula I is in one or more forms and at least one form is Form III, Form IIIa, Form 01, or Form 02,
b) 2-4% by weight of polymeric dispersant based on the total weight of the composition,
c) 4-6% by weight of polymer based on the total weight of the composition,
d) 5-7% by weight of sodium dioctyl sulfosuccinate based on the total weight of the composition,
e) 5-7% by weight of block copolymer of ethylene oxide and propylene oxide based on the total weight of the composition,
f) 4-6% by weight of ethoxylated isotridecyl alcohol based on the total weight of the composition,
g) 4-6% by weight of tetraethyl orthosilicate based on the total weight of the composition,
h) 0.1-0.3% by weight of decamethylcyclopentasiloxane based on the total weight of the composition,
i) 3-4% by weight of polysorbates based on the total weight of the composition, and
j) 40-45% by weight of C16-C18 unsaturated fatty acid methyl esters based on the total weight of the composition.

The present invention also provides an OD composition comprising:
a) 26% by weight of the compound of Formula I based on the total weight of the composition, wherein the compound of Formula I is in one or more forms and at least one form is Form III, Form IIIa, Form 01, or Form 02,
b) 3% by weight of polymeric dispersant based on the total weight of the composition,
c) 5% by weight of polymer based on the total weight of the composition,
d) 6% by weight of sodium dioctyl sulfosuccinate based on the total weight of the composition,
e) 6% by weight of block copolymer of ethylene oxide and propylene oxide based on the total weight of the composition,
l) 5% by weight of ethoxylated isotridecyl alcohol based on the total weight of the composition,
g) 5% by weight of tetraethyl orthosilicate based on the total weight of the composition,
h) 0.2% by weight of decamethylcyclopentasiloxane based on the total weight of the composition,
i) 3.25% by weight of polysorbates based on the total weight of the composition,
j) 41% by weight of C16-C18 unsaturated fatty acid methyl esters based on the total weight of the composition.

The present invention also provides an OD composition comprising:
a) 23-27% by weight of the compound of Formula I based on the total weight of the composition, wherein the compound of Formula I is in one or more forms and at least one form is Form III, Form IIIa, Form 01, or Form 02,
b) 2-4% by weight of polymeric dispersant based on the total weight of the composition,
c) 4-6% by weight of polymer based on the total weight of the composition,
d) 5-7% by weight of sodium dioctyl sulfosuccinate based on the total weight of the composition,
e) 5-7% by weight of block copolymer of ethylene oxide and propylene oxide based on the total weight of the composition,
f) 4-6% by weight of ethoxylated isotridecyl alcohol based on the total weight of the composition,
g) 4-6% by weight of epoxidized soybean oil based on the total weight of the composition,
h) 2-3% by weight of decamethylcyclopentasiloxane based on the total weight of the composition,
i) 3-4% by weight of polysorbates based on the total weight of the composition, and
j) 35-45% by weight of C16-C18 unsaturated fatty acid methyl esters based on the total weight of the composition.

The present invention also provides an OD composition comprising:
a) 26% by weight of the compound of Formula I based on the total weight of the composition, wherein the compound of Formula I is in one or more forms and at least one form is Form III, Form IIIa, Form 01, or Form 02,
b) 3% by weight of polymeric dispersant based on the total weight of the composition,
c) 5% by weight of polymer based on the total weight of the composition,
d) 6% by weight of sodium dioctyl sulfosuccinate based on the total weight of the composition,
e) 6% by weight of block copolymer of ethylene oxide and propylene oxide based on the total weight of the composition,
f) 5% by weight of ethoxylated isotridecyl alcohol based on the total weight of the composition,
g) 5% by weight of epoxidized soybean oil based on the total weight of the composition,
h) 2.5% by weight of decamethylcyclopentasiloxane based on the total weight of the composition,
i) 3.25% by weight of polysorbates based on the total weight of the composition,
j) 39% by weight of C16-C18 unsaturated fatty acid methyl esters based on the total weight of the composition.

The present invention also provides a stable, liquid composition comprising an admixture of the following components:
(a) a fungicidally effective and substantially pure amount of a compound of Formula I or a fungicidally effective amount of a mixture containing a compound of Formula I:

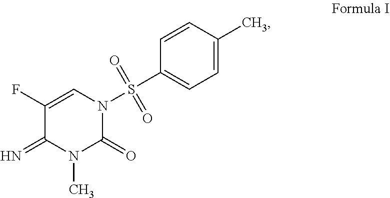 [SMILES: Cc1ccc(S(=O)(=O)n2cc(F)c(=N)n(C)c2=O)cc1]

in one or more forms and at least one form is Form III, Form IIIa, Form 01, or Form 02,
wherein 95% or more by weight of the mixture is the compound of Formula I, and
(b) a liquid carrier.

Any one of the compositions described herein comprising a list of components may also be described as a composition comprising an admixture comprising the same list of components.

In some embodiments, the fungicidally effective amount of a compound of Formula I is comprised in a mixture wherein 95% or more by weight of the mixture is the compound of Formula I.

In some embodiments of the invention herein, the composition is stable. In some embodiments of the invention herein, there is less than 10% degradation of the amount of the compound of Formula I in the composition after 2 weeks of storage at 54° C. In some embodiments of the invention herein, there is less than 5% degradation of the amount of the compound of Formula I in the composition after 2 weeks of storage at 54° C.

### (i) Compound of Formula I

The compound of Formula I of the present invention refers to any solid form including but not limited to amorphous, crystalline, solvate or hydrate.

The compound of Formula I includes crystalline forms of the compound of Formula I.

Crystalline Forms III, IIIa, 01 and 02 are defined hereinabove. Forms III, IIIa, 01 and 02 exhibit distinct spectral characteristics as depicted by their X-ray diffraction patterns. In some embodiments, the crystalline form is Form III. In some embodiments, the crystalline form is Form IIIa. In some embodiments, the crystalline form is Form 01. In some embodiments, the crystalline form is Form 02.

Other forms of the compound of Formula I are described below.

In some embodiments, the crystalline form is an anhydrous crystalline form. In some embodiments, the anhydrous crystalline form is a polymorph. In some embodiments, the anhydrous crystalline form is a pseudopolymorph.

Polymorphs of the compound of Formula I is described in PCT International Application Publication No. WO/2019/038583 (published Feb. 28, 2019), the entire content of which is hereby incorporated by reference into this application.

In some embodiments, the crystalline form is Form I polymorph. In some embodiments, the crystalline form is Form II polymorph. In some embodiments, the crystalline form is a hydrate.

In some embodiments, the crystalline form is a solvate. In some embodiments, the solvate contains 1,4-dioxane. In some embodiments, the solvate contains tetrahydrofuran. In some embodiments, the solvate contains ethyl acetate.

In some embodiments, the crystalline polymorphic form (Form I polymorph) exhibits an X-ray powder diffraction pattern having characteristic peaks at 2-theta angles of 9.08, 10.98, 14.05, 17.51, 18.75, 21.63, 23.33, 24.70, 24.83, 25.37, 26.51 and 29.23. In one embodiment, the powder X-ray diffraction pattern of Form I comprises characteristic peaks at 2-theta angles of 14.05, 17.51, 18.75, 21.63 and 26.51. In one embodiment, the powder X-ray diffraction pattern of Form I comprises characteristic peaks at 2-theta angles of 14.05, 17.51, 18.75 and 21.63.

In some embodiments, the crystalline polymorphic form (Form I polymorph) is characterized by decomposition beginning at a temperature greater than 210° C.

In some embodiments, the crystalline polymorphic form (Form I polymorph) exhibits a Differential Scanning Calorimetry (DSC) thermogram characterized by a predominant endothermic peak with a peak temperature of about 160° C., a predominant endothermic peak with an onset temperature of about 159° C., and a predominant endothermic peak with a melting enthalpy of about 110 J/g.

In one embodiment, the crystalline polymorphic form (Form II polymorph) exhibits an X-ray powder diffraction pattern having characteristic peaks at 2-theta angles of 7.98, 9.20, 9.96, 11.88, 15.99, 18.49, 21.23, 22.33, 22.59, 26.73. In one embodiment, the powder X-ray diffraction pattern of Form II comprises characteristic peaks at 2-theta angles of 9.20, 9.96, 11.88, 22.33 and 22.59. In one embodiment, the powder X-ray diffraction pattern of Form II comprises characteristic peaks at 2-theta angles of 9.20, 11.88, 22.33 and 22.59.

In one embodiment, the crystalline polymorphic form (Form II polymorph) exhibits a TG-FTIR thermogram characterized by decomposition beginning at a temperature greater than 210° C.

In one embodiment, the crystalline polymorphic form (Form II polymorph) exhibits a Differential Scanning Calorimetry (DSC) thermogram characterized by a predominant endothermic peak with a peak temperature of about 157° C., a predominant endothermic peak with an onset temperature of about 156° C., and a predominant endothermic peak with a melting enthalpy of about 112 J/g.

In one embodiment, the crystalline hydrate form (Hydrate) exhibits an X-ray powder diffraction pattern having characteristic peaks at 2-theta 5.34, 7.48, 10.68, 16.05, 21.79, 22.99, 23.19, 24.95, 26.95, 27.63. In one embodiment, the powder X-ray diffraction pattern of Hydrate comprises characteristic peaks at 2-theta angles of 5.34, 7.48, 10.68, 16.05 and 21.79. In one embodiment, the powder X-ray diffraction pattern of Hydrate comprises characteristic peaks at 2-theta angles of 5.34, 7.48, 10.68 and 16.05.

In one embodiment, the crystalline hydrate form (Hydrate) exhibits a TG-FTIR thermogram characterized by decomposition beginning at a temperature greater than 190° C.

In one embodiment, the crystalline hydrate form (Hydrate) exhibits a Differential Scanning Calorimetry (DSC) thermogram characterized by a predominant endothermic peak with a peak temperature of about 139.5° C., a predominant endothermic peak with an onset temperature of about 139° C., and a predominant endothermic peak with a melting enthalpy of about 115 J/g, wherein the DSC is measured in a sealed pan.

In one embodiment, the crystalline hydrate form (Hydrate) exhibits a Differential Scanning Calorimetry (DSC) thermogram characterized by a predominant endothermic peak with a peak temperature of about 160° C., a predominant endothermic peak with an onset temperature of about 159° C., and a predominant endothermic peak with a melting enthalpy of about 98 J/g, wherein the DSC is measured in an open pan.

In one embodiment, the crystalline solvate form (Form S5) exhibits an X-ray powder diffraction pattern having characteristic peaks at 2-theta 5.42, 7.50, 10.06, 10.82, 12.80, 16.91, 21.55, 23.13, 24.83, 26.81, 27.77. In one embodiment, the powder X-ray diffraction pattern of Form S5 comprises characteristic peaks at 2-theta angles of 5.42, 7.50, 10.06, 10.82, and 16.91. In one embodiment, the powder X-ray diffraction pattern of Form S5 comprises characteristic peaks at 2-theta angles of 5.42, 7.50, 10.82 and 16.91.

In one embodiment, the crystalline solvate form (Form S5) exhibits a TG-FTIR thermogram characterized by decomposition beginning at a temperature greater than 180° C.

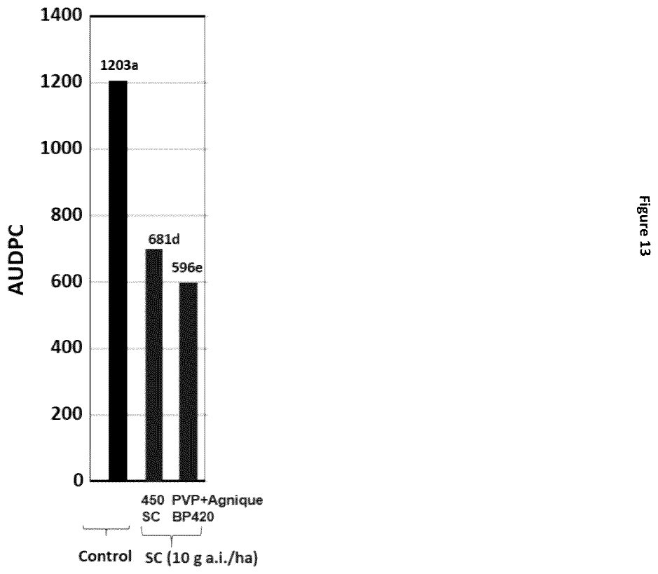

In one embodiment, the crystalline solvate form (Form S8) exhibits an X-ray powder diffraction pattern as shown in FIG. 13 of PCT/IB2018/000875, having characteristic peaks at 2-theta 4.7, 5.00, 5.38, 6.26, 9.66, 15.93, 21.05, 23.97, 24.69. In one embodiment, the powder X-ray diffraction pattern of Form S8 comprises characteristic peaks at 2-theta angles of 4.7, 5.00, 5.38, 6.26, 9.66 and 23.97. In one embodiment, the powder X-ray diffraction pattern of Form S8 comprises characteristic peaks at 2-theta angles of 4.7, 5.00, 9.66 and 23.97.

In one embodiment, the crystalline solvate form (Form S8) exhibits a TG-FTIR thermogram characterized by decomposition beginning at a temperature greater than 180° C.

In one embodiment, the crystalline solvate form (Form S1) exhibits an X-ray powder diffraction pattern having characteristic peaks at 2-theta 5.34, 7.48, 10.10, 10.68, 12.90, 16.07, 21.83, 23.09, 24.91, 26.93. In one embodiment, the powder X-ray diffraction pattern of Form S1 comprises characteristic peaks at 2-theta angles of 5.34, 7.48, and 10.68. In one embodiment, the powder X-ray diffraction pattern of Form S1 comprises characteristic peaks at 2-theta angles of 5.34, 7.48, 10.68 and 21.83. In one embodiment, the powder X-ray diffraction pattern of Form S1 comprises characteristic peaks at 2-theta angles of 5.34, 7.48, 10.68, 16.07 and 21.83.

In one embodiment, the crystalline solvate form (Form S1) exhibits a TG-FTIR thermogram characterized by decomposition beginning at a temperature greater than 200° C.

In some embodiments, the compound of Formula I is a mixture of crystalline forms of the compound of Formula I.

In some embodiments, the mixture is a mixture of one or more anhydrous crystalline forms.

### (ii) Suitable Stabilizing Surfactants

In some embodiments, the composition comprises at least one stabilizing surfactant. In some embodiments, the composition comprises at least two stabilizing surfactants. In some embodiments, the composition comprises a stabilizing system.

In some embodiments, the composition comprises a non-ionic stabilizing surfactant. In some embodiments, the composition comprises an anionic stabilizing surfactant. In some embodiments, the composition comprises a combination of a non-ionic stabilizing surfactant and an anionic stabilizing surfactant.

In some embodiments, the suspension concentrate (SC) composition comprises at least one stabilizing surfactant. In some embodiments, the suspension concentrate (SC) composition comprises at least two stabilizing surfactants. In some embodiments, the suspension concentrate (SC) composition comprises two stabilizing surfactants.

In some embodiments, the composition is a suspoemulsion (SE) composition. In some embodiments, the SE composition comprises at least one stabilizing surfactant. In some embodiments, the SE composition comprises at least two stabilizing surfactants. In some embodiments, the SE composition comprises two stabilizing surfactants.

In some embodiments, the stabilizing surfactant is a physical stabilizer.

In some embodiments, the stabilizing surfactant affects the crystals growth rate of the compound of Formula I in the liquid carrier. In some embodiments, the stabilizing surfactant decreases the crystals growth rate of the compound of Formula I in the liquid carrier. In some embodiments, the stabilizing surfactant have a crystal growth inhibiting property. In some embodiments, the stabilizing surfactant is a crystal growth inhibitor.

In some embodiments, one of the stabilizing surfactants is a non-ionic stabilizing surfactant. In some embodiments, the non-ionic stabilizing surfactant is selected from the group consisting of polymers, ester alkoxylated amine, ester of alkoxylated diethylethanolamine, poly alkylene oxide alcohol ether, and alcohols.

In some embodiments, the polymer is a block polymer of random polymer. In some embodiments, the polymer is a tri-block polymer. In some embodiments, the tri-block polymer is an ABA block polymer. In some embodiments, the polymer has a low HLB (hydrophile-lpophile balance) value, preferably an HLB value of 5. In some embodiments, the polymer is Atlox™ 4912 (manufactured and sold by Croda). In some embodiments, the polymer is Atlox™ 4916 (manufactured and sold by Croda).

In some embodiments, the amount of polymer in the composition is 1-10% by weight based on the total weight of the composition. In some embodiments, the amount of polymer in the composition is 4-6% by weight based on the total weight of the composition. In some embodiments, the amount of polymer in the composition is about 5% by weight based on the total weight of the composition.

In some embodiments, the non-ionic stabilizing surfactant is an ester alkoxylated amine. In some embodiments, the ester alkoxylated amine is Atlox™ 4915 (manufactured and sold by Croda). In some embodiments, the non-ionic stabilizing surfactant is Atlox™ 4915 (manufactured and sold by Croda). In some embodiments, the non-ionic stabilizing surfactant is alkoxylated diethylethanolamine. In some embodiments, the non-ionic stabilizing surfactant is di-ethyl ethanol amine mono-trimerate. In some embodiments, the non-ionic stabilizing surfactant is Atlox™ 4915 (manufactured and sold by Croda).

In some embodiments, the poly alkylene oxide alcohol ether is a fatty alcohol ether and/or a non-fatty alcohol ether.

In some embodiments, the non-ionic stabilizing surfactant is an alkoxylated fatty alcohol.

In some embodiments, the alkoxylated fatty alcohol is Genapol® X080 (manufactured and sold by Clariant), Genapol® X 050 (manufactured and sold by Clariant), tridecyl alcohol polyglycol ether, Rhodasurf® LA 30 (manufactured and sold by Solvay), Aerosol® OT-SE or Aerosol® OT-100 (manufactured and sold by Solvay), Rhodacal® 70/B (manufactured and sold by Solvay), Arlatone™ TV (manufactured and sold by Croda), Alkamuls® A (manufactured and sold by Solvay), or Alkamuls® BR (manufactured and sold by Solvay).

In some embodiments, the alkoxylated fatty alcohol is Genapol® X080 (manufactured and sold by Clariant), Genapol® X 050 (manufactured and sold by Clariant), tridecyl alcohol polyglycol ether, or Rhodasurf® LA 30 (manufactured and sold by Solvay).

In some embodiments, the alkoxylated fatty alcohol is Atlas' 5002L.

In some embodiments, the alcohol has a short carbon chain of C1-C6. In some embodiments, the alcohol has a long carbon chain of C7-C20.

In some embodiments, the non-ionic stabilizing surfactant is a non-ionic derivative of polyalkylene oxide polyaryl ether.

In some embodiments, one of the stabilizing surfactants is an ionic surfactant. In some embodiments, one of the stabilizing surfactants is an ionic stabilizing surfactant.

In some embodiments, the ionic stabilizing surfactant is selected from the group consisting of Aerosol® OT-SE or Aerosol® OT-100 (manufactured and sold by Solvay), Rhodacal® 70/B (manufactured and sold by Solvay), and a combination thereof.

In some embodiments, the ionic stabilizing surfactant is an anionic stabilizing surfactant. Anionic stabilizing surfactant refers to compounds which have an anionic group such as phosphonic salt and sulfonic salt. An example of an ionic surfactant that may be used is sodium dioctyl sulfosuccinate which is manufactured and sold by Solvay as Aerosol® OT-SE.

In some embodiments, the anionic stabilizing surfactant is anionic derivative of polyalkylene oxide polyaryl ether.

In some embodiments, the composition comprises at least one non-ionic stabilizing surfactant and at least one anionic stabilizing surfactant.

In some embodiments, the stabilizing system comprises at least one non-ionic stabilizing surfactant and at least one anionic stabilizing surfactant.

In some embodiments, the composition comprising a non-ionic stabilizing surfactant and an anionic stabilizing surfactant is a SC composition.

In some embodiments, the composition comprising a non-ionic stabilizing surfactant and an anionic stabilizing surfactant is a SE composition.

In some embodiments, one of the stabilizing surfactants is a derivative of polyalkylene oxide polyaryl ether. In some embodiments, the derivative of polyalkylene oxide polyaryl ether is a nonionic derivative of polyalkylene oxide polyaryl ether. In some embodiments, the derivative of polyalkylene oxide polyaryl ether surfactant is an anionic derivative of polyalkylene oxide polyaryl ether.

In some embodiments, the composition comprises at least two stabilizing surfactants. In some embodiments, the two stabilizing surfactants comprise two derivatives of polyalkylene oxide polyaryl ether. In some embodiments, the two stabilizing surfactants comprise a non-ionic derivative of polyalkylene oxide polyaryl ether and an anionic derivative of polyalkylene oxide polyaryl ether.

In some embodiments, the non-ionic derivative of polyalkylene oxide polyaryl ether is a compound having an aryl group substituted with at least two aromatic groups.

In some embodiments, the non-ionic derivative of polyalkylene oxide polyaryl ether has the following structure:

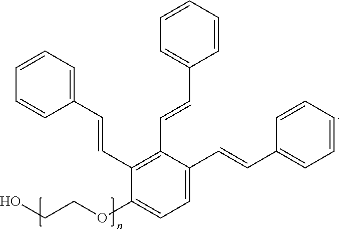

In some embodiments, the non-ionic derivative of polyalkylene oxide polyaryl ether has the following structure:

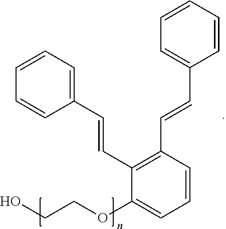

In some embodiments, the anionic derivative of polyalkylene oxide polyaryl ether is a compound having an aryl group substituted with at least two aromatic groups.

In some embodiments, the anionic derivative of polyalkylene oxide polyaryl ether comprises an anionic group selected from phosphate (PO₄), phosphonate (PO₃), sulfonate (SO₃), and sulfate (SO₄). In some embodiment, the anionic group of the anionic derivative of polyalkylene oxide polyaryl ether has an anionic group selected from phosphate (PO₄), phosphonate (PO₃), sulfonate (SO₃), and sulfate (SO₄).

In some embodiments, the polyalkylene oxide polyaryl ether comprises a polyalkylene oxide group selected from the group consisting of polyethylene oxide group, polypropylene oxide, polybutylene oxide and any combination thereof. In some embodiments, the polyalkylene oxide group is a polyethylene oxide. In some embodiments, the polyalkylene oxide group is a polypropylene oxide.

Polyalkylene oxides may include but are not limited to copolymers and homogenous polymers. Copolymers may include but are not limited to random polymer and block polymer. In some embodiments, the polyalkylene oxide group is a di block copolymer. In some embodiments, the polyalkylene oxide group is a tri block copolymer.

In some embodiments, the polyalkylene oxide polyaryl ether is a polyalkylene oxide styryl phenyl ether. In some embodiments, the polyalkylene oxide polyaryl ether is a polyalkylene oxide benzyl phenyl ether. In some embodiments, the polyalkylene oxide polyaryl ether is a polyalkylene oxide bisphenyl ether. In some embodiments, the polyalkylene oxide polyaryl ether is a polyalkylene oxide tristyryl phenyl ether. In some embodiments, the polyalkylene oxide polyaryl ether is a polyalkylene oxide distyryl phenyl ether. In some embodiments, the polyalkylene oxide distyryl phenyl ether is polyoxyethylene distyryl phenyl ether.

In some embodiments, the polyalkylene oxide polyaryl ether is an anionic stabilizing surfactant. Anionic stabilizing surfactant refers to compounds which have an anionic group such as phosphonic salt and sulfonic salt.

In some embodiments, the salt comprises a cation. In some embodiments, the cation is selected from a group consisting of sodium, potassium, ammonium, calcium, magnesium and combinations thereof.

In some embodiments, the anionic derivative of polyalkylene oxide polyaryl ether has the following structure:

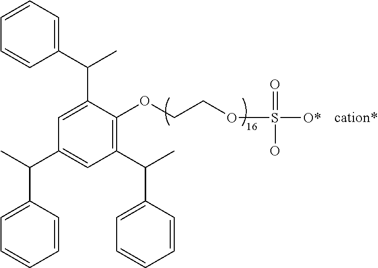

In some embodiments, the anionic derivative of polyalkylene oxide polyaryl ether is tristyrylphenol ethoxylate phosphate ester.

In some embodiments, the polyalkylene oxide polyaryl ether is tristyrylphenol ethoxylate phosphate ester. Preferably, the tristyrylphenol ethoxylate phosphate ester is Soprophor® 3D33 manufactured and sold by Solvay. In some embodiments, the amount of Soprophor® 3D33 in the SC composition is 3-5% by weight based on the total weight of the composition. In some embodiments, the amount of Soprophor® 3D33 in the SC composition is about 4% by weight based on the total weight of the composition.

In some embodiments, the polyalkylene oxide polyaryl ether is 2,4,6-Tri-(1-phenylethyl)-phenol polyglycol ether with 54 EQ. Preferably, the 2,4,6-Tri-(1-phenylethyl)-phenol polyglycol ether with 54 EQ is Emulsogen® TS 540 manufactured and sold by Clariant. In some embodiments, the amount of Emulsogen® TS 540 in the SC composition is 1-2% by weight based on the total weight of the composition. In some embodiments, the amount of Emulsogen® TS 540 in the SC composition is about 1.6% by weight based on the total weight of the composition.

In some embodiments, the polyalkylene oxide polyaryl ether is ethoxylated tristyrylphenol. Preferably, the ethoxylated tristyrylphenol is Soprophor® TS/54 manufactured and sold by Solvay.

In some embodiments, the salt comprises at least one cation selected from group consisting of sodium, potassium, ammonium, calcium, magnesium and combination thereof.

Polyalkylene oxide polyaryl ether surfactants may include but is not limited to poly phenyl ethyl phenol and tristyrylphenol.

Polyalkylene oxide polyaryl ethers surfactant may include but is not limited to non-capped surfactants, end-capped surfactants or combination thereof.

In some embodiments, the composition comprises a combination of stabilizing surfactants and the combination of stabilizing surfactants comprises a mixture of a nonionic polyalkylene oxide polyaryl ether surfactant and an anionic polyalkylene oxide polyaryl ether surfactant.

In some embodiments, the nonionic surfactant is tristyrylphenol ethoxylate. In some embodiments, the anionic surfactant is tristyrylphenol ethoxylate phosphate ether.

In some embodiments, the combination of stabilizing surfactants comprises tristyrylphenol ethoxylate and tristyrylphenol ethoxylate phosphate ether.

In some embodiments, the nonionic polyalkylene oxide polyaryl ether is a compound having an ether group substituted with at least two groups comprising aromatic rings.

In some embodiments, the polyalkylene oxide group is a polyoxyethylene.

In some embodiments, the polyalkylene oxide group is a polyoxypropylene.

In some embodiments, the polyalkylene oxide group is a block copolymer of polyoxyethylene. In some embodiments, the polyalkylene oxide group is a block copolymer of polyoxypropylene.

Polyalkylene oxides may include but are not limited to poly ethoxylated group, poly propoxylated group, poly butoxylated group and any combination thereof.

Polyalkylene oxides may include but are not limited to copolymers and homogenous polymers.

Copolymers may include but are not limited to random polymer and block polymer.

In some embodiments, the polyalkylene oxide polyaryl ether is a polyalkylene oxide tristyryl phenyl ether. In some embodiments the polyalkylene oxide tristyryl phenyl ether is polyoxyethylene tristyryl phenyl ether. In some embodiments, the polyalkylene oxide tristyryl phenyl ether is polyoxyethylene polyoxypropylene tristyryl phenyl ether.

In some embodiments, the polyalkylene oxide polyaryl ether is a polyalkylene oxide distyryl phenyl ether. In some embodiments, the polyalkylene oxide distyryl phenyl ether is polyoxyethylene distyryl phenyl ether.

In some embodiments, non-ionic derivative of a polyalkylene oxide polyaryl ether is tristyrylphenol ethoxylate phosphate ester.

In some embodiments, the stabilizing surfactant is a derivative of tristyryl phenol-polyethylene glycol ether.

In some embodiments, the stabilizing surfactant is an anionic derivative of tristyryl phenol-polyethylene glycol ether.

In some embodiments, the stabilizing surfactant is a non-ionic derivative of tristyryl phenol-polyethylene glycol ether.

In some embodiments, the composition comprises two stabilizing surfactants and the two stabilizing surfactants are Soprophor® 3D33 and Soprophor® TS/54 (TSP 54).

In some embodiments, the composition comprises two stabilizing surfactants and both stabilizing surfactants are derivatives of polyalkylene oxide polyaryl ether. In some embodiments, the composition comprises two stabilizing surfactants wherein one stabilizing surfactant is a non-ionic derivative of polyalkylene oxide polyaryl ether and one stabilizing surfactant is an anionic derivative of polyalkylene oxide polyaryl ether.

In some embodiments, the composition comprises at least two stabilizing surfactants wherein at least one stabilizing surfactant is a non-ionic derivative of polyalkylene oxide polyaryl ether and at least one stabilizing surfactant is an anionic derivative of polyalkylene oxide polyaryl ether.

In some embodiments, the SC composition comprises two stabilizing surfactants and the two stabilizing surfactants are Soprophor® 3D33 and Soprophor® TS/54 (TSP 54).

In some embodiments, the SC composition comprises two stabilizing surfactants and both stabilizing surfactants are derivatives of polyalkylene oxide polyaryl ether. In some embodiments, the composition comprises two stabilizing surfactants wherein one stabilizing surfactant is a non-ionic derivative of polyalkylene oxide polyaryl ether and one stabilizing surfactant is an anionic derivative of polyalkylene oxide polyaryl ether.

In some embodiments, the SC composition comprises at least two stabilizing surfactants wherein at least one stabilizing surfactant is a non-ionic derivative of polyalkylene oxide polyaryl ether and at least one stabilizing surfactant is an anionic derivative of polyalkylene oxide polyaryl ether.

In some embodiments, the SE composition comprises two stabilizing surfactants and the two stabilizing surfactants are Soprophor® 3D33 and Soprophor® TS/54 (TSP 54).

In some embodiments, the SE composition comprises two stabilizing surfactants and both stabilizing surfactants are derivatives of polyalkylene oxide polyaryl ether. In some embodiments, the composition comprises two stabilizing surfactants wherein one stabilizing surfactant is a non-ionic derivative of polyalkylene oxide polyaryl ether and one stabilizing surfactant is an anionic derivative of polyalkylene oxide polyaryl ether.

In some embodiments, the SE composition comprises at least two stabilizing surfactants wherein at least one stabilizing surfactant is a non-ionic derivative of polyalkylene oxide polyaryl ether and at least one stabilizing surfactant is an anionic derivative of polyalkylene oxide polyaryl ether.

In some embodiments, stabilizing surfactant is Soprophor® 3D33.

In some embodiments, stabilizing surfactant is tristyrylphenol ethoxylate phosphate ester.

In some embodiments, the polyalkylene oxide polyaryl ether is Soprophor® 3D 33 from Solvay.

In some embodiments, the polyalkylene oxide polyaryl ether is Emulsogen® TS 540 from Clariant.

In some embodiments, the polyalkylene oxide polyaryl ether is Soprophor® TS/54 from Solvay.

In some embodiments, the salt comprising cation is selected from group consisting of sodium, potassium ammonium, calcium, magnesium and combination thereof.

Polyaryl may refer to but is not limited to poly phenyl ethyl phenol and tristyrylphenol.

Polyalkylene oxide polyaryl ethers surfactant refer to non-capped surfactants, end-capped surfactants or combination thereof.

In some embodiments, the combination of surfactants comprises a mixture of a nonionic polyalkylene oxide polyaryl ether surfactant and an anionic polyalkylene oxide polyaryl ether surfactant. In some embodiments, the nonionic surfactant is tristyrylphenol ethoxylate. In some embodiments, the anionic surfactant is tristyrylphenol ethoxylate phosphate ether.

In some embodiments, the combination of surfactants comprises tristyrylphenol ethoxylate and tristyrylphenol ethoxylate phosphate ether.

In some embodiments, the nonionic polyalkylene oxide polyaryl ether is a compound having an ether group substituted with at least two groups comprising aromatic rings.

In some embodiments, the polyalkylene oxide group is a polyoxyethylene.

In some embodiments, the polyalkylene oxide group is a polyoxypropylene. In some embodiments, the polyalkylene oxide group is a block copolymer of polyoxyethylene. In some embodiments, the polyalkylene oxide group is a block copolymer of polyoxypropylene.

Polyalkylene oxides may include but are not limited to poly ethoxylated group, poly propoxylated group, poly butoxylated group and any combination thereof.

Polyalkylene oxides may include but ae not limited to copolymers and homogenous polymers.

Copolymers may include but are not limited to random polymer and block polymer.

In some embodiments, the polyalkylene oxide polyaryl ether is a polyalkylene oxide tristyryl phenyl ether. In some embodiments the polyalkylene oxide tristyryl phenyl ether is polyoxyethylene tristyryl phenyl ether. In some embodiments, the polyalkylene oxide tristyryl phenyl ether is polyoxyethylene polyoxypropylene tristyryl phenyl ether.

In some embodiments, the polyalkylene oxide polyaryl ether is a polyalkylene oxide distyryl phenyl ether. In some embodiments, the polyalkylene oxide distyryl phenyl ether is polyoxyethylene distyryl phenyl ether.

In some embodiments, nonionic derivative of a polyalkylene oxide polyaryl ether is tristyrylphenol ethoxylate phosphate ester In some embodiments, stabilizing surfactant is Emulsogen® TS 540.

In some embodiments, nonionic derivative of surfactant is Emulsogen® TS 540.

In some embodiments, stabilizing surfactant is Soprophor® TS/54.

In some embodiments, nonionic derivative of a polyalkylene oxide polyaryl ether is Soprophor® TS/54.

In some embodiments, stabilizing surfactant is anionic derivative of tristyryl phenol-polyethylene glycol ether.

In some embodiments, stabilizing surfactant is nonionic derivative of tristyryl phenol-polyethylene glycol ether.

In some embodiments, the composition comprises a stabilizing system.

In some embodiments, the stabilizing surfactant is a dispersant. In some embodiments, the stabilizing surfactant is an emulsifier.

In some embodiments, the weight ratio of the non-ionic derivative of polyalkylene oxide polyaryl ether and the anionic derivative of polyalkylene oxide polyaryl ether is in the range of 0.25:1 to 1:1. In some embodiments, the weight ratio—of the non-ionic derivative of polyalkylene oxide polyaryl ether and the anionic derivative of polyalkylene oxide polyaryl ether is in the range of 0.25:1 to 0.5:1.

In some embodiments, the weight ratio of the non-ionic derivative of polyalkylene oxide polyaryl ether and the anionic derivative of polyalkylene oxide polyaryl ether is about 0.36:1.

In some embodiments, the stable composition comprises at least 0.5% by weight based on the total weight of the composition of the polyalkylene oxide polyaryl ether stabilizing surfactant(s). In some embodiments, the stable composition comprises from 0.5% to 7% by weight based on the total weight of the composition of the polyalkylene oxide polyaryl ether stabilizing surfactant(s). In some embodiments, the stable composition comprises from 0.5% to 15% by weight based on the total weight of the composition of the polyalkylene oxide polyaryl ether stabilizing surfactant(s). In some embodiments, the stable composition comprises from 0.5% to 25% by weight based on the total weight of the composition of the polyalkylene oxide polyaryl ether stabilizing surfactant(s).

In some embodiments, the weight ratio of the compound of Formula I to the non-ionic derivative of polyalkylene oxide polyaryl ether is from 25:1 to 10:1. In some embodiments, the weight ratio of the compound of Formula I to the anionic derivative of polyalkylene oxide polyaryl ether is from 25:1 to 10:1.

In embodiments, the stabilizing surfactant(s) is effective for increasing stability of the compound of Formula I in the compositions described herein compared to liquid composition wherein the compound of Formula I is soluble. In some embodiments, the stability is chemical stability. In some embodiments, the stability is physical stability.

(iii) Suitable pH Adjusters

In some embodiments, the composition comprises a pH adjuster.

In some embodiments, the pH adjusters may include but are not limited to buffers, bases and/or acidifiers.

In some embodiments the pH adjuster is an acid. In some embodiments the pH adjuster is a base.

In some embodiments the pH adjuster is a mixture of at least one base and at least one acid.

In some embodiments the pH adjuster is a buffer.

Buffers refer to combinations of acids and bases. Acids include but are not limited to organic and inorganic acids. Bases include but are not limited to organic and inorganic bases.

Organic acids may include but are not limited to citric acid, formic acid, acetic acid, propionic acid, butyric acid, oxalic acid, lactic acid, malic acid, and benzoic acid.

Inorganic acids may include but are not limited to hydrochloric acid, nitric acid, phosphoric acid, sulfuric acid and boric acid.

Organic bases may include but are not limited to primary and secondary amines, pyridines, imidazole and any combination thereof.

In some embodiments, the pH adjuster is potassium hydrogen phosphate.

In some embodiments, the pH adjuster is potassium dihydrogen phosphate. In some embodiments, the amount of potassium dihydrogen phosphate in the composition is 0.1-0.2% by weight based on the total weight of the composition. In some embodiments, the amount of potassium dihydrogen phosphate in the composition is about 0.14% by weight based on the total weight of the composition.

In some embodiments, the pH adjuster is disodium mono hydrogen phosphate. In some embodiments, the amount of disodium mono hydrogen phosphate in the composition is 0.5-0.6% by weight based on the total weight of the composition. In some embodiments, the amount of disodium mono hydrogen phosphate in the composition is about 0.57% by weight based on the total weight of the composition.

In some embodiments, the pH adjuster is combination of disodium mono hydrogen phosphate and potassium hydrogen phosphate. In some embodiments, the pH adjuster is combination of disodium mono hydrogen phosphate and potassium dihydrogen phosphate.

In some embodiments, the stable liquid composition further comprises a buffer. In some embodiments, the amount of the buffer in the stable composition is 1 g/L to 20 g/L. In some embodiments, the stable liquid composition further comprises a buffer. In some embodiments, the amount of the buffer in the stable composition is 6 g/L to 15 g/L. In some embodiments, the stable liquid composition further comprises a buffer.

In some embodiments, the amount of the buffer in the stable composition is 7 g/L to 10 g/L. In some embodiments, the concentration of the buffer in the stable composition is about 8.6 g/L.

In some embodiments the buffer is potassium dihydrogenorthophosphate. In some embodiments, the concentration of potassium dihydrogenorthophosphate in the stable liquid composition is 1 g/L to g/L. In some embodiments, the concentration of potassium dihydrogenorthophosphate in the stable liquid composition is 1 g/L to 3 g/L. In some embodiments, the concentration of potassium dihydrogenorthophosphate in the stable liquid composition is about 1.7 g/L.

In some embodiments the buffer is disodium phosphate anhydrous. In some embodiments, the concentration of disodium phosphate anhydrous in the stable liquid composition is 1 g/L to 10 g/L. In some embodiments, the concentration of disodium phosphate anhydrous in the stable liquid composition is 5 g/L to 10 g/L. In some embodiments, the concentration of disodium phosphate anhydrous in the stable liquid composition is 5 g/L to 8 g/L. In some embodiments, the concentration of disodium phosphate anhydrous in the stable liquid composition is about 6.9 g/L.

### (iv) Suitable Non-Aqueous Liquid Carriers

In some embodiments, the non-aqueous liquid carrier comprises one organic solvent.

In some embodiments, the non-aqueous liquid carrier comprises at least two organic solvents.

In some embodiments, the organic solvent is a non-aromatic solvent. In some embodiments, non-aromatic solvent is an aprotic solvent.

In some embodiments, organic solvent refers to co-solvent.

The solubility of the compound of Formula I in the solvent depends on the polarity of the solvent. In some embodiments, the polarity of the solvent between 25-50 (if water is 100). Solvents (non-aqueous liquid carriers) can be combined if the polarity of the combination of solvents is between 25-50. In some embodiments, the solubility of water in the solvent less than 25 g/l. In some embodiments, the solvent has a dipole (D) at 20° C. of less than 10, preferably less than 5. In some embodiments, the solvent has a Log P value of higher than 1.

In some embodiments, the non-aqueous liquid carrier is selected from a group consisting of aromatic hydrocarbons, paraffins, petroleum, diesel, mineral oil, ester and/or amide of fatty acids, tall oil fatty acids, and any combination thereof.

In some embodiments, the non-aqueous liquid carrier is an aromatic hydrocarbon.

In some embodiments, the aromatic hydrocarbon is selected from a group consisting of toluene, o-xylene, m-xylene, p-xylene, ethylbenzene, isopropylbenzene, tert-butylbenzene, naphthalenes, and mono- or polyalkyl-substituted naphthalenes.

In some embodiments, the organic solvent is a paraffin.

In some embodiments, the non-aqueous liquid carrier is a vegetable oil.

In some embodiments, the vegetable oil is selected from a group consisting of olive oil, kapok oil, castor oil, papaya oil, camellia oil, Canola oil, palm oil, sesame oil, corn oil, rice bran oil, peanut oil, cotton seed oil, soybean oil, rapeseed oil, linseed oil, tung oil, sunflower oil, safflower oil, and tall oil.

In some embodiments, the non-aqueous liquid carrier is an ester of a fatty acid. In some embodiments, the alkyl ester of the fatty acid is C18 methyl canolate ester. In some embodiments, the C18 methyl canolate ester is Agnique® ME 18 RD-F (manufactured and sold by BASF). In some embodiments, the non-aqueous liquid carrier is a C16-18 unsaturated fatty acid methyl ester. In some embodiments, the C16-18 unsaturated fatty acid methyl ester is Agnique® ME 18 RD-F (manufactured and sold by BASF). In some embodiments, the amount of Agnique® ME 18 RD-F in the OD composition is between 35-45% by weight based on the total weight of the composition. In some embodiments, the amount of Agnique® ME 18 RD-F in the OD composition is between 35-40% by weight based on the total weight of the composition. In some embodiments, the amount of Agnique® ME 18 RD-F in the OD composition is about 39% by weight based on the total weight of the composition. In some embodiments, the amount of Agnique® ME 18 RD-F in the OD composition is between 40-45% by weight based on the total weight of the composition. In some embodiments, the amount of Agnique® ME 18 RD-F in the OD composition about 41% by weight based on the total weight of the composition.

In some embodiments, the non-aqueous liquid carrier is an amide of a fatty acid. In some embodiments, the amide of the fatty acid is selected from a group consisting of C₁-C₃ amines, alkylamines and alkanolamines with C₆-C₁₈ carboxylic acids.

In some embodiments, the non-aqueous liquid carrier is an alkyl ester of a fatty acid. In some embodiments, the alkyl ester of the fatty acid is selected from a group consisting of C₁-C₄ monohydric alcohol esters of C₈ to C₂₂ fatty acids such as methyl oleate and ethyl oleate.

Other examples of non-aqueous liquid carriers are methyl fatty acid ester, plant oil alkyl ester, xylene, octanol, acetophenone, cyclohexanone, Solvesso™ (manufactured and sold by ExxonMobil Chemical), N-methyl pyrrolidone, tributyl sulphate (TBP), ethyl hexyl lactate (EHL), alkyl (linear or cyclic) amide of fatty acid (natural or synthetic), aryl acetate (benzyl acetate), polyethylene carbonate, benzyl acetate, and propylene carbonate. In some embodiments, the non-aqueous liquid carrier is cyclohexanone. In some embodiments, the non-aqueous liquid carrier is acetophenone. In some embodiments, the non-aqueous liquid carrier is benzyl acetate. In some embodiments, the non-aqueous liquid carrier is propylene carbonate.

### (v) Other Additives

Compositions of the present invention may further comprise one or more additional agriculturally acceptable inert additives, as known in the art, including but not limited to solid diluents, liquid diluents, wetting agents, adhesives, thickening agents, anti-foaming agent, preservative, wetting agent, anti-oxidation agent, binders, fertilizers, or anti-freeze agents. In addition, the present composition may also further comprise additional crop protection agents known in the art, for example pesticides, safeners, agents for controlling phytopathogenic fungi or bacteria, and the like.

In some embodiments, the liquid stable liquid composition further comprises a rheology modifier. Rheology modifiers may be used to reduce phases separation, to increase the physical stability, and to increase the viscosity which affect the chemical stability.

In some embodiments, the rheology modifier is Bentone SD®-1 (modified bentonite) or Bentone SD®-3 (modified hectorite) (manufactured by Elementis). In some embodiments, the amount of Bentone SD®-1 or Bentone SD®-3 in the composition is between 0.5 to 1.0% by weight. In some embodiments, the rheology modifier is Attagel® 50 (manufactured by BASF) and Bentone SD®-1. In some embodiments, the amount of Attagel® 50 in the composition is 0.5% by weight and the amount of Bentone SD®-1 in the composition is 0.5% by weight. Use of Attagel® 50 (0.5% by weight based on the total weight of the composition) and Bentone SD®-1 (0.5% by weight based on the total weight of the composition) decreased degradation of the compound of Formula I from 7-8% to 4% after 8 weeks of storage at 40° C. The water concentration of the composition should be maintained at less than 0.5%, including when Bentone SD®-1 is used as rheology modifier.

In some embodiments, the rheology modifier is xanthan gum.

In some embodiments, the rheology modifier is a thickener.

In some embodiments, the composition comprises one thickener. In some embodiments, the composition comprises two thickeners.

In some embodiments, the thickener is a silica thickener.

In some embodiments, the amount of thickener in the composition is less than or equal to 1% by weight based on the total weight of the composition. In some embodiments, the amount of thickener in the composition is less than or equal to 0.75% by weight based on the total weight of the composition. In some embodiments, the amount of thickener in the composition is less than or equal to 0.5% by weight based on the total weight of the composition. In some embodiments, the amount of thickener in the composition is less than or equal to 0.25% by weight based on the total weight of the composition. In some embodiments, the amount of thickener in the composition is less than or equal to 0.1% by weight based on the total weight of the composition.

In some embodiments, the amount of thickener in the SC composition is less than or equal to 1% by weight based on the total weight of the composition. In some embodiments, the amount of thickener in the SC composition is less than or equal to 0.75% by weight based on the total weight of the composition. In some embodiments, the amount of thickener in the aqueous composition is less than or equal to 0.5% by weight based on the total weight of the composition. In some embodiments, the amount of thickener in the SC composition is less than or equal to 0.25% by weight based on the total weight of the composition. In some embodiments, the amount of thickener in the SC composition is less than or equal to 0.1% by weight based on the total weight of the composition.

In some embodiments, the amount of thickener in the aqueous composition is less than or equal to 1% by weight based on the total weight of the composition. In some embodiments, the amount of thickener in the aqueous composition is less than or equal to 0.75% by weight based on the total weight of the composition. In some embodiments, the amount of thickener in the aqueous composition is less than or equal to 0.5% by weight based on the total weight of the composition. In some embodiments, the amount of thickener in the aqueous composition is less than or equal to 0.25% by weight based on the total weight of the composition. In some embodiments, the amount of thickener in the aqueous composition is less than or equal to 0.1% by weight based on the total weight of the composition.

In some embodiments, the amount of thickener in the non-aqueous composition is less than or equal to 1% by weight based on the total weight of the composition. In some embodiments, the amount of thickener in the non-aqueous composition is less than or equal to 0.75% by weight based on the total weight of the composition. In some embodiments, the amount of thickener in the non-aqueous composition is less than or equal to 0.5% by weight based on the total weight of the composition. In some embodiments, the amount of thickener in the non-aqueous composition is less than or equal to 0.25% by weight based on the total weight of the composition. In some embodiments, the amount of thickener in the non-aqueous composition is less than or equal to 0.1% by weight based on the total weight of the composition.

In some embodiments, the amount of thickener in the composition wherein the liquid carrier is non-aqueous is less than or equal to 5%—by weight based on the total weight of the composition. In some embodiments, the amount of thickener in the composition wherein the liquid carrier is non-aqueous is less than or equal to 2.5% by weight based on the total weight of the composition. In some embodiments, the amount of thickener in the composition wherein the liquid carrier is non-aqueous is less than or equal to 1% by weight based on the total weight of the composition. In some embodiments, the amount of thickener in the composition wherein the liquid carrier is non-aqueous is less than or equal to 0.75% by weight based on the total weight of the composition. In some embodiments, the amount of thickener in the composition wherein the liquid carrier is non-aqueous is less than or equal to 0.5% by weight based on the total weight of the composition. In some embodiments, the amount of thickener in the composition wherein the liquid carrier is non-aqueous is less than or equal to 0.25% by weight based on the total weight of the composition. In some embodiments, the amount of thickener in the composition wherein the liquid carrier is non-aqueous is less than or equal to 0.1% by weight based on the total weight of the composition.

In some embodiments, the thickener is a silica. In some embodiments, the thickener is a hydrophilic fumed silica. In some embodiments, the hydrophilic fumed silica is Aerosil® 200. In some embodiments, the hydrophilic fumed silica is Aerosil® R972.

In some embodiments, the thickener is a fumed silica surface-treated with polydimethylsiloxane. In some embodiments, the thickener is decamethylcyclopentasiloxane. In some embodiments, the fumed silica surface-treated with polydimethylsiloxane is Aerosil® R202. In some embodiments, the decamethylcyclopentasiloxane is Aerosil® R202. In some embodiments, the amount of Aerosil® R202 in the composition is between 0.1-5% by weight based on the total weight of the composition. In some embodiments, the amount of Aerosil® R202 in the composition is between 0.2-2.5% by weight based on the total weight of the composition. In some embodiments, the amount of Aerosil® R202 in the composition is between 0.1-0.3% by weight based on the total weight of the composition. In some embodiments, the amount of Aerosil® R202 in the composition is about 0.2% by weight based on the total weight of the composition. In some embodiments, the amount of Aerosil® R202 in the composition is between 2-3% by weight based on the total weight of the composition. In some embodiments, the amount of Aerosil® R202 in the composition is about 2.5% by weight based on the total weight of the composition.

In some embodiments, the thickener is selected from the group consisting of Aerosil® 200, Aerosil® R972, Aerosil® R202 and any combination thereof.

In some embodiments, the silica thickener is selected from the group consisting of Aerosil® R202, Aerosil® R812 and any combination thereof.

In some embodiments, the silica thickener is selected from the group consisting of Aerosil® R202, Aerosil® R812, magnesium aluminum silicate (VAN GEL® B) and any combination thereof.

In some embodiments, the amount of Aerosil® R202 in the composition is between 1% to 5% by weight based on the total weight of the composition. In some embodiments, the amount of Aerosil® R202 in the composition is between 1.7% to 2.5% by weight based on the total weight of the composition. In some embodiments, the amount of Aerosil® R202 in the composition is about 2.5% by weight based on the total weight of the composition. In some embodiments, the amount of Aerosil® R202 in the composition is less than 0.5% by weight based on the total weight of the composition. In some embodiments, the amount of Aerosil® R202 in the non-aqueous composition is less than 0.5% by weight based on the total weight of the composition.

In some embodiments, the amount of thickener in the OD composition is less than or equal to 0.5% by weight based on the total weight of the composition. In some embodiments, the amount of Aerosil® R202 in the OD composition is less than or equal to 0.5% by weight based on the total weight of the composition. In some embodiments, the amount of thickener in the OD composition is less than 0.5% by weight based on the total weight of the composition. In some embodiments, the amount of Aerosil® R202 in the OD composition is less than 0.5% by weight based on the total weight of the composition.

In some embodiments, the amount of thickener in the OD composition is 1-5% by weight based on the total weight of the composition. In some embodiments, the amount of thickener in the OD composition is 2-3% by weight based on the total weight of the composition. In some embodiments, the amount of thickener in the OD composition is about 2.5% by weight based on the total weight of the composition.

Viscosity of the composition is affected by the purity of the compound of Formula I, the form of the compound of Formula I, and the concentration of the compound of Formula I.

Physical stability, such as phase separation, of the composition is affected by the purity of the compound of Formula I, the form of the compound of Formula I, and the concentration of the compound of Formula I.

In some embodiments, the OD composition comprises a thickener such as Aerosil® R202 in an amount of 0.5% by weight based on the total weight of the composition.

In some embodiments, the OD composition comprises a thickener such as Aerosil® R202 in an amount of 2.5% by weight based on the total weight of the composition.

In some embodiments, the amount of Aerosil® R812 in the composition is between 1% to 5% by weight based on the total weight of the composition.

In some embodiments, the amount of Aerosil® R812 in the composition is between 3.0% to 3.5% by weight based on the total weight of the composition.

In some embodiments, the amount of Bentone SD®-1 or Bentone SD®-3 in the composition is between 0.5 to 1.0% by weight based on the total weight of the composition.

In some embodiments, the concentration of rheology modifier in the stable liquid composition is 1 g/L to 150 g/L. In some embodiments, the concentration of rheology modifier in the stable liquid composition is 1 g/L to 5 g/L. In some embodiments, the concentration of rheology modifier in the stable liquid composition is 2.3 g/L. In some embodiments, the concentration of rheology modifier in the stable liquid composition is from 0.5 g/L to 130 g/L. In some embodiments, the concentration of rheology modifier in the stable liquid composition is 3 g/L.

In some embodiments, the composition further comprises at least one adjuvant.

In some embodiments, the composition is mixed with at least one adjuvant.

In some embodiments, the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof; and
(v) sugar-based surfactants.

In some embodiments, the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii).

In some embodiments, the adjuvant is polyalkylene oxide alkyl ether.

In some embodiments, the adjuvant is siloxane polyalkyleneoxide copolymer.

In some embodiments, the adjuvant is esters of fatty acid.

In some embodiments, the adjuvant is vinylpyrrolidones and derivatives thereof.

In some embodiments, the adjuvant is sugar-based surfactants.

In some embodiments, the adjuvant is lignin.

In some embodiments, the adjuvant is terpene.

Preferred adjuvants are described in more detail below.

In some embodiments, the present invention composition further comprises additionally acceptable inert additives. In some embodiments, the agriculturally acceptable inert additives refer but are not limited to anti-oxidation agents, de-foaming agents, dye, pigment, flavoring agent, dispersing agent, synergists, encapsulates, photo-stabilizer, Binder, sticker, water soluble fertilizers, repellents and sensitizers.

In some embodiments, the agriculturally acceptable inert additive is a dispersant. In some embodiments, the agriculturally acceptable inert additive is an emulsifier.

In some embodiments, the agriculturally acceptable inert additive comprises water in an amount of less than 1% by weight based on the weight of the agriculturally acceptable inert additive.

In some embodiments, the agriculturally acceptable inert additive, when used in compositions with non-aqueous liquid carrier, comprises water in amount of less than 1% by weight based on the weight of the agriculturally acceptable inert additive.

In some embodiments, the water content is evaluated at the time the agriculturally acceptable inert additive is obtained. In some embodiments, the water content is evaluated at the time immediately prior to addition of the agriculturally acceptable inert additive to the admixture.

In some embodiments, the process further comprises adding least one dispersant.

In some embodiments, the stable liquid composition further comprises a dispersant agent. In some embodiments, the concentration of the dispersing agent in the stable liquid composition is from 1 g/L to 200 g/L.

In some embodiments, the stable liquid composition further comprises a wetting agent. In some embodiments, the wetting agent is sodium diisopropylnaphthalene sulphonate. In some embodiments, the concentration of the wetting agent in the stable composition is from 1 g/L to 10 g/L. In some embodiments, the concentration of the wetting agent in the stable composition is 5.5 g/L.

In some embodiments the wetting agent is polyalkyleneoxide modified heptamethyltrisiloxane. In some embodiments, the polyalkyleneoxide modified heptamethyltrisiloxane is Silwet L-77, manufactured and sold by Momentive. In some embodiments, the amount of Silwet L-77 in the SC composition is 0.1-0.5% by weight based on the total weight of the composition. In some embodiments, the amount of Silwet L-77 in the SC composition is 0.2-0.3% by weight based on the total weight of the composition. In some embodiments, the amount of Silwet L-77 in the SC composition is 0.25% by weight based on the total weight of the composition.

In some embodiments, the stable liquid composition further comprises a thickener agent. In some embodiments, the thickener agent is Xanthan gum. In some embodiments, the concentration of the thickener agent in the stable composition is from 0.25 g/L to 10 g/L. In some embodiments, the concentration of the thickener agent in the stable composition is 2 g/L.

In some embodiments, the stable liquid composition is an OD and the thickener agent is fumed silica.

In some embodiments, the stable liquid composition is an aqueous composition and the thickener agent is Xanthan gum. In some embodiments, the xanthan gum is AGRH 23 2% solution. In some embodiments, the amount of AGRH 23 2% solution in the SC composition is 7-8% by weight based on the total weight of the composition. In some embodiments, the amount of AGRH 23 2% solution in the SC composition is about 7.4% by weight based on the total weight of the composition.

In some embodiments, the stable liquid composition further comprises an anti-freeze agent. In some embodiments, the anti-freeze agent is 1,2-propanediol. In some embodiments, the concentration of anti-freeze agent in the stable composition is from 20 g/L to 70 g/L. In some embodiments, the concentration of antifreeze agent in the composition is 57.5 g/L.

In some embodiments, the anti-freeze agent is propylene glycol. In some embodiments, the amount of propylene glycol in the SC composition is 1-3% by weight based on the total weight of the composition. In some embodiments, the amount of propylene glycol in the SC composition is about 2% by weight based on the total weight of the composition.

In some embodiments, the stable liquid composition further comprises an anti-foaming agent. In some embodiments, the concentration of antifoaming agent in the stable composition is from 1 g/L to 5 g/L. In some embodiments the concentration of antifoaming agent in the composition is 2 g/L.

In some embodiment, the anti-foaming agent is a polydimethylsiloxane antifoam emulsion. In some embodiments, the polydimethylsiloxane antifoam emulsion is SAG 1572, manufactured and sold by Momentive. In some embodiments, the amount of SAG 1572 in the SC composition is 0.5-1.5% by weight based on the total weight of the composition. In some embodiments, the amount of SAG 1572 in the SC composition is about 1% by weight based on the total weight of the composition.

In some embodiments, the stable liquid composition further comprises an anti-oxidation agents. Anti-oxidation agents include but are not limited to clay, BHA, BHT, TBH, Propyl gallate, Sodium thiosulphate, Tocopherol, Pyrogallol and Epichlorohydrin.

In some embodiments, the stable liquid composition further comprises a defoaming agent. Defoaming agents include but are not limited to organosilicones, EO/PO based defoamers, alkyl polyacrylates.

In some embodiments, the stable liquid composition further comprises a dyes. Dyes include but are not limited to acid dye, basic dye, natural dye, synthetic dye and azo dye.

In some embodiments, the stable liquid composition further comprises a wetting agent. Examples of a wetting agent include but are not limited to di alkyl naphthalene sulfonate, di alkyl sulfosuccinate, metal salt of alkyl ether sulfonate, alpha olefin sulfonate, N-acyl N-alkyl taurate, linear alkyl benzene sulfonates, carboxylates, sulphates, phosphate esters, polyoxyethylene surfactants, ethoxylated alkyl phenols, ethoxylated aliphatic alcohols, anhydrosorbitol esters and cetyltrimethylammonium bromide.

In some embodiments, the stable liquid composition further comprises a surfactant.

Surfactants may include but are not limited to alcohol polyglycol ether, alkyl-end-capped ethoxylate glycol, alkyl-end-capped alkyl block alkoxylate glycol, dialkyl sulfosuccinate, phosphated esters, alkyl sulfonates, alkyl aryl sulfonates, tristyrylphenol alkoxylates, natural or synthetic fatty acid alkoxylates, natural or synthetic fatty alcohols alkoxylates, alkoxylated alcohols (such as n-butyl alcohol poly glycol ether), block copolymers (such as ethylene oxide-propylene oxide block copolymers and ethylene oxide-butylene oxide block copolymers) or combinations thereof.

In some embodiments, the surfactant is an alkyl-end-capped alkoxylate. In some embodiments, the adjuvant is a methyl-end-capped ethoxylate. In some embodiments, the adjuvant is a methyl-end-capped tridecyl ethoxylate. In some embodiments, the adjuvant is a methyl-end-capped tridecyl ethoxylate with six ethylene oxides.

In some embodiments the surfactant is di isopropyl naphthalene sulfonate.

In some embodiments, the composition comprises a biocide. In some embodiments, the amount of biocide in the SC composition is 0.05-0.1% by weight based on the total weight of the composition. In some embodiments, the amount of biocide in the SC composition is about 0.08% by weight based on the total weight of the composition.

In some embodiments, the amount of water in the SC composition is 35-45% by weight based on the total weight of the composition. In some embodiments, the amount of water in the SC composition is about 40% by weight based on the total weight of the composition.

In some embodiments the composition disclosed herein may include additional pesticide.

The disclosed compositions may optionally include combinations that can comprise at least 1% by weight of one or more of the compositions with another pesticidal compound. Such additional pesticidal compounds may be fungicides, insecticides, nematocides, miticides, arthropodicides, bactericides or combinations thereof that are compatible with the synergistic compositions of the present disclosure in the medium selected for application, and not antagonistic to the activity of the present compounds. Accordingly, in such embodiments the other pesticidal compound of Formula I s employed as a supplemental toxicant for the same or for a different pesticidal use. The pesticidal compound and the synergistic composition can generally be mixed together in a weight ratio of from 1:100 to 100:1.

In some embodiments, the composition is a formulation.

### Mixtures Comprising Crystalline Forms of the Compound of Formula I and Adjuvant(s)

Adjuvants are inert chemicals which are added for increasing performance of the active ingredient and composition thereof. Enhancing the activity of the compound of Formula I is particularly challenging because many drawbacks were observed such as rapidly drifting, high surface tension of the drops on the leaf, which dramatically affected and limited penetration into the plant.

It was found that applying at least one of the selected adjuvants with the compound of Formula (I) enhances efficacy of the compound of Formula (I) in controlling fungal attack on a plant. The selected adjuvant(s) may be built-into the compositions comprising the compound for Formula I. The selected adjuvant(s) may also be added into a tank mix comprising the compound for Formula I. Additionally, if more than one adjuvant is used, one or more of the adjuvant(s) may be built-into the composition while other adjuvant(s) are added to the tank mix.

The present invention provides a fungicidal mixture comprising:
(a) a fungicidally effective amount of a compound of Formula I:

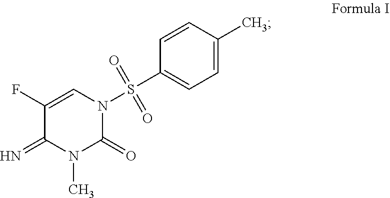 [SMILES: Cc1ccc(S(=O)(=O)n2cc(F)c(=N)n(C)c2=O)cc1]

wherein the compound of Formula I is in one or more forms and at least one form is Form III, IIIa, 01 or 02, and
(b) an adjuvant selected from the group consisting of:
   (i) polyalkylene oxide alkyl ether;
   (ii) siloxane polyalkyleneoxide copolymer;
   (iii) esters of fatty acid;
   (iv) vinylpyrrolidones and derivatives thereof;
   (v) sugar-based surfactants; and
   (vi) any combination of (i), (ii), (iii), (iv) and (v).

Any combination of (i), (ii), (iii), (iv) and (v) includes any combination of two, three, four, or five of (i), (ii), (iii), (iv) and (v), for example, (i) and (ii); (i) and (iii); (i) and (iv); (i) and (v); (ii) and (iii); (ii) and (iv); (ii) and (v); (iii) and (iv); (iii) and (v); (iv) and (v); (i), (ii), and (iii); (i), (ii), and (iv); (i), (ii), and (v); (i), (iii), and (iv); (i), (iii), and (v); (i), (iv), and (v); (i), (ii), (iii), and (iv); (i), (ii), (iii), and (v); (i), (ii), (iv), and (v); (i), (iii), (iv), and (v); (i), (ii), (iii), (iv), and (v); etc.

The present invention provides a fungicidal mixture comprising the following components:
(a) a fungicidally effective and substantially pure amount of a compound of Formula I or a fungicidally effective amount of a mixture containing a compound of Formula I:

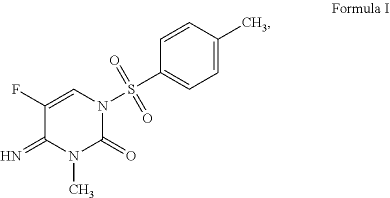 [SMILES: Cc1ccc(S(=O)(=O)n2cc(F)c(=N)n(C)c2=O)cc1]

wherein the compound of Formula I is in one or more forms and at least one form is Form III, IIIa, 01 or 02, and wherein 95% or more by weight of the mixture is the compound of Formula I; and
(b) an adjuvant selected from the group consisting of:
   (i) polyalkylene oxide alkyl ether;
   (ii) siloxane polyalkyleneoxide copolymer;
   (iii) esters of fatty acid;
   (iv) vinylpyrrolidones and derivatives thereof;
   (v) sugar-based surfactants; and
   (vi) any combination of (i), (ii), (iii), (iv) and (v).

The present invention provides a fungicidal mixture comprising:
(a) a fungicidally effective amount of a compound of Formula I:

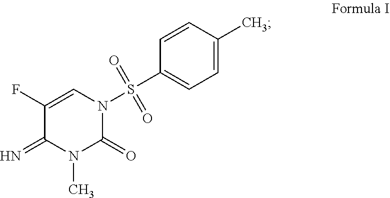 [SMILES: Cc1ccc(S(=O)(=O)n2cc(F)c(=N)n(C)c2=O)cc1]

wherein the compound of Formula I is in one or more forms and at least one form is Form III, IIIa, 01 or 02, and:
(b) an adjuvant selected from the group consisting of:
   (i) polyalkylene oxide alkyl ether;
   (ii) siloxane olyalkyleneoxide copolymer;
   (iii) esters of fatty acid;
   (iv) vinylpyrrolidones and derivatives thereof;
   (v) sugar-based surfactants; and
   (vi) any combination of (i), (ii), (iii), (iv), and (v),
wherein 94% or more of the amount of the compound of Formula I is in the form of Form III, Form IIIa, Form 01, Form 02, or a mixture thereof.

The present invention provides a fungicidal mixture comprising:
(a) a fungicidally effective amount of a compound of Formula I:

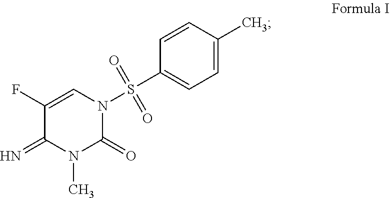 [SMILES: Cc1ccc(S(=O)(=O)n2cc(F)c(=N)n(C)c2=O)cc1]

wherein the compound of Formula I is in one or more forms and at least one form is Form III, IIIa, 01 or 02, and
(b) an adjuvant selected from the group consisting of:
   (i) polyalkylene oxide alkyl ether;
   (ii) siloxane polyalkyleneoxide copolymer;
   (iii) esters of fatty acid;
   (iv) vinylpyrrolidones and derivatives thereof;
   (v) sugar-based surfactants;
   (vi) lignins;
   (vii) terpens; and
   (viii) any combination of (i), (ii), (iii), (iv), (v), (vi) and (vii).

Any combination of (i), (ii), (iii), (iv), (v), (vi) and (vii) includes any combination of two, three four, five, six or seven of (i), (ii), (iii), (iv), (v), (vi) and (vii), for example, (i) and (ii); (i) and (iii); (i) and (iv); (i) and (v); (i) and (vi); (i) and (vii); (ii) and (iii); (ii) and (iv); (ii) and (v); (ii) and (vi); (ii) and (vii); (iii) and (iv); (iii) and (v); (iii) and (vi); (iii) and (vii); (iv) and (v); (iv) and (vi); (iv) and (vii); (v) and (vi); (v) and (vii); (vi) and (vii); (i), (ii), and (iii); (i), (ii), and (iv); (i), (ii), and (v); (i), (ii), (vi); (i), (ii), (vii); (i), (iii), and (iv); (i), (iii), and (v); (i), (iii), and (vi); (i), (iii), and (vii); (i), (iv), and (v); (i), (iv), and (vi); (i), (iv), and (vii); (i), (ii), (iii), and (iv); (i), (ii), (iii), and (v); (i), (ii), (iii), and (vi); (i), (ii), (iii), and (vii); (i), (ii), (iv), and (v); (i, (ii), (iv), and (vi); (i), (ii), (iv), and (vii), etc.

The present invention provides a fungicidal mixture comprising the following components:
(a) a fungicidally effective and substantially pure amount of a compound of Formula I or a fungicidally effective amount of a mixture containing a compound of Formula I:

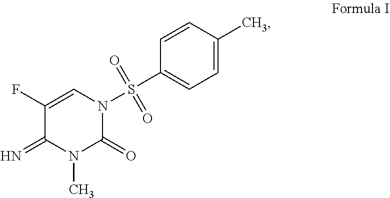 [SMILES: Cc1ccc(S(=O)(=O)n2cc(F)c(=N)n(C)c2=O)cc1]

wherein the compound of Formula I is in one or more forms and at least one form is Form III, IIIa, 01 or 02, and wherein 95% or more by weight of the mixture is the compound of Formula I; and
(b) an adjuvant selected from the group consisting of:
   (i) polyalkylene oxide alkyl ether;
   (ii) siloxane polyalkyleneoxide copolymer;
   (iii) esters of fatty acid;
   (iv) vinylpyrrolidones and derivatives thereof;
   (v) sugar-based surfactants;
   (vi) lignins;
   (vii) terpenes; and
   (viii) any combination of (i), (ii), (iii), (iv), (v), (vi) and (vii).

The present invention provides a fungicidal mixture comprising:
(a) a fungicidally effective amount of a compound of Formula I:

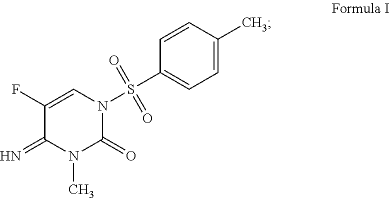 [SMILES: Cc1ccc(S(=O)(=O)n2cc(F)c(=N)n(C)c2=O)cc1]

wherein the compound of Formula I is in one or more forms and at least one form is Form III, IIIa, 01 or 02, and
(b) an adjuvant selected from the group consisting of:
   (i) polyalkylene oxide alkyl ether;
   (ii) siloxane polyalkyleneoxide copolymer;
   (iii) esters of fatty acid;
   (iv) vinylpyrrolidones and derivatives thereof;
   (v) sugar-based surfactants;
   (vi) lignins;
   (vii) terpenes; and
   (viii) any combination of (i), (ii), (iii), (iv), (v), (vi) and (vii),
      wherein 940 or more of the amount of the compound of Formula I is in the form of Form III, Form IIIa, Form 01, Form 02, or a mixture thereof.

In some embodiments, 95% or more by weight of the compound of Formula I in the mixture is in the form of Form III, Form IIIa, Form 01, Form 02, or a mixture thereof. In some embodiments, 96% or more by weight of the compound of Formula I in the mixture is in the form of Form III, Form IIIa, Form 01, Form 02, or a mixture thereof. In some embodiments, 97% or more by weight of the compound of Formula I in the mixture is in the form of Form III, Form IIIa, Form 01, Form 02, or a mixture thereof. In some embodiments, 98% or more by weight of the compound of Formula I in the mixture is in the form of Form III, Form IIIa, Form 01, Form 02, or a mixture thereof. In some embodiments, 99% or more by weight of the compound of Formula I in the mixture is in the form of Form III, Form IIIa, Form 01, Form 02, or a mixture thereof. In some embodiments, 99.5% or more by weight of the compound of Formula I in the mixture is in the form of Form III, Form IIIa, Form 01, Form 02, or a mixture thereof. In some embodiments, 99.9% by weight of the compound of Formula I in the mixture is in the form of Form III, Form IIIa, Form 01, Form 02, or a mixture thereof.

In some embodiments, the fungicidal mixture is a composition. In some embodiments, the fungicidal mixture is a tank mix.

In some embodiments, the compound of Formula I is in a composition. In some embodiments, the compound of Formula I is in a stable, liquid composition. The stable, liquid composition of the compound of Formula I includes, but is not limited to, the stable, liquid compositions disclosed herein. In some embodiments, the stable, liquid composition is a suspension concentrate (SC) composition. In some embodiments, the stable, liquid composition is a suspoemulsion (SE) composition. In some embodiments, the stable, liquid composition is an oil dispersion (OD) composition. In some embodiments, the stable, liquid composition is an emulsifiable concentrate (EC) composition.

In some embodiments, the polyalkylene oxide alkyl ether is poly alkoxylated alcohol.

In some embodiments, the alkyl of the polyalkylene oxide alkyl ether comprises, but is not limited to, carbohydrate chain comprising C1-C26.

In some embodiments, the alcohol of the poly alkoxylated alcohol comprises, but is not limited to, carbohydrate chain of C1-C26.

In some embodiments, the alkyl of the polyalkylene oxide alkyl ethers comprises, but is not limited to, short carbohydrate chain and long carbohydrate chain.

Carbohydrate chains may refer, but are not limited, to saturated, unsaturated, branched and unbranched chains.

In some embodiments, short chain refers to C1-C8. In some embodiments, long chain refers to C9-C26.

In some embodiments, the polyalkylene oxide refers but is not limited to polyethylene oxide, polypropylene oxide, polybutylene oxide or combinations thereof.

In some embodiments, the polyalkylene oxide includes but is not limited to copolymers. Copolymer refers to block co-polymers, such as polyethylene oxide-polypropylene oxide, and/or random co-polymers, such as ethylene oxide-propylene oxide. In some embodiments, the polyalkylene oxide block copolymer is di block copolymer. In some embodiments, the polyalkylene oxide block copolymer is tri block copolymer.

In some embodiments, the tri block copolymer is polyethylene oxide/polypropylene oxide/polyethylene oxide.

In some embodiments, the polyalkylene oxide alkyl ether is alkyl end capped. In some embodiments, the alkyl includes but is not limited to short carbohydrate chain and long carbohydrate chain. Carbohydrate chains may refer but are not limited to saturated, unsaturated, branched and unbranched chains. In some embodiments, short chain refers to C1-C8.

In some embodiments, the polyalkylene oxide alkyl ether is isotridecyl alcohol polyglycol ether.

In some embodiments, the polyalkylene oxide alkyl ether is C16-C18 alcohol ethoxylate propoxylate ether.

In some embodiments, the C16-C18 alcohol ethoxylate propoxylate ether is Ethylan™ 995 manufactured and sold by Akzo Nobel Agrochemicals. In some embodiments, the C16-C18 alcohol ethoxylate propoxylate ether is Agnique® BP420 manufactured and sold by BASF. In some embodiments, the amount of Agnique® BP420 in the EC composition is 5-6% by weight based on the total weight of the compositing. In some embodiments, the amount of Agnique® BP420 in the EC composition is about 5.5% by weight based on the total weight of the compositing.

In some embodiments, the polyalkylene oxide alkyl ether is ethoxylate propoxylate alcohol.

In some embodiments, the ethoxylate propoxylate alcohol is Synperonic-13/9 manufactured and sold by Croda. In some embodiments, the ethoxylate propoxylate alcohol is Atplus' PFA manufactured and sold by Croda.

In some embodiments, the polyalkylene oxide alkyl ether is iso-tridecyl alcohol polyglycol ether.

In some embodiments, the iso-tridecyl alcohol polyglycol ether is Genapol® X80 manufactured and sold by Clariant. In some embodiments, the iso-tridecyl alcohol polyglycol ether is Trycol® manufactured and sold by BASF.

In some embodiments, the polyalkylene oxide alkyl ether is ethoxylated isotridecyl alcohol. In some embodiments, the ethoxylated isotridecyl alcohol is Genapol®×050 manufactured and sold by Clariant. In some embodiments, the amount of ethoxylated isotridecyl alcohol in the composition is 1-10% by weight based on the total weight of the composition. In some embodiments, the amount of ethoxylated isotridecyl alcohol in the composition is 4-6% by weight based on the total weight of the composition. In some embodiments, the amount of ethoxylated isotridecyl alcohol in the composition is about 5% by weight based on the total weight of the composition.

In some embodiments, the polyalkylene oxide alkyl ether is effective for reducing surface tension of the composition and improving spreading of the compound of Formula I on plant leaf. Reducing the surface tension leads to reduced drifting from the leaf.

In some embodiments, the siloxane polyalkylene oxide copolymer as adjuvant comprises at least one organo modified trisiloxane. In some embodiments, the siloxane polyalkylene oxide copolymer as adjuvant comprises at least two organo modified trisiloxane.

In some embodiments, the siloxane polyalkylene oxide copolymer refers to organo modified trisiloxane.

In some embodiments, the siloxane polyalkylene oxide copolymer is Break-Thru® S233 from Evonik. In some embodiments, the siloxane polyalkylene oxide copolymer is Silwett® 077 from Momentive.

In some embodiments, the siloxane polyalkylene oxide copolymer is effective for reducing surface tension of the composition. Silicone surfactant was found efficient agent for reducing surface tension and rapidly spread on of the composition over lipophilic surfaces.

In some embodiments, the ester of fatty acid may include but is not limited to alkyl ester of fatty acid and plant oil. In some embodiments, esters of fatty acid is vegetable oil methyl esters.

In some embodiments, the alky ester comprising carbohydrate chain comprising C10-C20.

In some embodiments, the alkyl includes but is not limited to short carbohydrate chain.

Carbohydrate chains may refer but are not limited to saturated, unsaturated, branched and unbranched chains.

In some embodiments, short chain refers to C1-C8. In some embodiments, fatty acid alkyl ester is Rhodaphac® PA/23 from Solvay (phosphate ester of ethoxylated fatty alcohol) or Alkamuls® VO/2003 (ethoxylated (18EO) fatty acid) from Solvay.

In some embodiments, the adjuvant is tridecyl alcohol ethoxylated or polyoxyethylene (9) isotridecanol.

In some embodiments, plant oil includes but is not limited to vegetable oil and derivatives thereof.

In some embodiments, vegetable oil includes but is not limited to seed oil, coconut oil, rape seed oil, castor oil, soybean oil, palm oil and corn oil.

In some embodiments, derivative of vegetable oil refers to alkyl ester, poly alkylene oxide.

Polyalkylene oxide refers to polyethylene oxide, polypropylene oxide, polybutylene oxide and combination thereof.

In some embodiments, vegetable oil and derivatives thereof include but is not limited to rapeseed oil methylated ester and coconut fatty acid ester of polyglycerol ether.

In some embodiments, the adjuvant is a mixture of methylated seed oil and polyglycerol ester.

In some embodiments, the rapeseed oil methylated ester is Agnique® ME 18 RDF manufactured and sold by BASF.

In some embodiments, the polyalkylene oxide derivative of vegetable oil is coconut fatty acid ester of polyglycerol ether.

In some embodiments, the coconut fatty acid ester of polyglycerol ether is Synergen® GL5 manufactured and sold by Clariant.

In some embodiments, the ester of fatty acid soften the leaf's surface properties for better and efficient penetration of the compound of Formula I.

In some embodiments, the derivative of vinylpyrrolidones is a block copolymer of vinylpyrrolidone and vinyl acetate (VP/VA).

In some embodiments, the block copolymer of vinylpyrrolidone and vinyl acetate is Sokalan® VA 64 P manufactured and sold by Ashland.

In some embodiments, the block copolymer of vinylpyrrolidone and vinyl acetate is Agrimer™ VA 6 manufactured and sold by Ashland.

In some embodiments, the derivative of vinylpyrrolidones is a vinyl pyrrolidone copolymers like alkyl grafted PVP. In some embodiments, the vinyl pyrrolidone copolymers like alkyl grafted PVP is Agrimer AL-22 manufactured and sold by Ashland.

In some embodiments, the amount of the derivative of vinylpyrrolidones in the composition is 1-5% by weight based on the total weight of the composition. In some embodiments, the amount of the derivative of vinylpyrrolidones in the composition is 2-4% by weight based on the total weight of the composition. In some embodiments, the amount of the derivative of vinylpyrrolidones in the composition is about 3% by weight based on the total weight of the composition.

In some embodiments, the vinylpyrrolidones (PVP) and derivatives thereof are effective for increasing adherence of the compound of Formula I to plant leaves, for improvement of adhesive and retention properties (e.g. for rain fastness).

Sugar-based surfactants may include but are not limited to sorbitan esters, sucrose esters, alkyl polyglycosides, and fatty acid glucamides.

In some embodiments, the sugar-based surfactant is alkyl or fatty acid derivative of lglucamides.

In some embodiments, the sugar-based surfactant is alkylglucamides.

In some embodiments, the fatty acid glucamide is C8/C10 fatty acid glucose amide.

In some embodiments, the C8/C10 fatty acid glucose amide is Synergen® GA from Clariant.

In some embodiments, the sugar-based surfactant is sorbitan and derivatives thereof.

In some embodiments, the derivative of sorbitan is poly ethylene oxide derivative and fatty acid ester.

In some embodiments, the sorbitan is di or tri fatty acid ester. In some embodiments, the derivative of sorbitan is poly ethylene oxide derivative comprising 20 to 80 groups of ethylene oxide.

In some embodiments, the derivative of sorbitan is Tween® 80. In some embodiments, the derivative of sorbitan is Tween® 24 LM. In some embodiments, the amount of the derivative of sorbitan in the composition is 1-5% by weight based on the total weight of the composition. In some embodiments, the amount of the derivative of sorbitan in the composition is 3-4% by weight based on the total weight of the composition. In some embodiments, the amount of the derivative of sorbitan in the composition is 3.25% by weight based on the total weight of the composition.

Tween® 24 LM may also be used in the composition as a surfactant.

In some embodiments, the sugar-based surfactant affects the leaf surface for improving the penetration of the compound of Formula I through the leaf surface.

In some embodiments, the adjuvant is vegetable oil methyl esters.

In some embodiments, the lignins and terpenes are effective for increasing adherence of the compound of Formula I to plant leaves, for improvement of adhesive and retention properties (e.g. for rain fastness).

In some embodiments, the lignins and terpenes are wood-based products.

In some embodiments, the wood-based products are wood oil-based products. In some embodiments, the wood oil-based product is pine oil.

In some embodiments, the adjuvant is pine tree based lignins and terpenes. In some embodiments, the adjuvant is pine oil based lignins and terpenes. In some embodiments, the pine oil based lignins and terpenes comprises 50-60% oleic and linoleic acids, 34-40% rosins, and 5-10% long chain alcohols and sterols.

In some embodiments, the fungicidal mixture comprises a multi adjuvants system. Multi adjuvants system refers to blend or any combination of adjuvants.

In some embodiments, the fungicidal mixture comprises at least two adjuvants. In some embodiments, the fungicidal mixture comprises fatty acid esters and fatty alcohol alkoxylates.

In some embodiments, the fungicidal mixture comprises at least three adjuvants. In some embodiments, the fungicidal mixture comprises methylated seed oil, polyglycerol ester and alkoxylated alcohols.

In some embodiments, the adjuvants affect the penetration in different manner. In some embodiments, the adjuvants affect the penetration in the same manner.

In some embodiments, blend of adjuvant includes but is not limited to combination of alkyl fatty acid ester and fatty alcohol alkoxyklate.

In some embodiments, the combination of alkyl fatty acid ester and fatty alcohol alkoxylate is Synergen® SOC manufactured and sold by Clariant. In some embodiments, the amount of Synergen® SOC in the EC composition is 5-6% by weight based on the total weight of the composition. In some embodiments, the amount of Synergen® SOC in the EC composition is 5.5% by weight based on the total weight of the composition.

In some embodiments, the combination of alkyl fatty acid ester and fatty alcohol alkoxylate is FOP manufactured and sold by Clariant.

In some embodiments, a blend of adjuvants is used with Compound of Formula I. In some embodiments, at least two adjuvants for at least two improvements are used are used with Compound of Formula I.

In some embodiments, a blend of adjuvant includes but is not limited to combination of plant oil and/or derivative thereof and sugar-based surfactant. In some embodiments, a blend of adjuvant includes a combination of adjuvants for improvement of adhesive and retention properties (e.g. for rain fastness), improving spreading of the compound of Formula I on plant leaf, for reducing the surface tension which leads to reduced drifting from the leaf and for improving the penetration of the compound of Formula I through the leaf surface.

In some embodiments, more than one adjuvant is used for the same use such as improvement of adhesive and retention properties (e.g. for rain fastness), improving spreading of the compound of Formula I on plant leaf, reducing the surface tension which leads to reduced drifting from the leaf and/or for improving the penetration of the compound of Formula I through the leaf surface.

In some embodiments, the amount of compound (I) in the mixture is between 1-99.99% by weight.

In some embodiments, the amount of the adjuvant(s) in the mixture is between 0.01-95% by weight.

In some embodiments, the range of weight ratio of the compound of Formula I to the adjuvant(s) is 50:1 to 1:50. In some embodiments, the range of the weight ratio of the compound of Formula I to the adjuvant(s) is 10:1 to 1:10. In some embodiments, the range of the weight ratio of the compound of Formula I to the adjuvant(s) is 5:1 to 1:5. In some embodiments, the weight ratio of the compound of Formula I to the adjuvant(s) is 1:1.

In some embodiments, the range of the volume ratio of the compound of Formula I to the adjuvant(s) is 50:1 to 1:50. In some embodiments, the range of the volume ratio of the compound of Formula I to the adjuvant(s) is 10:1 to 1:10. In some embodiments, the range of the volume ratio of the compound of Formula I to the adjuvant(s) is 5:1 to 1:5. In some embodiments, the volume ratio of the compound of Formula I to the adjuvant(s) is 1:1.

In some embodiments, the weight ratio of the compound of Formula I to the adjuvant having the vinylpyrrolidones and derivative thereof structure is 25:1.

In some embodiments, the weight ratio of the compound of Formula I to the adjuvant having the siloxane polyalkyleneoxide copolymer structure is 50:1.

In one embodiment, the weight ratio between the polyalkylene oxide alkyl ether and compound of Formula I in the mixture is 1:90.

In one embodiment, the weight ratio between the plant oils and derivatives thereof and compound of Formula I in the mixture is 1:90.

In one embodiment, the weight ratio between the vinylpyrrolidones and derivative thereof and compound of Formula I in the mixture is 1:90.

In one embodiment, the weight ratio between the sugar-based surfactants and compound of Formula I in the mixture is 1:90.

In some embodiments, the range of the weight ratio between the two adjuvants is 5:1 to 1:5. In some embodiments, the weight ratio of between the two adjuvants is 2:1 to 1:2. In some embodiments, the weight ratio of between the two adjuvants is 1:1.

In some embodiments, the range of the weight ratio between the adjuvant having the vinylpyrrolidones and derivative thereof structure to the adjuvant having the siloxane polyalkyleneoxide copolymer structure is 5:1 to 1:5, In some embodiments, the weight ratio between the adjuvant having the vinylpyrrolidones and derivative thereof structure to the adjuvant having the siloxane polyalkyleneoxide copolymer structure is 2:1. In some embodiments, the weight ratio between the adjuvant having the vinylpyrrolidones and derivative thereof structure to the adjuvant having the siloxane polyalkyleneoxide copolymer structure is 1.4:1.

In some embodiments, the range of the weight ratio between the adjuvant having the vinylpyrrolidones and derivative thereof structure to the adjuvant having the polyalkylene oxide alkyl ether structure is 10:1 to 1:10. In some embodiments, the weight ratio between the adjuvant having the vinylpyrrolidones and derivative thereof structure to the adjuvant having the polyalkylene oxide alkyl ether structure is 1:5.5.

In some embodiments, the range of the weight ratio between the adjuvant having the vinylpyrrolidones and derivative thereof structure to the adjuvant having the ester of fatty acid structure is 5:1 to 1:5. In some embodiments, the weight ratio between the adjuvant having the vinylpyrrolidones and derivative thereof structure to the adjuvant having the ester of fatty acid structure is 1:3.7.

In some embodiments, the range of the weight ratio between the adjuvant having the polyalkylene oxide alkyl ether structure to the adjuvant having the ester of fatty acid structure is 5:1 to 1:5. In some embodiments, the range of the weight ratio between the adjuvant having the polyalkylene oxide alkyl ether structure to the adjuvant having the ester of fatty acid structure is 1.5:1.

In some embodiments, the range of the weight ratio between the adjuvant having the polyalkylene oxide alkyl ether structure to the adjuvant having the ester of fatty acid to the adjuvant having the vinylpyrrolidones and derivative thereof structure is 10:5:1 to 1:5:10.

In some embodiments, the range of the weight ratio between the adjuvant having the polyalkylene oxide alkyl ether structure to the adjuvant having the ester of fatty acid to the adjuvant having the vinylpyrrolidones and derivative thereof structure is 5.7:3.76:1.

In some embodiments, the weight ratio range of the two adjuvants in the multi adjuvants system is between 5:1 to 1:5, or 1:3 to 3:1, or 1:2 to 2:1 or 1:1.

In some embodiments, the weight ratio of the compound of Formula I and the adjuvant(s) is 5:1 to 1:5, or 1:3 to 3:1, or 1:2 to 2:1 or 1:1.

In some embodiments, the weight ratio of the compound of Formula I and the adjuvant(s) in the mixture is 5:1 to 1:5, or 1:3 to 3:1, or 1:2 to 2:1 or 1:1.

In some embodiments, in built-in the adjuvant is present in an amount of at least 0.1% by weight based on the total weight of the composition.

In some embodiments, the adjuvant is present in an amount of at least 10% by weight based on the total weight of the composition. In some embodiments, the adjuvant is present in an amount of at least 15% by weight based on the total weight of the composition. In some embodiments, the adjuvant is present in an amount of up to 30% by weight based on the total weight of the composition.

In some embodiments, the mixture of the present invention formulated as one composition, called built-in composition. In some embodiments, the mixture is formulated into two separate compositions and the composition are added in tank mix.

In some embodiments the ratio of adjuvant to compound (I) in tank mix is from 50:1 to 1:50.

In some embodiments, the range of the volume ratio of the compound of formula I to the adjuvant(s) is 50:1 to 1:50. In some embodiments, the range of the volume ratio of the compound of formula I to the adjuvant(s) is 10:1 to 1:10. In some embodiments, the range of the volume ratio of the compound of formula I to the adjuvant(s) is 5:1 to 1:5. In some embodiments, the volume ratio of the compound of formula I to the adjuvant(s) is 1:1.

In some embodiments, the concentration of the adjuvant having the structure of polyalkylene oxide alkyl ether in the composition/mixture is at least 3% by weight based on the total weight of the composition.

In some embodiments, the concentration of the adjuvant having the structure of siloxane polyalkyleneoxide copolymer in the composition/mixture is at least 5% by weight based on the total weight of the composition.

In some embodiments, the concentration of the adjuvant having the structure of ester of fatty acid in the composition/mixture is at least 3% by weight based on the total weight of the composition.

In some embodiments, the concentration of the adjuvant having the structure of vinylpyrrolidones and derivative thereof in the composition/mixture is between 0.1% to 2.5% by weight based on the total weight of the composition.

In some embodiments, the concentration of the adjuvant having the structure of sugar-based surfactant in the composition/mixture is at least 3% by weight based on the total weight of the composition.

In some embodiments, when a polyalkylene oxide alkyl ether concentration in the composition is less than 3% by weight based on the total weight of the composition, the polyalkylene oxide alkyl ether is used as the surfactant/emulsifier.

In this connection, when a siloxane polyalkyleneoxide copolymer concentration in the composition less than 5% by weight based on the total weight of the composition, the siloxane polyalkyleneoxide copolymer is used as the surfactant/emulsifier.

In this connection, when an ester of fatty acid concentration in the composition is less than 3% by weight based on the total weight of the composition, the ester of fatty acid is used as the surfactant/emulsifier.

In this connection, when a sugar-based surfactant concentration in the composition less than 3% by weight based on the total weight of the composition, the sugar-based surfactant is used as the surfactant/emulsifier.

In some embodiments, when the polyalkylene oxide alkyl ether, siloxane polyalkyleneoxide copolymer, ester of fatty acid and/or sugar-based surfactant is used as surfactant/emulsifier it is also used/functioned as adjuvant.

In some embodiment the compositions of compound (I) and/or adjuvant are liquid compositions, solid composition or combination thereof.

Example for liquid composition is a suspension concentration (SC) composition, an oil dispersion (OD) composition or an emulsifiable concentrate (EC) composition.

In one embodiment, the amount of polyalkylene oxide alkyl ether in the mixture of compound of Formula I with adjuvant(s) or in composition, ranges from about 1% to about 5% by weight based on the total weight of the composition.

In one embodiment, the amount of siloxane polyalkyleneoxide copolymer in the mixture of compound of Formula I with adjuvant(s) or in composition, ranges from about 1% to about 5% by weight based on the total weight of the composition.

In one embodiment, the amount of fatty acid alkyl esters in the mixture of compound of Formula I with adjuvant(s) or in composition, ranges from about 1% to about 5% by weight based on the total weight of the composition.

In one embodiment, the amount of plant oils and derivatives thereof in the mixture of compound of Formula I with adjuvant(s) or in composition, ranges from about 1% to about 5% by weight based on the total weight of the composition.

In one embodiment, the amount of vinylpyrrolidones and derivative thereof in the mixture of compound of Formula I with adjuvant(s) or in composition, ranges from about 1% to about 5% by weight based on the total weight of the composition.

In one embodiment, the amount of sugar-based surfactants in the mixture of compound of Formula I with adjuvant(s) or in composition, ranges from about 1% to about 5% by weight based on the total weight of the composition.

In one embodiment, the amount of polyalkylene oxide alkyl ether in the mixture of compound of Formula I with adjuvant(s) or in composition, ranges from about 1% to about 5% by weight based on the total weight of the composition.

In one embodiment, the amount of siloxane polyalkyleneoxide copolymer in the mixture of compound of Formula I with adjuvant(s) or in composition, ranges from about 1% to about 5% by weight based on the total weight of the composition.

In one embodiment, the amount of fatty acid alkyl esters in the mixture of compound of Formula I with adjuvant(s) or in composition, ranges from about 1% to about 5% by weight based on the total weight of the composition.

In one embodiment, the amount of plant oils and derivatives thereof in the mixture of compound of Formula I with adjuvant(s) or in composition, ranges from about 1% to about 5% by weight based on the total weight of the composition.

In one embodiment, the amount of vinylpyrrolidones and derivative thereof in the mixture of compound of Formula I with adjuvant(s) or in composition, ranges from about 1% to about 5% by weight based on the total weight of the composition.

In one embodiment, the amount of sugar-based surfactants in the mixture of compound of Formula I with adjuvant(s) or in composition, ranges from about 1% to about 5% by weight based on the total weight of the composition.

In one embodiment, the concentration of polyalkylene oxide alkyl ether in composition comprising compound of Formula I is 5% by weight based on the total weight of the composition.

In one embodiment, the concentration of siloxane polyalkyleneoxide copolymer in composition comprising compound of Formula I is 0.1% by weight based on the total weight of the composition.

In one embodiment, the concentration of fatty acid alkyl esters in composition of compound of Formula I is 5% by weight based on the total weight of the composition.

In one embodiment, the concentration of plant oils and derivatives thereof in composition comprising compound of Formula I is 6% by weight based on the total weight of the composition.

In one embodiment, the concentration of vinylpyrrolidones and derivative thereof in composition comprising compound of Formula I is 1.5% by weight based on the total weight of the composition.

In one embodiment, the concentration of sugar-based surfactant in composition comprising compound of Formula I is 5% by weight based on the total weight of the composition.

In one embodiment, the amount of polyalkylene oxide alkyl ether in the mixture of compound of Formula I with adjuvant(s) or in composition, ranges from about 1% to about 5% by weight based on the total weight of the composition.

In one embodiment, the amount of siloxane polyalkyleneoxide copolymer in the mixture of compound of Formula I with adjuvant(s) or in composition, ranges from about 1% to about 5% by weight based on the total weight of the composition.

In one embodiment, the amount of fatty acid alkyl esters in the mixture of compound of Formula I with adjuvant(s) or in composition, ranges from about 1% to about 5% by weight based on the total weight of the composition.

In one embodiment, the amount of plant oils and derivatives thereof in the mixture of compound of Formula I with adjuvant(s) or in composition, ranges from about 1% to about 5% by weight based on the total weight of the composition.

In one embodiment, the amount of vinylpyrrolidones and derivative thereof in the mixture of compound of Formula I with adjuvant(s) or in composition, ranges from about 1% to about 5% by weight based on the total weight of the composition.

In one embodiment, the amount of sugar-based surfactants in the mixture of compound of Formula I with adjuvant(s) or in composition, ranges from about 1% to about 5% by weight based on the total weight of the composition

In some embodiments, the concentration of VP/VA in the composition is about 1-3% by weight based on the total weight of the composition. In some embodiments, the concentration of VP/VA in the composition is about 1.5% by weight based on the total weight of the composition.

In some embodiments, the concentration of PVP in the composition is about 0.5-1.5% % by weight based on the total weight of the composition.

In some embodiments, the concentration of PVP in the composition is about 0.75-1.25% by weight based on the total weight of the composition.

In some embodiments, the concentration of siloxane polyalkyleneoxide copolymer in the composition is about 0.25-2.5% by weight based on the total weight of the composition. In some embodiments, the concentration of VP/VA in is about 0.1-2.0% by weight based on the total weight of the composition.

In some embodiments, the adjuvants in the multi adjuvant system have similar properties.

In some embodiments, the adjuvants in the multi adjuvant system have different properties.

In some embodiments, the adjuvant affects the leaf's surface properties.

In some embodiments, the adjuvant affects the composition's physical properties.

In some embodiments, one adjuvant or multi adjuvant system/blend affect the surface tension of the drop/composition/composition after dilution; acts as a sticking agent; improve the spreading of the compound of Formula I on the leaf.

In some embodiment, the penetration of compound of Formula I is increased by reducing the surface tension of the composition, thus spreads the composition on the leaf's surface and enhances penetration.

In some embodiments, the adjuvant used may also function as a solvent, surfactant, wetting agent, and/or dispersant.

In some embodiments, the solvent, surfactant, wetting agent, and/or dispersant used may also function as an adjuvant.

In some embodiments, Agnique® ME 18 RD-F (fatty acids, C16-18 and C18-unsaturated, methyl esters) is a solvent and a built-in adjuvant.

In some embodiments, Genapol® x80 (isotridecyl alcohol polyglycol ether nonionic surfactant) is an emulsifier/surfactant and a built-in adjuvant.

In some embodiments, Agnique® ME 18 RD-F (fatty acids, C16-18 and C18-unsaturated, methyl esters) is a solvent and a built-in adjuvant in an OD composition.

In some embodiments, Genapol® x80 (isotridecyl alcohol polyglycol ether nonionic surfactant) is an emulsifier/surfactant and a built-in adjuvant in an OD composition.

In some embodiments, solvent Agnique® ME 18 RD-F (fatty acids, C16-18 and C18-unsaturated, methyl esters) is also a built-in adjuvant.

In some embodiments, emulsifier/surfactant Genapol® x80 (isotridecyl alcohol polyglycol ether nonionic surfactant) is also a built-in adjuvant.

In some embodiments, solvent Agnique® ME 18 RD-F (Fatty acids, C16-18 and C18-unsaturated, methyl esters) in an OD composition is also a built-in adjuvant.

In some embodiments, emulsifier/surfactant Genapol® x80 (isotridecyl alcohol polyglycol ether nonionic surfactant) in an OD composition is also a built-in adjuvant.

The compositions are prepared according to procedures which are conventional in the agricultural chemical art, but which are novel and important because of the presence therein of the disclosed mixture of compound (I) and adjuvant.

Concentrated compositions of the disclosed mixture can be dispersed in water, or another liquid, for application, or compositions can be dust-like or granular, which can then be applied without further treatment or can be dilute before application.

The compositions that are applied most often are aqueous suspensions or emulsions. Either such water-soluble, water-suspendable, or emulsifiable compositions are solids, usually known as wettable powders, or liquids, usually known as emulsifiable concentrates, aqueous suspensions, suspension concentrates or suspoemulsions. The present disclosure contemplates all vehicles by which the mixture can be formulated for delivery and use as a fungicide.

In some embodiments, the stable liquid composition is an EC composition comprising a non-aqueous liquid carrier wherein the non-aqueous liquid carrier is acetophenone. In some embodiments, the amount of acetophenone in the EC composition is 55-65% by weight based on the total weight of the composition. In some embodiments, the amount of acetophenone in the EC composition is about 61% by weight based on the total weight of the composition.

Acetophenone is used the composition to improve stability of the composition at cold temperature, and particularly to minimize crystallization.

In some embodiments, the stable liquid composition is an EC composition comprising two adjuvants. In some embodiments, the two adjuvants are Agnique® BP420 and Synergen® SOC.

In some embodiments, the stable liquid composition is an EC composition comprising a fatty alcohol alkoxy ether. In some embodiments, the EC composition comprises fatty alcohol alkoxy ether. In some embodiments, the EC composition comprises at least one liquid carrier which is acetophenone and the composition comprises fatty alcohol alkoxy ether.

In some embodiments, the fatty alcohol alkoxy ether is fatty alcohol alkoxy ether. In some embodiments, the fatty alcohol alkoxy ether is Agnique® BP420. In some embodiments, the EC composition is mixed with fatty alcohol alkoxy ether. In some embodiments, the EC composition comprises at least one liquid carrier which is acetophenone and is mixed with fatty alcohol alkoxy ether. In some embodiments, the fatty alcohol alkoxy ether is fatty alcohol alkoxy ether. In some embodiments, the fatty alcohol alkoxy ether is Agnique® BP420.

As used herein, the term “mixed with” refers to as tank mix or separate applications.

In some embodiments, the EC composition comprises at least one fatty alcohol alkoxy ether. In some embodiments, the EC composition comprises at least one liquid carrier which is acetophenone and at least one fatty alcohol alkoxy ether. In some embodiments, the fatty alcohol alkoxy ether is fatty alcohol alkoxy ether. In some embodiments, the fatty alcohol alkoxy ether is Agnique® BP420. In some embodiments, the EC composition comprise at least one fatty alcohol alkoxy ether and/or at least one alkyl fatty acid ester. In some embodiments, the EC composition comprises Synergen® SOC. In some embodiments, the EC composition comprises Synergen® SOC and Agnique® BP420. In some embodiments, the EC composition comprises at least one liquid carrier which is acetophenone.

In some embodiments, the EC composition has a concentration of the compound of Formula I of 25-75 g/L. In some embodiments, the EC composition has a concentration of the compound of Formula I of 50 g/L.

In some embodiments, the EC composition is mixed with at least one fatty alcohol alkoxy ether and/or at least one alkyl fatty acid ester.

In some embodiments, the EC composition is mixed with Synergen® SOC.

In some embodiments, the EC composition is mixed with Synergen® SOC and Agnique® BP420.

As used herein, the term “mixed with” refers to as tank mix or separate applications.

In some embodiments, the stable liquid composition is an OD composition comprising one adjuvant. In some embodiments, the adjuvant is Genapol® X050. In some embodiments, the adjuvant is iso-tridecyl alcohol polyglycol ether (5 EO).

In some embodiments, the OD composition comprises at least one fatty alcohol alkoxy ether.

In some embodiments, at least one of the fatty alcohol alkoxy ether is iso-tridecyl alcohol polyglycol ether (5 EO).

In some embodiments, the iso-tridecyl alcohol polyglycol ether (5 EO) is Genapol® X50.

In some embodiments, the OD composition comprises a fatty acid ester.

In some embodiments, the fatty acid ester is Agnique® RD-F18.

In some embodiments, the Agnique® RD-F18 functions both as a solvent and as an adjuvant.

In some embodiments, the fatty acid ester is C16-18 and C18-unsaturated, methyl esters.

In some embodiments, the OD composition comprises at least one fatty alcohol alkoxy ether and at least one fatty acid ester.

In some embodiments, the OD composition comprises C16-18 and C18-unsaturated, methyl esters and Genapol® X50 (iso-tridecyl alcohol polyglycol ether (5 EO)).

In some embodiments, the OD composition comprises at least one dispersant. In some embodiments, the dispersant is polymeric. In some embodiments, the dispersant is selected from the group consisting of Atlox™ 4912, Atlox™ 4915, Atlox™ 4916, Agrimer™ AL22, and any combination thereof.

In some embodiments, the OD composition comprises at least one polymeric dispersant.

Polymeric dispersants that may be used in connection with the present invention include, but are not limited to, Atlox™ 4912, Atlox™ 4915, Atlox™ 4916 and Agrimer™ AL22. In some embodiments, the polymeric dispersant is selected from the group consisting of Atlox™ 4912, Atlox™ 4915, Atlox™ 4916, Agrimer™ AL22, and any combination thereof.

In some embodiments, the dispersant is other than Soprophor® 3D33. In some embodiments, the dispersant is other than Soprophor® 4D384. In some embodiments, the dispersant is Soprophor® 3D33. In some embodiments, the dispersant is Soprophor® 4D384. In some embodiments, wherein the composition is OD, the dispersant is other than Soprophor® 3D33. In some embodiments, wherein the composition is OD, the dispersant is other than Soprophor® 4D384. In some embodiments, wherein the liquid carrier in the composition is an aqeuous liquid carrier, the dispersant is Soprophor® 3D33. In some embodiments, wherein the liquid carrier in the composition is an aqeuous liquid carrier, the dispersant is Soprophor® 4D384.

In some embodiments, the OD composition comprises at least one emulsifier.

Emulsifiers that may be used in connection with present invention include, but is not limited to, Aerosol® OT-SE ULA, Rhodocal® 70/B, Synperonic™ PE/L 64, Atlas' G5002, Genapol® X50, Genapol® X80 and Alkamils AP. In some embodiments, the emulsifier is selected from the group consisting of Aerosol® OT-SE ULA, Rhodocal® 70/B, Synperonic™ PE/L 64, Atlas' G5002, Genapol® X50, Genapol® X80, Alkamils AP, and any combination thereof.

In some embodiments, the emulsifier is Aerosol® OT-SE. In some embodiments, the amount of Aerosol® OT-SE in the composition is 1-10% by weight based on the total weight of the composition. In some embodiments, the amount of Aerosol® OT-SE in the composition is 5-7% by weight based on the total weight of the composition. In some embodiments, the amount of Aerosol® OT-SE in the composition is about 6% by weight based on the total weight of the composition.

In some embodiments, the emulsifier is Synperonic™ PE/L 64. In some embodiments, the amount of Synperonic™ PE/L 64 in the composition is 1-10% by weight based on the total weight of the composition. In some embodiments, the amount of Synperonic™ PE/L 64 in the composition is 5-7% by weight based on the total weight of the composition. In some embodiments, the amount of Synperonic™ PE/L 64 in the composition is about 6% by weight based on the total weight of the composition.

In some embodiments, the emulsifier comprises castor oil. In some embodiments, the emulsifier comprises TSP 16. In some embodiments the emulsifier comprises sorbitan trioleate (span type). In some embodiments, the emulsifier is tristyrylphenol ethoxylate. In some embodiments, the tristyrylphenol ethoxylate is Soprophor® TS/16. In some embodiments, the amount of Soprophor® TS/16 in the EC composition is 20-25% by weight based on the total weight of the composition. In some embodiments, the amount of Soprophor® TS/16 in the EC composition is 22-24% by weight based on the total weight of the composition. In some embodiments, the amount of Soprophor® TS/16 in the EC composition is about 23% by weight based on the total weight of the composition.

In some embodiments, the OD composition comprises ethylene oxide/propylene oxide block copolymer. The ethylene oxide/propylene oxide block copolymer can function as a surfactant and an emulsifier. In some embodiments, the ethylene oxide/propylene oxide block copolymer is Synperonic™ PE/L 64.

In some embodiments, the OD composition comprises a water scavenger. In some embodiments, the OD composition comprises 0.25-0.75% by weight of the water scavenger based on the total weight of the composition.

In some embodiments, the OD composition comprises 0.5% by weight of the water scavenger based on the total weight of the composition. In some embodiments, the water scavenger is tetraethyl orthosilicate.

In some embodiments, the OD composition has a concentration of the compound of Formula I of 200-300 g/L. In some embodiments, the OD composition has a concentration of the compound of Formula I of 250 g/L. In some embodiments, the OD composition has a concentration of the compound of Formula I of at least 200 g/L.

The present invention also provides an oil dispersion (OD) composition comprising:
(a) a fungicidally effective amount of a compound of Formula I:

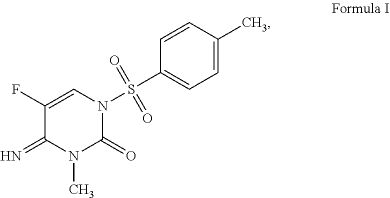 [SMILES: Cc1ccc(S(=O)(=O)n2cc(F)c(=N)n(C)c2=O)cc1]

(b) a non-aqueous liquid carrier, and
wherein:
   a) the composition further comprises 5% by weight of tetraethyl orthosilicate based on the total weight of the composition, and/or epoxidized soybean oil, and
   b) the concentration of the compound of Formula I in the composition is 250 g/L.

In some embodiments, the stable liquid composition is a SC composition. In some embodiments, the SC composition comprises one adjuvant. In some embodiments, the adjuvant is Silwett® 077.

In some embodiments, the stable liquid composition is an SC composition and the amount of the compound of Formula I in the SC composition is 400 g/L to 600 g/L. In some embodiments, the stable liquid composition is an SC composition and the amount of the compound of Formula I in the SC composition is 500 g/L. In some embodiments, the SC composition does not comprise VP/VA. In some embodiments, the SC composition does not comprise Van Gel® B. In some embodiments, the SC composition does not comprise magnesium aluminum silicate.

In some embodiments, the SC composition is formulated free of adjuvant.

In some embodiments, the SC composition comprises at least one adjuvant.

In some embodiments, the adjuvant is siloxane polyalkyleneoxide copolymer.

In some embodiments, the siloxane is Silwett® 177.

In some embodiments, the SC composition is mixed with one adjuvant.

In some embodiments, the adjuvant is siloxane polyalkyleneoxide copolymer.

In some embodiments, the siloxane polyalkyleneoxide copolymer comprises at least one derivative of siloxane polyalkyleneoxide copolymer.

In some embodiments, the siloxane polyalkyleneoxide copolymer comprises at least two derivatives of siloxane polyalkyleneoxide copolymer

In some embodiments, the siloxane adjuvant is Silwett® 1-77.

In some embodiments, the SC composition comprises siloxane polyalkyleneoxide copolymer and xanthan gum. In some embodiments, the SC composition comprises siloxane polyalkyleneoxide copolymer and disodium phosphate anhydrous. In some embodiments, the SC composition comprises siloxane polyalkyleneoxide copolymer and tristyryl phenol-polyethylene glycol ether. In some embodiments, the SC composition comprises siloxane polyalkyleneoxide copolymer and potassium dihydrogene phosphate. In some embodiments, the SC composition comprises siloxane polyalkyleneoxide copolymer and 1,2-propanediol. In some embodiments, the SC composition comprises siloxane polyalkyleneoxide copolymer and Proxel GXL. In some embodiments, the SC composition comprises siloxane polyalkyleneoxide copolymer and polydimethylsiloxane emulsion. In some embodiments, the SC composition comprises siloxane polyalkyleneoxide copolymer and Soprophor® 3D33. In some embodiments, the SC composition comprises siloxane polyalkyleneoxide copolymer and Supragil WP.

In some embodiments, the SC composition comprises sodium diisopropylnaphthalenesulphonate. In some embodiments, the sodium diisopropylnaphthalenesulphonate is Supragil WP. In some embodiments, the amount of Supragil WP in the composition is 0.1-1% by weight based on the total weight of the composition. In some embodiments, the amount of Supragil WP in the composition is about 0.5% by weight based on the total weight of the composition.

In some embodiments, the SC composition comprises two adjuvants.

In some embodiments, the two adjuvants are at least one siloxane polyalkyleneoxide copolymer and VP/VA.

In some embodiments, the SC composition comprises at least one siloxane polyalkyleneoxide copolymer and is mixed with at least one additional adjuvant.

In some embodiments, the SC composition is mixed with at least one adjuvant.

In some embodiments, the SC composition is mixed with at least two adjuvants.

In some embodiments, the adjuvant is VP/VA.

In some embodiments, the SC composition is mixed with Trycol®.

As used herein, the term “mixed with” refers to as tank mix or separate applications. In some embodiments, the SC composition comprises VP/VA.

In some embodiments, the SC composition comprise VP/VA as an adjuvant.

In some embodiments, the SC composition comprises the compound of Formula I at a concentration of at least 40% by weight based on the total weight of the composition. In some embodiments, the SC composition comprises the compound of Formula I at a concentration of at least 50% by weight based on the total weight of the composition.

In some embodiments, the SC composition comprises the compound of Formula I and VP/VA wherein the concentration of the compound of Formula I is at least 40% by weight based on the total weight of the composition.

In some embodiments, the SC composition comprises the compound of Formula I and is formulated in the absence of VP/VA, wherein the concentration of the compound of Formula I is at least 40% by weight based on the total weight of the composition.

In some embodiments, the SC composition comprises the compound of Formula I and VP/VA, wherein the concentration of the compound of Formula I is at least 50% by weight based on the total weight of the composition.

In some embodiments, the SC composition comprises the compound of Formula I and is formulated in the absence of VP/VA, wherein the concentration of the compound of Formula I is at least 50% by weight based on the total weight of the composition.

In some embodiments, the SC composition comprises the compound of Formula I and is formulated in the absence of VP/VA, wherein the concentration of the compound of Formula I is at least 45% by weight based on the total weight of the composition.

In some embodiments, the concentration of the compound of Formula I in the SC composition is greater than or equal to 45% by weight based on the total weight of the composition. In some embodiments, the concentration of the compound of Formula I in the SC composition is greater than or equal to 50% by weight based on the total weight of the composition. In some embodiments, the concentration of the compound of Formula I in the SC composition is greater than or equal to 55% by weight based on the total weight of the composition. In some embodiments, the concentration of the compound of Formula I in the SC composition is greater than or equal to 60% by weight based on the total weight of the composition. In some embodiments, the concentration of the compound of Formula I in the SC composition is greater than 65% by weight based on the total weight of the composition.

In some embodiments, 95% or more of the amount of the compound of Formula I used in the SC composition is in Form III, Form IIIa, Form 01, Form 02 or a mixture thereof. In some embodiments, 96% or more of the amount of the compound of Formula I used in the SC composition is in Form III, Form IIIa, Form 01, Form 02 or a mixture thereof. In some embodiments, 97% or more of the amount of the compound of Formula I used in the SC composition is in Form III, Form IIIa, Form 01, Form 02 or a mixture thereof. In some embodiments, 98% or more of the amount of the compound of Formula I used in the SC composition is in Form III, Form IIIa, Form 01, Form 02 or a mixture thereof. In some embodiments, 99% or more of the amount of the compound of Formula I used in the SC composition is in Form III, Form IIIa, Form 01, Form 02 or a mixture thereof. In some embodiments, 99.5% or more of the amount of the compound of Formula I used in the SC composition is in Form III, Form IIIa, Form 01, Form 02 or a mixture thereof. In some embodiments, 100% of the amount of the compound of Formula I used in the SC composition is in Form III, Form IIIa, Form 01, Form 02 or a mixture thereof.

The present invention also provides a suspension concentrate (SC) composition comprising:
(a) a fungicidally effective amount of a compound of Formula I:

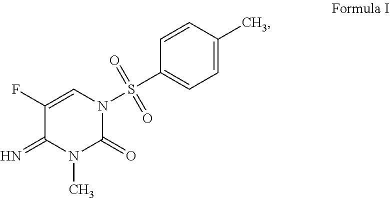 [SMILES: Cc1ccc(S(=O)(=O)n2cc(F)c(=N)n(C)c2=O)cc1]

and
(b) an aqueous liquid carrier,
wherein the composition is free of the block copolymer of vinylpyrrolidone and vinyl acetate (VP/VA).

The present invention also provides a suspension concentrate (SC) composition comprising:
(a) a fungicidally effective amount of a compound of Formula I:

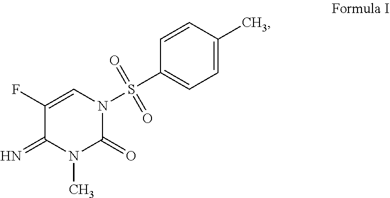 [SMILES: Cc1ccc(S(=O)(=O)n2cc(F)c(=N)n(C)c2=O)cc1]

and
(b) an aqueous liquid carrier,
wherein the composition is free of magnesium aluminum silicate.

The present invention also provides a suspension concentrate (SC) composition comprising:
(a) a fungicidally effective amount of a compound of Formula I:

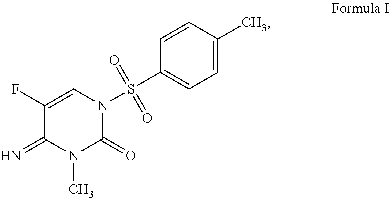 [SMILES: Cc1ccc(S(=O)(=O)n2cc(F)c(=N)n(C)c2=O)cc1]

and
(b) an aqueous liquid carrier,
wherein the composition comprises siloxane polyalkyleneoxide copolymer at a concentration of 0.01-0.8% by weight based on the total weight of the composition.

In some embodiments, the concentration of siloxane polyalkyleneoxide copolymer in the composition is 0.01-0.5% by weight based on the total weight of the composition.

In some embodiments, the concentration of siloxane polyalkyleneoxide copolymer in the composition is 0.1-0.3% by weight based on the total weight of the composition. In some embodiments, the concentration of siloxane polyalkyleneoxide copolymer in the composition is about 0.2% by weight based on the total weight of the composition.

In some embodiments, the stable liquid composition is an EC composition and greater than 96% of the amount of the compound of Formula I is in Form III, Form IIIa, Form 01, Form 02 or a mixture thereof. In some embodiments, the stable liquid composition is an OD composition and greater than 96% of the amount of the compound of Formula I is in Form III, Form IIIa, Form 01, Form 02 or a mixture thereof. In some embodiments, the stable liquid composition is a SC composition and greater than 96% of the amount of the compound of Formula I is in Form III, Form IIIa, Form 01, Form 02 or a mixture thereof. In some embodiments, the stable liquid composition is a SE composition and greater than 96% of the amount of the compound of Formula I is in Form III, Form IIIa, Form 01, Form 02 or a mixture thereof.

The present invention also provides an emulsifiable concentrate (EC) composition comprising:
(a) a fungicidally effective amount of a compound of Formula I:

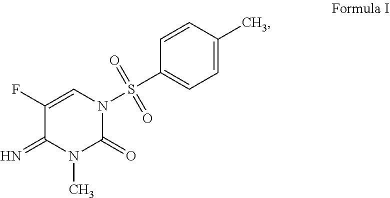 [SMILES: Cc1ccc(S(=O)(=O)n2cc(F)c(=N)n(C)c2=O)cc1]

and
wherein:
a) the composition comprises at least one fatty alcohol alkoxy ether and/or at least one alkyl fatty acid ester, and/or
b) the concentration of the compound of Formula I in the composition is 50 g/L.

The present invention provides an EC composition comprising (i) a compound of Formula I, (ii) acetophenone, (iii) Agnique® BP420, and (iv) Synergen® SOC.

The present invention provides an OD composition comprising (i) a compound of Formula I and (ii) Genapol® X050.

The present invention provides an OD composition comprising (i) a compound of Formula I and (ii) iso-tridecyl alcohol polyglycol ether (5 EO).

The present invention provides a SC composition comprising (i) a compound of Formula I, and (ii) Silwett® 077, wherein the concentration of the compound of Formula I in the composition is 500 g/L and wherein the composition is free of VP/VA and van gel.

The present invention also provides a composition comprising a compound of Formula I and a non-aqueous liquid carrier, wherein the non-aqueous liquid carrier has a water content equal to or less than 0.2% by weight based on the weight of the non-aqueous liquid carrier.

The present invention also provides a composition comprising a compound of Formula I, a non-aqueous liquid carrier, and at least one agriculturally acceptable inert additive, wherein (1) the non-aqueous liquid carrier has a water content equal to or less than 0.2% by weight based on the weight of the non-aqueous liquid carrier, and (2) the agriculturally acceptable inert additive has a water content of less than 1% by weight based on the weight of the agriculturally acceptable inert additive. In some embodiments, the water content is evaluated at the time the agriculturally acceptable inert additive is obtained. In some embodiments, the water content is evaluated at the time immediately prior to addition of the agriculturally acceptable inert additive to the admixture.

The present invention also provides a mixture comprising (i) any one of the SC compositions described herein, and (ii) Trycol®.

The present invention also provides a method for reducing crystallization of a compound of Formula I in a composition under cold temperature comprising using acetophenone as a solvent in the composition.

As used herein, the term “cold temperature” means a temperature below room temperature, i.e. below 20° C.

In some embodiments, the temperature is between −10° C. to 20° C. In some embodiments, the temperature is between 0° C. to 20° C. In some embodiments, the temperature is between 0° C. to 15° C. In some embodiments, the temperature is between 5° C. to 15° C. In some embodiments, the temperature is 0° C.

The present invention also provides a method for reducing viscosity of an SC composition comprising a compound of Formula I, wherein the method comprises formulating the composition to be free of VP/VA.

The present invention also provides a method for reducing viscosity of an SC composition comprising a compound of Formula I, wherein the method comprises formulating the composition to be free of Van Gel® B or magnesium aluminum silicate.

### Additional Agrochemicals

The mixtures and compositions of the present invention may further comprise one or more additional agrochemicals.

In some embodiments, the composition of the present invention further comprises at least one additional pesticide. In some embodiments, the pesticide is a fungicide, herbicide, insecticide, or nematicide.

In some embodiments, the composition of the present invention further comprises at least one additional fungicide. In some embodiments, the fungicidal mixture of the present invention further comprises at least one additional fungicide.

In some embodiments, the at least one additional fungicide is a fungicidal sterol biosynthesis inhibitor.

In some embodiments, the sterol biosynthesis inhibitor is selected from the group consisting of prothioconazole, epoxiconazole, cyproconazole, myclobutanil, prochloraz, metconazole, difenoconazole, tebuconazole, tetraconazole, fenbuconazole, propiconazole, fluquinconazole, flusilazole, flutriafol, and fenpropimorph.

In some embodiments, the sterol biosynthesis inhibitor is selected from the group consisting of prothioconazole, epoxiconazole, metconazole, difenoconazole, propiconazole, prochloraz, tetraconazole, tebuconazole, fenpropimorph, fenpropidin, ipconazole, triticonazole, spiroxamine, fenhexamid, and fenpyrazamine.

In some embodiments, the sterol biosynthesis inhibitor is prothioconazole. In some embodiments, the sterol biosynthesis inhibitor is epoxiconazole. In some embodiments, the sterol biosynthesis inhibitor is cyproconazole. In some embodiments, the sterol biosynthesis inhibitor is myclobutanil. In some embodiments, the sterol biosynthesis inhibitor is metconazole. In some embodiments, the sterol biosynthesis inhibitor is difenoconazole. In some embodiments, the sterol biosynthesis inhibitor is propiconazole. In some embodiments, the sterol biosynthesis inhibitor is prochloraz. In some embodiments, the sterol biosynthesis inhibitor is tetraconazole. In some embodiments, the sterol biosynthesis inhibitor is tebuconazole. In some embodiments, the sterol biosynthesis inhibitor is fluquinconazole. In some embodiments, the sterol biosynthesis inhibitor is flusilazole. In some embodiments, the sterol biosynthesis inhibitor is flutriafol. In some embodiments, the sterol biosynthesis inhibitor is fenpropimorph. In some embodiments, the sterol biosynthesis inhibitor is fenpropidin. In some embodiments, the sterol biosynthesis inhibitor is ipconazole. In some embodiments, the sterol biosynthesis inhibitor is triticonazole. In some embodiments, the sterol biosynthesis inhibitor is spiroxamin. In some embodiments, the sterol biosynthesis inhibitor is fenhexamid. In some embodiments, the sterol biosynthesis inhibitor is fenpyrazamine. In some embodiments, the sterol biosynthesis inhibitor is fenbuconazole.

In some embodiments, the at least one additional fungicide is a succinate dehydrogenase inhibitor.

In some embodiments, the succinate dehydrogenase inhibitor is selected from the group consisting of benzovindiflupyr, penthiopyrad, isopyrazam, fluxapyroxad, boscalid, fluopyram, bixafen, and penflufen.

In some embodiments, the succinate dehydrogenase inhibitor is benzovindiflupyr. In some embodiments, the succinate dehydrogenase inhibitor is penthiopyrad. In some embodiments, the succinate dehydrogenase inhibitor is isopyrazam. In some embodiments, the succinate dehydrogenase inhibitor is fluxapyroxad. In some embodiments, the succinate dehydrogenase inhibitor is boscalid. In some embodiments, the succinate dehydrogenase inhibitor is fluopyram. In some embodiments, the succinate dehydrogenase inhibitor is bixafen. In some embodiments, the succinate dehydrogenase inhibitor is penflufen.

In some embodiments, the at least one additional fungicide is a strobilurin fungicide.

In some embodiments, the strobilurin fungicide is selected from the group consisting of azoxystrobin, pyraclostrobin, picoxystrobin, fluoxastrobin, trifloxystrobin, kresoxim-methyl, dimoxystrobin, and orysastrobin.

In some embodiments, the strobilurin fungicide is selected from the group consisting of azoxystrobin, pyraclostrobin, picoxystrobin, fluoxastrobin, and trifloxystrobin.

In some embodiments, the strobilurin fungicide is azoxystrobin. In some embodiments, the strobilurin fungicide is pyraclostrobin. In some embodiments, the strobilurin fungicide is picoxystrobin. In some embodiments, the strobilurin fungicide is fluoxastrobin. In some embodiments, the strobilurin fungicide is trifloxystrobin. In some embodiments, the strobilurin fungicide is kresoxim-methyl. In some embodiments, the strobilurin fungicide is dimoxystrobin. In some embodiments, the strobilurin fungicide is orysastrobin.

In some embodiments, the at least one additional fungicide is a fungicidal multisite inhibitor.

In some embodiments, the fungicidal multisite inhibitor is selected from a group consisting of mancozeb, chlorothalonil, folpet, captan, metiram, maneb, propineb, copper hydroxide, copper octanoate, copper oxychloride, copper sulfate, copper sulfate (tribasic), mancopper, oxine-copper, copper bis(3-phenlsalicylate), copper zinc chromate, cuprous oxide, cupric hydrazinium sulfate, and cuprobam.

In some embodiments, the fungicidal multisite inhibitor is mancozeb. In some embodiments, the fungicidal multisite inhibitor is chlorothalonil. In some embodiments, the fungicidal multisite inhibitor is folpet. In some embodiments, the fungicidal multisite inhibitor is captan. In some embodiments, the fungicidal multisite inhibitor is metiram. In some embodiments, the fungicidal multisite inhibitor is maneb. In some embodiments, the fungicidal multisite inhibitor is propineb. In some embodiments, the fungicidal multisite inhibitor is copper hydroxide, copper octanoate, copper oxychloride, copper sulfate, copper sulfate (tribasic), mancopper, oxine-copper, copper bis(3-phenlsalicylate), copper zinc chromate, cuprous oxide, cupric hydrazinium sulfate, or cuprobam.

In some embodiments, the additional fungicide is selected from the group consisting of 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminum, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isoprothiolane, isopyrazam, isotianil, kasugamycin, kasugamycin hydrochloride hydrate, kresoxium-methyl, laminarin, mancopper, mancozeb, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril, benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, cufraneb, cupric hydrazinium sulfate, cuprobam, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, prothiocarb, prothiocarb hydrochloride, pyracarbolid, pyridinitril, pyroxychlor, pyroxyfur, quinacetol, quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, zarilamid, and any combinations thereof.

Synergistic compositions comprising the compound of Formula I, and uses thereof, are described in U.S. Pat. No. 9,526,245 (issued Dec. 27, 2016), 10,045,533 (issued Aug. 14, 2018), 9,532,570 (issued Jan. 3, 2017), 10,045,534 (issued Aug. 14, 2018), 9,538,753 (issued Jan. 10, 2017), and 10,051,862 (issued Aug. 21, 2018), the entire content of each of which is hereby incorporated by reference.

In some embodiments, the composition of the present invention further comprises at least one plant health stimulator. In some embodiments, the fungicidal mixture of the present invention further comprises at least one plant health stimulator.

In some embodiments, the plant health stimulator is selected from the group consisting of organic compounds, inorganic fertilizers or micronutrient donors, biocontrol agents and inoculants.

In some embodiments, the mixture comprises azoxystrobin. In some embodiments, the composition comprises azoxystrobin.

In some embodiments, the mixture comprises fluxapyroxad. In some embodiments, the composition comprises fluxapyroxad.

### Uses and Applications of the Mixtures and Compositions Described Herein

The present invention also provides a method for the control and/or prevention of fungal pathogen attack on a plant comprising applying any one of the compositions or mixtures described herein to soil, plant, root, foliage, seed, locus of the fungus, and/or a locus in which the infestation is to be prevented so as to thereby control and/or prevent fungal pathogen attack on a plant.

The present invention also provides any one of the compositions or mixtures described herein for use in controlling and/or preventing fungal attack on a plant.

The present invention also provides use of any one of the compositions or mixtures described herein for controlling and/or preventing fungal attack on a plant.

The present invention also provides a method for the control and/or prevention of plant and/or soil fungal diseases comprising applying any one of the compositions or mixtures described herein to soil, plant, root, foliage, seed, locus of the fungus, and/or a locus in which the infestation is to be prevented so as to thereby control and/or prevent plant and/or soil fungal diseases.

The present invention also provides any one of the compositions or mixtures described herein for use in controlling and/or preventing plant and/or soil fungal diseases.

The present invention also provides use of any one of the compositions or mixtures described herein for controlling and/or preventing plant and/or soil fungal diseases.

In some embodiments, the composition or mixture is applied to a portion of a plant, an area adjacent to a plant, soil in contact with a plant, soil adjacent to a plant, any surface adjacent to a plant, any surface in contact with a plant, a seed, and/or equipment used in agriculture.

In some embodiments, the composition or mixture is applied at an amount in the range of 5 g/ha to 150 g/ha of the compound of Formula I. In some embodiments, the composition or mixture is applied at an amount of 6.25 g/ha of the compound of Formula I. In some embodiments, the composition or mixture is applied at an amount of 10 g/ha of the compound of Formula I. In some embodiments, the composition or mixture is applied at an amount of 12.5 g/ha of the compound of Formula I. In some embodiments, the composition or mixture is applied at an amount of 20 g/ha of the compound of Formula I. In some embodiments, the composition or mixture is applied at an amount of 25 g/ha of the compound of Formula I. In some embodiments, the composition or mixture is applied at an amount of 50 g/ha of the compound of Formula I. In some embodiments, the composition or mixture is applied at an amount of 75 g/ha of the compound of Formula I. In some embodiments, the composition or mixture is applied at an amount of 100 g/ha of the compound of Formula I. In some embodiments, the composition or mixture is applied at an amount of 125 g/ha of the compound of Formula I. In some embodiments, the composition or mixture is applied at the time of planting.

In some embodiments, the composition or mixture is applied 1 to 60 day(s) after planting.

In some embodiments, the composition or mixture is applied 1 to 9 month(s) after planting.

In some embodiments, the composition or mixture is applied once during a growth season.

In some embodiments, the composition or mixture is applied at least one time during a growth season.

In some embodiments, the composition or mixture is applied two or more times during a growth season.

In some embodiments, the composition or mixture is applied as a foliar, seed treatment and/or a soil application.

The present invention also provides a method of controlling and/or preventing fungal pathogen attack on a plant comprising applying a fungicidally effective amount of a compound having Formula (I):

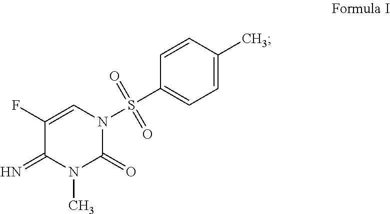 [SMILES: Cc1ccc(S(=O)(=O)n2cc(F)c(=N)n(C)c2=O)cc1]

and at least one adjuvant to soil, plant, root, foliage, seed, locus of the fungus, and/or a locus in which the infestation is to be prevented so as to thereby control and/or prevent fungal pathogen attack on the plant, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof; and
(v) sugar-based surfactants,
   wherein the compound of Formula I is in one or more forms and at least one form is Form III, Form IIIa, Form 01, or Form 02.

The present invention also provides a method of controlling and/or preventing plant and/or soil fungal diseases comprising applying a fungicidally effective amount of a compound having Formula (I):

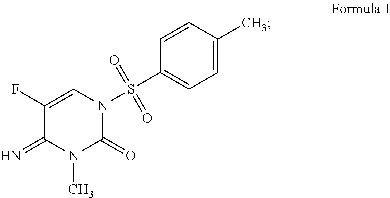 [SMILES: Cc1ccc(S(=O)(=O)n2cc(F)c(=N)n(C)c2=O)cc1]

and at least one adjuvant to soil, plant, root, foliage, seed, locus of the fungus, and/or a locus in which the infestation is to be prevented so as to thereby control and/or prevent plant and/or soil fungal diseases, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof; and
(v) sugar-based surfactants,
   wherein the compound of Formula I is in one or more forms and at least one form is Form III, Form IIIa, Form 01, or Form 02.

The present invention provides a method for improving biological activity of a compound of Formula I against fungal pathogen, the method comprising applying the compound of Formula I:

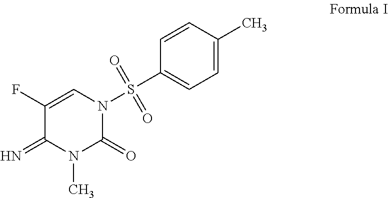 [SMILES: Cc1ccc(S(=O)(=O)n2cc(F)c(=N)n(C)c2=O)cc1]

in presence of at least one adjuvant, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof; and
(v) sugar-based surfactants
so as to thereby improve biological activity of the compound of Formula I,
   wherein the compound of Formula I is in one or more forms and at least one form is Form III, Form IIIa, Form 01, or Form 02.

In some embodiments, the compound of Formula I is applied in the presence of at least two adjuvants.

The present invention also provides use of a compound having Formula (I):

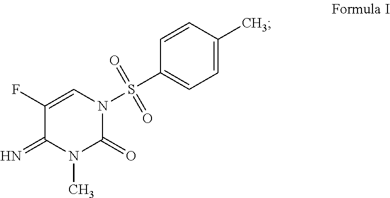 [SMILES: Cc1ccc(S(=O)(=O)n2cc(F)c(=N)n(C)c2=O)cc1]

and an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants; and
(vi) any combination of (i), (ii), (iii), (iv) and (v),
   for (a) controlling an/or preventing fungal pathogen attack on a plant and/or (b) controlling and/or preventing plant and/or soil fungal diseases,
   wherein the compound of Formula I is in one or more forms and at least one form is Fr III, Form IIIa, Form 01, or Form 02.

The present invention also provides a compound having Formula (I):

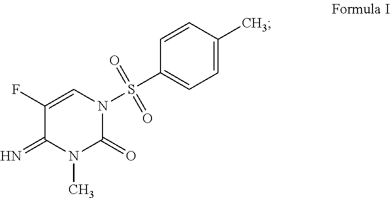 [SMILES: Cc1ccc(S(=O)(=O)n2cc(F)c(=N)n(C)c2=O)cc1]

and an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants; and
(vi) any combination of (i), (ii), (iii), (iv) and (v),
   for use in (a) controlling and/or preventing fungal pathogen attack on a plant and/or (b) controlling and/or preventing plant and/or soil fungal diseases,
   wherein the compound of Formula I is in one or more forms and at least one form is Form III, Form IIIa, Form 01, or Form 02.

The present invention provides use of an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants; and
(vi) any combination of (i), (ii), (iii), (iv) and (v),
   for improving the biological activity of the compound of Formula (I):

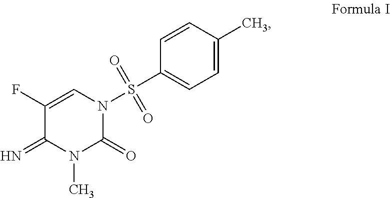 [SMILES: Cc1ccc(S(=O)(=O)n2cc(F)c(=N)n(C)c2=O)cc1]

wherein the compound of Formula I is in one or more forms and at least one form is Form III, Form IIIa, Form 01, or Form 02

The present invention provides an adjuvant selected from the group consisting of:
(i) polyalkylene oxide alkyl ether;
(ii) siloxane poly-akyleneoxide copolymer;
(iii) esters of fa-t acid;
(iv) vinylpyrrolidones and derivatives thereof;
(v) sugar-based surfactants; and
(vi) any combination of (i), (ii), (iii), (iv) and (v)
   for use in improving the biological activity of the compound of Formula (I)

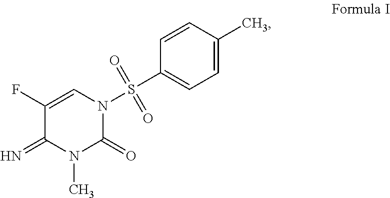 [SMILES: Cc1ccc(S(=O)(=O)n2cc(F)c(=N)n(C)c2=O)cc1]

wherein the compound of Formula I is in one or more forms and at least one form is Form III, Form IIIa, Form 01, or Form 02.

The present invention also provides a method of controlling and/or preventing fungal pathogen attack on a plant comprising applying a fungicidally effective amount of a compound having Formula (I):

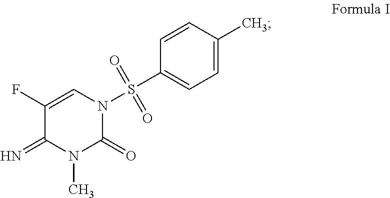 [SMILES: Cc1ccc(S(=O)(=O)n2cc(F)c(=N)n(C)c2=O)cc1]

and at least one adjuvant to soil, plant, root, foliage, seed, locus of the fungus, and/or a locus in which the infestation is to be prevented so as to thereby control and/or prevent fungal pathogen attack on the plant, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
   wherein the compound of Formula I is in one or more forms and at least one form is Form III, Form IIIa, Form 01, or Form 02.

The present invention also provides a method of controlling and/or preventing plant and/or soil fungal diseases comprising applying a fungicidally effective amount of a compound having Formula (I):

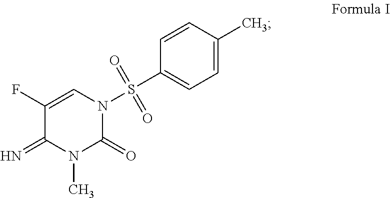 [SMILES: Cc1ccc(S(=O)(=O)n2cc(F)c(=N)n(C)c2=O)cc1]

and at least one adjuvant to soil, plant, root, foliage, seed, locus of the fungus, and/or a locus in which the infestation is to be prevented so as to thereby control and/or prevent plant and/or soil fungal diseases, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
   wherein the compound of Formula I is in one or more forms and at least one form is Form III, Form IIIa, Form 01, or Form 02.

The present invention provides a method for improving biological activity of a compound of Formula I against fungal pathogen, the method comprising applying the compound of Formula I:

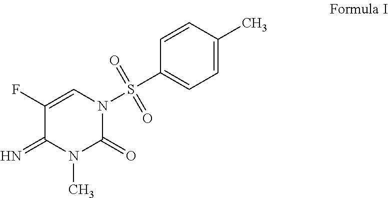 [SMILES: Cc1ccc(S(=O)(=O)n2cc(F)c(=N)n(C)c2=O)cc1]

in presence of at least one adjuvant, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
   so as to thereby improve biological activity of the compound of Formula I,
   wherein the compound of Formula I is in one or more forms and at least one form is Form III, Form IIIa, Form 01, or Form 02.

In some embodiments, the compound of Formula I is applied in the presence of at least two adjuvants.

The present invention also provides use of a compound having Formula (I)

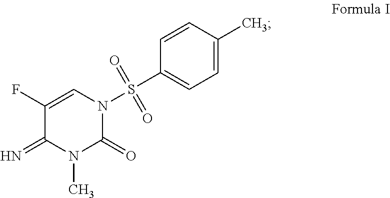 [SMILES: Cc1ccc(S(=O)(=O)n2cc(F)c(=N)n(C)c2=O)cc1]

and an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
   for (a) controlling and/or preventing fungal pathogen attack on a plant and/or (b) controlling and/or preventing plant and/or soil fungal diseases,
   wherein the compound of Formula I is in one or more forms and at least one form is Form III, Form IIIa, Form 01, or Form 02.

The present invention also provides a compound having Formula (I):

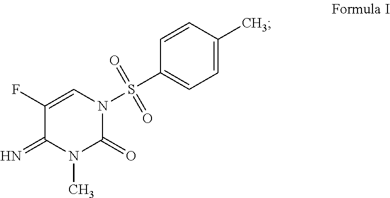 [SMILES: Cc1ccc(S(=O)(=O)n2cc(F)c(=N)n(C)c2=O)cc1]

and an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(vi) sugar-based surfactants;
(vii) lignins;
(viii) terpenes; and
(ix) any combination of (i), (ii), (iii), (iv), (v), (vi) and (vii),
   for use in (a) controlling and/or preventing fungal pathogen attack on a plant and/or (b) controlling and/or preventing plant and/or soil fungal diseases,
   wherein the compound of Formula I is in one or more forms and at least one form is Form III, Form IIIa, Form 01, or Form 02.

The present invention provides use of an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii).
for improving the biological activity of the compound of Formula (I)

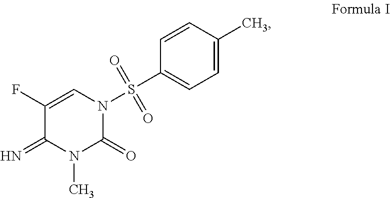 [SMILES: Cc1ccc(S(=O)(=O)n2cc(F)c(=N)n(C)c2=O)cc1]

wherein the compound of Formula I is in one or more forms and at least one form is Form III, Form IIIa, Form 01, or Form 02.

The present invention provides an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
   for use in improving the biological activity of the compound of Formula (I):

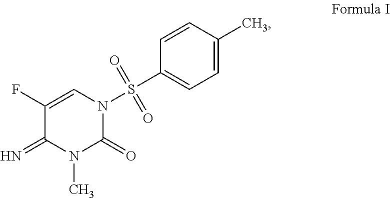 [SMILES: Cc1ccc(S(=O)(=O)n2cc(F)c(=N)n(C)c2=O)cc1]

wherein the compound of Formula I is in one or more forms and at least one form is Form III, Form IIIa, Form 01, or Form 02.

In some embodiments, the method or use comprises applying a tank mix comprising the compound of Formula I.

In some embodiments, the compound of Formula I is added to the tank mix in the form of any one of the mixtures or compositions described herein.

In some embodiments, 95% or more by weight of the amount of the compound of Formula applied is in the form of Form III, Form IIIa, Form 01, Form 02, or a mixture thereof.

Preferred adjuvants are described herein above.

In some embodiments, the compound of Formula I and the adjuvant are applied simultaneously. In some embodiments, the compound of Formula I and the adjuvant are applied sequentially.

In some embodiments, the compound of Formula I and the adjuvant(s) are applied separately. In some embodiments, the compound of Formula I and the adjuvant are applied together. In some embodiments, the compound of Formula I and the adjuvant are applied together as a tank mix. In some embodiments, the compound of Formula I and the adjuvant are formulated as a single composition. In some embodiments. Adjuvants that are formulated with the compound of Formula I in a composition are built-in adjuvants. Adjuvants that are tank mixed with the compound of Formula I or applied separately, for example via separate spraying, are add-on adjuvants.

In some embodiments, two or more adjuvants are applied wherein at least one of the adjuvants is a built-in adjuvant and at least one of the adjuvants is an add-on adjuvant.

In some embodiments, the compound of Formula I is applied at an amount in the range of 5 g/ha to 150 g/ha. In some embodiments, the compound of Formula I is applied at an amount of 6.25 g/ha. In some embodiments, the compound of Formula I is applied at an amount of 10 g/ha. In some embodiments, the compound of Formula I is applied at an amount of 12.5 g/ha. In some embodiments, the compound of Formula I is applied at an amount of 20 g/ha. In some embodiments, the compound of Formula I is applied at an amount of 25 g/ha. In some embodiments, the compound of Formula I is applied at an amount of 50 g/ha. In some embodiments, the compound of Formula I is applied at an amount of 75 g/ha. In some embodiments, the compound of Formula I is applied at an amount of 100 g/ha. In some embodiments, the compound of Formula I is applied at an amount of 125 g/ha.

The compound of Formula I and compositions and mixtures comprising the compound of Formula I may be applied to control and/or prevent a variety of fungal pathogen and diseases associated therewith. In some embodiments, the fungal pathogen is one of Leaf Blotch of Wheat (Mycosphaerella graminicola; anamorph: Zymoseptoria tritici), Wheat Brown Rust (Puccinia triticina), Stripe Rust (Puccinia striiformis f. sp. tritici), Scab of Apple (Venturia inaequalis), Blister Smut of Maize (Ustilago maydis), Powdery Mildew of Grapevine (Uncinula necator), Barley scald (Rhynchosporium secalis), Blast of Rice (Magnaporthe grisea), Rust of Soybean (Phakopsora pachyrhizi), Glume Blotch of Wheat (Leptosphaeria nodorum), Powdery Mildew of Wheat (Blumeria graminis f. sp. tritici), Powdery Mildew of Barley (Blumeria graminis f. sp. hordei), Powdery Mildew of Cucurbits (Erysiphe cichoracearum), Anthracnose of Cucurbits (Glomerella lagenarium), Leaf Spot of Beet (Cercospora beticola), Early Blight of Tomato (Alternaria solani), and Net Blotch of Barley (Pyrenophora teres).

In some embodiments, the fungal pathogen is one of Leaf Blotch of Wheat (Mycosphaerella graminicola; anamorph: Zymoseptoria tritici), Wheat Brown Rust (Puccinia triticina), Stripe Rust (Puccinia striiformis f. sp. tritici), Scab of Apple (Venturia inaequalis), Blister Smut of Maize (Ustilago maydis), Powdery Mildew of Grapevine (Uncinula necator), Barley scald (Rhynchosporium secalis), Blast of Rice (Magnaporthe grisea), Rust of Soybean (Phakopsora pachyrhizi), Glume Blotch of Wheat (Leptosphaeria nodorum), Powdery Mildew of Wheat (Blumeria graminis f. sp. tritici), Powdery Mildew of Barley (Blumeria graminis f. sp. hordei), Powdery Mildew of Cucurbits (Erysiphe cichoracearum), Anthracnose of Cucurbits (Glomerella lagenarium), Leaf Spot of Beet (Cercospora beticola), Early Blight of Tomato (Alternaria solani), and Net Blotch of Barley (Pyrenophora teres).

In some embodiments, the fungal pathogen is Zymoseptoria tritici.

In some embodiments, the plant or soil disease is one of Septoria, Brown rust, Yellow rust, Powdery Mildew, Rhyncosporium, Pyrenophora, Microduchium majus, Sclerotinia, Downy mildew, Phythopthora, Cercosporea beticola, Ramularia, ASR. Sigatoka negra.

The methods of the present invention refer to any crop plants, including but not limited to monocotyledons such as sugar cane cereals, rice, maize (corn), and/or; or dicotyledon crop such as beets (such as sugar beet or fodder beet); fruits (such as pomes, stone fruits, or soft fruits, for example apples, pears, plums, peaches, almonds, cherries, strawberries, raspberries, or blackberries); leguminous plants (such as beans, lentils, peas, or soybeans); oil plants (such as rape, mustard, poppy, olives, sunflowers, coconut, castor oil plants, cocoa beans, or groundnuts); cucumber plants (such as marrows, cucumbers or melons); fiber plants (such as cotton, flax, hemp, or jute); citrus fruits (such as oranges, lemons, grapefruit, or mandarins); vegetables (such as spinach, lettuce, cabbages, carrots, tomatoes, potatoes, cucurbits, or paprika); lauraceae (such as avocados, cinnamon, or camphor); tobacco; nuts; coffee; tea; vines; hops; durian; bananas; natural rubber plants; and ornamentals (such as flowers, shrubs, broad-leaved trees, or evergreens, for example conifers).

In some embodiments, the plants are monocotyledonous plants, more preferably, cereals. In a specific embodiment, the cereal crop is wheat. In another specific embodiment, the cereal crop is triticale. In another specific embodiment, the cereal crop is rye. In another specific embodiment, the cereal crop is oat. In a further embodiment, the cereal crop is barley. In another embodiment, the crop plants are rice plants. In still another embodiment, the crop plants are sugar cane plants. In yet another embodiment, the crop plants are corn plants.

In another embodiment, the crop plants are dicotyledonous plants.

In one embodiment, the crop plants are oil seed rape plants.

The compound of Formula I and compositions therefor may also be used as seed treatment to prevent or control phytopathogenic fungi as described in U.S. Patent Application Publication No. 2018-0000082 (published Jan. 4, 2018), the entire content of which is hereby incorporated by reference into this application.

The subject invention also provides a method for the control or prevention of fungal attack on a plant or protecting a plant from fungal attack, the method comprising applying any one of the compositions or mixtures disclosed herein to a seed adapted to produce the plant.

The subject invention also provides a method of treating a plant seed or seedling to produce a plant resistant to fungal attack, the method comprising applying any one of the compositions or mixtures disclosed herein to the plant seed or seedling.

The subject invention also provides a method of protecting a plant from fungal attack, the method comprising applying any one of the compositions or mixtures disclosed herein to the seedling environment.

The subject invention also provides a plant resistant to fungal attack, wherein the plant seed is treated with any one of the compositions or mixtures disclosed herein.

The subject invention also provides a plant seed or seedling adapted to produce a plant resistant to fungal attack, wherein the plant seed or seedling is treated with any one of the compositions or mixtures disclosed herein.

The subject invention also provides a package comprising any one of the compositions or mixtures disclosed herein.

The subject invention also provides use of any one of the mixtures disclosed herein for manufacturing a fungicidal composition. The subject invention also provides use of any one of the mixtures disclosed herein for manufacturing any one of the compositions disclosed herein.

### Method of Increasing Stability of Liquid Compositions Comprising Crystalline Form(s) of the Compound of Formula I

The present invention also provides a method for increasing stability of a liquid composition comprising a compound of Formula I:

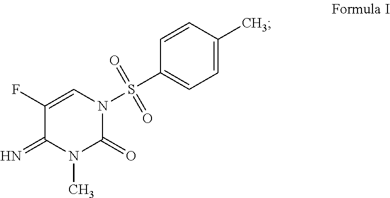 [SMILES: Cc1ccc(S(=O)(=O)n2cc(F)c(=N)n(C)c2=O)cc1]

and a liquid carrier, wherein the method comprises:
a) selecting a liquid carrier wherein the solubility of the compound of Formula I in the liquid carrier is less than 5000 ppm,
b) maintaining the pH value of the composition in the range of 5 to 7.5,
c) maintaining the water content of the composition to less than 0.5% by weight based on the total weight of the composition,
d) adding (i) at least one stabilizing surfactant having crystal growth inhibiting property or (ii) a stabilizing system having a crystal growth inhibiting property to the liquid composition, and/or
e) formulating the composition to have a viscosity of at least 500 cP,
   so as to thereby increase stability of the composition comprising the compound of Formula I,
   wherein the compound of Formula I is in one or more forms and at least one form is Form III, Form IIIa, Form 01, or Form 02.

The present invention also provides a method for increasing stability of a liquid composition comprising a compound of Formula I:

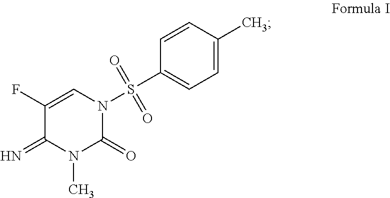 [SMILES: Cc1ccc(S(=O)(=O)n2cc(F)c(=N)n(C)c2=O)cc1]

and a liquid carrier, wherein the method comprises selecting a liquid carrier wherein the solubility of the compound of Formula I in the liquid carrier is less than 5000 ppm, and
wherein the compound of Formula I is in one or more forms and at least one form is Form III, Form IIIa, Form 01, or Form 02.

In some embodiments, the solubility of compound of Formula I in the liquid carrier is less than 1000 ppm. In some embodiments, the solubility of compound of Formula I in the liquid carrier is about 200 ppm. In some embodiments, the solubility of compound of Formula I in the liquid carrier is about 80 ppm.

The present invention also provides a method for increasing stability of a liquid composition comprising a compound of Formula I:

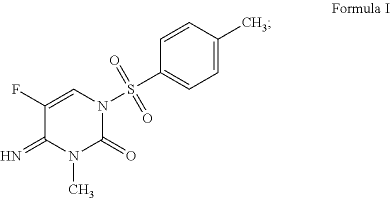 [SMILES: Cc1ccc(S(=O)(=O)n2cc(F)c(=N)n(C)c2=O)cc1]

and a liquid carrier, wherein the method comprises maintaining the pH value of the composition in the range of 5 to 7.5, and wherein the compound of Formula I is in one or more forms and at least one form is Form III, Form IIIa, Form 01, or Form 02.

In some embodiments, the pH of the composition is measured without further dilution or wetting. In some embodiments, the pH is measured after dilution or wetting with water.

In some embodiment, the pH of the composition is about 5, In some embodiments, the pH of the composition is about 5.5, in some embodiments, the pH of the composition is about 5.8, In some embodiments, the pH of the composition is about 6, In some embodiments, the pH of the composition is about 6.5, In some embodiments, the pH of the composition is about 7. In some embodiments, the pH of the composition is about 7.5.

In some embodiments, the method comprises adding a pH adjuster to the liquid composition.

The present invention also provides use of pH adjuster for increasing the stability of a suspension concentrate (SC) composition comprising a compound of Formula I:

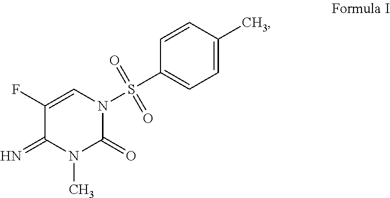 [SMILES: Cc1ccc(S(=O)(=O)n2cc(F)c(=N)n(C)c2=O)cc1]

wherein the compound of Formula I is in one or more forms and at least one form is Form III, Form IIIa, Form 01, or Form 02.

The present invention also provides use of pH adjuster for increasing the stability of a suspoemulsion (SE) composition comprising a compound of Formula I:

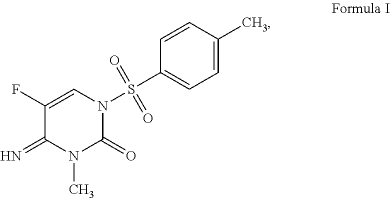 [SMILES: Cc1ccc(S(=O)(=O)n2cc(F)c(=N)n(C)c2=O)cc1]

wherein the compound of Formula I is in one or more forms a at least one form is Form III, Form IIIa, Form 01, or Form 2.

The present invention also provides a method for increasing stability of a liquid composition comprising a compound of Formula I:

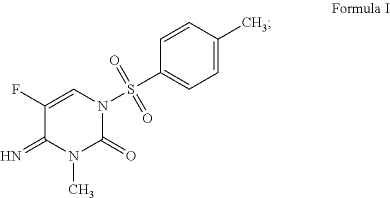 [SMILES: Cc1ccc(S(=O)(=O)n2cc(F)c(=N)n(C)c2=O)cc1]

and a liquid carrier, wherein the method comprises maintaining the water content of the composition to less than 0.5% by weight based on the total weight of the composition, and
wherein the compound of Formula I is in one or more forms and at least one form is Form III, Form IIIa, Form 01, or Form 02.

The present invention also provides a method for increasing stability of a liquid composition comprising a compound of Formula I:

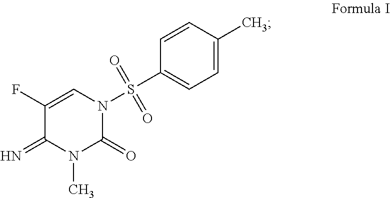 [SMILES: Cc1ccc(S(=O)(=O)n2cc(F)c(=N)n(C)c2=O)cc1]

and a liquid carrier wherein the method comprises maintaining the water content of the composition to less than or equal to 0.09% by weight based on the total weight of the composition, and
wherein the compound of Formula I is in one or more forms and at least one form is Form III, Form IIIa, Form 01, or Form 02.

The present invention also provides a method for increasing stability of a liquid composition comprising a compound of Formula I:

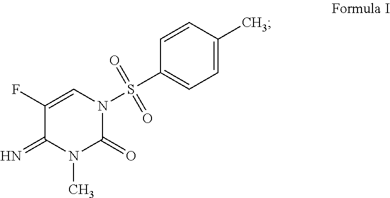 [SMILES: Cc1ccc(S(=O)(=O)n2cc(F)c(=N)n(C)c2=O)cc1]

and a liquid carrier, wherein the method comprises adding (i) at least one stabilizing surfactant having crystal growth inhibiting property or (ii) a stabilizing system having a crystal growth inhibiting property to the liquid composition, and
wherein the compound of Formula I is in one or more forms and at least one form is Form III, Form IIIa, Form 01, or Form 02.

In some embodiments, the stabilizing surfactant is a nonionic derivative of polyalkylene oxide polyaryl ether. In some embodiments, the stabilizing surfactant is an anionic derivative of polyalkylene oxide polyaryl ether.

In some embodiments, at least two stabilizing surfactants are added. In some embodiments, the at least two stabilizing surfactants comprise at least one nonionic derivative of polyalkylene oxide polyaryl ether and at least one anionic derivative of polyalkylene oxide polyaryl ether.

The present invention also provides use of at least one stabilizing surfactant having structure of polyalkylene oxide polyaryl ether for controlling solubility and/or degradation of compound of Formula I:

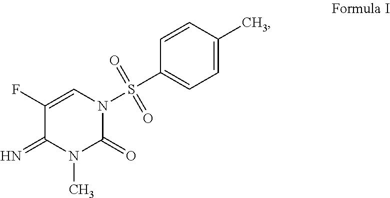 [SMILES: Cc1ccc(S(=O)(=O)n2cc(F)c(=N)n(C)c2=O)cc1]

wherein the compound of Formula I in Form III, Form IIIa, Form 01, or Form 02.

In some embodiments, the stabilizing surfactant having structure of polyalkylene oxide polyaryl ether is a non-ionic derivative of polyalkylene oxide polyaryl ether. In some embodiments, the stabilizing surfactant having structure of polyalkylene oxide polyaryl ether is an anionic derivative of polyalkylene oxide polyaryl ether.

In some embodiments, the method further comprises selecting a liquid carrier wherein the solubility of the compound of Formula I in the liquid carrier is less than 5000 ppm.

In some embodiments, the method further comprises maintaining the pH value of the composition in the range of 5 to 7.5.

In some embodiments, the method further comprises maintaining the water content of the composition to less than 0.5% by weight based on the total weight of the composition.

In some embodiments, the method further comprises adding (i) at least one stabilizing surfactant having crystal growth inhibiting property or (ii) a stabilizing system having a crystal growth inhibiting property to the liquid composition.

The present invention also provides a method for increasing stability of a liquid composition comprising a compound of Formula I:

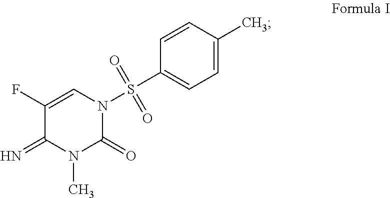 [SMILES: Cc1ccc(S(=O)(=O)n2cc(F)c(=N)n(C)c2=O)cc1]

and a liquid carrier, wherein the method comprises formulating the composition to have a viscosity of at least 500 cP, and
wherein the compound of Formula I is in one or more forms and at least one form is Form III, Form IIIa, Form 01, or Form 02.

In some embodiments, the stable liquid composition is a suspension concentrate (SC) composition.

In some embodiments, the stable composition is a suspoemulsion (SE) composition.

In some embodiments, the stable liquid composition is an oil dispersion (OD) composition.

In some embodiments, the stable liquid composition is an emulsifiable concentrate (EC) composition.

In some embodiments, the mixture or composition is diluted before application. In some embodiments, the mixture or composition is diluted with water. The rate of application of the diluted mixture or composition depends on the concentration of active ingredient(s) in the mixture or composition prior to dilution. Generally, the diluted mixture or composition is applied at a rate of about 5 L/ha to about 120 L/ha.

In some embodiments, the method comprises using a batch of the compound of Formula I that comprises 95% or more by weight of the compound of Formula I to prepare the stable, liquid composition.

The present invention also provides a method for increasing stability of a liquid composition comprising a compound of Formula I:

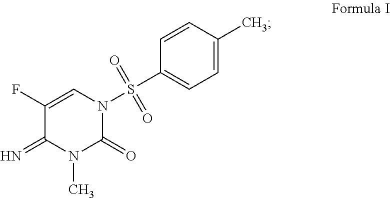 [SMILES: Cc1ccc(S(=O)(=O)n2cc(F)c(=N)n(C)c2=O)cc1]

and a liquid carrier, wherein the method comprises using a batch of the compound of Formula I that comprises 95% or more by weight of the compound of Formula I to prepare the stable, liquid composition, and
wherein the compound of Formula I is in one or more forms and at least one form is Form III, Form IIIa, Form 01, or Form 02.

In some embodiments, the method comprises using a batch of the compound of Formula I that comprises 96% or more by weight of the compound of Formula I to prepare the stable, liquid composition. In some embodiments, the method comprises using a batch of the compound of Formula I that comprises 97% or more by weight of the compound of Formula I to prepare the stable, liquid composition. In some embodiments, the method comprises using a batch of the compound of Formula I that comprises 98% or more by weight of the compound of Formula I to prepare the stable, liquid composition. In some embodiments, the method comprises using a batch of the compound of Formula I that comprises 99% or more by weight of the compound of Formula I to prepare the stable, liquid composition. In some embodiments, the method comprises using a batch of the compound of Formula I that comprises 99.5% or more by weight of the compound of Formula I to prepare the stable, liquid composition. In some embodiments, the method comprises using a batch of the compound of Formula I that comprises 99.9% by weight of the compound of Formula I to prepare the stable, liquid composition. In some embodiments, the method comprises using a batch of the compound of Formula I that is substantially pure of the compound of Formula I to prepare the stable, liquid composition. In some embodiments, the method comprises using a batch of the compound of Formula I that comprises 100% by weight of the compound of Formula I to prepare the stable, liquid composition.

### Process of Preparing Composition Comprising the Compound of Formula I

The present invention provides a process for preparing the suspension concentrate (SC) composition disclosed herein, the process comprises the steps:
(1) mixing the agriculturally acceptable inert additives and an aqueous liquid carrier to obtain a premix;
(2) adding the compound of Formula I to the premix obtained in step (1) to obtain a mixture; and
(3) milling the resulting mixture of step (2) to obtain the desired composition,
   wherein the compound of Formula I is in one or more forms and at least one form is Form III, Form IIIa, Form 01, or Form 02.

The present invention provides a process for preparing the suspension concentrate (SC) composition disclosed herein, the process comprises the steps:
(1) mixing the agriculturally acceptable inert additives and an aqueous liquid carrier to obtain a premix;
(2) adding the compound of Formula I to the premix obtained in step (1) to obtain a mixture; and
(3) milling the resulting mixture of step (2) to obtain the desired composition,
   wherein the compound of Formula I is in one or more forms and at least one form is Form IIIa, Form 01, or Form 02.

In some embodiments, the process comprises adding additional additive to the mixture of step (2) prior to milling the mixture.

In some embodiments, the premix of step (1) is heated before step (2).

The present invention provides a process for preparing the suspoemulsion (SE) composition disclosed herein, the process comprises the steps:
(1) mixing the agriculturally acceptable inert additives and an aqueous liquid carrier to obtain a premix;
(2) adding the compound of Formula I and at least one adjuvant to the premix obtained in step (1) to obtain a mixture; and
(3) milling the resulting mixture of step (2) to obtain the desired composition,
   wherein the compound of Formula I is in one or more forms and at least one form is Form III, Form IIIa, Form 01, or Form 02.

In some embodiments, step (1) comprises adding a non-aqueous liquid carrier. In some embodiments, step (2) comprises adding a non-aqueous liquid carrier. In some embodiments, the adjuvant added in step (2) is a non-aqueous liquid carrier.

The present invention provides a process for preparing the oil dispersion (OD) composition disclosed herein, the process comprises the steps:
(1) mixing the agriculturally acceptable inert additives and a non-aqueous liquid carrier to obtain a premix;
(2) adding the compound of Formula I to the premix obtained in step (1) to obtain a mixture; and
(3) milling the resulting mixture of step (2) to obtain the desired composition,
   wherein the compound of Formula I is in one or more forms and at least one form is Form III, Form IIIa, Form 01, or Form 02.

The present invention provides a process for preparing the emulsifiable concentrate (EC) composition disclosed herein, the process comprises the steps:
(1) mixing the agriculturally acceptable inert additives and a non-aqueous liquid carrier to obtain a premix;
(2) adding the compound of Formula I to the premix obtained in step (1) to obtain a mixture; and
(3) filtering the solution of step (2) to obtain the desired composition,
   wherein the compound of Formula I is in one or more forms and at least one form is Form III, Form IIIa, Form 01, or Form 02.

In some embodiments, step (2) of the process comprises obtaining a batch of the compound of Formula I comprising 95% or more by weight of the compound of Formula I and adding the batch of the compound of Formula I to the premix obtained in step (1) to obtain a mixture.

In some embodiments, the batch of the compound of Formula I comprises 96% or more by weight of the compound of Formula I. In some embodiments, the batch of the compound of Formula I comprises 97% or more by weight of the compound of Formula I. In some embodiments, the batch of the compound of Formula I comprises 98% or more by weight of the compound of Formula I. In some embodiments, the batch of the compound of Formula I comprises 99% or more by weight of the compound of Formula I. In some embodiments, the batch of the compound of Formula I comprises 99.5% or more by weight of the compound of Formula I. In some embodiments, the batch of the compound of Formula I comprises 99.9% by weight of the compound of Formula I. In some embodiments, the batch of the compound of Formula I is substantially pure of the compound of Formula I. In some embodiments, the batch of the compound of Formula I is 100% by weight of the compound of Formula I.

The present invention also provides a process of preparing a stable, liquid composition comprising an admixture of a compound of Formula I:

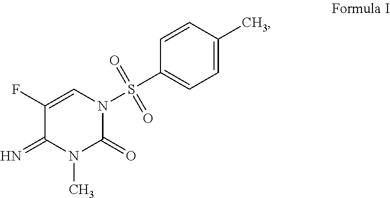 [SMILES: Cc1ccc(S(=O)(=O)n2cc(F)c(=N)n(C)c2=O)cc1]

and a liquid carrier,
wherein the process comprises the steps:
(1) obtaining a batch of the compound of Formula I that is substantially pure of the compound of Formula I or comprises 95% or more by weight of the compound of Formula I, and
(2) mixing the batch of the compound of Formula I from step (1) with the liquid carrier to obtain the desired composition,
wherein the compound of Formula I is in one or more forms and at least one form is Form III, Form IIIa, Form 01, or Form 02.

The present invention also provides a process of preparing a stable, liquid composition comprising a compound of Formula I:

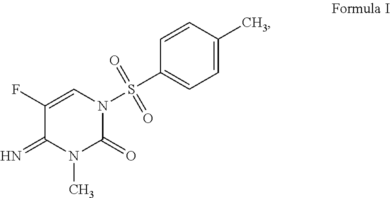 [SMILES: Cc1ccc(S(=O)(=O)n2cc(F)c(=N)n(C)c2=O)cc1]

and a liquid carrier,
wherein the process comprises the steps:
   (1) obtaining a batch of the compound of Formula I wherein 95% or more of the compound of Formula I in the batch is in Form III, Form IIIa, Form 01, Form 02 or a mixture thereof, and
   (2) mixing the batch of the compound of Formula I from step (1) with the liquid carrier to obtain the composition,
wherein the compound of Formula I is in one or more forms and at least one form is Form III, Form IIIa, Form 01, or Form 02.

In some embodiments, the batch of the compound of Formula is substantially pure of the compound of Formula I. In some embodiments, the batch of the compound of Formula I comprises 95% or more by weight of the compound of Formula I. In some embodiments, the batch of the compound of Formula I comprises 96% or more by weight of the compound of Formula I. In some embodiments, the batch of the compound of Formula I comprises 97% or more by weight of the compound of Formula I. In some embodiments, the batch of the compound of Formula I comprises 98% or more by weight of the compound of Formula I. In some embodiments, the batch of the compound of Formula I comprises 99% or more by weight of the compound of Formula I. In some embodiments, the batch of the compound of Formula I comprises 99.5% or more by weight of the compound of Formula I.

In some embodiments, 95% or more of the compound of Formula I in the batch is in the form of Form III, Form IIIa, Form 01, Form 02, or a mixture thereof. In some embodiments, 96% or more of the compound of Formula I in the batch is in the form of Form III, Form IIIa, Form 01, Form 02, or a mixture thereof. In some embodiments, 97% or more of the compound of Formula I in the batch is in the form of Form III, Form IIIa, Form 01, Form 02, or a mixture thereof. In some embodiments, 98% or more of the compound of Formula I in the batch is in the form of Form III, Form IIIa, Form 01, Form 02, or a mixture thereof. In some embodiments, 99% or more of the compound of Formula I in the batch is in the form of Form III, Form IIIa, Form 01, Form 02, or a mixture thereof. In some embodiments, 99.5% or more of the compound of Formula I in the batch is in the form of Form III, Form IIIa, Form 01, Form 02, or a mixture thereof.

In some embodiments, the process comprises preparing a premix by combining at least one agriculturally acceptable inert additives and a liquid carrier and combining the premix with the batch of the compound of Formula I from step (1) to obtain the desired composition.

Agriculturally acceptable inert additives that may be combined with the liquid carrier are described herein above.

The compound of Formula I exists in many forms. Forms III, IIIa, 01 and 02 are described herein. Other forms may be any solid form of the compound of Formula I including but not limited to amorphous, crystalline, solvate, hydrate or any mixture thereof.

The present invention also provides a composition comprising an admixture of a compound of Formula I:

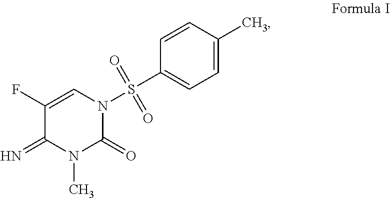 [SMILES: Cc1ccc(S(=O)(=O)n2cc(F)c(=N)n(C)c2=O)cc1]

and a liquid carrier,
wherein the composition is prepared using the process described herein, and
wherein the compound of Formula I is in one or more forms and at least one form is Form III, Form IIIa, Form 01, or Form 02.

The present invention also provides a composition comprising a compound of Formula I:

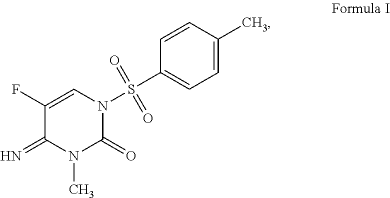 [SMILES: Cc1ccc(S(=O)(=O)n2cc(F)c(=N)n(C)c2=O)cc1]

and a liquid carrier,
wherein the composition is prepared using the process described herein, and
wherein the compound of Formula I is in one or more forms and at least one form is Form III, Form IIIa, Form 01, or Form 02.

The present invention provides a suspension concentrate (SC) composition prepared using the process described herein.

The present invention provides a suspoemulsion (SE) composition prepared using the process described herein.

The present invention provides an emulsifiable concentrate (EC) composition prepared using the process described herein. The present invention provides an oil dispersion (OD) composition prepared using the process described herein.

The crystalline forms Form III, Form IIIa, Form 01 and Form 02 of the present invention may be prepared, for example, using the processes disclosed herein. The crystalline forms Form III, Form IIIa, Form 01 and Form 02 of the present invention are not limited to only those that are prepared using the processes disclosed herein.

When a crystalline form of compound A is used as a starting material for preparing Form III, Form IIIa, Form 01 or Form 02 of the present invention, the starting crystalline form may be prepared using any process, including but not limited those that are described in International Patent Application No. PCT/IB2018/000875. Similarly, when compound A is used as a starting material for preparing crystalline forms, compound A may be prepared using any process, including but not limited to those that are described in PCT International Application Nos. PCT/US2014/072566, PCT/US2014/072569, PCT/IB2020/058893 and PCT/IB2021/051957.

Each embodiment disclosed herein is contemplated as being applicable to each of the other disclosed embodiments. Thus, all combinations of the various elements described herein are within the scope of the invention. In addition, when lists are provided, the list is to be considered as a disclosure of any one member of the list.

While the present subject matter has been shown and described with reference to preferred embodiments thereof, it will be understood by those skilled in the art that many alternatives, modifications and variations may be made thereto without departing from the spirit and scope thereof. Accordingly, it is intended to embrace all such alternatives, modifications, and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference.

The following examples illustrate the practice of the present subject matter in some of its embodiments but should not be construed as limiting the scope of the present subject matter. Other embodiments will be apparent to one skilled in the art from consideration of the specification and examples. It is intended that the specification, including the examples, is considered exemplary only, without limiting the scope and spirit of the present subject matter.

### EXPERIMENTAL DETAILS

### Section I (Examples 1-4)—Preparation of Crystalline Forms

XRPD: Rugaku MiniFlex600 with MD/teX ultra height speed 1D detector; Cu-Ka1 radiation; 40 kV and 40 mA tube power; curved Ge monochromator; 0.005° 2θ step size, 12 s step time, 2-140° 2θ scanning range; detector mode: step scan; 1° 2θ detector step. The sample (20-40 mg) was placed either between two acetate foils (dry samples) or between two Kapton foils (wet samples) and clamped in a Stoe transmission sample holder; the sample holder was rotated during the measurement.

DSC: Mettler Toledo DSC1; hermetically sealed with aluminum cover; 10° C./min heating rate, from 25 to 320° C. The melting point is understood as the peak onset.

TGA: Mettler Toledo TGA/SDC1. hermetically sealed with aluminum cover; 10° C./min heating rate, from 25 to 320° C.

Solvents: For all experiments, Fluka, Merck or ABCR analytical grade solvents were used. For experiments under dry conditions and preparing water-organic solvent mixtures with accurate water activity the organic solvents were previously dried over molecular sieves (4A) for a few days.

### Preparation of Form I Polymorph of Compound A (Form I):

Form I is defined in PCT/IB2018/000875. Form I exhibits an X-ray powder diffraction pattern as shown in FIG. 1 of PCT/IB2018/000875, having characteristic peaks at 2-theta angles of 9.08, 10.98, 14.05, 17.51, 18.75, 21.63, 23.33, 24.70, 24.83, 25.37, 26.51 and 29.23. In some embodiments, the powder X-ray diffraction pattern of Form I comprises characteristic peaks at 2-theta angles of 14.05, 17.51, 18.75, 21.63 and 26.51. In some embodiments the powder X-ray diffraction pattern of Form I comprises characteristic peaks at 2-theta angles of 14.05, 17.51, 18.75 and 21.63.

6 gr of 5-fluoro-3-methylcytosine 90.3% were added to 3 bottle neck 500 mL flask equipped with pH meter, dropping funnel and thermometer. 40 gr of CPME were added to the flask and mixed. The solution was heated to 50° C. 0.43 gr of TBAB were added to the flask during heating. 8.83 gr of TsCl were added to the reaction mixture in one portion during heating. NaOH (8M) was added dropwise to reaction mixture using addition funnel. The pH was kept between 9-10 during the course of the reaction. The reaction was monitored using HPLC. When the reaction finished, 130 gr of CPME was added at 50° C. The solution becomes clear. 100 gr preheated water added at 50° C. and mix for few minutes, then separate the phases at 50° C. The organic phase was washed with warm water and then the CPME was partially evaporated to ratio of 1:3 (product vs. solvent) at vacuum of 30 mbar at 50° C.

The mixture was cooled to 15° C. at rate of 1° C./min while stirring. The product was filtered and dried overnight at vacuum oven in 70° C. resulting on Form I polymorph (96.6% purity).

Compound A can also be prepared by any process, including but not limited to the processes described in PCT International Application Nos. PCT/US2014/072566, PCT/US2014/072569, PCT/IB2020/058893 and PCT/IB2021/051957.

Form I may be prepared using any process, including but not limited to the process of crystallization of compound A for receiving Form I as disclosed in International Patent Application No. PCT/IB2018/000875.

In the following examples, where it is mentioned that the starting material is Form III, it can be prepared according to any one of examples 1.1-1.14, or any other process that results in Form III. Where it is mentioned that the starting material includes hydrate, it can be prepared according to the procedure disclosed in PCT/IB2018/000875.

### Example 1. Form III

Examples 1.1-1.14 resulted in Form III.

### Example 1.1

50 mg of Form I was dissolved in 5 mL isobutyl acetate and the solution was stirred at room temperature (25° C.) for approx. 60 minutes. The solution was filtered with a Whatman 0.45 μm filter and left to evaporate at 50° C. When the solvent was completely evaporated, by visual inspection, the solid was analyzed by XRPD.

### Example 1.2

50 mg of Form I was dissolved in 5 mL cyclohexanone and the solution was stirred at room temperature (25° C.) for approx. 60 minutes. The solution was filtered with a Whatman 0.45 μm filter and left to evaporate at 50° C. When the solvent was completely evaporated, by visual inspection, the solid was analyzed by XRPD.

### Example 1.3

50 mg of Form I was dissolved in 5 mL propylacetate and the solution was stirred at room temperature (25° C.) for approx. 60 minutes. The solution was filtered with a Whatman 0.45 μm filter and left to evaporate at high temperature: 50° C. When the solvent was completely evaporated, by visual inspection, the solid was analyzed by XRPD.

### Example 1.4

50 mg of Form I was dissolved in 5 mL isopropylacetate and the solution was stirred at room temperature (25° C.) for approx. 60 minutes. The solution was filtered with a Whatman 0.45 μm filter and left to evaporate at high temperature: 50° C. When the solvent was completely evaporated, by visual inspection, the solid was analyzed by XRPD.

### Example 1.5

50 mg of Form I was dissolved in 5 mL butylacetate and the solution was stirred at room temperature (25° C.) for approx. 60 minutes. The solution was filtered with a Whatman 0.45 μm filter and left to evaporate at high temperature: 50° C. When the solvent was completely evaporated, by visual inspection, the solid was analyzed by XRPD.

### Example 1.6

50 mg of Form I was dissolved in 5 mL diethyl carbonate and the solution was stirred at room temperature (25° C.) for approx. 60 minutes. The solution was filtered with a Whatman 0.45 μm filter and left to evaporate at high temperature: 50° C. When the solvent was completely evaporated, by visual inspection, the solid was analyzed by XRPD.

### Example 1.7

50 mg of Form I was dissolved in 5 mL 1,2 dimethoxyethane and the solution was stirred at room temperature (25° C.) for approx. 60 minutes. The solution was filtered with a Whatman 0.45 μm filter and left to evaporate at high temperature: 50° C. When the solvent was completely evaporated, by visual inspection, the solid was analyzed by XRPD.

### Example 1.8

50 mg of Form I was dissolved in 5 mL 1,2 methyl ethyl ketone and the solution was stirred at room temperature (25° C.) for approx. 60 minutes. The solution was filtered with a Whatman 0.45 μm filter and left to evaporate at 50° C. When the solvent was completely evaporated, by visual inspection, the solid was analyzed by XRPD.

### Example 1.9

50 mg of Form I was dissolved in 5 mL 2-methyltetrahydrofuran and the solution was stirred at room temperature (25° C.) for approx. 60 minutes. The solution was filtered with a Whatman 0.45 μm filter and left to evaporate at 50° C. When the solvent was completely evaporated, by visual inspection, the solid was analyzed by XRPD.

### Example 1.10

A solution of 50 mg of Form I in 5 mL of Methyl ethyl ketone was heated at 75° C. under stirring. The solution was filtered with a Whatman 0.45 μm filter and allowed to evaporate to dryness at 60° C. When the solvent was completely evaporated, by visual inspection, the solid was analyzed by XRPD.

### Example 1.11

50 mg of Form I was dissolved in 5 mL of anisole and the solution was stirred at room temperature (25° C.) for approx. 60 minutes. The solution was filtered with a Whatman 0.45 μm filter and left to evaporate at 60° C. When the solvent was completely evaporated, by visual inspection, the solid was analyzed by XRPD.

### Example 1.12

50 mg of Form I was dissolved in 5 mL of toluene and the solution was stirred at room temperature (25° C.) for approx. 60 minutes. The solution was filtered with a Whatman 0.45 μm filter and left to evaporate at 60° C. When the solvent was completely evaporated, by visual inspection, the solid was analyzed by XRPD.

### Example 1.13

50 mg of Form I was suspended in 5 mL of water (10 mg/mL) and left under magnetic stirring at approx. 350 rpm for 3 days at room temperature (25° C.). The suspensions were recovered, filtered under vacuum and analyzed by XRPD.

### Example 1.14

5 gr Form I (97.4% purity) and 40 mL acetonitrile added to 100 mL round bottom flask equipped with magnetic stirrer and heated under stirring to 60° C. until full dissolution. After 25 minutes at this temperature 50 mL of water was added. The temperature dropped to 40° C. and then heated back to 65° C. Then the mixture cooled to room temperature slowly and the product filtered off and dried at 80° C. at 10 mbar overnight

### Example 2. Form IIIa

Examples 2.1-2.8 resulted in the crystalline Form IIIa.

### Example 2.1

100 mg of Form I was suspended in 10 mL of Water (10 mg/mL) and left under magnetic stirring at 50° C. at approx. 350 rpm for 7 days. The suspension was recovered, filtered under vacuum and analyzed by XRPD.

### Example 2.2

50 mg of Form I was suspended in 5 mL of Water (10 mg/mL) and left under magnetic stirring at room temperature at approx. 350 rpm for 15 days. The suspension was recovered, filtered under vacuum and analyzed by XRPD.

### Example 2.3

50 mg of Form III was suspended in 5 mL of Water (10 mg/mL) and left under magnetic stirring at room temperature at approx. 350 rpm for 15 days. The suspension was recovered, filtered under vacuum and analyzed by XRPD.

### Example 2.4

50 mg of hydrate form, was suspended in 5 mL of Water (10 mg/mL) and left under magnetic stirring at room temperature at approx. 350 rpm for 15 days. The suspension was recovered, filtered under vacuum and analyzed by XRPD.

### Example 2.5

50 mg of Form III was suspended in 0.625 mL of a Methyl Ethyl Ketone (80 mg/mL) and left under magnetic stirring at approx. 350 rpm for 3 days at room temperature (25° C.). The suspension was recovered, filtered under vacuum and analyzed by XRPD.

### Example 2.6

50 mg of Form I was suspended in a mixture of 5 mL water plus 1% of N,N-Dimethylformamide and left under magnetic stirring at 50° C. for four days. After four days, Form IIIa was collected by vacuum filtration using Buchner funnel, vacuum flask, filter paper Whatman® W42

### Example 2.7

50 mg of Form I was suspended in a mixture of 5 mL water plus 1% of N,N-Dimethylformamide and left under magnetic stirring at 50° C. for seven days. After seven days, Form IIIa was collected by vacuum filtration using Buchner funnel, vacuum flask, filter paper Whatman® W42

### Example 2.8

50 mg of Form I was suspended in a mixture of 5 mL water plus 1% of N,N-Dimethylformamide and left under magnetic stirring at 50° C. for two days. After two days, Form IIIa was collected by vacuum filtration using Buchner funnel, vacuum flask, filter paper Whatman® W42.

### Example 3. Form 02

Examples 3.1-3.2 resulted in the crystalline Form 02.

### Example 3.1

50 mg of Form I was dissolved in 5 mL p-xylene and the solution was stirred at room temperature (25° C.) for approx. 60 minutes. The solution was filtered with a Whatman 0.45 μm filter and left to evaporate at 50° C. When the solvent was completely evaporated, by visual inspection, the solid was analyzed by XRPD.

### Example 3.2

50 mg of Form I was dissolved in 5 mL mesitylene and the solution was stirred at room temperature (25° C.) for approx. 60 minutes. The solution was filtered with a Whatman 0.45 μm filter and left to evaporate at 50° C. When the solvent was completely evaporated, by visual inspection, the solid was analyzed by XRPD.

### Example 4. Form 01

The example below resulted in crystalline Form 01.

50 mg of Form I was dissolved in 5 mL of Chloroform and the solution was stirred at room temperature (25° C.) for approx. 60 minutes. The solution was filtered with a Whatman 0.45 μm filter and left to evaporate at room temperature.

### Section II (Examples 5-29)—Compositions and Mixtures

In this section, the compound of Formula I refers to a compound having the following structure:

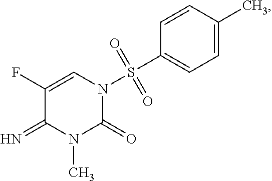 [SMILES: Cc1ccc(S(=O)(=O)n2cc(F)c(=N)n(C)c2=O)cc1]

and does not refer to crystalline forms Form III, Form IIIa, Form 01 and Form 02 of the present invention.

Compound of Formula I can be prepared as described, for example, in WO2015/103144 and WO2015/103142.

Preparing a stable composition comprising the compound of Formula I is challenging due to the high sensitivity of the compound. Numerous attempts were made to stabilize the composition. Some of the results are described below.

The compound of Formula I was combined with adjuvant(s) as tank mixes and/or as built-in compositions. Different types of adjuvants with different compositions were tested.

The adjuvants which were tested were polyvinylpyrrolidone (PVP), vinylpyrrolidone and vinyl acetate block copolymer (VP/VA), siloxane polyalkyleneoxide copolymer (Silwet® L-077), tridecyl alcohol ethoxylated 13/9 (Trycol®), alkoxylated alcohol (Agnique® BP420) and fatty acid methyl ester (Agnique® ME 18 RDF).

### Example 5: 450 SC Composition with No Adjuvant

A suspension concentrate (SC) composition containing 450 g/L of the compound of Formula I and no adjuvant was prepared as follow:

### Step I: Preparation of Agriculturally Acceptable Inert Additives Premix

Soft water and Van Gel® B were charged to the vessel and mixed (high shear) to form a solution. The content of the vessel was heated to 60° C. SOPROPHOR® TS/54 (TSP 54) was heated to 50-60° C. and added gradually to the vessel. Supragil® WP, Soprophor® 3D33, KH₂PO₄, Na₂HPO₄ and SAG 1572 were then added to the homogeneous solution.

### Step II: Preparation of the Compound of Formula I

The compound of Formula I (40% W/W) was added to the premix with the surfactants inside, the suspension was milled in a bead mill (0.8-1.2 mm beads) until a particle size distribution of d90<5 μm was reached. The milled suspension was drained from the reactor to new vessel.

Step III: Finalization of the Composition Propylene glycol was added to the milled suspension and mixed until a uniform suspension was obtained. Soft water and AgRH 23 2% solution were added to the suspension while mixing until a viscosity of 1600-2200 cP was reached. Mixing was continued until a homogenous solution was obtained. Viscosity was measured by viscometer according to CIPAC method MT 192.

The composition is summarized in Table 1.

TABLE 1
450 SC composition of the compound of Formula I and no adjuvant

| | | Amount for | |
|---|---|---|---|
| Ingredient trade name | Chemical name and CAS No. | 1000 L (kg) | |
| Compound of Formula I | 5-fluoro-4-imino-3methyl-1- | 450 kg | |
| tech. as 100% of ADAMA | [4-methylphenyl)sulfonyl]- | (469 kg | |
| Makhteshim LTD. | 3,4-dohydropyrimidin-2(1H)- | for 96%) | |
| | one | | |
| | CAS No. N/A | | |
| Supragil ® WP of Solvay or | Sodium | 5.9 | Kg |
| SURFOM ® HRB of OXITENO | Diisopropylnaphthalene | | |
| | sulphonate | | |
| | CAS No. 1322-93-6 | | |
| Soprophor ® 3 D 33 of | POLYARYLPHENYL ETHER | 47.0 | kg |
| Solvay | PHOSPHATE | | |
| | CAS No. 90093-37-1, 99734- | | |
| | 09-5, 7664-38-2 | | |
| Emulsogen ® TS 540 of | Tristyryl phenol- | 17.5 | Kg |
| Clariant or SOPROPHOR ® | polyethylene glycol ether | | |
| TS/54 of Solvay | CAS No. 70559-25-0 (of | | |
| | Clariant) | | |
| | CAS No. 104376-75-2 (of | | |
| | Solvay) | | |
| SAG ™ 1572 of Momentive | Polydimethylsiloxane | 2.0 | Kg |
| Performance Materials | emulsion | | |
| GmbH | CAS No. N/A, 56-81-5 | | |
| | (Glycerine) | | |
| MPG - MPG USP of INEOS | 1,2-propanediol | 23.5 | Kg |
| OXIDE or | CAS No. 57-55-6 | | |
| MPG Industrial TG of | | | |
| SHELL EASTERN | | | |
| CHEMICALS or | | | |
| Propylene Glycol | | | |
| Industrial Grade of | | | |
| DOW | | | |
| MKP (Mono potassium | KH₂PO₄ (Potassium | 1.7 | Kg |
| phosphate) of Rotem | dihydrogenorthophosphate) | | |
| Amfert Negev Ltd., | CAS No. 7778-77-0 | | |
| Disodium Phosphate | Na₂HPO₄ (Disodium Phosphate | 6.9 | Kg |
| Anhydrous of Haifa | Anhydrous) | | |
| Chemicals Ltd. | CAS No. 7558-79-4 | | |
| Van Gel ® B of | Magnesium aluminum | 5.9 | kg |
| Vanderbilt minerals, | silicate, Smectite clay; | | |
| LLC. | CAS No. 12199-37-0 | | |
| AgRH 23 of Ametech | Xanthan gum | 2.8 | Kg |
| | CAS No. 11138-66-2 | | |

| | |
|---|---|
| Soft Water | Up to 1000 L |
| | (about |
| | 616 kg) |

Appearance: Off-white homogeneous suspension
Density, g/ml: 1.15-1.25

### Stability Results:

The physical and chemical stability of composition of Example 5 was tested under various conditions including CIPAC conditions. The stability results are summarized in Table 2.

TABLE 2
Stability results of the composition of Example 1

| | Start | Room | Oven 54° C. |
|---|---|---|---|
| Appearance | Off-white | Off-white | Off-white |
| | homogeneous | homogeneous | homogeneous |
| | suspension | suspension | suspension |
| Concentration of | 40.80% | 40.4% | 40.2% |
| compound of | | | |
| Formula I | | | |
| Density, g/ml | 1.18 | 1.21 | — |
| pH | 6.3 | 6.0 | 6.6 |
| Viscosity, sp. 62 at | 1790 (After | 1000 | — |
| 12 rpm (cP) | 1 day) | | |
| Particle size (D₉₀) | 6.326 | 6.326 | 10.14 |
| foaming 0.2% | — | 10 ml | 10 ml |
| foaming 1% | — | 25 ml | 25 ml |
| WSR (45 u/75 u) 10% | ok | ok | ok |
| Suspensibility, 0.2% | — | 101 | 99.7 |
| Suspensibility, 1% | — | 102.8 | 101.6 |

The composition of Example 5 was stored for 2 weeks at 54° C. No crystal growth was observed. The concentration of the compound of Formula I was measured, and the concentration was higher than 95.

### Example 6: 450 SC Composition with a Mixture of Vinylpyrrolidone and Vinyl Acetate Copolymer (VA/VP)

A suspension concentrate (SC) composition containing 450 g/L of compound of Formula I with 1 built-in adjuvant (VP/VA) was prepared as follow:

### Step I: Preparation of Agriculturally Acceptable Inert Additives Premix

Soft water and Van Gel® B were charged to the vessel and mixed (high shear) to form a solution. The content of the vessel was heated to 60° C. SOPROPHORR TS/54 (TSP 54) was heated to 50-60° C. and added gradually to the vessel. SupragilR WP, SoprophorR 3D33, KH₂PO₄, Na₂HPO₄ and SAG 1572 were added to the homogeneous solution.

### Step II: Preparation of Compound of Formula I

Compound of Formula I (40% W/W) was added to the premix with the surfactants inside. The suspension was milled in a bead mill (0.8-1.2 mm beads) until a particle size of d90<5 μm was reached. The milled suspension was drained from the reactor to a new vessel.

### Step III: Finalization of the Composition

Propylene glycol and VP/VA were added to the milled suspension until a uniform suspension was obtained. Soft water and AgRH 23 2% solution were added to the suspension while mixing until a viscosity of 1600-2200 cP was reached. Mixing was continued until a homogenous solution was obtained. Viscosity was measured by viscometer according to CIPAC method MT 192.

The composition is summarized in Table 3.

TABLE 3
450 SC composition with VP/VA

| | | Amount for | |
|---|---|---|---|
| Ingredient trade name | Chemical name and CAS | 1000 L (kg) | |
| Compound of Formula I | 5-fluoro-4-imino-3methyl- | 450 kg | |
| tech. as 100% of ADAMA | 1-[4- | (469 kg | |
| Makhteshim LTD. | methylphenyl)sulfonyl]- | for 96%) | |
| | 3,4-dohydropyrimidin- | | |
| | 2(1H)-one | | |
| | CAS No. N/A | | |
| Supragil ® WP of Solvay or | Sodium | 5.9 | Kg |
| SURFOM HRB of OXITENO | Diisopropylnaphthalene | | |
| | sulphonate | | |
| | CAS No. 1322-93-6 | | |
| Soprophor ® 3 D 33 of | POLYARYLPHENYL | 47.0 | kg |
| Solvay | ETHER PHOSPHATE | | |
| | CAS No. 90093-37-1, | | |
| | 99734-09-5, 7664-38-2 | | |
| Emulsogen ® TS 540 of | Tristyryl phenol- | 17.5 | Kg |
| Clariant or | polyethylene glycol ether | | |
| SOPROPHOR ® TS/54 of | CAS No. 70559-25-0 (of | | |
| Solvay | Clariant), | | |
| | 104376-75-2 (of Solvay) | | |
| Agrimer ™ VA 6 of | Mixture of | 17.5 | Kg |
| Ashland or | vinylpyrrolidone and | | |
| Sokalan ® VA 64 P of | vinyl acetate copolymer | | |
| BASF | CAS No. 25086-89-9 | | |
| SAG ™ 1572 of Momentive | Polydimethylsiloxane | 2.0 | Kg |
| Performance Materials | emulsion | | |
| GmbH | CAS No. N/A, 56-81-5 | | |
| | (Glycerine) | | |
| MPG - MPG USP of INEOS | 1,2-propanediol | 23.5 | Kg |
| OXIDE or | CAS No. 57-55-6 | | |
| MPG Industrial TG of | | | |
| SHELL EASTERN | | | |
| CHEMICALS or | | | |
| Propylene Glycol | | | |
| Industrial Grade of | | | |
| DOW | | | |
| MKP (Mono potassium | KH₂PO₄ (Potassium | 1.7 | Kg |
| phosphate) of Rotem | dihydrogenorthophosphate) | | |
| Amfert Negev Ltd., | CAS No. 7778-77-0 | | |
| Disodium Phosphate | Na₂HPO₄ (Disodium | 6.9 | Kg |
| Anhydrous of Haifa | Phosphate Anhydrous) | | |
| Chemicals Ltd. | CAS No. 7558-79-4 | | |
| Van Gel ® B of | Magnesium aluminum | 5.9 | kg |
| Vanderbilt minerals, | silicate, Smectite clay; | | |
| LLC. | CAS No. 12199-37-0 | | |
| AgRH 23 of Ametech | Xanthan gum | 2.8 | Kg |
| | CAS No. 11138-66-2 | | |

| | |
|---|---|
| Soft Water | Up to |
| | 1000 L |
| | (about |
| | 599 kg) |

Appearance: Off-white homogeneous suspension
Density, g/ml: 1.15-1.25

### Stability Results:

The composition of Example 6 was stored for 2 weeks at 54° C. No crystal growth was observed. The concentration of the compound of Formula I was measured, and the concentration was slightly less than 95%.

### Example 7: 450 SC and 660 SC Compositions with a Mixture of Vinylpyrrolidone/Vinyl Acetate Copolymer and Silwet® L-077

Suspension concentrate (SC) compositions, one containing 450 g/L of the compound of Formula I and two built-in adjuvants (VP/VA and Silwet® L-077), and the other containing 660 g/L of the compound of Formula I and two built-in adjuvants (VP/VA and Silwet® L-077) were prepared as Follow:

Step I: Preparation of agriculturally acceptable inert additives premix Soft water (and Van Gel® B for the 450 SC composition) were charged to the vessel and mixed (high shear) to form a solution. The content of the vessel was heated to 60° C. SOPROPHOR® TS/54 (TSP 54) was heated to 50-60° C. and added gradually to the vessel. Supragil® WP, Soprophor® 3D33, KH₂PO₄, Na₂HPO₄ and SAG 1572 were added to the homogeneous solution.

### Step II: Preparation of Compound of Formula I

Compound of Formula I (40% W/W) was added to the premix with the surfactants inside. The suspension was milled in a bead mill (0.8-1.2 mm beads) until a particle size of d90<5 μm was reached. The milled suspension was drained from the reactor to a new vessel.

### Step III: Finalization of the Composition

Propylene glycol and Silwet® L-077 were added to the milled suspension until a uniform suspension was obtained. Soft water and AgRH 23 2% solution were added to the suspension while mixing until a viscosity of 1600-2200 cP was reached. Mixing was continued until a homogenous solution was obtained. Viscosity was measured by viscometer according to CIPAC method MT 192.

The 450 SC composition is summarized in Table 4 and the 660 SC composition is summarized in Table 5.

TABLE 4
450 SC composition with VP/VA and Silwet ® L-077

| Ingredient | | Amount for | |
|---|---|---|---|
| trade name | Chemical name and CAS | 1000 L (kg) | |
| Compound of | 5-fluoro-4-imino-3methyl-1- | 450 kg (469 | |
| Formula I tech. as | [4-methylphenyl)sulfonyl]- | kg for 96%) | |
| 100% of ADAMA | 3,4-dohydropyrimidin-2(1H)- | | |
| Makhteshim LTD. | one | | |
| | CAS No. N/A | | |
| Supragil ® WP of | Sodium | 5.9 | Kg |
| Solvay or | Diisopropylnaphthalene | | |
| SURFOM ® HRB of | sulphonate | | |
| OXITENO | CAS No. 1322-93-6 | | |
| Soprophor ® 3 D 33 | POLYARYLPHENYL ETHER | 47.0 | kg |
| of Solvay | PHOSPHATE | | |
| | CAS No. 90093-37-1, 99734-09- | | |
| | 5, 7664-38-2 | | |
| Emulsogen ® TS 540 | Tristyryl phenol-polyethylene | 17.5 | Kg |
| of Clariant or | glycol ether | | |
| SOPROPHOR ® TS/54 | CAS No. 70559-25-0 (of | | |
| of Solvay | Clariant), | | |
| | 104376-75-2 (of Solvay) | | |
| Agrimer ™ VA 6 of | Mixture of vinylpyrrolidone | 17.5 | Kg |
| Ashland or | and vinyl acetate copolymer | | |
| Sokalan ® VA 64 P | CAS No. 25086-89-9 | | |
| of BASF | | | |
| SAG ™ 1572 of | Polydimethylsiloxane emulsion | 2.0 | Kg |
| Momentive | CAS No. N/A, 56-81-5 | | |
| Performance | (Glycerine) | | |
| Materials GmbH | | | |
| Silwet ® L-077 of | Polyalkyleneoxide modified | 9.0 | kg |
| Momentive | Heptamethyltrisiloxane, CAS | | |
| | No. | | |
| | 27306-78-1 | | |
| MPG - MPG USP of | 1,2-propanediol | 23.5 | Kg |
| INEOS OXIDE or | CAS No. 57-55-6 | | |
| MPG Industrial TG | | | |
| of SHELL EASTERN | | | |
| CHEMICALS or | | | |
| Propylene Glycol | | | |
| Industrial Grade | | | |
| of DOW | | | |
| MKP (Mono | KH₂PO₄ (Potassium | 1.7 | Kg |
| potassium | dihydrogenorthophosphate) | | |
| phosphate) of | CAS No. 7778-77-0 | | |
| Rotem Amfert Negev | | | |
| Ltd., | | | |
| Disodium Phosphate | Na₂HPO₄ (Disodium Phosphate | 6.9 | Kg |
| Anhydrous of Haifa | Anhydrous) | | |
| Chemicals Ltd. | CAS No. 7558-79-4 | | |
| Van Gel ® B of | Magnesium aluminum silicate, | 5.9 | kg |
| Vanderbilt | Smectite clay; | | |
| minerals, LLC. | CAS No. 12199-37-0 | | |
| AgRH 23 of Ametech | Xanthan gum | 2.8 | Kg |
| | CAS No. 11138-66-2 | | |

| | |
|---|---|
| Soft Water | Up to 1000 |
| | L (about |
| | 590 kg) |

Appearance: Off-white homogeneous suspension
Density, g/ml: 1.15-1.25

### Stability Results:

The composition of Table 4 was stored for 2 weeks at 54° C. No crystal growth was observed. The concentration of the compound of Formula I was measured, and the concentration was slightly less than 95.

TABLE 5
660 SC composition with VP/VA and Silwet ® L-077

| Ingredient | | Amount for | |
|---|---|---|---|
| trade name | Chemical name and CAS | 1000 L (kg) | |
| Compound of Formula | 5-fluoro-4-imino-3methyl- | 660 kg | |
| I tech. as 100% of | 1-[4- | (687.5 kg | |
| ADAMA Makhteshim | methylphenyl)sulfonyl]- | for 96%) | |
| LTD. | 3,4-dohydropyrimidin- | | |
| | 2(1H)-one | | |
| | CAS No. N/A | | |
| Supragil ® WP of | Sodium | 10.0 | Kg |
| Solvay or | Diisopropylnaphthalene | | |
| SURFOM ® HRB of | sulphonate | | |
| OXITENO | CAS No. 1322-93-6 | | |
| Soprophor ® 3 D 33 | POLYARYLPHENYL | 56.25 | kg |
| of Solvay | ETHER PHOSPHATE | | |
| | CAS No. 90093-37-1, 99734- | | |
| | 09-5, 7664-38-2 | | |
| Emulsogen ® TS 540 of | Tristyryl phenol- | 21.25 | Kg |
| Clariant or | polyethylene glycol ether | | |
| SOPROPHOR ® TS/54 | CAS No. 70559-25-0 (of | | |
| of Solvay | Clariant), | | |
| | 104376-75-2 (of Solvay) | | |
| Agrimer ™ VA 6 of | Mixture of | 13.5 | Kg |
| Ashland or | vinylpyrrolidone and vinyl | | |
| Sokalan ® VA 64 P of | acetate copolymer | | |
| BASF | CAS No. 25086-89-9 | | |
| SAG ™ 1572 of | Polydimethylsiloxane | 5.0 | Kg |
| Momentive | emulsion | | |
| Performance | CAS No. N/A, 56-81-5 | | |
| Materials GmbH | (Glycerine) | | |
| Silwet ® L-077 of | Polyalkyleneoxide modified | 9.5 | kg |
| Momentive | Heptamethyltrisiloxane, | | |
| | CAS No. | | |
| | 27306-78-1 | | |
| MPG - MPG USP of | 1,2-propanediol | 37.5 | Kg |
| INEOS OXIDE or | CAS No. 57-55-6 | | |
| MPG Industrial TG of | | | |
| SHELL EASTERN | | | |
| CHEMICALS or | | | |
| Propylene Glycol | | | |
| Industrial Grade of | | | |
| DOW | | | |
| MKP (Mono potassium | KH₂PO₄ (Potassium | 2.5 | Kg |
| phosphate) of Rotem | dihydrogenorthophosphate) | | |
| Amfert Negev Ltd., | CAS No. 7778-77-0 | | |
| Disodium Phosphate | Na₂HPO₄ (Disodium | 11.25 | Kg |
| Anhydrous of Haifa | Phosphate Anhydrous) | | |
| Chemicals Ltd. | CAS No. 7558-79-4 | | |
| AgRH 23 of Ametech | Xanthan gum | 0.25 | Kg |
| | CAS No. 11138-66-2 | | |

| | |
|---|---|
| Soft Water | Up to 1000 L |
| | (about 423 |
| | kg) |

Appearance: Off-white homogeneous suspension
Density, g/ml: 1.15-1.35

### Stability Results:

Stability results of the composition of Table 5 are summarized in Table 6 below.

TABLE 6
Stability Results

| | | | Oven | Cold | Cold | Oven |
|---|---|---|---|---|---|---|
| | | Room | 54° C. | 4° C. | 10° C. | 40° C. |
| | | after 2 | after 2 | after 2 | after 2 | after 8 |
| | Start | weeks | weeks | weeks | weeks | weeks |
| Appearance | Off white | Off white | Light phase | Off white | Off white | Phase |
| | suspension | suspension | separation | suspension | suspension | separation |
| Concentration | 57.10% | 56.5% | 55.2% | 56.3% | 56.3% | — |
| of compound | | | | | | |
| of Formula I | | | | | | |
| Density, g/ml | 1.314 | 1.275 | 1.215 | 1.27 | 1.252 | 1.279 |
| pH | 6.6 | 6.6 | 6.5 | 6.5 | 6.6 | 6.36 |
| Viscosity, | 1400 | 1800 | 1040 | 690 | 1460 | 1410 |
| 12 rpm, | | | | | | |
| sp.62 (cP) | | | | | | |
| Particle | 4.7 | 4.95 | 5.76 | 4.8 | 4.84 | 5.43 |
| size (D₉₀) | | | | | | |

### Example 8: 450 SC Composition with Two Built-In Adjuvants

A suspension concentrate (SC) composition containing 450 g/L of compound of Formula I and two built-in adjuvants (PVP and Silwet® L-077) was prepared as follow:

### Step I: Preparation Agriculturally Acceptable Inert Additives Premix

Soft water and Van Gel® B were charged to a vessel, and the solutions were mixed (high shear). The content of the vessel was heated to 60° C. SOPROPHOR® TS/54 (TSP 54) was heated to 50-60° C. and added gradually to the vessel. Supragil® WP, Soprophor® 3D33, KH₂PO₄, Na₂HPO₄ and SAG were then added.

### Step II: Preparation of the Compound of Formula I

The compound of Formula I (40% W/W) was added to the premix with the surfactants inside to form a suspension. The suspension was milled in a bead mill (0.8-1.2 mm beads) until a particle size of d90<5 μm was reached. The suspension was drain from the reactor to a new vessel.

### Step III: Finalization of the Composition

Propylene glycol and PVP were added to the milled suspension and mixed until a uniform suspension was obtained. Soft water and Ag RH 23 2% solution were added while mixing until a viscosity of 1600-2200 cP was reached. Mixing was continued until the solution was homogenous. Viscosity was measured by viscometer according to CIPAC method MT 192.

The composition is summarized in Table 7 below.

TABLE 7
450 SC composition with PVP and Silwet ® L-077

| | | Amount for | |
|---|---|---|---|
| Chemical name and CAS | CAS No. | 1000 L (kg) | |
| Compound of Formula I | | 450 kg (469 kg | |
| | | for 96%) | |
| Sodium | CAS No. 1322- | 5.9 | Kg |
| Diisopropylnaphthalene | 93-6 | | |
| sulphonate | (Supragil WP | | |
| | of Solvay or | | |
| | SURFOM HRB | | |
| | of OXITENO | | |
| Soprophor ® 3 D 33 of Solvay | CAS No. | 47.0 | kg |
| POLYARYLPHENYL ETHER | 90093-37-1, | | |
| PHOSPHATE | 99734-09-5, | | |
| | 7664-38-2 | | |
| Emulsogen ® TS 540 of Clariant | CAS No. | 17.5 | Kg |
| or | 70559-25-0 | | |
| SOPROPHOR ® TS/54 of Solvay | (of Clariant), | | |
| Tristyryl phenol-polyethylene | 104376-75-2 | | |
| glycol ether | (of Solvay) | | |
| PVP K-30 of Ashland | CAS No. 9003- | 17.5 | Kg |
| Vinylpyrrolidone polymer | 39-8 | | |
| SAG ™ 1572 of Momentive | CAS No. N/A, | 2.0 | Kg |
| Performance Materials GmbH | 6-81-5 | | |
| Polydimethylsiloxane emulsion | (Glycerine) | | |
| Silwet ® L-077 of Momentive | CAS No. | 9.0 | kg |
| Polyalkyleneoxide modified | 27306-78-1 | | |
| Heptamethyltrisiloxane, | | | |
| MPG - MPG USP of INEOS OXIDE | CAS No. 57- | 23.5 | Kg |
| or MPG Industrial TG of SHELL | 55-6 | | |
| EASTERN CHEMICALS or | | | |
| Propylene Glycol Industrial | | | |
| Grade of DOW | | | |
| 1,2-propanediol | | | |
| MKP (Mono potassium phosphate) | CAS No. 7778- | 1.7 | Kg |
| of Rotem Amfert Negev Ltd., | 77-0 | | |
| KH₂PO₄ (Potassium | | | |
| dihydrogenorthophosphate) | | | |
| Disodium Phosphate Anhydrous of | CAS No. 7558- | 6.9 | Kg |
| Haifa Chemicals Ltd | 79-4 | | |
| Na₂HPO₄ (Disodium Phosphate | | | |
| Anhydrous) | | | |
| Van Gel ® B of Vanderbilt | CAS No. | 5.9 | kg |
| minerals, LLC. Magnesium | 12199-37-0 | | |
| aluminum silicate, Smectite | | | |
| clay; | | | |
| AgRH 23 of Ametech | CAS No. | 2.8 | Kg |
| Xanthan gum | 11138-66-2 | | |

| | |
|---|---|
| Soft water | Up to 1000 L |
| | (about 590 kg) |

Appearance: Off-white homogeneous suspension
Density, g/ml: 1.15-1.25

### Example 9: 300 SE Composition with VP/VA, Agnique® BP 420 and Agnique® ME 18 RD-F

A suspoemulsion (SE) composition containing 300 g/L of compound of Formula I and three built-in adjuvants (VP/VA, Agnique® BP 420 and Agnique® ME 18 RD-F) was prepared as follow:

Step I: Preparation of agriculturally acceptable inert additives premix

Soft water and Van Gel® B were charged to the vessel and mix (high shear). The content of the vessel was heated to 60° C. SOPROPHOR® TS/54 (TSP 54) was heated to 50-60° C. and added gradually to the vessel. Supragil® WP, Soprophor® 3D33, KH₂PO₄, Na₂HPO₄ and SAG were added to the solution.

### Step II: Preparation of Compound of Formula I

Compound of Formula I (40% W/W) was added to the premix with the surfactants inside to form a suspension. The suspension was milled in a bead mill (0.8-1.2 mm beads) until a particle size of d90<5 μm was reached. The milled suspension was drained from the reactor to new vessel.

### Step III: Preparation of the Organic Phase

Agnique® ME RDF, Atlox™ 4914, Atlas™ G5002L, Genapol® X80 and Agnique® BP 420 were charged to the vessel and mixed until a homogeneous solution was obtained. Before adding the SE to the suspension, the content of the vessel was mixed (high shear) for at least 10 min until a droplet size of D90=10 μm was reached.

### Step IV: Finalization of the Composition

Propylene glycol and VP/VA were added to the milled suspension and mixed until a uniform suspension was obtained. The SE solution was added gradually to the milled suspension in three doses. Between each dose, Atlox™ 4913, soft water and Ag RH 23 2% solution were added while mixing until a viscosity of 1600-2200 cP was reached. Mixing was continued until a homogenous solution was obtained. Viscosity was measured by viscometer according to CIPAC method MT 192.

The compositions are summarized in Table 8 below.

TABLE 8
300 SE composition with VP/VA, Agnique ® BP
420 and Agnique ® ME 18 RD- F

| Ingredient | | Func- | Amount for | |
|---|---|---|---|---|
| trade name | Chemical name and CAS | tion | 1000 L (kg) | |
| Compound of | 5-fluoro-4-imino-3methyl- | | 300 kg | |
| Formula I | 1-[4- | | (312.5 kg | |
| | methylphenyl)sulfonyl]- | | for 96%) | |
| | 3,4-dohydropyrimidin- | | | |
| | 2(1H)-one | | | |
| | CAS No. N/A | | | |
| Supragil ® WP of | Sodium | | 4.0 | Kg |
| Solvay or | Diisopropylnaphthalene | | | |
| SURFOM ® HRB | sulphonate | | | |
| of OXITENO | CAS No. 1322-93-6 ( ) | | | |
| Soprophor ® 3 D | POLYARYLPHENYL | | 31.5 | kg |
| 33 of Solvay | ETHER PHOSPHATE | | | |
| | CAS No. 90093-37-1, | | | |
| | 99734-09-5, 7664-38-2 | | | |
| Emulsogen ® TS | Tristyryl phenol- | | 12.0 | Kg |
| 540 of Clariant or | polyethylene glycol ether | | | |
| SOPROPHOR ® | CAS No. 70559-25-0 (of | | | |
| TS/54 of Solvay | Clariant), | | | |
| | 104376-75-2 (of Solvay) | | | |
| Agrimer ™ VA 6 of | Mixture of vinylpyrrolidone | | 17.5 | Kg |
| Ashland or | and vinyl acetate copolymer | | | |
| Sokalan ® VA 64 P | CAS No. 25086-89-9 | | | |
| of BASF | | | | |
| SAG ™ 1572 of | Polydimethylsiloxane | | 2.0 | Kg |
| Momentive | emulsion | | | |
| Performance | CAS No. N/A, 56-81-5 | | | |
| Materials GmbH | (Glycerine) | | | |
| MPG - MPG USP | 1,2-propanediol | | 23.5 | Kg |
| of INEOS OXIDE | CAS No. 57-55-6 | | | |
| or MPG Industrial | | | | |
| TG of SHELL | | | | |
| EASTERN | | | | |
| CHEMICALS or | | | | |
| Propylene Glycol | | | | |
| Industrial Grade | | | | |
| of DOW | | | | |
| MKP (Mono | KH₂PO₄ (Potassium | | 1.2 | Kg |
| potassium | dihydrogenorthophosphate) | | | |
| phosphate) of | CAS No. 7778-77-0 | | | |
| Rotem Amfert | | | | |
| Negev Ltd. | | | | |
| Disodium | Na₂HPO₄ (Disodium | | 4.6 | Kg |
| Phosphate | Phosphate Anhydrous) | | | |
| Anhydrous of | CAS No. 7558-79-4 | | | |
| Haifa Chemicals | | | | |
| Ltd. | | | | |
| Van Gel ® B of | Magnesium aluminum | | 4.0 | kg |
| Vanderbilt | silicate, Smectite clay; | | | |
| minerals, LLC. | CAS No. 12199-37-0 | | | |
| Agnique ® ME 18 | Rapeseed oil fatty acid | | 64.5 | kg |
| RD-F | methyl ester. | | | |
| | CAS No. 67762-38-3 | | | |
| Atlox ™ 4914 of | Random copolymer (alkyd- | | 9.5 | kg |
| Croda | PEG resin). | | | |
| | CAS No. N/A | | | |
| Atlas ™ G5002L of | Polyalkylene oxide block | | 29.0 | kg |
| Croda | copolymer. | | | |
| | CAS No. N/A | | | |
| Genapol ® X80 of | A fatty alcohol polyglycol | | 13.0 | kg |
| Croda | ether. | | | |
| | CAS No. 9043-30-5 | | | |
| Agnique ® BP 420 | Alcohols, C16-18, | | 97.0 | kg |
| of BASF | ethoxylated propoxylated. | | | |
| | CAS No. 68002-96-0 | | | |
| Atlox ™ 4913 of | Methyl ether methacrylate | | 35.0 | kg |
| Croda | copolymer. | | | |
| | CAS No. N/A. | | | |
| | Propylene glycol. CAS No. | | | |
| | 57-55-6 | | | |
| AgRH 23 of | Xanthan gum | | 1.0 | Kg |
| Ametech | CAS No. 11138-66-2 | | | |

| | |
|---|---|
| Soft Water | Up to 1000 L |
| | (about 511.0 |
| | kg) |

Appearance: Off-white homogeneous suspension
Density, g/ml: 1.14-1.25

### Example 10: 300 SE Composition with PVP, AgniqueR BP 420 and Agnique® ME 18 RD-F

SE composition containing 300 g/L of compound of Formula I and three built-in adjuvants (PVP, AgniqueR BP 420 and AgniqueR ME 18 RD-F) was prepared as follow:

### Step I: Preparation of Agriculturally Acceptable Inert Additives Premix

Soft water and Van Gel® B were charged to the vessel and mixed (high shear). The content of the vessel was heated to 60° C. SOPROPHOR® TS/54 (TSP 54) was heated to 50-60° C. and added gradually to the vessel. Supragil® WP, Soprophor® 3D33, KH₂PO₄, Na₂HPO₄ and SAG were added to the solution.

### Step II: Preparation of Compound of Formula I

Compound of Formula I (40% W/W) was added to the premix with the surfactants inside to form a suspension. The suspension was milled in a bead mill (0.8-1.2 mm beads) until a particle size of d90<5 μm was reached. The milled suspension was drained from the reactor to new vessel.

### Step III: Preparation of the Organic Phase

Agnique® ME RDF, Atlox™ 4914, Atlas® G5002L, Genapol® X80 and Agnique® BP 420 were charged to the vessel and mixed until a homogeneous solution was obtained. Before adding the SE to the suspension, the content of the vessel was mixed (high shear) for at least 10 min until a droplet size of D90=10 μm was reached.

### Step IV: Finalization of the Composition

Propylene glycol and PVP were added to the milled suspension and mixed until a uniform suspension was obtained. The SE solution was added gradually to the milled suspension in three doses. Between each dose, Atlox™ 4913, soft water and Ag RH 23 2% solution were added while mixing until a viscosity of 1600-2200 cP was reached. Mixing was continued until a homogenous solution was obtained. Viscosity was measured by viscometer according to CIPAC method MT 192.

The compositions are summarized in Table 9 below.

TABLE 9
300 SE Composition with PVP, Agnique ® BP 420
and Agnique ® ME 18 RD- F

| | | Amount for | |
|---|---|---|---|
| Ingredient trade name | Chemical name and CAS | 1000 L (kg) | |
| Compound of Formula I. | 5-fluoro-4-imino-3methyl-1- | 300 kg | |
| | [4-methylphenyl)sulfonyl]- | (312.5 kg | |
| | 3,4-dohydropyrimidin-2(1H)- | for 96%) | |
| | one | | |
| | CAS No. N/A | | |
| Supragil ® WP of Solvay | Sodium | 4.0 | Kg |
| or | Diisopropylnaphthalene | | |
| SURFOM ® HRB of | sulphonate | | |
| OXITENO | CAS No. 1322-93-6 | | |
| Soprophor ® 3 D 33 of | POLYARYLPHENYL | 31.5 | kg |
| Solvay | ETHER PHOSPHATE | | |
| | CAS No. 90093-37-1, 99734- | | |
| | 09-5, 7664-38-2 | | |
| Emulsogen ® TS 540 of | Tristyryl phenol- | 12.0 | Kg |
| Clariant or | polyethylene glycol ether | | |
| SOPROPHOR ® TS/54 of | CAS No. 70559-25-0 (of | | |
| Solvay | Clariant), | | |
| | 104376-75-2 (of Solvay) | | |
| PVP K-30 of Ashland or | Vinylpyrrolidone polymer | 17.5 | Kg |
| | CAS No. 9003-39-8 | | |
| SAG ™ 1572 of | Polydimethylsiloxane | 2.0 | Kg |
| Momentive | emulsion | | |
| Performance Materials | CAS No. N/A, 56-81-5 | | |
| GmbH | (Glycerine) | | |
| MPG - MPG USP of | 1,2-propanediol | 23.5 | Kg |
| INEOS OXIDE or | CAS No. 57-55-6 | | |
| MPG Industrial TG of | | | |
| SHELL EASTERN | | | |
| CHEMICALS or | | | |
| Propylene Glycol | | | |
| Industrial Grade of DOW | | | |
| MKP (Mono potassium | KH₂PO₄ (Potassium | 1.2 | Kg |
| phosphate) of Rotem | dihydrogenorthophosphate) | | |
| Amfert Negev Ltd., | CAS No. 7778-77-0 | | |
| Disodium Phosphate | Na₂HPO₄ (Disodium | 4.6 | Kg |
| Anhydrous of Haifa | Phosphate Anhydrous) | | |
| Chemicals Ltd. | CAS No. 7558-79-4 | | |
| Van Gel ® B of Vanderbilt | Magnesium aluminum | 4.0 | kg |
| minerals, LLC. | silicate, Smectite clay; | | |
| | CAS No. 12199-37-0 | | |
| Agnique ® ME 18 RD-F | Rapeseed oil fatty acid | 64.5 | kg |
| | methyl ester. | | |
| | CAS No. 67762-38-3 | | |
| Atlox ™ 4914 of Croda | Random copolymer (alkyd- | 9.5 | kg |
| | PEG resin). | | |
| | CAS No. N/A | | |
| Atlas ™ G5002L of Croda | Polyalkylene oxide block | 29.0 | kg |
| | copolymer. | | |
| | CAS No. N/A | | |
| Genapol ® X80 of Croda | A fatty alcohol polyglycol | 13.0 | kg |
| | ether. | | |
| | CAS No. 9043-30-5 | | |
| Agnique ® BP 420 of | Alcohols, C16-18, | 97.0 | kg |
| BASF | ethoxylated propoxylated. | | |
| | CAS No. 68002-96-0 | | |
| Atlox ™ 4913 of Croda | Methyl ether methacrylate | 35.0 | kg |
| | copolymer. | | |
| | CAS No. N/A. | | |
| | Propylene glycol. CAS No. | | |
| | 57-55-6 | | |
| AgRH 23 of Ametech | Xanthan gum | 1.0 | Kg |
| | CAS No. 11138-66-2 | | |

| | |
|---|---|
| Soft Water | Up to 1000 |
| | L (about |
| | 511.0 kg) |

Appearance: Off-white homogeneous suspension
Density, g/ml: 1.14-1.25

### Example 11: Suspension Concentrate (SC) Composition without Stabilizing Surfactant at pH of Approx. 7

A suspension concentrate (SC) composition containing 450 g/L of the compound of Formula I and no stabilizing surfactant was prepared as follows:

In water, 4% Atlox™ 4913, 20 Ethylan™ NS 500 LQ/Antarox® B 848, 0.50 Supragil® WP and 0.1% antifoam (SAG™ 1572) were added and mixed until a homogeneous solution was obtained.

Compound of Formula I was added while mixing (high sheer). Mixing was performed for 5 minutes. The mixture was put in the Tinky with a few beads for 20 minutes.

The rest of the materials were inserted into the suspension and mixed, divided to vials and put in the room and oven.

The composition is summarized in Table 10.

TABLE 10
450 SC composition of the compound
of Formula I with pH approx. = 7

| | | Quantity for |
|---|---|---|
| | | 1000 Liters |
| | | (up to 1180 kg |
| | | with 1. 18 |
| Raw Material | CAS No. | gr/ml density) |
| Compound of Formula I, tech. | | 450 kg (469 kg |
| as 100% | | for 96%) |
| 4% Atlox ™ 4913 | 1322-93-6 | 47 |
| 2% Ethylan ™ NS 500 LQ/ | 90093-37-1 | 24 |
| Antarox ® B 848 | | |
| 0.5% Supragil ® WP | 70559-25-0 | 59 |
| Propylene Glycol | 57-55-6 | 24 |
| SAG ™ 1572 | Trade secret | 30 |
| (Polydimethylsiloxane antifoam | | |
| emulsion) | | |
| Xantan Gum AG RH 2.0% | 11138-66-2 | 118 |
| KH₂PO₄ (Monopotassium | 7778-77-0 | 1.7 |
| phosphate) | | |
| Na₂HPO₄ (Disodium phosphate) | 7558-79-4 | 6.9 |
| water DI | | Up to 1000 L |
| | | (about 419.4 |
| | | kg) |

### Stability Results

The SC composition was placed in room temperature and in oven (54° C.) for 24 hours and the concentration was checked. Crystalline particle was observed and the degradation of the compound of Formula I was measured. The results showed that there was more than 5% decrease in concentration of the compound of Formula I.

### Example 12: Suspension Concentrate Composition with Stabilizing Surfactants at pH 3.5

A suspension concentrate (SC) composition containing 450 g/L of compound of Formula I and two stabilizing surfactants (Soprophor® 3D33 and Soprophor® TS/54) was prepared as follow:

### Step I: Preparation of Agriculturally Acceptable Inert Additives Premix

Soft water was added gradually to a vessel containing preheated SOPROPHOR® TS/54 (TSP 54). The content of the vessel was mixed and heated to 50-65° C. until a homogeneous solution was obtained. Supragil® WP, Soprophor® 3D33, KH₂PO₄, Na₂HPO₄ and SAG™ 1572 were added to the solution.

### Step II: Preparation of the Compound of Formula I

Compound of Formula I (40% W/W) was added to the premix with the surfactants inside to form a suspension. The suspension was milled in a bead mill (0.8-1.2 mm beads) until a particle size of d90<5 μm was reached. The milled suspension was drained from the reactor to a new vessel.

### Step III: Finalization of the Composition

Propylene glycol was added to the milled suspension and mixed until a uniform suspension was obtained. Soft water and AgRH 23 2% solution were added to the suspension while mixing until a viscosity of 1600-2200 cP was reached. Mixing was continued until a homogenous solution was obtained. Viscosity was measured by viscometer according to CIPAC method MT 192.

The composition is summarized in Table 11.

TABLE 11
450 SC composition of compound Formula I with pH = 3.5

| | Chemical name | Amount for | |
|---|---|---|---|
| Ingredient trade name | and CAS No. | 1000 L (kg) | |
| Compound of Formula I, | 5-fluoro-4-imino-3methyl- | 450 kg (469 kg | |
| tech. as 100% of | 1-[4- | for 96%) | |
| ADAMA | methylphenyl)sulfonyl]-3,4- | | |
| Makhteshim LTD. | dohydropyrimidin-2(1H)- | | |
| | one | | |
| | CAS No. N/A | | |
| Supragil ® WP of | Sodium | 5.9 | Kg |
| Solvay or | Diisopropylnaphthalene | | |
| SURFOM ® HRB of | sulphonate | | |
| OXITENO | CAS No. 1322-93-6 | | |
| Soprophor ® 3 D 33 of | POLYARYLPHENYL | 47.0 | kg |
| Solvay | ETHER PHOSPHATE | | |
| | CAS No. 90093-37-1, 99734- | | |
| | 09-5, 7664-38-2 | | |
| Emulsogen ™ TS 540 of | Tristyryl phenol- | 17.5 | Kg |
| Clariant or | polyethylene glycol ether | | |
| SOPROPHOR ® | CAS No. 70559-25-0 (of | | |
| TS/54 of Solvay | Clariant), | | |
| | 104376-75-2 (of Solvay) | | |
| SAG ™ 1572of | Polydimethylsiloxane | 2.0 | Kg |
| Momentive | emulsion | | |
| Performance Materials | CAS No. N/A, 56-81-5 | | |
| GmbH | (Glycerine) | | |
| MPG - MPG USP of | 1,2-propanediol | 23.5 | Kg |
| INEOS OXIDE or | CAS No. 57-55-6 | | |
| MPG Industrial TG of | | | |
| SHELL EASTERN | | | |
| CHEMICALS or | | | |
| Propylene Glycol | | | |
| Industrial Grade of | | | |
| DOW | | | |
| AgRH 23 of Ametech | Xanthan gum | 2.8 | Kg |
| | CAS No. 11138-66-2 | | |

| | |
|---|---|
| Soft Water | Up to 1000 L |
| | (about 621 kg) |

### Stability Results:

The concentration in the accelerated storage after 2 weeks in the oven at 54° C. decreased in 60. A small amount of crystalline particles was observed. However, the concentration of compound of Formula I was reduced.

Accordingly, the pH of the composition must be maintained within the range of 5.0-7.5 in order to have a stable SC composition.

### Example 13: Suspension Concentrate Compositions with Stabilizing Surfactants at pH 4 and 8

Table 12 shows two SC compositions (BN 161213-5-Sop3d_TSP54_PG and BN 161213-6-Sop3d_TSP54_PG) each containing two stabilizing surfactants (Soprophor® 3D33 and Soprophor® TS/54). BN 161213-5-Sop3d_TSP4 PG has a low pH of 4 and BN 161213-6-Sop3d_TSP54_PG has a high pH of 8.

Neither composition was stable. The concentration decreased in the oven in the first composition and major viscosity elevation was observed for the second composition. Viscosity was measured by viscometer according to CIPAC method MT 192.

TABLE 12
Comparison of SC compositions at pH 4 and 8

| | BN 161213-5- | | | BN 161213-6- | | |
|---|---|---|---|---|---|---|
| | Sop3d TSP54 PG | | | Sop3d_TSP54 PG | | |
| | PH 4 | | | PH 8 | | |
| Composition: | % W/W | g/1 L | g/2.5 | % W/W | g/1 L | g/2.5 |
| Compound of | 44.51 | 489.58 | 1223.96 | 44.51 | 489.58 | 1223.96 |
| formula I | | | | | | |
| Supragil ® WP | 0.50 | 5.50 | 13.75 | 0.50 | 5.50 | 13.75 |
| Soprophor ® 3d33 | 4.00 | 44.00 | 110.00 | 4.00 | 44.00 | 110.00 |
| TSP54 | 1.50 | 16.50 | 41.25 | 1.50 | 16.50 | 41.25 |
| SAGTM1572 | 0.15 | 1.65 | 4.13 | 0.15 | 1.65 | 4.13 |
| Water | 32.24 | 354.67 | 886.67 | 31.84 | 350.27 | 875.67 |
| sub total | 82.90 | 911.90 | 2279.75 | 82.50 | 907.50 | 2268.75 |

Milling parameters: 5 cycles, the first two at 2500-3000 rpm,
the third at 3100 rpm and the final two at 3500-3600 rpm
(temp. 24-28° C.)

| After milling, the milled | | | | | | |
|---|---|---|---|---|---|---|
| solution is split into batches | | | | | | |
| Total milled | | | | | | |
| composition: | | | | | | |
| 2000 gr | | | | | | |
| | | | 200.00 | | | 200.00 |
| Factor | | | 0.09 | | | 0.09 |
| Xantan gum Ag/RH | 12.00 | 132.00 | 28.95 | 12.00 | 132.00 | 28.95 |
| 2% | | | | | | |
| Propylene glycol | 5.00 | 55.00 | 12.06 | 5.00 | 55.00 | 12.06 |
| Citric acid | 0.10 | 1.10 | 0.24 | 0.00 | 0.00 | 0.00 |
| Trisodium citrate | | | | 0.50 | 5.50 | 1.21 |
| Urea | 0.00 | 0.00 | 0.00 | 0.00 | 0.00 | 0.00 |
| Total | 100.0 | 1100.00 | 241.25 | 100.0 | 1100.00 | 242.22 |

| | BN 161213-5- | BN 161213-6- |
|---|---|---|
| | Sop3d_TSP54_PG | Sop3d_TSP54_PG |
| | PH 4 | PH 8 |
| PH | 4 | 8 |
| Viscosity (cP) 12 rpm S62 | 1440 | 1610 |
| Concentration of compound of | 44.4 | 37.7 |
| formula I (%) | | |
| Concentration of compound of | 36.4 | 38 |
| formula I (%) | | |
| oven 2W 54° C. (%) | | |
| Comments | Viscosity elevation | Viscosity elevation, |
| | occurred and | aggregation and |
| | crystallization | crystallization |
| | | occurred. |

When pH is low, i.e. pH 4, there is degradation of the compound for Formula I. In Table 12, the amount of the compound of Formula I in the composition decreased from 44.4% by weight based on the total weight of the composition to 31.1% by weight based on the total weight of the composition. When pH is high, the physical stability of the composition decreases. In Table 12, the composition became inhomogeneous and viscosity elevated.

As a conclusion, there is a need to maintain the pH of the composition in the range of 5.0-7.5 in order to have a stable SC composition.

### Example 14: 250 OD Composition with Agnique ME 18 RD-F (OD Composition A)

An oil dispersion (OD) composition containing 250 g/L of compound of Formula I was prepared as follows:

Step I: Preparation of Agriculturally Acceptable Inert Additives Premix in Non-Aqueous Liquid Carrier (Fatty Acid Ester) Bentone SD®-1 was added to Agnique® ME 18 RD-F under high shear (3000 rpm) using a large round hole Silverson mixer and mixed for 5 minutes.

The mixture was milled in a colloid mill (IKA MagicLab) until the viscosity at 10 s⁻¹ was no longer significantly increasing (50 passes). Viscosity was measured by viscometer according to CIPAC method MT 192.

Agnique® ME 18 RD-F was added to a suitable vessel and the pre-gel was added and mixed for 5 minutes.

Emulsifiers and dispersants were added and mixed for 15 minutes until homogenous.

### Step II: Preparation of Compound of Formula I

Compound of Formula I was added under agitation and mixed until homogenous. Once all active ingredients were added, it was mixed for 15 minutes.

### Step III: Finalization of the Composition

The sample was milled in Eiger mini motor mill (80% 0.75 mm-1.0 mm bead charge at 4000 rpm) for 15 minutes.

The composition is summarized in Table 13.

TABLE 13
250 OD composition of the compound of Formula I (A)

| Ingredient | % w/w |
|---|---|
| compound of Formula I | 25 |
| Atlox ™ 4912 | 5 |
| Atlox ™ 4915 | 2 |
| Rhodocal ® 70/B | 5 |
| Genapol ® X 080 | 4 |
| Genapol ® X 050 | 8 |
| Bentone SD ®-1 | 1.5 |
| Agnique ® ME 18 RD-F | up to 100% (49.5) |

Density 1 gr/ml

### Example 15: 250 OD Composition with Agnique® ME 18 RD-F (OD Composition B)

An oil dispersion (OD) composition containing 250 g/L of compound of Formula I was prepared as follows:

### Step I: Preparation of Agriculturally Acceptable Inert Additives Premix in Non-Aqueous Liquid Carrier (Fatty Acid Ester)

Bentone SD®-1 was added to Agnique® ME 18 RD-F under high shear (3000 rpm) using a large round hole Silverson mixer and mixed for 5 minutes.

The mixture was milled in a colloid mill (IKA MagicLab) until the viscosity at 10 s⁻¹ was no longer significantly increasing (50 passes). Viscosity was measured by viscometer according to CIPAC method MT 192.

Agnique® ME 18 RD-F was added to a suitable vessel and the pre-gel was added and mixed for 5 minutes.

Emulsifiers and dispersants were added and mixed for 15 minutes until homogenous.

### Step II: Preparation of Compound of Formula I

Compound of Formula I was added under agitation and mixed until homogenous. Once all active ingredients were added, it was mixed for 15 minutes.

### Step III: Finalization of the Composition

The sample was milled in Eiger mini motor mill (80% 0.75 mm-1.0 mm bead charge at 4000 rpm) for 15 minutes.

The composition is summarized in Table 14.

TABLE 14
250 OD composition of the compound of Formula I (B)

| Ingredient | % w/W |
|---|---|
| Compound of Formula I | 25 |
| Atlox ™ 4912 | 2 |
| Atlox ™ 4915 | 1.25 |
| Aerosol ® OT-SE | 5 |
| Genapol ® x80 | 5 |
| Rhodasurf ® LA30 | 5 |
| Bentone SD ®-1 | 1.25 |
| Agnique ® ME 18-RD-F | 55.5 |
| (solvent /adjuvant) | |

The composition of Example 15 was physically stable (phase separation was observed, however it was homogeneous after mixing). Chemical degradation was observed after 2 weeks of storage at 54° C. (<10% of the compound of Formula I degraded).

### Example 16: 250 OD Composition with Agnique® ME 18 RD-F (OD Composition C)

An oil dispersion (OD) composition containing 250 g/L of compound of Formula I was prepared using a process similar to Example 14 and 15.

The composition is summarized in Table 15.

TABLE 15
250 OD composition of the compound of Formula I (C)

| Component | WEIGHT |
|---|---|
| Compound of Formula I | 250 |
| (100% purity) | |
| Atlox ™ 4912 | 30 |
| Atlox ™ 4915 | 20 |
| Genapol ® X080 | 37.5 |
| Genapol ® X050 | 75 |
| Bentone SD ®-1 | 5 |
| Attagel ® 50 | 5 |
| Rhodacal ® 70/B | 50 |
| Agnique ® ME 18 RD-F | TO 1 LITER (~527.5) |

The composition of Example 16 was stored for 2 weeks at 54° C. and no significant degradation of the compound of Formula I was observed.

### Example 17: 250 OD Composition with Agnique® ME 18 RD-F (OD Composition D)

### Step I: Preparation of Agriculturally Acceptable Inert Additives Premix in Non-Aqueous Liquid Carrier (Fatty Acid Ester)

Agnique® ME 18 RD-F was added to a suitable vessel and the pre-gel was added and mixed for 5 minutes.

Emulsifiers and dispersants were added and mixed for 15 minutes until homogenous.

### Step II: Preparation of Compound of Formula I

Compound of Formula I was added under agitation and mixed until homogenous. Once all active ingredients were added, it was mixed for 15 minutes.

The mixture was milled in a colloid mill (IKA MagicLab) until the viscosity at 10 s⁻ was no longer significantly increasing (50 passes). Viscosity was measured by viscometer according to CIPAC method MT 192.

### Step III: Finalization of the Composition

The sample was milled in Eiger mini motor mill (80% 0.75 mm-1.0 mm bead charge at 4000 rpm) for 15 minutes.

TABLE 16
250 OD composition of the compound of Formula I (D)

| Ingredient | % w/w |
|---|---|
| compound of Formula I | 25 |
| Atlox ™ 4912 | 3 |
| Atlox ™ 4915 | 2 |
| Rhodocal ® 70/B or Aerosol ® OT-SE* | 6 |
| Alkamuls ® BR or Atlas ™ G5002L* | 6 |
| Genapol ® X 050 | 3 |
| Agnique ® ME 18 RD-F | up to 100% (55) |

*The actual composition was prepared with Rhodocal ® 70/B and Alkamuls ® BR. The Aerosol ® OT-SE and Atlas ™ G5002 are written as an additional option, however they were not tested.

TABLE 17
Compound of Formula I - 250 g/L OD composition specification

| Characteristic | Specification | |
|---|---|---|
| Appearance | Brown to White, homogenous | |
| | liquid | |
| Persistent Foam (CIPAC MT 47.3) | <60 mL after 1 minute | |
| pH (1% deionized water, CIPAC MT | 4-9 | |
| 75.3) | | |
| Viscosity at 10 s⁻¹ | >500 | mPa · s |
| Yield Stress | >1 | Pa |

| | |
|---|---|
| Dispersion Stability (CIPAC MT 180) | <2 mL separation after 30 |
| | minutes |
| Wet Sieve (CIPAC MT 185) | <2% reside on 75 μm sieve |
| Particle Size (CIPAC MT 187) | D(50) μm: <4, D(90) μm: <20 |
| Density (gr/ml) | 0.96-1.04 |

### Example 18: EC Compositions Comprising the Compound of Formula I

Three emulsifiable concentrate (EC) compositions (A, B and C) each containing 50 g/L of the compound of Formula I were prepared.

The process of preparing composition C is summarized below. (Compositions A and B may be prepared using a similar process.)
1. Alkamuls® 14/R (CO60) was melted in a hot bath/oven at 50° C.;
2. benzyl acetate and propylene carbonate were charged to the vessel and heated to 50° C.;
3. the compound of Formula I was added to the reactor while mixing until a clear solution was obtained;
4. Ninate® 60, TSP16 and molten Alkamuls® 14/R (4) were added gradually to the reactor while mixing until clear solution was obtained;
5. mixing was continued for 1 hour while the reactor was cooled to room temperature; and
6. the solution was discharged from the reactor through a filter (5 μm).

Compositions A, B and C are summarized in Tables 18, 19 and 20, respectively. The stability results for compositions B and C are summarized in Tables 21 and 22.

TABLE 18
EC Composition A

| | | | | Quantity for | |
|---|---|---|---|---|---|
| Raw Material | Manufacture | CAS No. | Function | 1000 Liters | |
| Compound of | | | A.I. | 50 kg (52 kg | |
| Formula I | | | | for 96%) | |
| JEFFSOL ® AG | Hunstman | 140-11-4 | Solvent | 730 | kg |
| 1705 benzyl | | | | | |
| acetate | | | | | |
| Nansa ® EVM | Hunstman | 90194-25-6 | Surfactant | 78 | kg |
| 70/2E Calcium | | | | | |
| dodecyl benzene | | | | | |
| sulphonate | | | | | |
| Toximul ® 8320 | Stepan | 9038-95-5 | Surfactant | 46.8 | kg |
| eo/po block | | | | | |
| copolymer | | | | | |
| Synperonic ® | Croda | 24938-91-8 | Surfactant | 31.2 | |
| 13/10 tridecyl | | | | | |
| alcohol | | | | | |
| ethoxylated | | | | | |
| Hallcomid ® | Stepan | 45280-17 | Solvent | 104 | |
| M-8-10 dimethyl | | | | | |
| fatty acid | | | | | |
| amide | | | | | |

Density, g/ml: 1.00-1.08

Crystal formation was observed in this composition after a week in 0° C. and after a few weeks at room temperature.

TABLE 19
EC Composition B

| | | | | Quantity | |
|---|---|---|---|---|---|
| | Manufac- | | | for 1000 | |
| Raw Material | turer | CAS No. | Function | Liters | |
| Compound of | | | A.I. | 50 kg (52 | |
| Formula I | | | | kg for | |
| | | | 96%) | | |
| JEFFSOL ® AG | Hunstman | 140-11-4 | Solvent | 834.0 | kg |
| 1705 benzyl | | | | | |
| acetate | | | | | |
| Genagen ® NBP N | Clariant | 3470-98-2 | Co-solvent | 104 | kg |
| butyl pyrrolidone | | | | | |
| Castor oil | Akzo Nobel | 61791-12-6 | Surfactant | 26 | kg |
| ethylene oxide | | | | | |
| (20) | | | | | |
| Castor oil | Akzo Nobel | 61791-12-6 | Surfactant | 26 | kg |
| ethylene oxide | | | | | |
| (60) | | | | | |

TABLE 20
Stability Results of Composition B

| | |
|---|---|
| Appearance | Yellowish solution |
| Density, g/ml | 1.04 |
| pH (1%) | 5.2 |
| Emulsion stability | Max. 0.2 ml separation |
| (0.2%, 1%) | after 2 hours. |
| Cold test | no crystal growth |

TABLE 21
EC Composition C

| | | Amount for |
|---|---|---|
| Ingredient trade name | Chemical name and CAS No. | 1000 L (kg) |
| Compound of Formula I | 5-fluoro-4-imino-3methyl-1- | 50 kg (52.1 |
| tech. as 100% of ADAMA | [4-methylphenyl)sulfonyl]- | kg for 96%) |
| Makhteshim LTD. | 3,4-dohydropyrimidin-2 (1H) - | |
| | one CAS No. N/A | |
| ALKAMULS ® 14/R of | ETHOXYLATED CASTOR OIL | 10.4 Kg |
| Solvay | CAS No. 61791-12-6 | |
| RHODACAL ® 60/BE of | Benzenesulfonic acid, C10-13- | 34.6 Kg |
| Solvay or | (linear)alkyl derivs., calcium salt | |
| AGNIQUE ® ABS 60 C-EH | CAS No. 104-76-7 (2- | |
| of Cognis or | ethylhexan-1-ol), 57-55-6 | |
| NANSA ® EVN 70/2E of | (Propylene glycol, only for | |
| Innospec | NANSA) | |
| SUPROPHOR ® TS/16 of | Tristyryl phenol-polyethylene | 69.2 Kg |
| Solvay or | glycol ether | |
| EMULSOGEN ® TS 160 of | CAS No. 104376-75-2 (Only for | |
| Clariant or | Solvay), 99734-09-5 (Only for | |
| Agnique ® TSP 16 of | Solvay and BASF), 70559-25-0 | |
| BASF | (Only for Clariant) | |
| Propylene carbonate | Propylene carbonate | 87.3 Kg |
| of BASF | CAS No. 108-32-7 | |
| Benzyl acetate of | ACETIC ACID PHENYL METHYL | Up to 1000 L |
| Hunstman or | ESTER | (about 788.5 kg) |
| Benzyl acetate of | CAS No. 140-11-4 | |
| Tennants fine | | |
| chemicals LTD | | |

Density, g/ml: 1.00-1.08

TABLE 22
Stability Results of Composition C

| | | | Oven . | Oven | Cold | Cold |
|---|---|---|---|---|---|---|
| | Start | Room | 54° C | 40° C. | 10° C. | 4° C. |
| Appearance | Clear | Clear | Clear | | no | Not |
| | yellowish | yellowi | yellowish | | crystal | crystal |
| | solution | sh | solution, | | growth | growth |
| | | solutio | little | | | |
| | | n | turbid at | | | |
| | | | the | | | |
| | | | bottom | | | |
| Concentration | 5.00% | 4.869% | 4.814% | | | |
| Compound of | | (reduction | (reduction | | | |
| Formula I | | 2.62%) | 3.72%) | | | |
| Emulsion | | ok | ok | | | |
| stability, | | | | | | |
| 0.2% (Water D, | | | | | | |
| 2 hr.) | | | | | | |
| Emulsion | | ok | ok | | | |
| stability | | | | | | |
| 1.0% (Water | | | | | | |
| D, 2 hr.) | | | | | | |
| Density | | 1.0821 | 1.0823 | | | |
| (gr/ml) | | | | | | |
| PH (1%) | | 6.65 | 6.64 | | | |
| Foaming 0.2% | | 60 ml | 60 ml | | | |
| (Water D, | | | | | | |
| 2 hr.) | | | | | | |
| Foaming 1% | | 10 ml | 10 ml | | | |
| (Water D, | | | | | | |
| 2 hr.) | | | | | | |

### Example 19: Tank Mix of Compositions Comprising the Compound of Formula I and Adjuvant(s)

### Example 15(a): SC Composition Tank Mixed with Adjuvant

Wheat crop (Winter wheat plants cv. Alixan (Limagrain) at the BBCH 12 growth stage) was treated with compositions comprising the compound of Formula I and adjuvants in different concentrations as built-in composition and/or as tank mix application.

All the tested compositions and mixtures were prepared in a volume of water corresponding to 200 L/ha and used 3 hours after preparation.

The SC composition of Example 5 was mixed with Trycol® (0.2 or 0.4 L per hectare) or Silwet (0.01 L per hectare) which were added as tank mix in 200 L volume of water.

The compositions and mixtures were applied with a hand sprayer at operating pressure of 2 bars. Three replicates (pots) of 6 wheat plants each were used for each condition tested.

After treatment, wheat plants were left to dry at room temperature for 1 hour and then incubated in a climatic chamber: Temperature of 24° C. day/18° C. night—Photoperiod of 16 h light/8 h dark and a Relative Humidity of 65%.

Fragments of the first leaf are cut and transferred in Petri dish containing adapted water agar (6 leaf fragments per Petri dish). Fragments are inoculated with a calibrated pycnospores suspension of Z. tritici strain Mg Tri-R6.

After inoculation, the Petri dishes are incubated in a climatic chamber: Temperature of 20° C. day/17° C. night—Photoperiod of 16 h light/8 h dark and controlled Relative Humidity.

After incubation time of 21 days, the intensity of the infection, which is the surface of colonized leaf by Z. tritici strain, is assessed (quantitative criteria). The fungicidal efficacy on each composition is then determined in percent of the untreated control. All data are treated by statistical software (XL STAT). The expected output of this step is the biological fungicidal efficiency ranking of the compound of Formula I in presence of different adjuvants.

Disease assessments were carried out 21 days post inoculation (dpi) by measuring the length of the necrosis of the leaf fragment. The intensity of infection was then determined in percent of the total length of the leaf fragment.

The efficacy was calculated based on the Area Under the Disease Progress Curve (AUDPC) which is a quantitative measure of disease intensity over time. The most commonly used method for estimating the AUDPC, the trapezoidal method, was performed by multiplying the average disease intensity between each pair of adjacent time points by the time interval corresponding and this for each interval time.

The fungicide efficacies were determined from the AUDPC values and expressed in percent of the untreated control.

### Results and Discussion

All adjuvants were tested alone towards Zymoseptoria tritici strain MG Tri-R6. None of the adjuvants tested alone had any significant fungicidal activity against Z. tritici strain Mg Tri-R6 in controlled conditions.

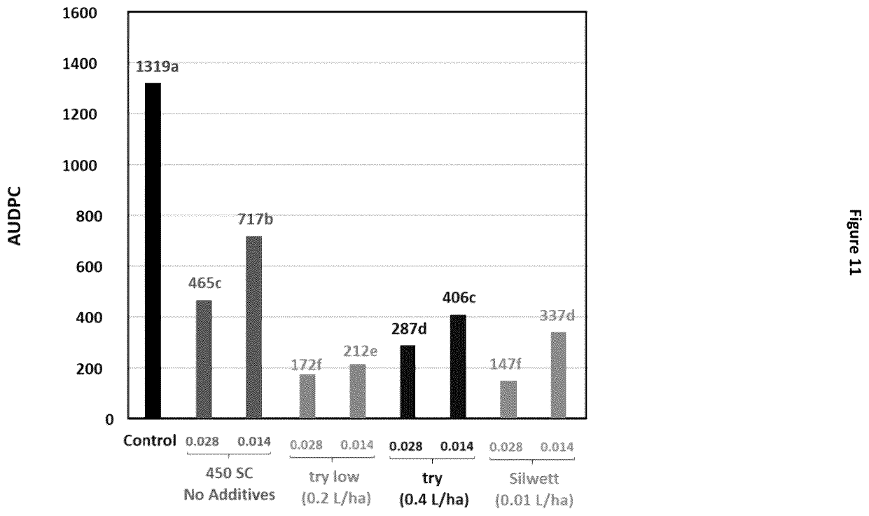

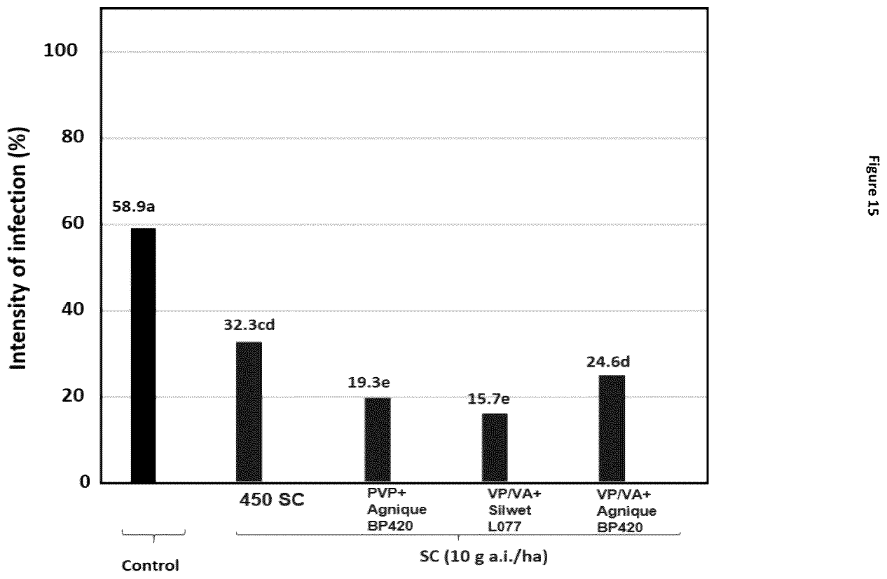

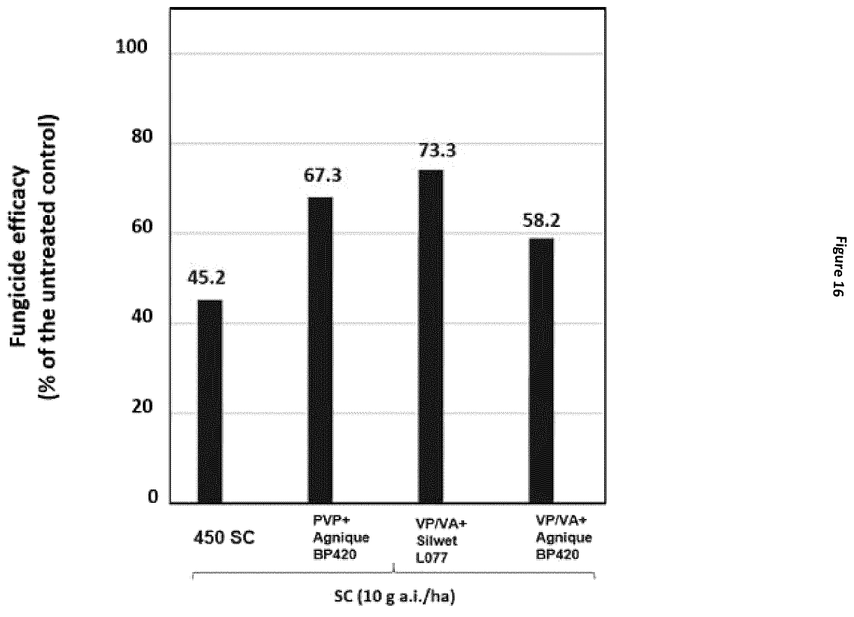

Results are summarized in FIGS. 11 to 16.

Results show that adding adjuvant(s) increase the efficacy of compound of Formula I towards Zymoseptoria tritici strain MG Tri-R6.

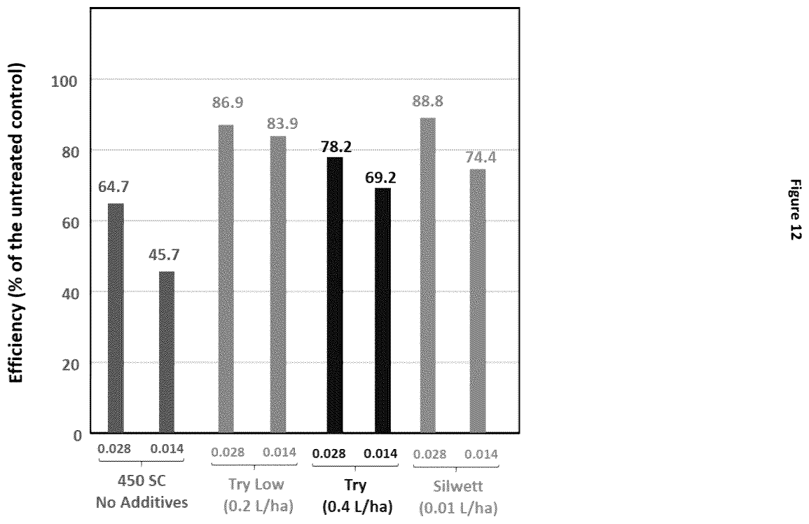

As shown in FIGS. 11 and 12, adding one adjuvant, Trycol® or Silwet® (even in low amount) increased the efficacy and the effective fungicidal activity of compound of Formula I compared to the application without adjuvant.

The results showed that the addition of Trycol® or Silwet® to compound of Formula I SC in a tank mix significantly improved fungicidal efficacy of the composition.

Without wishing to be bound by any theory, it is hypothesized that Silwet® L-077 and Trycol improved fungicidal efficacy of the composition (this adjuvant's concentration is up to 3% by weight based on the total weight of the composition) by lowering the surface tension of the leaf's surface.

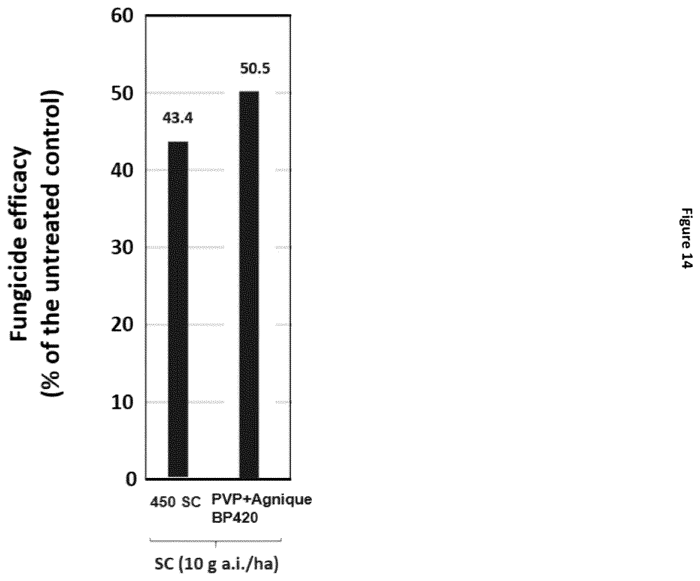

As shown in FIGS. 14, 15 and 16, adding two adjuvants, Silwet® or Agnique® BP 420 with PVP or VP/VA increased the efficacy and the effective fungicidal activity of compound of Formula I compared to the application without adjuvant.

The efficacy of compound of Formula I in presence of PVP or VP/VA in combination with Agnique® BP 420 (Alcohol ethoxylate propoxylate C16 C18) or Silwet® was increased compared to application of the compound of Formula I without adjuvant.

Without wishing to be bound by any theory, it is hypothesized that Silwet® L-077 (Siloxane polyalkyleneoxide copolymer) in combination with VP/VA (this adjuvant's concentration is up to 2% by weight based on the total weight of the composition) increased the efficacy and the effective fungicidal activity of compound of Formula I by lowering the surface tension, thus, spreading the composition on leaf's surface. In other words, the deposit of the composition on the leaf surface is more spread out and stays more time on the leaf, thus has rain fastness properties.

### Example 15 (b): EC Composition Tank Mixed with Adjuvant

The EC composition and the adjuvants were mixed only prior the experiment (e.g. as tank mix).

All the fungicides are prepared in a volume of water or S-solutions (S-solution refers to the diluted solution of the compositions) corresponding to 200 L/ha and used 3 hours after preparation.

TABLE 23
Compound of Formula I 50 EC Composition

| A.I/ha | Number of | | Composition | Adjuvant | Water |
|---|---|---|---|---|---|
| (gr) | treatment | Treatment | (L/ha) | (L/ha) | (L/ha) |
| 12.5 | 1 | Composition | 0.25 | 0 | 200 |
| | | A − No | | | |
| | | adjuvant | | | |
| 6.25 | 2 | Composition | 0.125 | 0 | 200 |
| | | A − No | | | |
| | | adjuvant | | | |
| 12.5 | 3 | Composition | 0.25 | 0.4 | 200 |
| | | A + Trycol ® | | | |
| 6.25 | 4 | Composition | 0.125 | 0.4 | 200 |
| | | A + Trycol ® | | | |
| 12.5 | 5 | Composition | 0.25 | 0 | 200 |
| | | B − No | | | |
| | | adjuvant | | | |
| 6.25 | 6 | Composition | 0.125 | 0 | 200 |
| | | B − No | | | |
| | | adjuvant | | | |
| 12.5 | 7 | Composition | 0.25 | 0.2 | 200 |
| | | B + Trycol ® | | | |
| | | low | | | |
| 6.25 | 8 | Composition | 0.125 | 0.2 | 200 |
| | | B + Trycol ® | | | |
| | | low | | | |
| 12.5 | 9 | Composition | 0.25 | 0.4 | 200 |
| | | B + Trycol ® | | | |
| 6.25 | 10 | Composition | 0.125 | 0.4 | 200 |
| | | B + Trycol ® | | | |
| 12.5 | 11 | Composition | 0.25 | 1 | 200 |
| | | B + SOC | | | |
| 6.25 | 12 | Composition | 0.125 | 1 | 200 |
| | | B + SOC | | | |

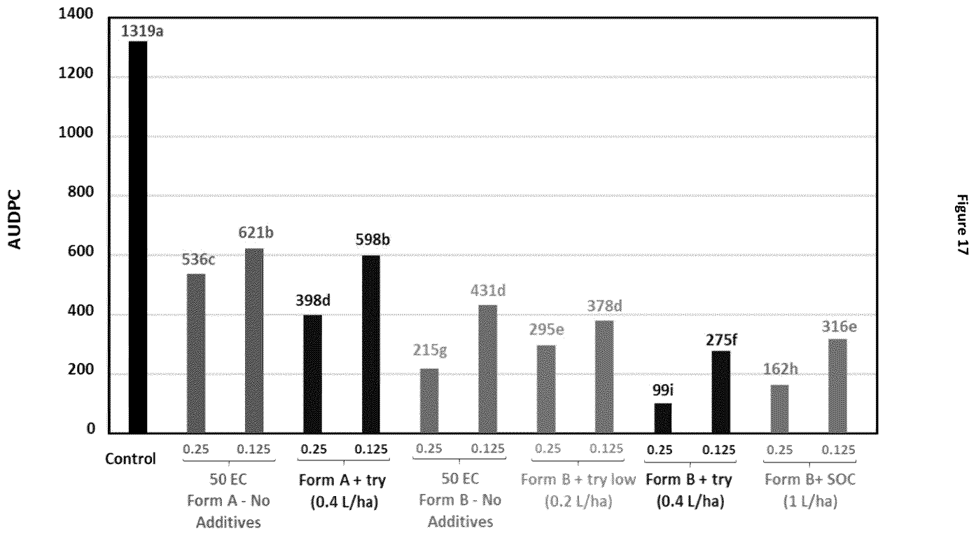

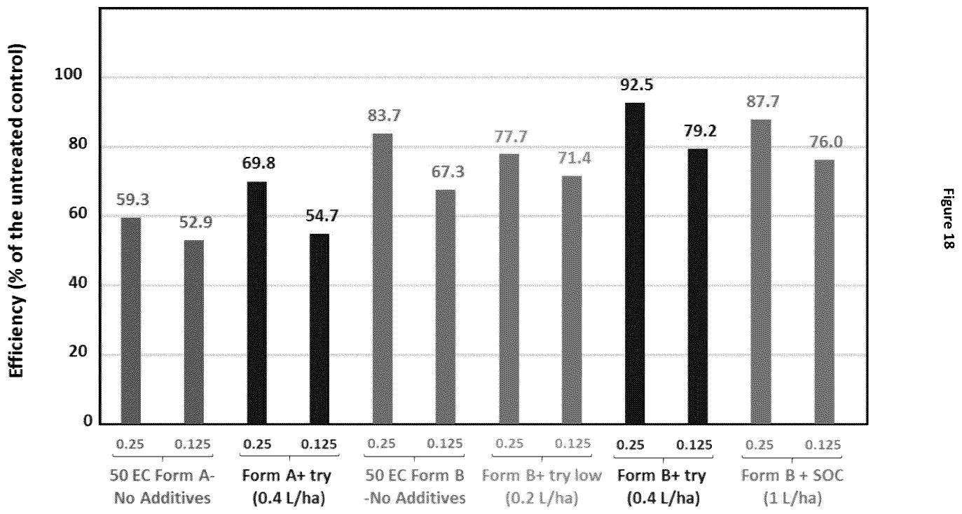

The results are summarized in FIGS. 17 and 18.

### Example 20: OD Composition

To assess the fungicidal activity of OD composition containing compound of Formula I, an OD composition (from Example 14, Table 13) was prepared in a volume of water or S-solutions (S-solution refers to the diluted solution of the compositions) corresponding to 200 L/ha and used 3 hours after preparation.

The first leaf of wheat plantlets cv. Alixan were untreated or treated with the OD composition of Compound of Formula I Prototype A (from Example 10, Table 13) at 10 g a.i./ha and 20 g a.i./ha at 21 days post inoculation with pycnospores of the Zymoseptoria tritici strain Mg Tri-R6 (moderately resistant to DMI and highly resistant to QoI fungicides in controlled conditions). Disease was assessed using intensity of infection.

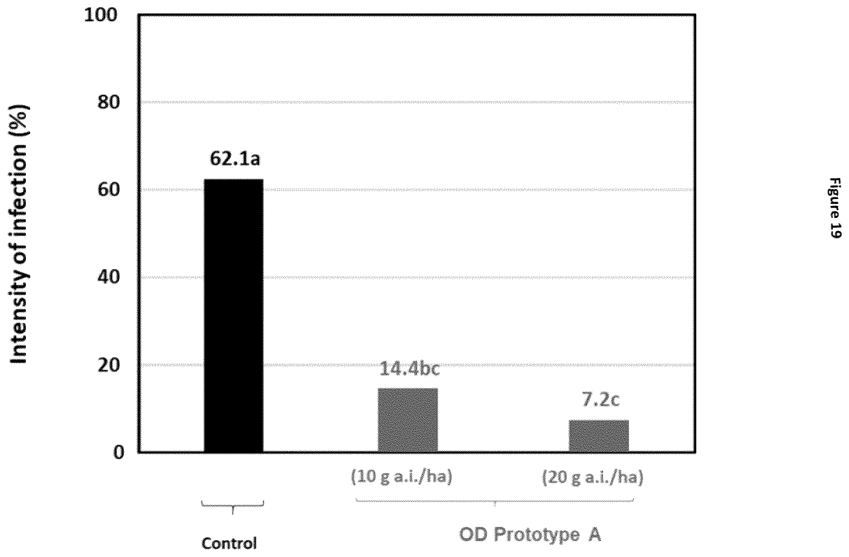

The results are shown in FIG. 19.

### Example 21: OD Composition and EC Composition with Adjuvant

To assess the fungicidal activity of OD composition and EC composition with adjuvant containing the compound of Formula I on potato late blight (Phytophthora infestans), an OD composition (from Example 14, Table 13) was prepared in a volume of in a volume of water or S-solutions corresponding to 300 L/ha and was applied by knap-sack sprayer: with horizontal boom flat fan nozzles (six weekly applications). The EC composition used is EC Composition C (Example 18, Table 21).

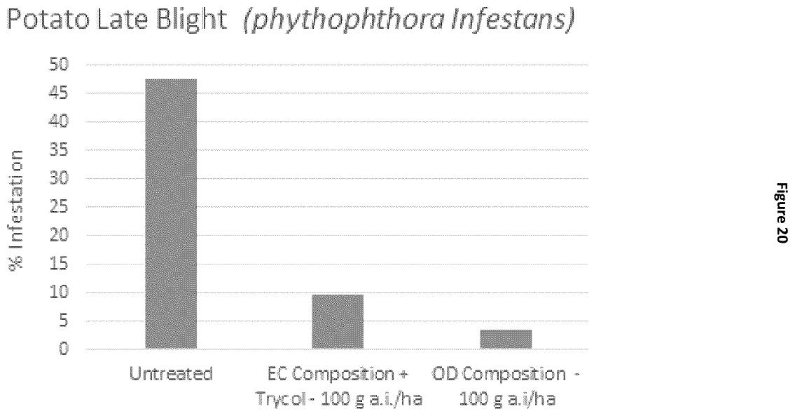

Disease was assessed using percent of infection. The results are shown in FIG. 20.

### Example 22: OD Compositions

Processing details (1-liter batches):
Tetraethyl orthosilicate (TEOS) was added to Agnique® ME 18 RD-F and mixed with a high shear mixer for 5 mins.
Aerosil® was added whilst shearing and mixed until fully dispersed (approximately 5 mins).
Agrimer™ AL-22, Atlox® 4916, Aerosol® OT-SE, Synperonic® PE/L 64 and Genapol® X 050 were added and mixed until homogenous (approximately 5-10 mins).
The compound of Formula I was added slowly whilst shearing and mixed for 15 mins.
The resulting batch was milled in Eiger mini motor mill for 15 mins at 4500 rpm (75% 0.75-1.0 mm glass bead charge). D(50) was approximately 2-3 μm.

The composition should remain below 30° C. throughout the processing.

Two OD compositions as summarized in Tables 24 and 25 were prepared.

TABLE 24
OD Composition

| Ingredient | % w/w |
|---|---|
| Compound of Formula I | 25.0 |
| Atlox ® 4912 | 3.0 |
| Atlas ™ G5002 L | 3.0 |
| Aerosol ® OT-SE | 6.0 |
| Synperonic ® PE/L 64 | 6.0 |
| Genapol ® X 050 | 3.0 |
| TEOS | 5.0 |
| Aerosil ® R202 | 0.5 |
| Agnique ® ME 18 RD-F | To 100 |

TABLE 25
OD Composition

| Ingredient | % w/w |
|---|---|
| Compound of Formula I | 25.00 |
| Agrimer ™ AL-22 | 3.00 |
| Atlox ® 4916 | 3.00 |
| Aerosol ® OT-SE | 6.00 |
| Synperonic ® PE/L 64 | 6.00 |
| Genapol ® X 050 | 3.00 |
| TEOS | 5.00 |
| Aerosil ® R202 | 2.60 |
| Agnique ® ME 18 RD-F | 46.40 |

During fumed silica screening, 100 mL caused aerated and thickening of samples. Any changes in dispersant will likely cause thickening in the composition (Atlas™ G5002L and Atlox® 4912 causes slight thickening, Atlox® 4915 caused extreme gelling of the silica).

### Initial Properties

Appearance: White, free flowing, homogenous liquid.
pH (1% deionised water): 7.3
Persistent Foam (1% tap water): 6 mL after 1 min, 2 mL after 12 mins.
Water Content: 0.03%
Wet sieve residue (<75 μm): 0.03%
Dispersion properties: Homogenous after 10 inversions. Trace sediment/cream after 30 mins and 2 hours.
Viscosity at 10s−1: 609 mPa·s, Viscosity at 100s−1: 176 mPa·s

### Example 23: EC50 Composition

TABLE 26
EC50 Composition

| | | | | | | | Assay |
|---|---|---|---|---|---|---|---|
| | Cas | Chemical | | | | | (purity |
| Ingredient | No. | Name | w/w | Function | Supplier | v/w | level) |
| Compound | N/A | | 4.74 | Fungicide | Adama | 50 | 0.978 |
| of Formula I | | | | | Makhteshim | | |
| Agnique BP | 68002-96-0 | | 7.11 | Adjuvant | BASF | 75 | 1 |
| 420 | | | | | SE | | |
| Soprophor | 104376-75 | Poly (oxy- | 23.22 | Emulsifier | Solvay | 245 | 1 |
| TS/16 | | 1,2- | | | | | |
| | | ethanediyl).- | | | | | |
| | | alpha.- | | | | | |
| | | phenyl-.omega.- | | | | | |
| | | hydroxy-, | | | | | |
| | | styrenated | | | | | |
| | | (Tristyrylphenol | | | | | |
| | | ethoxylate) | | | | | |
| Synergen | Proprietary | Fatty | 3.79 | Adjuvant | Clariant | 40 | 1 |
| SOC | | alcohol | | | | | |
| | | alkoxylate | | | | | |
| | | blend in | | | | | |
| | | propylene | | | | | |
| | | glycol | | | | | |
| Acetophenone | 98-86-2 | Acetophenone | 61.14 | Solvent | Ruetgers | Up | 1 |
| 99% | | | | | | to 100 | |

TABLE 27
Properties

| Appearance | Clear white/brownish solution |
|---|---|
| Active ingredient content | compound of Formula I conc |
| Density gr/ml | 1.03-1.08 |

### Example 24: 500SC Composition

TABLE 28
500 SC Composition

| Ingredient | Cas No. | Chemical Name | w/w |
|---|---|---|---|
| Compound of | N/A | | 41.67 |
| Formula I | | | |
| Ag-RH 23 | 11138-66-2 | Xanthan Gum | 0.19 |
| Disodium | 7558-79-4 | Disodium Phosphate | 0.58 |
| Phosphate | | Anhydrous | |
| Anhydrous | | | |
| Emulsogen TS | 104376-75-2 | Tristyryl phenol- | 1.42 |
| 540 | | polyethylene glycol ether | |
| Potassium | 7778-77-0 | Potassium dihydrogene | 0.14 |
| dihydrogene | | phosphate | |
| phosphate | | | |
| Propylene | 57-55-6 | 1,2-propanediol | 1.96 |
| glycol | | | |
| Proxel GXL | (A)2634-33-5, | (A)1,2-Benzisothiazol-3(2H)-one, | 1.67E−2 |
| | (B) 1310-73-2 | (B)Sodium hydroxide | |
| SAG 1572 | N/A | Polydimethylsiloxane | 1.5 |
| | | emulsion | |
| Silwet L-77 | (A) 27306-78-1, | (A)Polyalkyleneoxide | 0.2 |
| | (B) 1310-73-2 | modified | |
| | | Heptamethyltrisiloxane, | |
| | | (B)Polyalkyleneoxide Modified | |
| | | Heptamethyltrisiloxane | |
| Soprophor 3 D | (A) 90093-37-1, | (A) 2,4,6-Tris(1- | 3.75 |
| 33 | (B) 99734-09-5 | phenylethyl)polyoxyethylenated | |
| | | phosphates, (B)Poly(oxy- | |
| | | 1,2-ethanediyl), .alpha.- | |
| | | [tris(1- | |
| | | phenylethyl)phenyl]- | |
| | | .omega.-hydroxy-, | |
| | | (C)orthophosphoric acid | |
| | | (POLYARYLPHENYL ETHER | |
| | | PHOSPHATE) | |
| Supragil WP | N/A | Reaction product of | 0.5 |
| | | naphthalene, propan-2-ol, | |
| | | sulfonated and neutralized | |
| | | by caustic soda | |
| Soft water | 7732-18-5 | water | 48.575 |

TABLE 29
Properties

| Appearance | White liquid suspension |
|---|---|
| Density, g/ml | 1.1-1.3 |
| pH | 5.5-7.0 |

### Example 25: Stability of Non-Aqueous Compositions Comprising the Compound of Formula I

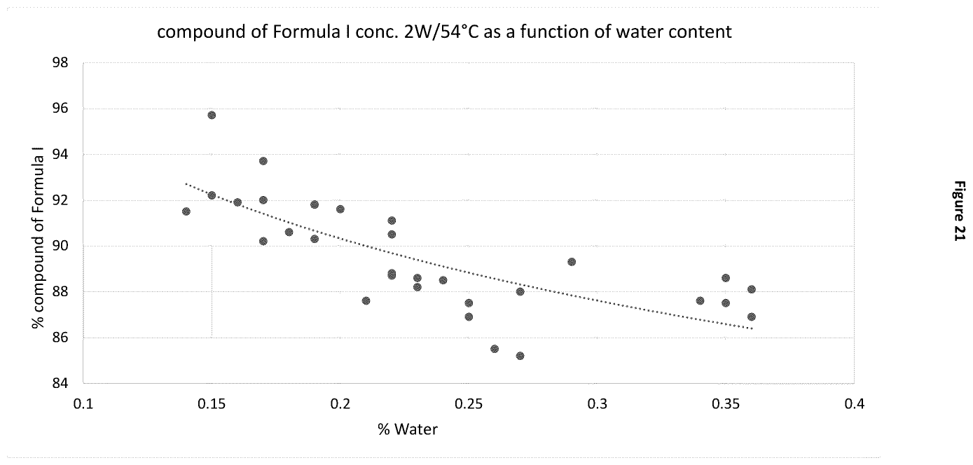

The stability of the compound of Formula I in compositions having varying water content was evaluated and the results are shown in Table 30 below and in FIG. 21.

TABLE 30

| 2W/54° C. | | | |
|---|---|---|---|
| analysis (% | % Water | Compound of | |
| of −10° C.) | Content | Formula I | |
| 93.7 | 0.17 | Form I | |
| 92.2 | 0.15 | Polymorph | |
| 88.5 | 0.24 | Hydrate I | 20% Hydrate, and |
| 88.7 | 0.22 | | 80% Form I Polymorph |
| 89.3 | 0.29 | Hydrate I | 45% Hydrate, and |
| 88.6 | 0.35 | | 55% Form I Polymorph |
| 95.7 | 0.15 | Form I | |
| | | Polymorph | |
| 96.3 | 0.17 | Hydrate I | |
| 95.9 | 0.17 | | |
| 96.8 | 0.15 | | |
| 97.4 | 0.16 | | |
| 96.9 | 0.16 | | |
| 96.4 | 0.15 | Form I | |
| 96.0 | 0.18 | Polymorph | |
| 95.6 | 0.17 | | |
| 96.3 | 0.17 | | |
| 93.1 | 0.19 | | |
| 94.8 | 0.18 | | |
| 95.1 | 0.18 | | |
| 95.2 | 0.18 | | |
| 94.7 | 0.19 | | |
| 96.5 | 0.16 | Form I | |
| | | Polymorph | |

* water content refer to the total water in the composition, including free water molecules and water molecules in the hydrate.

### Example 26: OD Composition Comprising the Compound of Formula I

TABLE 31

| Ingredient | | | |
|---|---|---|---|
| number | Ingredient trade name | g/2 L | % w/w |
| 1 | Compound of Formula I | 525.1 | 25.61 |
| | (TECH 800-09-131-0794-18, | | |
| | 0.976) | | |
| 2 | Agrimer AL-22 | 61.5 | 3.00 |
| 3 | Atlox 4916 | 102.5 | 5.00 |
| 4 | Aerosol OT-SE | 123 | 6.00 |
| 5 | Synperonic PE/L 64 | 123 | 6.00 |
| 6 | Genapol X 050 | 102.5 | 5.00 |
| 7 | TEOS | 102.5 | 5.00 |
| 8 | Aerosil R202 | 4.1 | 0.2 |
| 9 | Tween 24 L.M. | 66.6 | 3.25 |
| 10 | Agnique ME 18 RD-F | 838.5 | 40.90 |
| | TOTAL | 2049.3 | 100.0 |

Density = 1.025

### Example 27: OD Composition Comprising the Compound of Formula I

TABLE 32

| | | Density | 1.04 |
|---|---|---|---|
| | | 3750-070421-14 | |
| | | | g/1200 |
| | Chemical name | % w/w | mL |
| Compound of | 5-fluoro-4-imino-3-methyl- | 25.61 | 319.7 |
| Formula I | 1-tosyl-3,4- | | |
| | dihydropyrimidin-2(1H)-one | | |
| Agrimer AL-22 | Vinyl pyrrolidone copolymers | 3.00 | 37.4 |
| | like alkyl grafted PVP | | |
| Atlox 4916 | High molecular weight | 5.00 | 62.4 |
| | polymer | | |
| Aerosol OT-SE | Hydrocarbons, C12-C-15 | 6.00 | 74.9 |
| | alkanes, isoalkanes, | | |
| | cyclic <2% aromatics | | |
| | Docusate sodium | | |
| Synperonic PE/ | Block copolymer of ethylene | 6.00 | 74.9 |
| L 64 | oxide and propylene oxide | | |
| Genapol X 050 | Ethoxylated Isotridecyl | 5.00 | 62.4 |
| | Alcohol | | |
| EPOXOL D65 | Epoxidized soybean oil | 5.00 | 62.4 |
| Aerosil R202 | Decamethylcyclo- | 2.50 | 31.2 |
| Tween 24 L.M. | pentasiloxane | 3.25 | 40.6 |
| Agnique ME 18 | C16-18-unsaturated fatty | 38.64 | 482.2 |
| RD-F | acid methyl esters | | |
| Total | | 100.0 | 1248.0 |

* EPOXOL D65 (manufactured and sold by FACI SpA) may be replaced with Agnique ® ESO 81-G (manufactured and sold by BASF)

The properties of the OD composition shown in Table 32 is summarized below in Table 33.

TABLE 33

| Appearance | White/off white |
|---|---|
| Compound of Formula I | 237.5-262.5 |
| Density g/cm³ | 1.00-1.05 |
| pH (1% water) | 6.0-7.5 |
| Viscosity S-62 12 rpm | 500-3000 cp |

### Production Procedure:

1. Epoxol was added to the Agnique ME 18 RD-F and mixed with a high shear mixer for 5 mins.
2. Added Aerosil R202 whilst shearing and mixed until fully dispersed (approximately 5 mins).
3. Agrimer AL-22, Atlox 4916, Aerosol OT-SE, Synperonic PE/L 64, Tween 24 L.M and Genapol X 050 were added and mixed until homogenous.
4. The compound of Formula I was added slowly whilst shearing.
5. Batch milled.
6. D(50) approximately 2-3 μm.

### Example 28: SC Composition Comprising the Compound of Formula I

TABLE 34

| | | | | Quantity for | |
|---|---|---|---|---|---|
| Raw Material | Manufacturer | CAS No. | Function | 1000 Liters | |
| Compound of Formula I | Adama | | A.I. | 500 kg | |
| as 100% | Makhteshim | | | (512.3 kg | |
| | | | | for 97.6%) | |
| Supragil WP - (sodium | Solvay | 1322-93-6 | Wetting | 6.1 | kg |
| Diisopropylnaphthalenesulphonate) | | | agent | | |
| Soprophor 3 D 33 - | Croda | 90093-37-1 | Dispersant | 48.0 | kg |
| (2,4,6-Tris(1- | | | | | |
| phenylethyl)polyoxyethylenated | | | | | |
| phosphates) | | | | | |
| Emulsogen TS 54 | Clariant | 70559-25-0 | Dispersant | 19.2 | kg |
| (2,4,6-Tri-(1- | | | | | |
| phenylethyl)- | | | | | |
| phenol polyglycol | | | | | |
| ether with 54 EO) | | | | | |
| Propylene Glycol | INEOS | 57-55-6 | Anti- | 23.5 | kg |
| | OXIDE | | freeze | | |
| Silwet L-77 | Momentive | 27306-78-1 | Wetting | 3 | kg |
| (Polyalkyleneoxide | | | agent | | |
| modified | | | | | |
| Heptamethyltrisiloxane) | | | | | |
| SAG 1572 | Momentive | Trade | Antifoam | 12 | kg |
| (Polydimethylsiloxane | | secret | emulsion | | |
| antifoam emulsion) | | | | | |
| AGRH 23 2% Solution | | 11138-66-2 | Rheology | 89 | kg |
| (Xanthan gum) | | | modifier | | |

| | | | |
|---|---|---|---|
| KH₂PO₄ | 7778-77-0 | pH | 1.7 |
| | | modifier | |
| Na₂HPO₄ | 7558-79-4 | pH | 6.9 |
| | | modifier | |
| Proxyl GXL | (A)2634-33-5, | Biocide | 1.0 |
| | (B)1310-73-2 | | |
| Soft Water | | | Up to 1000 |
| | | | L (About |
| | | | 478.3 kg) |

*Check also after 8 weeks storage at 40° C.

### Specification:

| Appearance | White suspension | |
|---|---|---|
| Compound of Formula I conc. | 475-525 | g/l |
| Density | 1.1-1.3 | g/ml |
| Particle size | d₉₀ < 6.0 | μm |

| | |
|---|---|
| pH | 5.5-7.5 |
| Viscosity (12 rpm) | 1400 cP-2400 cP |
| WSR (75 μm) | Max 2% |
| Pourability (5%, 0.25%) | According to CIPAC |
| Suspensibility (0.2%, 1%) | >60% |
| Foaming (0.2%, 1%) | According to CIPAC |

### Production Procedure:

Stage 1: Mixing: Charged 356 Kg Soft Water to the vessel and heated to about 60-70° C., added TSP-54 and mixed by heating until the solution was homogenous, then cooled down to room temp. Then added KH₂PO₄, Na₂HPO₄, Supragil WP, Soprophor 3D33 Propylene Glycol, Silwet L-077 and 3 kg of SAG 1572 and mixed until the solution was homogenous.
Stage 2: Milling: Slowly charged the compound of Formula I tech. while shearing with high shear until the solution was homogenous. Added rest of SAG 1572 to reduce foaming after HS and milled the suspension until d₉₀<6 μm.
Stage 3: Add Ag/RH 23 2% sol. to get viscosity 1400-2400 cP and rest of water.

### Example 29: EC Composition Comprising the Compound of Formula I

TABLE 35

| | | | | Quantity |
|---|---|---|---|---|
| | | | | for 1 |
| Actives and | | | | Liters |
| co-formulants | Manufacturer | CAS No. | Function | (gr) |
| Compound of | Makhteshim | BN 8001-09- | Active | 50 (51.22 |
| Formula I tech. | | 131-0794-18 | ingredient | as 97.6%) |
| (as 97.6%) | | | | |
| Soprophor ® | Solvay | 104376-75-2 | Emulsifier | 239 |
| TS/16 (TSP-16, | | | | |
| Tristyrylphenol | | | | |
| ethoxylate 16) | | | | |
| Synergen SOC | Clariant | Proprietary | Adjuvant | 57 |
| (Fatty (alcohol | | | | |
| alkoxylate | | | | |
| blend in | | | | |
| propylene | | | | |
| glycol) | | | | |
| Agnique 420 | BASF | 68002-96-0 | Adjuvant | 57 |
| (Alcohols, C16- | | | | |
| 18, ethoxylated | | | | |
| propoxylated) | | | | |
| Acetophenone | RÜTGERS | 98-86-2 | Solvent | Up to 1 L |
| | | | | (about 635 |
| | | | | gr/L) |

### Specification:

| Appearance | Clear yellow/brownish solution | |
|---|---|---|
| Azoxystrobin conc. | 110-130 | g/L |
| Prothioconazole conc. | 80-100 | g/L |
| Density | 1.02-1.07 | g/ml |
| Cold test | as in WHO | |
| Emulsion stability (0.2%, 1%) | as in WHO | |
| pH (1%) | 5.5-7.5 | |
| Foaming (0.2%, 1%) | <60 ml after 1 min | |
| Flash point | >90° | C. |

### Preparation Procedure:

1. Charged acetophenone to the vessel and heated to 45° C.
2. Added the compound of Formula I tech. to the reactor while mixing until a clear solution was obtained.
3. Added gradually to the reactor TSP-16, Agnique 420 and Synergen SOC, while mixing until a clear solution was obtained.
4. Continued with 1 hour mixing while cooling the reactor to room temperature.
5. Filtered (5 μm) the solution when discharging it from the reactor.
6. Sent sample to QC.

1. A crystalline form of 5-fluoro-4-imino-3-methyl-1-tosyl-3,4-dihydropyrimidin-2 (1H)-one (compound A), which has the following structure: 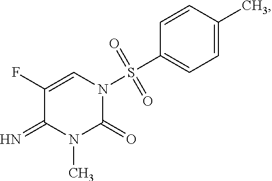 [SMILES: Cc1ccc(S(=O)(=O)n2cc(F)c(=N)n(C)c2=O)cc1]
wherein:
a. the crystalline form is Form III having powder X-ray diffraction pattern comprising peaks at 4.5, 5.1, 9.1, 10.1, 22.4, 23.5±0.2 deg 2-theta;
b. the crystalline form is Form IIIa having powder X-ray diffraction pattern comprising peaks at 4.5, 5.0, 9.3, 10.0, 22.3, 22.6±0.2 deg 2-theta;
c. the crystalline form is Form 02 having powder X-ray diffraction pattern comprising peaks at 7.5, 14.3, 15.8, 22.4, 24.7±0.2 deg 2-theta; or
d. the crystalline form is Form 01 having powder X-ray diffraction pattern comprising peaks at 5.1, 6.4, 7.5, 10.3, 16.1±0.2 deg 2-theta.

2. The crystalline form of claim 1,
wherein:
a) the crystalline form is Form III having X-ray powder diffraction pattern comprising characteristic peaks at 4.5, 5.1, 9.1, 10.1, 11.9, 14.2, 16.1, 22.4, 23.5±0.2 deg 2-theta,
b the crystalline form is Form IIIa having X-ray powder diffraction pattern comprising characteristic peaks at 4.5, 5.0, 9.3, 10.0, 11.9, 14.2, 16.0, 22.3, 22.6, 25.3±0.2 deg 2-theta,
c) the crystalline form is crystalline Form 01 having X-ray powder diffraction pattern comprising characteristic peaks at 5.1, 6.4, 7.5, 10.3, 12.2, 13.4, 14.9, 16.1, 18.4±0.2 deg 2-theta, or
d) the crystalline form is crystalline Form 02 having X-ray powder diffraction pattern comprising characteristic peaks at 7.5, 11.9, 14.3, 15.8, 18.7, 22.4, 24.7, 27.4, 31.1±0.2 deg 2-theta.

3-5. (canceled)

6. A combination comprising the crystalline form of claim 1 and at least one agriculturally acceptable carrier, optionally wherein:
a) the combination comprises an amount of compound A in two or more different forms and at least one agriculturally acceptable carrier, wherein at least one form of compound A is the crystalline form of claim 1,
b) the combination comprises at least one additional pesticide.

7-12. (canceled)

13. A composition comprising the combination of claim 6.

14-17. (canceled)

18. A method for treating a plant or locus against fungal infection, wherein the method comprises:
i) obtaining an effective amount of a compound having the following structure: 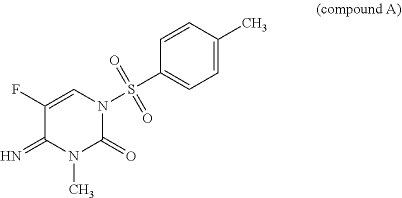 [SMILES: Cc1ccc(S(=O)(=O)n2cc(F)c(=N)n(C)c2=O)cc1]
in one or more forms, wherein at least one form of compound A is the crystalline Form III, IIIa, 01 or 02 of claim 1, and
ii) applying the amount of compound A to the plant or locus so as to thereby treat the plant or locus against fungal infection.

19-22. (canceled)

23. A process for preparing the crystalline form of claim 1,
wherein the process comprises:
a) combining an amount of a compound having the following structure: 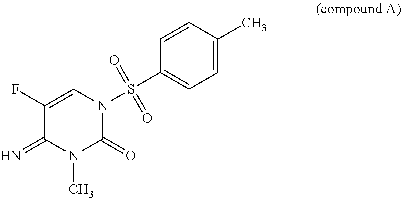 [SMILES: Cc1ccc(S(=O)(=O)n2cc(F)c(=N)n(C)c2=O)cc1]
and a suitable solvent to prepare a mixture;
b) optionally removing any precipitated solid from the mixture of step a); and
c) obtaining the crystalline form.

24-39. (canceled)

40. A process of manufacturing a fungicidal composition comprising obtaining the crystalline form of claim 1 and combining the crystalline form with at least one agriculturally acceptable carrier so as to thereby manufacture the fungicidal composition.

41. (canceled)

42. A stable, liquid composition comprising:
(a) a fungicidally effective amount of compound in one or more forms, wherein at least one form is the crystalline form of claim 1.

43. The stable, liquid composition of claim 42, wherein:
a) the composition comprises (a) a fungicidally effective and substantially pure amount of compound A, or a fungicidally effective amount of a mixture containing compound A,
wherein 95% or more by weight of the mixture is compound A, and (b) the liquid carrier,
b) 95% or more of the amount of compound A is in the form of Form III, Form IIIa, Form 01, or Form 02 of claim 1, or a mixture thereof,
c) the solubility of compound A in the liquid carrier is less than 5000 ppm,
d) the composition comprises at least one stabilizing surfactant,
e) the pH of the composition is in the range of 5 to 7.5,
f) the composition has a water content of less than 0.5% by weight, preferably less than 0.2% by weight, based on the total weight of the composition,
g) the composition has a viscosity of at least 500 cP, and/or
h) the composition further comprises at least one adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii).

44-49. (canceled)

50. A fungicidal mixture comprising the following components:
a fungicidally effective and substantially pure amount of compound A or a fungicidally effective amount of a mixture containing compound A; and
(b) an adjuvant selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants;
(vi) lignins;
(vii) terpenes; and
(viii) any combination of (i), (ii), (iii), (iv), (v), (vi) and (vii),
wherein compound A is in one or more forms and at least one form is Form III, Form IIIa, Form 01, or Form 02 of claim 1.

51. (canceled)

52. A method of controlling and/or preventing (i) fungal pathogen attack on a plant, or (ii) plant and/or soil fungal disease, wherein the method comprises applying a fungicidally effective amount of compound A
and at least one adjuvant to soil, plant, root, foliage, seed, locus of the fungus, and/or a locus in which the infestation is to be prevented so as to thereby control and/or prevent fungal pathogen attack on the plant or plant and/or fungal disease, wherein the adjuvant is selected from the group consisting of:
(i) polyalkylene oxide alkyl ether;
(ii) siloxane polyalkyleneoxide copolymer;
(iii) esters of fatty acid;
(iv) vinylpyrrolidones and derivatives thereof;
(v) sugar-based surfactants,
(vi) lignins;
(vii) terpens; and
(viii) any combination of (i), (ii), (iii), (iv), (v), (vi) and (vii),
wherein compound A is in one or more forms and at least one form is Form III, Form IIIa, Form 01, or Form 02 of claim 1.

53. A method for improving biological activity of compound A against fungal pathogen, the method comprising applying compound A
in the presence of at least one adjuvant, wherein the adjuvant is selected from the group consisting of:
(i) polyalkylene oxide alkyl ether;
(ii) siloxane polyalkyleneoxide copolymer;
(iii) esters of fatty acid;
(iv) vinvlpvrrolidones and derivatives thereof:
(v) sugar-based surfactants;
(vi) lignins;
(vii) terpenes; and
(viii) any combination of (i), (ii), (iii), (iv), (v), (vi) and (vii),
so as to thereby improve biological activity of compound A,
wherein compound A is in one or more forms and at least one form is Form III, Form IIIa, Form 01, or Form 02 of claim 1.

54. A method for increasing stability of a liquid composition comprising compound A, wherein compound A is in one or more forms and at least one form is Form III, Form IIIa, Form 01, or Form 02 of claim 1;
and a liquid carrier,
wherein the method comprises:
a) selecting a liquid carrier wherein the solubility of compound A in the liquid carrier is less than 5000 ppm,
b) maintaining the pH value of the composition in the range of 5 to 7.5,
c) maintaining the water content of the composition to less than 0.2% by weight based on the total weight of the composition,
d) adding (i) at least one stabilizing surfactant having crystal growth inhibiting property or (ii) a stabilizing system having a crystal growth inhibiting property to the liquid composition,
e) formulating the composition to have a viscosity of at least 500 cP, and/or
f) using a batch of compound A that is substantially pure of compound A or comprises 95% or more by weight of compound A to prepare the liquid composition,
so as to thereby increase stability of the composition comprising compound A.

55. (canceled)

56. Use of at least one stabilizing surfactant having structure of polyalkylene oxide polyaryl ether for controlling solubility and/or degradation of an amount of compound A,
wherein compound A is in one or more forms and at least one form is Form III, Form IIIa, Form 01, or Form 02 of claim 1.

57. A process for preparing a composition comprising an amount of compound A,
wherein compound A is in one or more forms and at least one form is Form III, IIIa, Form 01, or Form 02 of claim 1,
wherein:
a) the composition is a suspension concentrate (SC) composition and
the process comprises the steps:
(1) mixing agriculturally acceptable inert additives and an aqueous liquid carrier to obtain a premix;
(2) adding compound A to the premix obtained in step (1) to obtain a mixture; and
(3) milling the resulting mixture of step (2) to obtain the desired composition,
b) the composition is a suspoemulsion (SE) composition and the process comprises the steps:
(1) mixing agriculturally acceptable inert additives and an aqueous liquid carrier to obtain a premix;
(2) adding compound A and at least one adjuvant to the premix obtained in step (1) to obtain a mixture; and
(3) milling the resulting mixture of step (2) to obtain the desired composition, or
c) the composition is an oil dispersion (OD) composition and the process comprises the steps:
(1) mixing agriculturally acceptable inert additives and a non-aqueous liquid carrier to obtain a premix;
(2) adding compound A to the premix obtained in step (1) to obtain a mixture; and
(3) milling the resulting mixture of step (2) to obtain the desired composition,
d) the composition is an emulsifiable concentrate (EC) composition and the process comprises the steps:
(1) mixing the agriculturally acceptable inert additives and a non-aqueous liquid carrier to obtain a premix;
(2) adding compound A to the premix obtained in step (1) to obtain a mixture; and
(3) filtering the solution of step (2) to obtain the desired composition.

58-64. (canceled)

65. A composition comprising an admixture of compound A,
and a liquid carrier,
wherein the composition is prepared using the process of claim 57.

66. A method for:
a) (i) the control or prevention of fungal attack on a plant or (ii) protecting a plant from fungal attack comprising applying the composition of claim 42 to a seed adapted to produce the plant,
b) treating a plant seed or seedling to produce a plant resistant to fungal attack comprising applying the composition of claim 42 to the plant seed or seedling, or
c) protecting a plant from fungal attack comprising applying the composition of claim 42 to the seedling environment.

67-68. (canceled)

69. A plant resistant to fungal attack or a plant seed or seedling adapted to produce a plant resistant to fungal attack, wherein the seed or seedling of the plant is treated with the composition of claim 42.

70-77. (canceled)

78. A process of preparing a stable, liquid composition comprising an admixture of compound A,
and a liquid carrier,
wherein the process comprises the steps:
(1) obtaining a batch of compound A that is substantially pure of compound A or comprises 95% or more by weight of compound A, and
(2) mixing the batch of compound A from step (1) with the liquid carrier to obtain the composition,
wherein compound A is in one or more forms wherein at least one form is Form III, Form IIIa, Form 01, or Form 02 of claim 1.

79. A process of preparing a stable, liquid composition comprising compound A,
and a liquid carrier,
wherein the process comprises the steps:
obtaining a batch of compound A wherein 95% or more of compound A in the batch is in the form of Form III, Form IIIa, Form 01, or Form 02 of claim 1, or a mixture thereof, and
mixing the batch of compound A from step (1) with the liquid carrier to obtain the composition.

80. (canceled)
